SEQUENCE LISTING (314 sequences)

SEQ ID NO: 1 - protein, 10 aa, unidentified
GFTFSNYAMS

SEQ ID NO: 2 - protein, 17 aa, unidentified
AINSNGGSAYYPDTVKD

SEQ ID NO: 3 - protein, 11 aa, unidentified
RFYYDYGWFAY

SEQ ID NO: 4 - protein, 11 aa, unidentified
RASENIDSYLA

SEQ ID NO: 5 - protein, 7 aa, unidentified
AATLLAD

SEQ ID NO: 6 - protein, 9 aa, unidentified
QHYYSSPWT

SEQ ID NO: 7 - protein, 120 aa, unidentified
EVQLVESGGGLVKPGGSLKLSCAASGFTFSNYAMSWVRQTPEKRLEWVAAINSNGGSAYYPDTVKDRFTISRDNAKNTLYLQMSSLRSEDTALYYCARRFYYDYGWFAYWGQGTLVTVSS

SEQ ID NO: 8 - protein, 107 aa, unidentified
DIVLTQSPASLSASVGETVTITCRASENIDSYLAWYQQKQGKSPQLLVYAATLLADGVPSRFSGSGSGTQYSLKINSLQSEDVARYYCQHYYSSPWTFGGGTKLEIK

SEQ ID NO: 9 - protein, 10 aa, unidentified
GFSLSSYAMS

SEQ ID NO: 10 - protein, 16 aa, unidentified
IISSSGTTYYASWAKG

SEQ ID NO: 11 - protein, 11 aa, unidentified
VDYYSSGWGGL

SEQ ID NO: 12 - protein, 11 aa, unidentified
QASEDIYSNLA

SEQ ID NO: 13 - protein, 7 aa, unidentified
GASTLAS

SEQ ID NO: 14 - protein, 12 aa, unidentified
QCTYDTSSYGNT

SEQ ID NO: 15 - protein, 116 aa, unidentified
QSVEESGGRLVTPGTPLTLTCTVSGFSLSSYAMSWVRQAPGKGLEWIGIISSSGTTYYASWAKGRFTISKTSTTVDLKITSPTTEDTATYFCARVDYYSSGWGGLWGPGTLVTVSS

SEQ ID NO: 16 - protein, 110 aa, unidentified
AVEMTQTPASMSAAVGGTVTIKCQASEDIYSNLAWYQQKPGQPPKLLIYGASTLASGVPSRFKGSGSGTEYTLTISGVQCDDAATYYCQCTYDTSSYGNTFGGGTEMVVE

SEQ ID NO: 17 - protein, 10 aa, unidentified
GFSLSSYAMI

SEQ ID NO: 18 - protein, 16 aa, unidentified
IINSSDNTHYATWAKG

SEQ ID NO: 19 - protein, 13 aa, unidentified
DPYDYGYGWYFDL

SEQ ID NO: 20 - protein, 13 aa, unidentified
QASQSVFNNKNLA

SEQ ID NO: 21 - protein, 7 aa, unidentified
KASTLAS

SEQ ID NO: 22 - protein, 13 aa, unidentified
QGEFSCSSTDCVT

SEQ ID NO: 23 - protein, 118 aa, unidentified
QSVEESGGRLVTPGTPLTLTCTVSGFSLSSYAMIWVRQAPGKGLEWIGIINSSDNTHYATWAKGRFTISKTSTTVDLKITSPTTEDTATYFCARDPYDYGYGWYFDLWGPGTLVTVSS

SEQ ID NO: 24 - protein, 113 aa, unidentified
AQVLTQTPSPVSAAVGGTVTINCQASQSVFNNKNLAWYQQKPGQPPKLLIYKASTLASGVSSRFKGSGSGTQFALTISGVQCDDAATYYCQGEFSCSSTDCVTFGGGTEVVVK

SEQ ID NO: 25 - protein, 10 aa, unidentified
GFSLSTYAMS

SEQ ID NO: 26 - protein, 16 aa, unidentified
IISSSGSTYYASWAKG

SEQ ID NO: 27 - protein, 8 aa, unidentified
ETWYWLDL

SEQ ID NO: 28 - protein, 11 aa, unidentified
QASEDIYSNLA

SEQ ID NO: 29 - protein, 7 aa, unidentified
GASTLAS

SEQ ID NO: 30 - protein, 12 aa, unidentified
QCAYDSSSYGTP

SEQ ID NO: 31 - protein, 113 aa, unidentified
QSVEESGGRLVTPGTPLTLTCTASGFSLSTYAMSWVRQAPGKGLEWIGIISSSGSTYYASWAKGRFTISKTSTTVDLKITSPTTEDTATYFCARETWYWLDLWGQGTLVTVSS

SEQ ID NO: 32 - protein, 110 aa, unidentified
AVEMTQTPASVSAAVGGTVTINCQASEDIYSNLAWYQQKPGQPPKLLIYGASTLASGVPSRFKGSGSGTEYTLTISGVQCDDAATYYCQCAYDSSSYGTPFGGGTEVVVK

SEQ ID NO: 33 - protein, 10 aa, unidentified
GFSLSNYAMS

SEQ ID NO: 34 - protein, 16 aa, unidentified
IIYTTGFTYYASWVKG

SEQ ID NO: 35 - protein, 14 aa, unidentified
GLATYVSPPTRLDL

SEQ ID NO: 36 - protein, 13 aa, unidentified
QASQSIYNSKNLA

SEQ ID NO: 37 - protein, 7 aa, unidentified
SASTLAS

SEQ ID NO: 38 - protein, 13 aa, unidentified
QGEFSCSSVDCAT

SEQ ID NO: 39 - protein, 119 aa, unidentified
QSVEESGGRLVTPGTPLTLTCTVSGFSLSNYAMSWVRQAPGKGLEWIGIIYTTGFTYYASWVKGRFTISKTSTTVDLKITSPTTEDTATYFCARGLATYVSPPTRLDLWGQGTLVTVSS

SEQ ID NO: 40 - protein, 113 aa, unidentified
AQVLTQTPSPVSAAVGGTVTINCQASQSIYNSKNLAWYQQKPGQPPKLLIYSASTLASGVPSRFKGSGSGTQFTLTISDLECDDAATYYCQGEFSCSSVDCATFGGGTEVVVK

SEQ ID NO: 41 - protein, 444 aa, unidentified
EVQLVESGGGLVKPGGSLKLSCAASGFTFSNYAMSWVRQTPEKRLEWVAAINSNGGSAYYPDTVKDRFTISRDNAKNTLYLQMSSLRSEDTALYYCARRFYYDYGWFAYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 42 - protein, 214 aa, unidentified
DIVLTQSPASLSASVGETVTITCRASENIDSYLAWYQQKQGKSPQLLVYAATLLADGVPSRFSGSGSGTQYSLKINSLQSEDVARYYCQHYYSSPWTFGGGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 43 - protein, 440 aa, unidentified
QSVEESGGRLVTPGTPLTLTCTVSGFSLSSYAMSWVRQAPGKGLEWIGIISSSGTTYYASWAKGRFTISKTSTTVDLKITSPTTEDTATYFCARVDYYSSGWGGLWGPGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 44 - protein, 217 aa, unidentified
AVEMTQTPASMSAAVGGTVTIKCQASEDIYSNLAWYQQKPGQPPKLLIYGASTLASGVPSRFKGSGSGTEYTLTISGVQCDDAATYYCQCTYDTSSYGNTFGGGTEMVVERTDAAPTVSIFPPSSEQLTSGGASVVCFLNNFYPKDINVKWKIDGSERQNGVLNSWTDQDSKDCTYSMSSTLTLTKDEYERHNSYTCEATHKTSTSPIVKSFNRNEC

SEQ ID NO: 45 - protein, 442 aa, unidentified
QSVEESGGRLVTPGTPLTLTCTVSGFSLSSYAMIWVRQAPGKGLEWIGIINSSDNTHYATWAKGRFTISKTSTTVDLKITSPTTEDTATYFCARDPYDYGYGWYFDLWGPGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 46 - protein, 220 aa, unidentified
AQVLTQTPSPVSAAVGGTVTINCQASQSVFNNKNLAWYQQKPGQPPKLLIYKASTLASGVSSRFKGSGSGTQFALTISGVQCDDAATYYCQGEFSCSSTDCVTFGGGTEVVVKRTDAAPTVSIFPPSSEQLTSGGASVVCFLNNFYPKDINVKWKIDGSERQNGVLNSWTDQDSKDCTYSMSSTLTLTKDEYERHNSYTCEATHKTSTSPIVKSFNRNEC

SEQ ID NO: 47 - protein, 437 aa, unidentified
QSVEESGGRLVTPGTPLTLTCTASGFSLSTYAMSWVRQAPGKGLEWIGIISSSGSTYYASWAKGRFTISKTSTTVDLKITSPTTEDTATYFCARETWYWLDLWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 48 - protein, 217 aa, unidentified
AVEMTQTPASVSAAVGGTVTINCQASEDIYSNLAWYQQKPGQPPKLLIYGASTLASGVPSRFKGSGSGTEYTLTISGVQCDDAATYYCQCAYDSSSYGTPFGGGTEVVVKRTDAAPTVSIFPPSSEQLTSGGASVVCFLNNFYPKDINVKWKIDGSERQNGVLNSWTDQDSKDCTYSMSSTLTLTKDEYERHNSYTCEATHKTSTSPIVKSFNRNEC

SEQ ID NO: 49 - protein, 443 aa, unidentified
QSVEESGGRLVTPGTPLTLTCTVSGFSLSNYAMSWVRQAPGKGLEWIGIIYTTGFTYYASWVKGRFTISKTSTTVDLKITSPTTEDTATYFCARGLATYVSPPTRLDLWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 50 - protein, 220 aa, unidentified
AQVLTQTPSPVSAAVGGTVTINCQASQSIYNSKNLAWYQQKPGQPPKLLIYSASTLASGVPSRFKGSGSGTQFTLTISDLECDDAATYYCQGEFSCSSVDCATFGGGTEVVVKRTDAAPTVSIFPPSSEQLTSGGASVVCFLNNFYPKDINVKWKIDGSERQNGVLNSWTDQDSKDCTYSMSSTLTLTKDEYERHNSYTCEATHKTSTSPIVKSFNRNEC

SEQ ID NO: 51 - DNA, 429 bp, unidentified
  source 1..429: mol_type = other DNA
aagcttgccaccatggaatggagctgggtctttctcttcttcctgtcagtaactacaggagtccattctgaggtgcagctggtggagtctgggggaggcttagtgaagcctggagggtccctgaaactctcctgtgcagcctctggattcactttcagtaactatgccatgtcttgggttcgccagactccagagaagaggctggagtgggtcgcagccattaatagtaatggtggtagcgcctactatccagacactgtgaaggaccgattcaccatctccagagacaatgccaagaacaccctgtacctgcaaatgagcagtctgaggtctgaggacacagccttgtattactgtgcaagacgcttctactatgattacggctggtttgcttactggggccaagggactctggtcacagtctcgagc

SEQ ID NO: 52 - DNA, 399 bp, unidentified
  source 1..399: mol_type = other DNA
aagcttgccaccatgtctgtccccacccaagtcctcggactcctgctactctggcttacagatgccagatgcgacattgtgctgacccaatctccagcttccctgtctgcatctgtgggagaaactgtcaccatcacatgtcgagcaagtgagaatattgacagttatttagcatggtatcagcagaaacagggaaaatctcctcagctcctggtctatgctgcaacactcttagcagatggtgtgccatcaaggttcagtggcagtggatcaggcacacagtattctctcaagatcaacagcctgcagtctgaagatgttgcgagatattactgtcaacattattatagttctccgtggacgttcggtggaggcaccaagctggaaataaaacgtacg

SEQ ID NO: 53 - DNA, 417 bp, unidentified
  source 1..417: mol_type = other DNA
aagcttgccaccatggaatggagctgggtctttctcttcttcctgtcagtaactacaggagtccattctcagtcggtggaggagtccgggggtcgcctggtcacgcctgggacacccctgacactcacatgcacagtctctggattctccctcagtagctatgcgatgagctgggtccgccaggctccagggaaggggctggaatggatcggaatcattagtagcagtggtaccacatactacgcgagctgggcgaaaggccgattcaccatttccaaaacctcgaccacggtggatctgaaaatcaccagtccgacaaccgaggacacggccacctatttctgtgccagagtcgattactatagtagtggctggggtggcttgtggggcccaggcaccctggtcaccgtctcgagc

SEQ ID NO: 54 - DNA, 418 bp, unidentified
  source 1..418: mol_type = other DNA
aagcttcgaagccaccatggacacgagggcccccactcagctgctggggctcctgctgctctggctcccaggtgccacatttgccgttgaaatgacccagactccagcctcgatgtctgccgctgtgggaggcacagtcaccatcaagtgccaggccagtgaggacatttatagcaatttggcctggtatcagcagaaaccagggcagcctcccaagctcctgatctatggtgcatccactctggcttctggggtcccatcgcggttcaaaggcagtggatctgggacagagtacactctcaccatcagcggtgtgcagtgtgacgatgctgccacttactattgtcaatgcacttatgatactagtagttatggtaatactttcggcggagggaccgagatggtagtcgaacgtacg

SEQ ID NO: 55 - DNA, 423 bp, unidentified
  source 1..423: mol_type = other DNA
aagcttgccaccatggaatggagctgggtctttctcttcttcctgtcagtaactacaggagtccattctcagtcggtggaggagtccgggggtcgcctggtcacgcctgggacacccctgacactcacctgcacagtctctggattctccctcagtagctatgcaatgatctgggtccgccaggctccagggaaggggctggaatggatcggaatcattaatagtagtgataacacacactacgcgacctgggcgaaaggccgattcaccatctccaaaacctcgaccacggtggatctaaaaatcaccagtccgacaaccgaggacacggccacctatttctgtgccagagatccctacgactatggttatggttggtactttgacttgtggggcccaggcaccctggtcaccgtctcgagc

SEQ ID NO: 56 - DNA, 427 bp, unidentified
  source 1..427: mol_type = other DNA
aagcttcgaagccaccatggacacgagggcccccactcagctgctggggctcctgctgctctggctcccaggtgccacatttgcccaagtgctgacccagactccatcccctgtgtctgcagctgtgggaggcacagtcaccatcaactgccaggccagtcagagtgtttttaataacaaaaatttagcctggtatcagcagaaaccagggcagcctcccaagctcctgatctacaaggcatccactctggcatctggcgtctcatcgcggttcaaaggcagtggatctgggacacagttcgctctcaccatcagcggcgtgcagtgtgacgatgctgccacttactactgtcaaggcgaatttagttgtagtagtactgattgcgtgactttcggcggagggaccgaggtggtggtcaaacgtacg

SEQ ID NO: 57 - DNA, 408 bp, unidentified
  source 1..408: mol_type = other DNA
aagcttgccaccatggaatggagctgggtctttctcttcttcctgtcagtaactacaggagtccattctcagtcggtggaggagtccgggggtcgcctggtcacgcctgggacacccctgacactcacctgcacagcctctggattctccctcagtacctatgcaatgagttgggtccgccaggctccagggaaggggctggaatggatcggaatcattagtagtagtggtagcacatactacgcgagctgggcgaaaggccgattcaccatctccaaaacctcgaccacggtggatctgaaaatcaccagtccgacaaccgaggacacggccacctatttctgtgccagagagacttggtactggttggatctctggggccagggcaccctggtcaccgtctcgagc

SEQ ID NO: 58 - DNA, 418 bp, unidentified
  source 1..418: mol_type = other DNA
aagcttcgaagccaccatggacatgagggcccccactcagctgctggggctcctgctgctctggctcccaggtgccacatttgccgttgaaatgacccagactccagcctcggtgtctgccgctgtgggaggcacagtcaccatcaattgccaggccagtgaggacatttatagcaatttggcctggtatcagcagaaaccagggcagcctcccaagctcctgatctatggtgcatccactctggcatctggggtcccatcgcggttcaaaggcagtggatctgggacagagtacactctcaccatcagcggtgtgcagtgtgacgatgctgccacttactactgtcaatgcgcttatgatagtagtagttatggtacccctttcggcggagggaccgaggtggtggtcaaacgtacg

SEQ ID NO: 59 - DNA, 426 bp, unidentified
  source 1..426: mol_type = other DNA
aagcttgccaccatggaatggagctgggtctttctcttcttcctgtcagtaactacaggagtccattctcagtcggtggaggagtccgggggtcgcctggtcacgcctgggacacccctgacactcacctgcacagtctctggattctccctcagtaactatgcaatgagctgggtccgccaggctccagggaaggggctggaatggatcggaatcatttatactactggtttcacatactacgcgagctgggtgaaaggccgattcaccatctccaaaacctcgaccacggtggacctgaaaatcaccagtccgacaaccgaggacacggccacctatttctgtgccagagggctggctacttatgttagtcccccgactcggttggatctctggggccagggcaccctggtcaccgtctcgagc

SEQ ID NO: 60 - DNA, 427 bp, unidentified
  source 1..427: mol_type = other DNA
aagcttcgaagccaccatgaacatgagggcccccactcagctgctggggctcctgctgctctggctcccaggtgccacatttgcccaagtgctgacccagactccatcccctgtgtctgcagctgtgggaggcacagtcaccatcaactgccaggccagtcagagtatttataatagcaaaaatttagcctggtatcagcagaaaccagggcagcctcccaagctcctgatctattctgcatccactctggcatctggggtcccatcgcggttcaaaggcagtggatctgggacacagttcactctcaccatcagcgacctggagtgtgacgatgctgccacttactactgtcaaggcgaatttagttgtagtagtgttgattgcgccactttcggcggagggaccgaggtggtggtcaaacgtacg

SEQ ID NO: 61 - DNA, 30 bp, unidentified
  source 1..30: mol_type = other DNA
ggattcactttcagtaactatgccatgtct

SEQ ID NO: 62 - DNA, 51 bp, unidentified
  source 1..51: mol_type = other DNA
gccattaatagtaatggtggtagcgcctactatccagacactgtgaaggac

SEQ ID NO: 63 - DNA, 33 bp, unidentified
  source 1..33: mol_type = other DNA
cgcttctactatgattacggctggtttgcttac

SEQ ID NO: 64 - DNA, 33 bp, unidentified
  source 1..33: mol_type = other DNA
cgagcaagtgagaatattgacagttatttagca

SEQ ID NO: 65 - DNA, 21 bp, unidentified
  source 1..21: mol_type = other DNA
gctgcaacactcttagcagat

SEQ ID NO: 66 - DNA, 27 bp, unidentified
  source 1..27: mol_type = other DNA
caacattattatagttctccgtggacg

SEQ ID NO: 67 - DNA, 30 bp, unidentified
  source 1..30: mol_type = other DNA
ggattctccctcagtagctatgcgatgagc

SEQ ID NO: 68 - DNA, 48 bp, unidentified
  source 1..48: mol_type = other DNA
atcattagtagcagtggtaccacatactacgcgagctgggcgaaaggc

SEQ ID NO: 69 - DNA, 33 bp, unidentified
  source 1..33: mol_type = other DNA
gtcgattactatagtagtggctggggtggcttg

SEQ ID NO: 70 - DNA, 33 bp, unidentified
  source 1..33: mol_type = other DNA
caggccagtgaggacatttatagcaatttggcc

SEQ ID NO: 71 - DNA, 21 bp, unidentified
  source 1..21: mol_type = other DNA
ggtgcatccactctggcttct

SEQ ID NO: 72 - DNA, 36 bp, unidentified
  source 1..36: mol_type = other DNA
caatgcacttatgatactagtagttatggtaatact

SEQ ID NO: 73 - DNA, 30 bp, unidentified
  source 1..30: mol_type = other DNA
ggattctccctcagtagctatgcaatgatc

SEQ ID NO: 74 - DNA, 48 bp, unidentified
  source 1..48: mol_type = other DNA
atcattaatagtagtgataacacacactacgcgacctgggcgaaaggc

SEQ ID NO: 75 - DNA, 39 bp, unidentified
  source 1..39: mol_type = other DNA
gatccctacgactatggttatggttggtactttgacttg

SEQ ID NO: 76 - DNA, 39 bp, unidentified
  source 1..39: mol_type = other DNA
caggccagtcagagtgtttttaataacaaaaatttagcc

SEQ ID NO: 77 - DNA, 21 bp, unidentified
  source 1..21: mol_type = other DNA
aaggcatccactctggcatct

SEQ ID NO: 78 - DNA, 39 bp, unidentified
  source 1..39: mol_type = other DNA
caaggcgaatttagttgtagtagtactgattgcgtgact

SEQ ID NO: 79 - DNA, 30 bp, unidentified
  source 1..30: mol_type = other DNA
ggattctccctcagtacctatgcaatgagt

SEQ ID NO: 80 - DNA, 48 bp, unidentified
  source 1..48: mol_type = other DNA
atcattagtagtagtggtagcacatactacgcgagctgggcgaaaggc

SEQ ID NO: 81 - DNA, 24 bp, unidentified
  source 1..24: mol_type = other DNA
gagacttggtactggttggatctc

SEQ ID NO: 82 - DNA, 33 bp, unidentified; identical to SEQ ID NO: 70
  source 1..33: mol_type = other DNA

SEQ ID NO: 83 - DNA, 21 bp, unidentified
  source 1..21: mol_type = other DNA
ggtgcatccactctggcatct

SEQ ID NO: 84 - DNA, 36 bp, unidentified
  source 1..36: mol_type = other DNA
caatgcgcttatgatagtagtagttatggtacccct

SEQ ID NO: 85 - DNA, 30 bp, unidentified
  source 1..30: mol_type = other DNA
ggattctccctcagtaactatgcaatgagc

SEQ ID NO: 86 - DNA, 48 bp, unidentified
  source 1..48: mol_type = other DNA
atcatttatactactggtttcacatactacgcgagctgggtgaaaggc

SEQ ID NO: 87 - DNA, 42 bp, unidentified
  source 1..42: mol_type = other DNA
gggctggctacttatgttagtcccccgactcggttggatctc

SEQ ID NO: 88 - DNA, 39 bp, unidentified
  source 1..39: mol_type = other DNA
caggccagtcagagtatttataatagcaaaaatttagcc

SEQ ID NO: 89 - DNA, 21 bp, unidentified
  source 1..21: mol_type = other DNA
tctgcatccactctggcatct

SEQ ID NO: 90 - DNA, 39 bp, unidentified
  source 1..39: mol_type = other DNA
caaggcgaatttagttgtagtagtgttgattgcgccact

SEQ ID NO: 91 - protein, 10 aa, unidentified
GFSLTTYEIN

SEQ ID NO: 92 - protein, 16 aa, unidentified
VIWTGGSTNYNSALIS

SEQ ID NO: 93 - protein, 10 aa, unidentified
TLPYYYAMDY

SEQ ID NO: 94 - protein, 12 aa, unidentified
SVSSSISSSNLH

SEQ ID NO: 95 - protein, 7 aa, unidentified
GTSNLAS

SEQ ID NO: 96 - protein, 9 aa, unidentified
QQWSSYPPT

SEQ ID NO: 97 - protein, 118 aa, unidentified
QVQLQQSGPGLVAPSQSLFITCTVSGFSLTTYEINWVRQPPGKGLEWLGVIWTGGSTNYNSALISRLSISKDNSKSLVFLKMNSLQTDDTAIYYCVRTLPYYYAMDYWGQGTSVTVSS

SEQ ID NO: 98 - protein, 108 aa, unidentified
EIVLTQSPALMAASPGEKVTITCSVSSSISSSNLHWYQQKSGTSPKPWIYGTSNLASGVPVRFSGSGSGTSYSLTISSMEAEDAATYYCQQWSSYPPTFGSGTKLEIK

SEQ ID NO: 99 - protein, 215 aa, unidentified
EIVLTQSPALMAASPGEKVTITCSVSSSISSSNLHWYQQKSGTSPKPWIYGTSNLASGVPVRFSGSGSGTSYSLTISSMEAEDAATYYCQQWSSYPPTFGSGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 100 - protein, 442 aa, unidentified
QVQLQQSGPGLVAPSQSLFITCTVSGFSLTTYEINWVRQPPGKGLEWLGVIWTGGSTNYNSALISRLSISKDNSKSLVFLKMNSLQTDDTAIYYCVRTLPYYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 101 - protein, 302 aa, unidentified
METDTLLLWVLLLWVPGSTGDAQSVEESGGRLVTPGTPLTLTCTVSGFSLSNYAMSWVRQAPGKGLEWIGIIYTTGFTYYASWVKGRFTISKTSTTVDLKITSPTTEDTATYFCARGLATYVSPPTRLDLWGQGTLVTVSSGGGGSGGGGSGGGGSAQVLTQTPSPVSAAVGGTVTINCQASQSIYNSKNLAWYQQKPGQPPKLLIYSASTLASGVPSRFKGSGSGTQFTLTISDLESDDAATYYCQGEFSCSSVDCATFGGGTEVVVKSGLEVLFQGPGSGLNDIFEAQKIEWHEHHHHHH

SEQ ID NO: 102 - protein, 297 aa, unidentified
METDTLLLWVLLLWVPGSTGDAEVQLVESGGGLVKPGGSLKLSCAASGFTFSNYAMSWVRQTPEKRLEWVAAINSNGGSAYYPDTVKDRFTISRDNAKNTLYLQMSSLRSEDTALYYCARRFYYDYGWFAYWGQGTLVTVSSGGGGSGGGGSGGGGSDIVLTQSPASLSASVGETVTITCRASENIDSYLAWYQQKQGKSPQLLVYAATLLADGVPSRFSGSGSGTQYSLKINSLQSEDVARYYCQHYYSSPWTFGGGTKLEIKSGLEVLFQGPGSGLNDIFEAQKIEWHEHHHHHH

SEQ ID NO: 103 - protein, 301 aa, unidentified
METDTLLLWVLLLWVPGSTGDAQSVEESGGRLVTPGTPLTLTCTVSGFSLSSYAMIWVRQAPGKGLEWIGIINSSDNTHYATWAKGRFTISKTSTTVDLKITSPTTEDTATYFCARDPYDYGYGWYFDLWGPGTLVTVSSGGGGSGGGGSGGGGSAQVLTQTPSPVSAAVGGTVTINCQASQSVFNNKNLAWYQQKPGQPPKLLIYKASTLASGVSSRFKGSGSGTQFALTISGVQSDDAATYYCQGEFSCSSTDCVTFGGGTEVVVKSGLEVLFQGPGSGLNDIFEAQKIEWHEHHHHHH

SEQ ID NO: 104 - protein, 247 aa, unidentified
QSVEESGGRLVTPGTPLTLTCTVSGFSLSNYAMSWVRQAPGKGLEWIGIIYTTGFTYYASWVKGRFTISKTSTTVDLKITSPTTEDTATYFCARGLATYVSPPTRLDLWGQGTLVTVSSGGGGSGGGGSGGGGSAQVLTQTPSPVSAAVGGTVTINCQASQSIYNSKNLAWYQQKPGQPPKLLIYSASTLASGVPSRFKGSGSGTQFTLTISDLESDDAATYYCQGEFSCSSVDCATFGGGTEVVVK

SEQ ID NO: 105 - protein, 246 aa, unidentified
QSVEESGGRLVTPGTPLTLTCTVSGFSLSSYAMIWVRQAPGKGLEWIGIINSSDNTHYATWAKGRFTISKTSTTVDLKITSPTTEDTATYFCARDPYDYGYGWYFDLWGPGTLVTVSSGGGGSGGGGSGGGGSAQVLTQTPSPVSAAVGGTVTINCQASQSVFNNKNLAWYQQKPGQPPKLLIYKASTLASGVSSRFKGSGSGTQFALTISGVQSDDAATYYCQGEFSCSSTDCVTFGGGTEVVVK

SEQ ID NO: 106 - protein, 242 aa, unidentified
EVQLVESGGGLVKPGGSLKLSCAASGFTFSNYAMSWVRQTPEKRLEWVAAINSNGGSAYYPDTVKDRFTISRDNAKNTLYLQMSSLRSEDTALYYCARRFYYDYGWFAYWGQGTLVTVSSGGGGSGGGGSGGGGSDIVLTQSPASLSASVGETVTITCRASENIDSYLAWYQQKQGKSPQLLVYAATLLADGVPSRFSGSGSGTQYSLKINSLQSEDVARYYCQHYYSSPWTFGGGTKLEIK

SEQ ID NO: 107 - protein, 12 aa, unidentified
QSTYDTSSYGNT

SEQ ID NO: 108 - protein, 12 aa, unidentified
QATYDTSSYGNT

SEQ ID NO: 109 - protein, 12 aa, unidentified
QVTYDTSSYGNT

SEQ ID NO: 110 - protein, 13 aa, unidentified
EPYDYGYGWYFDL

SEQ ID NO: 111 - protein, 13 aa, unidentified
QGEFSSSSTDSVT

SEQ ID NO: 112 - protein, 13 aa, unidentified
QGEFSSSSTDAVT

SEQ ID NO: 113 - protein, 13 aa, unidentified
QGEFSSSSTDVVT

SEQ ID NO: 114 - protein, 13 aa, unidentified
QGEFSASSTDSVT

SEQ ID NO: 115 - protein, 13 aa, unidentified
QGEFSASSTDAVT

SEQ ID NO: 116 - protein, 13 aa, unidentified
QGEFSASSTDVVT

SEQ ID NO: 117 - protein, 13 aa, unidentified
QGEFSVSSTDSVT

SEQ ID NO: 118 - protein, 13 aa, unidentified
QGEFSVSSTDAVT

SEQ ID NO: 119 - protein, 13 aa, unidentified
QGEFSVSSTDVVT

SEQ ID NO: 120 - protein, 12 aa, unidentified
QSAYDSSSYGTP

SEQ ID NO: 121 - protein, 12 aa, unidentified
QAAYDSSSYGTP

SEQ ID NO: 122 - protein, 12 aa, unidentified
QVAYDSSSYGTP

SEQ ID NO: 123 - protein, 13 aa, unidentified
QGEFSSSSVDSAT

SEQ ID NO: 124 - protein, 13 aa, unidentified
QGEFSSSSVDAAT

SEQ ID NO: 125 - protein, 13 aa, unidentified
QGEFSSSSVDVAT

SEQ ID NO: 126 - protein, 13 aa, unidentified
QGEFSASSVDSAT

SEQ ID NO: 127 - protein, 13 aa, unidentified
QGEFSASSVDAAT

SEQ ID NO: 128 - protein, 13 aa, unidentified
QGEFSASSVDVAT

SEQ ID NO: 129 - protein, 13 aa, unidentified
QGEFSVSSVDSAT

SEQ ID NO: 130 - protein, 13 aa, unidentified
QGEFSVSSVDAAT

SEQ ID NO: 131 - protein, 13 aa, unidentified
QGEFSVSSVDVAT

SEQ ID NO: 132 - protein, 16 aa, unidentified
IIQSSDNTHYATWAKG

SEQ ID NO: 133 - protein, 16 aa, unidentified
IIDSSDNTHYATWAKG

SEQ ID NO: 134 - protein, 16 aa, unidentified
IISSSDNTHYATWAKG

SEQ ID NO: 135 - protein, 13 aa, unidentified
QASQSIYNTKNLA

SEQ ID NO: 136 - protein, 13 aa, unidentified
QASQSIYNAKNLA

SEQ ID NO: 137 - protein, 13 aa, unidentified
QASQSIYNNKNLA

SEQ ID NO: 138 - protein, 13 aa, unidentified
QASQSIYQSKNLA

SEQ ID NO: 139 - protein, 13 aa, unidentified
QASQSIYDSKNLA

SEQ ID NO: 140 - protein, 7 aa, unidentified
AATLLAE

SEQ ID NO: 141 - protein, 7 aa, unidentified
AATLLAS

SEQ ID NO: 142 - protein, 17 aa, unidentified
AINSNSGSAYYPDTVKD

SEQ ID NO: 143 - protein, 17 aa, unidentified
AINSNAGSAYYPDTVKD

SEQ ID NO: 144 - protein, 17 aa, unidentified
AINSNAGSAYYPDTVKD

SEQ ID NO: 145 - protein, 17 aa, unidentified
AINSQGGSAYYPDTVKD

SEQ ID NO: 146 - protein, 17 aa, unidentified
AINSDGGSAYYPDTVKD

SEQ ID NO: 147 - protein, 17 aa, unidentified
AINANGGSAYYPDTVKD

SEQ ID NO: 148 - protein, 17 aa, unidentified
AINTNGGSAYYPDTVKD

SEQ ID NO: 149 - protein, 17 aa, unidentified
AIQSNGGSAYYPDTVKD

SEQ ID NO: 150 - protein, 17 aa, unidentified
AIDSNGGSAYYPDTVKD

SEQ ID NO: 151 - protein, 17 aa, unidentified
AITSNGGSAYYPDTVKD

SEQ ID NO: 152 - protein, 17 aa, unidentified
AISSNGGSAYYPDTVKD

SEQ ID NO: 153 - protein, 110 aa, unidentified
AVEMTQSPSTLSASVGDRVTITCQASEDIYSNLAWYQQKPGKAPKLLIYGASTLASGVPSRFSGSGSGTEYTLTISSLQPDDFATYYCQCTYDTSSYGNTFGGGTKVEIK

SEQ ID NO: 154 - protein, 110 aa, unidentified
AVEMTQSPSTLSASVGDRVTITCQASEDIYSNLAWYQQKPGKAPKLLIYGASTLASGVPSRFSGSGSGTEYTLTISSLQPDDFATYYCQSTYDTSSYGNTFGGGTKVEIK

SEQ ID NO: 155 - protein, 110 aa, unidentified
AVEMTQSPSTLSASVGDRVTITCQASEDIYSNLAWYQQKPGKAPKLLIYGASTLASGVPSRFSGSGSGTEYTLTISSLQPDDFATYYCQATYDTSSYGNTFGGGTKVEIK

SEQ ID NO: 156 - protein, 110 aa, unidentified
AVEMTQSPSTLSASVGDRVTITCQASEDIYSNLAWYQQKPGKAPKLLIYGASTLASGVPSRFSGSGSGTEYTLTISSLQPDDFATYYCQVTYDTSSYGNTFGGGTKVEIK

SEQ ID NO: 157 - protein, 119 aa, unidentified
EVQLLESGGGLVQPGGSLRLSCAVSGFSLSSYAMSWVRQAPGKGLEWIGIISSSGTTYYASWAKGRFTISKDSSKNTVYLQMNSLRAEDTAVYYCARVDYYSSGWGGLWGQGTLVTVSS

SEQ ID NO: 158 - protein, 113 aa, unidentified
AQVLTQSPSSLSASVGDRVTITCQASQSVFNNKNLAWYQQKPGKAPKLLIYKASTLASGVPSRFSGSGSGTQFTLTISSLQPEDFATYYCQGEFSCSSTDCVTFGGGTKVEIK

SEQ ID NO: 159 - protein, 113 aa, unidentified
AQVLTQSPSSLSASVGDRVTITCQASQSVFNNKNLAWYQQKPGKAPKLLIYKASTLASGVPSRFSGSGSGTQFTLTISSLQPEDFATYYCQGEFSSSSTDSVTFGGGTKVEIK

SEQ ID NO: 160 - protein, 113 aa, unidentified
AQVLTQSPSSLSASVGDRVTITCQASQSVFNNKNLAWYQQKPGKAPKLLIYKASTLASGVPSRFSGSGSGTQFTLTISSLQPEDFATYYCQGEFSSSSTDAVTFGGGTKVEIK

SEQ ID NO: 161 - protein, 113 aa, unidentified
AQVLTQSPSSLSASVGDRVTITCQASQSVFNNKNLAWYQQKPGKAPKLLIYKASTLASGVPSRFSGSGSGTQFTLTISSLQPEDFATYYCQGEFSSSSTDVVTFGGGTKVEIK

SEQ ID NO: 162 - protein, 113 aa, unidentified
AQVLTQSPSSLSASVGDRVTITCQASQSVFNNKNLAWYQQKPGKAPKLLIYKASTLASGVPSRFSGSGSGTQFTLTISSLQPEDFATYYCQGEFSASSTDSVTFGGGTKVEIK

SEQ ID NO: 163 - protein, 113 aa, unidentified
AQVLTQSPSSLSASVGDRVTITCQASQSVFNNKNLAWYQQKPGKAPKLLIYKASTLASGVPSRFSGSGSGTQFTLTISSLQPEDFATYYCQGEFSASSTDAVTFGGGTKVEIK

SEQ ID NO: 164 - protein, 113 aa, unidentified
AQVLTQSPSSLSASVGDRVTITCQASQSVFNNKNLAWYQQKPGKAPKLLIYKASTLASGVPSRFSGSGSGTQFTLTISSLQPEDFATYYCQGEFSASSTDVVTFGGGTKVEIK

SEQ ID NO: 165 - protein, 113 aa, unidentified
AQVLTQSPSSLSASVGDRVTITCQASQSVFNNKNLAWYQQKPGKAPKLLIYKASTLASGVPSRFSGSGSGTQFTLTISSLQPEDFATYYCQGEFSVSSTDSVTFGGGTKVEIK

SEQ ID NO: 166 - protein, 113 aa, unidentified
AQVLTQSPSSLSASVGDRVTITCQASQSVFNNKNLAWYQQKPGKAPKLLIYKASTLASGVPSRFSGSGSGTQFTLTISSLQPEDFATYYCQGEFSVSSTDAVTFGGGTKVEIK

SEQ ID NO: 167 - protein, 113 aa, unidentified
AQVLTQSPSSLSASVGDRVTITCQASQSVFNNKNLAWYQQKPGKAPKLLIYKASTLASGVPSRFSGSGSGTQFTLTISSLQPEDFATYYCQGEFSVSSTDVVTFGGGTKVEIK

SEQ ID NO: 168 - protein, 121 aa, unidentified
EVQLVESGGGLVQPGGSLRLSCAVSGFSLSSYAMIWVRQAPGKGLEWIGIINSSDNTHYATWAKGRFTISKDSAKNSVYLQMNSLRDEDTAVYYCARDPYDYGYGWYFDLWGRGTLVTVSS

SEQ ID NO: 169 - protein, 121 aa, unidentified
EVQLVESGGGLVQPGGSLRLSCAVSGFSLSSYAMIWVRQAPGKGLEWIGIIQSSDNTHYATWAKGRFTISKDSAKNSVYLQMNSLRDEDTAVYYCARDPYDYGYGWYFDLWGRGTLVTVSS

SEQ ID NO: 170 - protein, 121 aa, unidentified
EVQLVESGGGLVQPGGSLRLSCAVSGFSLSSYAMIWVRQAPGKGLEWIGIIDSSDNTHYATWAKGRFTISKDSAKNSVYLQMNSLRDEDTAVYYCARDPYDYGYGWYFDLWGRGTLVTVSS

SEQ ID NO: 171 - protein, 121 aa, unidentified
EVQLVESGGGLVQPGGSLRLSCAVSGFSLSSYAMIWVRQAPGKGLEWIGIITSSDNTHYATWAKGRFTISKDSAKNSVYLQMNSLRDEDTAVYYCARDPYDYGYGWYFDLWGRGTLVTVSS

SEQ ID NO: 172 - protein, 121 aa, unidentified
EVQLVESGGGLVQPGGSLRLSCAVSGFSLSSYAMIWVRQAPGKGLEWIGIISSSDNTHYATWAKGRFTISKDSAKNSVYLQMNSLRDEDTAVYYCARDPYDYGYGWYFDLWGRGTLVTVSS

SEQ ID NO: 173 - protein, 121 aa, unidentified
EVQLVESGGGLVQPGGSLRLSCAVSGFSLSSYAMIWVRQAPGKGLEWIGIINSSDNTHYATWAKGRFTISKDSAKNSVYLQMNSLRDEDTAVYYCAREPYDYGYGWYFDLWGRGTLVTVSS

SEQ ID NO: 174 - protein, 110 aa, unidentified
AVEMTQSPSTLSASVGDRVTITCQASEDIYSNLAWYQQKPGKAPKLLIYGASTLASGVPSRFSGSGSGTEYTLTISSLQPDDFATYYCQCAYDSSSYGTPFGGGTKVEIK

SEQ ID NO: 175 - protein, 110 aa, unidentified
AVEMTQSPSTLSASVGDRVTITCQASEDIYSNLAWYQQKPGKAPKLLIYGASTLASGVPSRFSGSGSGTEYTLTISSLQPDDFATYYCQSAYDSSSYGTPFGGGTKVEIK

SEQ ID NO: 176 - protein, 110 aa, unidentified
AVEMTQSPSTLSASVGDRVTITCQASEDIYSNLAWYQQKPGKAPKLLIYGASTLASGVPSRFSGSGSGTEYTLTISSLQPDDFATYYCQAAYDSSSYGTPFGGGTKVEIK

SEQ ID NO: 177 - protein, 110 aa, unidentified
AVEMTQSPSTLSASVGDRVTITCQASEDIYSNLAWYQQKPGKAPKLLIYGASTLASGVPSRFSGSGSGTEYTLTISSLQPDDFATYYCQVAYDSSSYGTPFGGGTKVEIK

SEQ ID NO: 178 - protein, 116 aa, unidentified
EVQLLESGGGLVQPGGSLRLSCAASGFSLSTYAMSWVRQAPGKGLEWIGIISSSGSTYYASWAKGRFTISKDSSKNTVYLQMNSLRAEDTAVYYCARETWYWLDLWGRGTLVTVSS

SEQ ID NO: 179 - protein, 113 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSCSSVDCATFGGGTKVEIK

SEQ ID NO: 180 - protein, 113 aa, unidentified
AQVLTQSPSSLSASVGDRVTITCQASQSIYNTKNLAWYQQKPGKAPKLLIYSASTLASGVPSRFSGSGSGTQFTLTISSLQPEDFATYYCQGEFSCSSVDCATFGGGTKVEIK

SEQ ID NO: 181 - protein, 113 aa, unidentified
AQVLTQSPSSLSASVGDRVTITCQASQSIYNAKNLAWYQQKPGKAPKLLIYSASTLASGVPSRFSGSGSGTQFTLTISSLQPEDFATYYCQGEFSCSSVDCATFGGGTKVEIK

SEQ ID NO: 182 - protein, 113 aa, unidentified
AQVLTQSPSSLSASVGDRVTITCQASQSIYNNKNLAWYQQKPGKAPKLLIYSASTLASGVPSRFSGSGSGTQFTLTISSLQPEDFATYYCQGEFSCSSVDCATFGGGTKVEIK

SEQ ID NO: 183 - protein, 113 aa, unidentified
AQVLTQSPSSLSASVGDRVTITCQASQSIYQSKNLAWYQQKPGKAPKLLIYSASTLASGVPSRFSGSGSGTQFTLTISSLQPEDFATYYCQGEFSCSSVDCATFGGGTKVEIK

SEQ ID NO: 184 - protein, 113 aa, unidentified
AQVLTQSPSSLSASVGDRVTITCQASQSIYDSKNLAWYQQKPGKAPKLLIYSASTLASGVPSRFSGSGSGTQFTLTISSLQPEDFATYYCQGEFSCSSVDCATFGGGTKVEIK

SEQ ID NO: 185 - protein, 113 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSSSSVDSATFGGGTKVEIK

SEQ ID NO: 186 - protein, 113 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSSSSVDAATFGGGTKVEIK

SEQ ID NO: 187 - protein, 113 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSSSSVDVATFGGGTKVEIK

SEQ ID NO: 188 - protein, 113 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSASSVDSATFGGGTKVEIK

SEQ ID NO: 189 - protein, 113 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSASSVDAATFGGGTKVEIK

SEQ ID NO: 190 - protein, 113 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSASSVDVATFGGGTKVEIK

SEQ ID NO: 191 - protein, 113 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSVSSVDSATFGGGTKVEIK

SEQ ID NO: 192 - protein, 113 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSVSSVDAATFGGGTKVEIK

SEQ ID NO: 193 - protein, 113 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSVSSVDVATFGGGTKVEIK

SEQ ID NO: 194 - protein, 122 aa, unidentified
EVQLVESGGGLVQPGGSLRLSCAVSGFSLSNYAMSWVRQAPGKGLEWIGIIYTTGFTYYASWVKGRFTISKDSSKNTVYLQMNSLRAEDTAVYYCARGLATYVSPPTRLDLWGQGTLVTVSS

SEQ ID NO: 195 - protein, 107 aa, unidentified
DIQMTQSPSSLSASVGDRVTITCRASENIDSYLAWYQQKPGKAPKLLVYAATLLADGVPSRFSGSGSGTQYTLTISSLQPEDFARYYCQHYYSSPWTFGQGTKVEIK

SEQ ID NO: 196 - protein, 107 aa, unidentified
DIQMTQSPSSLSASVGDRVTITCRASENIDSYLAWYQQKPGKAPKLLVYAATLLAEGVPSRFSGSGSGTQYTLTISSLQPEDFARYYCQHYYSSPWTFGQGTKVEIK

SEQ ID NO: 197 - protein, 107 aa, unidentified
DIQMTQSPSSLSASVGDRVTITCRASENIDSYLAWYQQKPGKAPKLLVYAATLLASGVPSRFSGSGSGTQYTLTISSLQPEDFARYYCQHYYSSPWTFGQGTKVEIK

SEQ ID NO: 198 - protein, 120 aa, unidentified
EVQLLESGGGLVQPGGSLRLSCAASGFTFSNYAMSWVRQAPGKRLEWVAAINSNGGSAYYPDTVKDRFTISRDNSKNTLYLQMNSLRAEDTAVYYCARRFYYDYGWFAYWGQGTLVTVSS

SEQ ID NO: 199 - protein, 120 aa, unidentified
EVQLLESGGGLVQPGGSLRLSCAASGFTFSNYAMSWVRQAPGKRLEWVAAINSNSGSAYYPDTVKDRFTISRDNSKNTLYLQMNSLRAEDTAVYYCARRFYYDYGWFAYWGQGTLVTVSS

SEQ ID NO: 200 - protein, 120 aa, unidentified
EVQLLESGGGLVQPGGSLRLSCAASGFTFSNYAMSWVRQAPGKRLEWVAAINSNAGSAYYPDTVKDRFTISRDNSKNTLYLQMNSLRAEDTAVYYCARRFYYDYGWFAYWGQGTLVTVSS

SEQ ID NO: 201 - protein, 120 aa, unidentified
EVQLLESGGGLVQPGGSLRLSCAASGFTFSNYAMSWVRQAPGKRLEWVAAINSNVGSAYYPDTVKDRFTISRDNSKNTLYLQMNSLRAEDTAVYYCARRFYYDYGWFAYWGQGTLVTVSS

SEQ ID NO: 202 - protein, 120 aa, unidentified
EVQLLESGGGLVQPGGSLRLSCAASGFTFSNYAMSWVRQAPGKRLEWVAAINSQGGSAYYPDTVKDRFTISRDNSKNTLYLQMNSLRAEDTAVYYCARRFYYDYGWFAYWGQGTLVTVSS

SEQ ID NO: 203 - protein, 120 aa, unidentified
EVQLLESGGGLVQPGGSLRLSCAASGFTFSNYAMSWVRQAPGKRLEWVAAINSDGGSAYYPDTVKDRFTISRDNSKNTLYLQMNSLRAEDTAVYYCARRFYYDYGWFAYWGQGTLVTVSS

SEQ ID NO: 204 - protein, 120 aa, unidentified
EVQLLESGGGLVQPGGSLRLSCAASGFTFSNYAMSWVRQAPGKRLEWVAAINANGGSAYYPDTVKDRFTISRDNSKNTLYLQMNSLRAEDTAVYYCARRFYYDYGWFAYWGQGTLVTVSS

SEQ ID NO: 205 - protein, 120 aa, unidentified
EVQLLESGGGLVQPGGSLRLSCAASGFTFSNYAMSWVRQAPGKRLEWVAAINTNGGSAYYPDTVKDRFTISRDNSKNTLYLQMNSLRAEDTAVYYCARRFYYDYGWFAYWGQGTLVTVSS

SEQ ID NO: 206 - protein, 120 aa, unidentified
EVQLLESGGGLVQPGGSLRLSCAASGFTFSNYAMSWVRQAPGKRLEWVAAIQSNGGSAYYPDTVKDRFTISRDNSKNTLYLQMNSLRAEDTAVYYCARRFYYDYGWFAYWGQGTLVTVSS

SEQ ID NO: 207 - protein, 120 aa, unidentified
EVQLLESGGGLVQPGGSLRLSCAASGFTFSNYAMSWVRQAPGKRLEWVAAIDSNGGSAYYPDTVKDRFTISRDNSKNTLYLQMNSLRAEDTAVYYCARRFYYDYGWFAYWGQGTLVTVSS

SEQ ID NO: 208 - protein, 120 aa, unidentified
EVQLLESGGGLVQPGGSLRLSCAASGFTFSNYAMSWVRQAPGKRLEWVAAITSNGGSAYYPDTVKDRFTISRDNSKNTLYLQMNSLRAEDTAVYYCARRFYYDYGWFAYWGQGTLVTVSS

SEQ ID NO: 209 - protein, 120 aa, unidentified
EVQLLESGGGLVQPGGSLRLSCAASGFTFSNYAMSWVRQAPGKRLEWVAAISSNGGSAYYPDTVKDRFTISRDNSKNTLYLQMNSLRAEDTAVYYCARRFYYDYGWFAYWGQGTLVTVSS

SEQ ID NO: 210 - protein, 214 aa, unidentified
DIVLTQSPASLSASVGETVTITCRASENIDSYLAWYQQKQGKSPQLLVYAATLLADGVPSRFSGSGSGTQYSLKINSLQSEDVARYYCQHYYSSPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 223 aa, unidentified
EVQLVESGGGLVKPGGSLKLSCAASGFTFSNYAMSWVRQTPEKRLEWVAAINSNGGSAYYPDTVKDRFTISRDNAKNTLYLQMSSLRSEDTALYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 212 - protein, 450 aa, unidentified
EVQLVESGGGLVKPGGSLKLSCAASGFTFSNYAMSWVRQTPEKRLEWVAAINSNGGSAYYPDTVKDRFTISRDNAKNTLYLQMSSLRSEDTALYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 450 aa, unidentified
EVQLVESGGGLVKPGGSLKLSCAASGFTFSNYAMSWVRQTPEKRLEWVAAINSNGGSAYYPDTVKDRFTISRDNAKNTLYLQMSSLRSEDTALYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 220 aa, unidentified
AQVLTQTPSPVSAAVGGTVTINCQASQSVFNNKNLAWYQQKPGQPPKLLIYKASTLASGVSSRFKGSGSGTQFALTISGVQCDDAATYYCQGEFSCSSTDCVTFGGGTEVVVKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 215 - protein, 221 aa, unidentified
QSVEESGGRLVTPGTPLTLTCTVSGFSLSSYAMIWVRQAPGKGLEWIGIINSSDNTHYATWAKGRFTISKTSTTVDLKITSPTTEDTATYFCARDPYDYGYGWYFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 216 - protein, 448 aa, unidentified
QSVEESGGRLVTPGTPLTLTCTVSGFSLSSYAMIWVRQAPGKGLEWIGIINSSDNTHYATWAKGRFTISKTSTTVDLKITSPTTEDTATYFCARDPYDYGYGWYFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 448 aa, unidentified
QSVEESGGRLVTPGTPLTLTCTVSGFSLSSYAMIWVRQAPGKGLEWIGIINSSDNTHYATWAKGRFTISKTSTTVDLKITSPTTEDTATYFCARDPYDYGYGWYFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 220 aa, unidentified
AQVLTQTPSPVSAAVGGTVTINCQASQSIYNSKNLAWYQQKPGQPPKLLIYSASTLASGVPSRFKGSGSGTQFTLTISDLECDDAATYYCQGEFSCSSVDCATFGGGTEVVVKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 219 - protein, 222 aa, unidentified
QSVEESGGRLVTPGTPLTLTCTVSGFSLSNYAMSWVRQAPGKGLEWIGIIYTTGFTYYASWVKGRFTISKTSTTVDLKITSPTTEDTATYFCARGLATYVSPPTRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 220 - protein, 449 aa, unidentified
QSVEESGGRLVTPGTPLTLTCTVSGFSLSNYAMSWVRQAPGKGLEWIGIIYTTGFTYYASWVKGRFTISKTSTTVDLKITSPTTEDTATYFCARGLATYVSPPTRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 449 aa, unidentified
QSVEESGGRLVTPGTPLTLTCTVSGFSLSNYAMSWVRQAPGKGLEWIGIIYTTGFTYYASWVKGRFTISKTSTTVDLKITSPTTEDTATYFCARGLATYVSPPTRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 17 aa, unidentified
AINANSGSAYYPDTVKD

SEQ ID NO: 223 - protein, 17 aa, unidentified
AINTNSGSAYYPDTVKD

SEQ ID NO: 224 - protein, 17 aa, unidentified
AIQSNSGSAYYPDTVKD

SEQ ID NO: 225 - protein, 17 aa, unidentified
AIDSNSGSAYYPDTVKD

SEQ ID NO: 226 - protein, 17 aa, unidentified
AITSNSGSAYYPDTVKD

SEQ ID NO: 227 - protein, 17 aa, unidentified
AISSNSGSAYYPDTVKD

SEQ ID NO: 228 - protein, 17 aa, unidentified
AINANAGSAYYPDTVKD

SEQ ID NO: 229 - protein, 17 aa, unidentified
AINTNAGSAYYPDTVKD

SEQ ID NO: 230 - protein, 17 aa, unidentified
AIQSNAGSAYYPDTVKD

SEQ ID NO: 231 - protein, 17 aa, unidentified
AIDSNAGSAYYPDTVKD

SEQ ID NO: 232 - protein, 17 aa, unidentified
AITSNAGSAYYPDTVKD

SEQ ID NO: 233 - protein, 17 aa, unidentified
AISSNAGSAYYPDTVKD

SEQ ID NO: 234 - protein, 17 aa, unidentified
AINANVGSAYYPDTVKD

SEQ ID NO: 235 - protein, 17 aa, unidentified
AINTNVGSAYYPDTVKD

SEQ ID NO: 236 - protein, 17 aa, unidentified
AIQSNVGSAYYPDTVKD

SEQ ID NO: 237 - protein, 17 aa, unidentified
AIDSNVGSAYYPDTVKD

SEQ ID NO: 238 - protein, 17 aa, unidentified
AITSNVGSAYYPDTVKD

SEQ ID NO: 239 - protein, 17 aa, unidentified
AISSNVGSAYYPDTVKD

SEQ ID NO: 240 - protein, 17 aa, unidentified
AINAQGGSAYYPDTVKD

SEQ ID NO: 241 - protein, 17 aa, unidentified
AINTQGGSAYYPDTVKD

SEQ ID NO: 242 - protein, 17 aa, unidentified
AIQSQGGSAYYPDTVKD

SEQ ID NO: 243 - protein, 17 aa, unidentified
AIDSQGGSAYYPDTVKD

SEQ ID NO: 244 - protein, 17 aa, unidentified
AITSQGGSAYYPDTVKD

SEQ ID NO: 245 - protein, 17 aa, unidentified
AISSQGGSAYYPDTVKD

SEQ ID NO: 246 - protein, 17 aa, unidentified
AINADGGSAYYPDTVKD

SEQ ID NO: 247 - protein, 17 aa, unidentified
AINTDGGSAYYPDTVKD

SEQ ID NO: 248 - protein, 17 aa, unidentified
AIQSDGGSAYYPDTVKD

SEQ ID NO: 249 - protein, 17 aa, unidentified
AIDSDGGSAYYPDTVKD

SEQ ID NO: 250 - protein, 17 aa, unidentified
AITSDGGSAYYPDTVKD

SEQ ID NO: 251 - protein, 17 aa, unidentified
AISSDGGSAYYPDTVKD

SEQ ID NO: 252 - protein, 285 aa, unidentified
ATGLKEPLSFHVIWIASFYNHSWKQNLVSGWLSDLQTHTWDSNSSTIVFLWPWSRGNFSNEEWKELETLFRIRTIRSFEGIRRYAHELQFEYPFEIQVTGGCELHSGKVSGSFLQLAYQGSDFVSFQNNSWLPYPVAGNMAKHFCKVLNQNQHENDITHNLLSDTCPRFILGLLDAGKAHLQRQVKPEAWLSHGPSPGPGHLQLVCHVSGFYPKPVWVMWMRGEQEQQGTQRGDILPSADGTWYLRATLEVAAGEAADLSCRVKHSSLEGQDIVLYWEGSLVPRG

SEQ ID NO: 253 - protein, 215 aa, unidentified
EIVLTQSPALMAASPGEKVTITCSVSSSISSSNLHWYQQKSGTSPKPWIYGTSNLASGVPVRFSGSGSGTSYSLTISSMEAEDAATYYCQQWSSYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 254 - protein, 221 aa, unidentified
QVQLQQSGPGLVAPSQSLFITCTVSGFSLTTYEINWVRQPPGKGLEWLGVIWTGGSTNYNSALISRLSISKDNSKSLVFLKMNSLQTDDTAIYYCVRTLPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 255 - protein, 448 aa, unidentified
QVQLQQSGPGLVAPSQSLFITCTVSGFSLTTYEINWVRQPPGKGLEWLGVIWTGGSTNYNSALISRLSISKDNSKSLVFLKMNSLQTDDTAIYYCVRTLPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 256 - protein, 448 aa, unidentified
QVQLQQSGPGLVAPSQSLFITCTVSGFSLTTYEINWVRQPPGKGLEWLGVIWTGGSTNYNSALISRLSISKDNSKSLVFLKMNSLQTDDTAIYYCVRTLPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 16 aa, unidentified
VIWTGGSTNYNAALIS

SEQ ID NO: 258 - protein, 16 aa, unidentified
VIWTGGSTNYNTALIS

SEQ ID NO: 259 - protein, 16 aa, unidentified
VIWTGGSTNYQSALIS

SEQ ID NO: 260 - protein, 108 aa, unidentified
EIVLTQSPDFQSVTPKEKVTITCSVSSSISSSNLHWYQQKPDQSPKPWIYGTSNLASGVPVRFSGSGSGTSYTLTINSLEAEDAATYYCQQWSSYPPTFGQGTKLEIK

SEQ ID NO: 261 - protein, 118 aa, unidentified
EVQLQESGPGLVKPSETLSLTCTVSGFSLTTYEINWVRQPPGKGLEWLGVIWTGGSTNYNSALISRVTISKDNSKSQVSLKLSSVTAADTAVYYCVRTLPYYYAMDYWGQGTTVTVSS

SEQ ID NO: 262 - protein, 118 aa, unidentified
EVQLQESGPGLVKPSETLSLTCTVSGFSLTTYEINWVRQPPGKGLEWLGVIWTGGSTNYNAALISRVTISKDNSKSQVSLKLSSVTAADTAVYYCVRTLPYYYAMDYWGQGTTVTVSS

SEQ ID NO: 263 - protein, 118 aa, unidentified
EVQLQESGPGLVKPSETLSLTCTVSGFSLTTYEINWVRQPPGKGLEWLGVIWTGGSTNYNTALISRVTISKDNSKSQVSLKLSSVTAADTAVYYCVRTLPYYYAMDYWGQGTTVTVSS

SEQ ID NO: 264 - protein, 118 aa, unidentified
EVQLQESGPGLVKPSETLSLTCTVSGFSLTTYEINWVRQPPGKGLEWLGVIWTGGSTNYQSALISRVTISKDNSKSQVSLKLSSVTAADTAVYYCVRTLPYYYAMDYWGQGTTVTVSS

SEQ ID NO: 265 - protein, 225 aa, unidentified
EVQLVESGGGLVQPGGSLRLSCAVSGFSLSNYAMSWVRQAPGKGLEWIGIIYTTGFTYYASWVKGRFTISKDSSKNTVYLQMNSLRAEDTAVYYCARGLATYVSPPTRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 266 - protein, 220 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSCSSVDCATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - protein, 220 aa, unidentified
AQVLTQSPSSLSASVGDRVTITCQASQSIYNTKNLAWYQQKPGKAPKLLIYSASTLASGVPSRFSGSGSGTQFTLTISSLQPEDFATYYCQGEFSCSSVDCATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 220 aa, unidentified
AQVLTQSPSSLSASVGDRVTITCQASQSIYNAKNLAWYQQKPGKAPKLLIYSASTLASGVPSRFSGSGSGTQFTLTISSLQPEDFATYYCQGEFSCSSVDCATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 220 aa, unidentified
AQVLTQSPSSLSASVGDRVTITCQASQSIYNNKNLAWYQQKPGKAPKLLIYSASTLASGVPSRFSGSGSGTQFTLTISSLQPEDFATYYCQGEFSCSSVDCATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 270 - protein, 220 aa, unidentified
AQVLTQSPSSLSASVGDRVTITCQASQSIYQSKNLAWYQQKPGKAPKLLIYSASTLASGVPSRFSGSGSGTQFTLTISSLQPEDFATYYCQGEFSCSSVDCATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 220 aa, unidentified
AQVLTQSPSSLSASVGDRVTITCQASQSIYDSKNLAWYQQKPGKAPKLLIYSASTLASGVPSRFSGSGSGTQFTLTISSLQPEDFATYYCQGEFSCSSVDCATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - protein, 220 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSSSSVDSATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 273 - protein, 220 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSSSSVDAATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 274 - protein, 220 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSSSSVDVATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 275 - protein, 220 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSASSVDSATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 276 - protein, 220 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSASSVDAATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 277 - protein, 220 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSASSVDVATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 278 - protein, 220 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSVSSVDSATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 279 - protein, 220 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSVSSVDAATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 280 - protein, 220 aa, unidentified
AQVLTQSPSSLSASVGDRVTITCQASQSIYNSKNLAWYQQKPGKAPKLLIYSASTLASGVPSRFSGSGSGTQFTLTISSLQPEDFATYYCQGEFSVSSVDVATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 281 - protein, 223 aa, unidentified
EVQLLESGGGLVQPGGSLRLSCAASGFTFSNYAMSWVRQAPGKRLEWVAAINSNGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 282 - protein, 214 aa, unidentified
DIQMTQSPSSLSASVGDRVTITCRASENIDSYLAWYQQKPGKAPKLLVYAATLLADGVPSRFSGSGSGTQYTLTISSLQPEDFARYYCQHYYSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - protein, 214 aa, unidentified
DIQMTQSPSSLSASVGDRVTITCRASENIDSYLAWYQQKPGKAPKLLVYAATLLAEGVPSRFSGSGSGTQYTLTISSLQPEDFARYYCQHYYSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 284 - protein, 214 aa, unidentified
DIQMTQSPSSLSASVGDRVTITCRASENIDSYLAWYQQKPGKAPKLLVYAATLLASGVPSRFSGSGSGTQYTLTISSLQPEDFARYYCQHYYSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 285 - protein, 223 aa, unidentified
EVQLLESGGGLVQPGGSLRLSCAASGFTFSNYAMSWVRQAPGKRLEWVAAINSNSGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 286 - protein, 223 aa, unidentified
EVQLLESGGGLVQPGGSLRLSCAASGFTFSNYAMSWVRQAPGKRLEWVAAINSNAGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 287 - protein, 223 aa, unidentified
EVQLLESGGGLVQPGGSLRLSCAASGFTFSNYAMSWVRQAPGKRLEWVAAINSNVGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 288 - protein, 223 aa, unidentified
EVQLLESGGGLVQPGGSLRLSCAASGFTFSNYAMSWVRQAPGKRLEWVAAINSQGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 289 - protein, 223 aa, unidentified
EVQLLESGGGLVQPGGSLRLSCAASGFTFSNYAMSWVRQAPGKRLEWVAAINSDGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 290 - protein, 223 aa, unidentified
EVQLLESGGGLVQPGGSLRLSCAASGFTFSNYAMSWVRQAPGKRLEWVAAINANGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 291 - protein, 223 aa, unidentified
EVQLLESGGGLVQPGGSLRLSCAASGFTFSNYAMSWVRQAPGKRLEWVAAINTNGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 292 - protein, 223 aa, unidentified
EVQLLESGGGLVQPGGSLRLSCAASGFTFSNYAMSWVRQAPGKRLEWVAAIQSNGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 293 - protein, 223 aa, unidentified
EVQLLESGGGLVQPGGSLRLSCAASGFTFSNYAMSWVRQAPGKRLEWVAAIDSNGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 294 - protein, 223 aa, unidentified; identical to SEQ ID NO: 293

SEQ ID NO: 295 - protein, 223 aa, unidentified
EVQLLESGGGLVQPGGSLRLSCAASGFTFSNYAMSWVRQAPGKRLEWVAAITSNGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 296 - protein, 223 aa, unidentified
EVQLLESGGGLVQPGGSLRLSCAASGFTFSNYAMSWVRQAPGKRLEWVAAISSNGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 297 - protein, 227 aa, unidentified
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 298 - protein, 476 aa, unidentified
EVQLVESGGGLVQPGGSLRLSCAASGYSFTGYTMNWVRQAPGKGLEWVALINPYKGVTTYADSVKGRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 299 - protein, 450 aa, unidentified
EVQLVESGGGLVKPGGSLKLSCAASGFTFSNYAMSWVRQTPEKRLEWVAAINSNGGSAYYPDTVKDRFTISRDNAKNTLYLQMSSLRSEDTALYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 300 - protein, 452 aa, unidentified
EVQLVESGGGLVQPGGSLRLSCAVSGFSLSNYAMSWVRQAPGKGLEWIGIIYTTGFTYYASWVKGRFTISKDSSKNTVYLQMNSLRAEDTAVYYCARGLATYVSPPTRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 301 - protein, 450 aa, unidentified
EVQLLESGGGLVQPGGSLRLSCAASGFTFSNYAMSWVRQAPGKRLEWVAAINSNGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 302 - protein, 450 aa, unidentified
EVQLLESGGGLVQPGGSLRLSCAASGFTFSNYAMSWVRQAPGKRLEWVAAINSNSGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 303 - protein, 450 aa, unidentified
EVQLLESGGGLVQPGGSLRLSCAASGFTFSNYAMSWVRQAPGKRLEWVAAINSNAGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 304 - protein, 450 aa, unidentified
EVQLLESGGGLVQPGGSLRLSCAASGFTFSNYAMSWVRQAPGKRLEWVAAINSNVGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 305 - protein, 450 aa, unidentified
EVQLLESGGGLVQPGGSLRLSCAASGFTFSNYAMSWVRQAPGKRLEWVAAINSQGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - protein, 450 aa, unidentified
EVQLLESGGGLVQPGGSLRLSCAASGFTFSNYAMSWVRQAPGKRLEWVAAINSDGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 307 - protein, 450 aa, unidentified
EVQLLESGGGLVQPGGSLRLSCAASGFTFSNYAMSWVRQAPGKRLEWVAAINANGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - protein, 450 aa, unidentified
EVQLLESGGGLVQPGGSLRLSCAASGFTFSNYAMSWVRQAPGKRLEWVAAINTNGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 309 - protein, 450 aa, unidentified
EVQLLESGGGLVQPGGSLRLSCAASGFTFSNYAMSWVRQAPGKRLEWVAAIQSNGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 450 aa, unidentified
EVQLLESGGGLVQPGGSLRLSCAASGFTFSNYAMSWVRQAPGKRLEWVAAIDSNGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 450 aa, unidentified
EVQLLESGGGLVQPGGSLRLSCAASGFTFSNYAMSWVRQAPGKRLEWVAAITSNGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 312 - protein, 450 aa, unidentified
EVQLLESGGGLVQPGGSLRLSCAASGFTFSNYAMSWVRQAPGKRLEWVAAISSNGGSAYYPDTVKDRFTISRDNSKNTLYLQMNSLRAEDTAVYYCARRFYYDYGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 120 aa, unidentified; identical to SEQ ID NO: 7

SEQ ID NO: 314 - protein, 107 aa, unidentified; identical to SEQ ID NO: 8

(19) United States
(12) Patent Application Publication
(10) Pub. No.: US 2025/0382372 A1
(43) Pub. Date: Dec. 18, 2025

(54) ANTI-CD1A ANTIBODIES

(71) Applicant: Oxford University Innovation Limited, Oxford (GB)

(72) Inventors: Clare HARDMAN, Oxford (GB); Yi-Ling CHEN, Oxford (GB); Graham OGG, Oxford (GB); Marcin WEGRECKI, Oxford (GB); Jamie ROSSJOHN, Oxford (GB)

(21) Appl. No.: 19/221,384

(22) Filed: May 28, 2025

Related U.S. Application Data
(63) Continuation of application No. PCT/GB2023/053095, filed on Nov. 29, 2023.

(30) Foreign Application Priority Data
Nov. 29, 2022 (GB) 2217923.8

(51) Int. Cl. C07K 16/28; A61K 39/00; A61K 45/06; A61P 29/00; G01N 33/68

(52) U.S. Cl. CPC C07K 16/2833; A61K 45/06; A61P 29/00; G01N 33/6854; A61K 2039/505; C07K 2317/24; C07K 2317/52; C07K 2317/565; C07K 2317/622; C07K 2317/732; G01N 2333/70596; G01N 2800/52; G01N 2800/7095

(57) ABSTRACT

The invention relates to an antibody or antigen binding fragment thereof which is capable of binding to CD1a. The antibody or antigen binding fragment thereof may be chimeric or humanised, and may be used to treat one or more inflammatory skin or mucosal disorder, or disease or one or more associated systemic disease or disorder, or one or more inflammatory drug reaction which manifests systemically, or a CD1a-expressing malignancy.

### RELATED APPLICATIONS

The present application is a continuation of International PCT Application No. PCT/GB2023/053095, filed Nov. 29, 2023, which claims the benefit of British Application No. 2217923.8, filed Nov. 29, 2022, the content of which is hereby incorporated by reference in its entirety.

### REFERENCE TO AN ELECTRONIC SEQUENCE LISTING

The contents of the electronic sequence listing (COAB_002_01US_SubSeqList_ST26.xml; Size: 311,202 bytes; and Date of Creation: Aug. 19, 2025) are herein incorporated by reference in its entirety.

### FIELD OF INVENTION

The present invention relates to antibodies, and their use in treating, preventing, diagnosing or monitoring inflammatory skin and mucosal diseases or disorders, or associated systemic diseases or disorders, or inflammatory drug reactions, or CD1a-expressing malignancies.

### BACKGROUND

Antigen presentation is one of the fundamental pillars of host immunity, by which the immune system detects threats including infection, tissue damage and disease, and orchestrates a tailored defence. Antigen presentation encompasses antigen internalisation, processing and display by presentation molecules on the surface of specialised antigen-presenting cells (APCs). Presentation of antigen is organised to achieve optimal activation of the immune response targeted to the antigen source and eliminate the threat. Antigens encompass a broad range of molecules including peptides, lipids and metabolites and others. MHCI and MHCII are proteins expressed on the surface of APCs which bind to peptide antigens and largely present to CD8⁺ T cells and CD4+ T cells respectively. These T cell subsets are induced to exert their effector functions upon recognition of the MHC-bound peptide antigen by the cell surface T-cell receptor (TCR) enabling immunity to pathogens and to cancers. However, dysregulated presentation of innocuous antigens, such as allergens in allergic diseases, or self-proteins in autoimmunity causes host damage, inflammation and disease. Therefore, targeting of the antigen presentation pathway is a powerful means of modulating the ensuing immune response.

CD1 molecules constitute a family of antigen presentation molecules structurally akin to MHCI. In contrast, CD1 molecules are relatively non-polymorphic and the CD1 antigen binding groove is enriched in hydrophobic amino acids enabling presentation of lipid species. Lipids are important antigens forming vital components of host and pathogen cell membranes and are less subject to mutation than protein-derived peptide antigens. The CD1 family is made up of cell surface group-1 molecules CD1a/b/c and group-2 CD1d and group-3 CD1e. Most of the understanding of CD1 lipid presentation and T cell responses has come from study of invariant Natural Killer T cell recognition of glycolipid bound CD1d, partly because CD1d is the only CD1 normally expressed by mice. CD1d and MHCI molecules are broadly expressed whereas MHCII and group 1 CD1 expression is relatively restricted to APCs. However, CD1a unique among these molecules is highly specific to the skin and mucosae. CD1a is constitutively expressed by Langerhans cells (LCs) in the epidermis of skin and mucosae (1) and is commonly used as an identifying marker for LCs, in addition to langerin. Additionally, CD1a is expressed at lower levels on subsets of dermal dendritic cells (2-4) and can be expressed and upregulated on skin innate lymphoid cells (ILCs), in particular ILC2 (5). Importantly, CD1a was first described on the surface of immature thymocytes, but expression is typically lost upon T cell maturation (6). The high level of constitutive expression of CD1a in the skin is indicative of an important physiological role for CD1a-dependent surveillance and T cell activation in healthy and diseased human skin. Moreover, the increase in CD1a expression in atopic dermatitis skin may underlie the increased activation of CD1a-reactive T cell populations in inflammatory skin disease.

T cell responses directed by CD1a, CD1b, or CD1c molecules presenting mycobacterial lipid-based antigens have been implicated in human immune responses to Mycobacterium tuberculosis and Mycobacterium leprae infections. Recognition of other, more common pathogenic or commensal bacterial lipids by CD1a-restricted T cells is the subject of ongoing studies, with some data presented herein. Whereas TCR recognition of peptide antigens by MHC-restricted T cells is generally highly specific for the peptide antigen, the CD1 mode of TCR recognition is more diverse with highly lipid-specific responses (7) and cross-reactive or even apparently lipid independent signalling mediated by direct TCR-CD1 interaction (8-10), as is the case for CD1a-autoreactive T cells. CD1a-autoreactive T cells are activated in some cases upon recognition of CD1a carrying small hydrophobic host-derived lipids that nest within the antigen binding groove and do not protrude, allowing the TCR to interact with the CD1a protein itself, rather than with the lipid. In this case binding of lipids with large or charged headgroups would prevent the interaction between an autoreactive TCR and CD1a, thereby preventing T cell activation (11, 12).

CD1a is relatively non-polymorphic, and so there is therefore population-wide potential in prevention and/or treatment of inflammatory skin and mucosal diseases and disorders, such as atopic dermatitis, psoriasis, lupus erythematosus, or associated systemic diseases or disorders, or inflammatory drug reactions which manifest systemically, where the frequency of CD1a-expressing dendritic cell subsets is altered, and migratory patterns of LCs or responding T cells are altered (13-15). Furthermore, CD1a has been linked to other systemic disorders including inflammatory bowel disease, multiple sclerosis, Guillain-Barre syndrome, thyroiditis, and neurodegeneration (Al-amodi Inflammatory Bowel Diseases 2018 24: 1225-1236; Caporale J Neuroimmunol 2006 177:112-8; Jamshidian Immunological Investigations 2010 3:874-889; Roura-Mir J Immunol 2005 174:3773-80; Wang Aging 2019 11: 4521-4535). In addition, CD1a can be expressed by certain malignancies including Langerhans cell histiocytosis, Langerhans cell sarcoma, subsets of T cell lymphomas, subsets of thymomas and rare descriptions of other malignancies, such as subsets of mastocytosis.

It is an object of the invention to provide anti-CD1a antibodies. Such antibodies are particularly useful in treating or preventing inflammatory diseases or disorders of the skin or mucosa, such as psoriasis, dermatitis, lupus erythematosus or drug reactions which manifest as an inflammatory skin or mucosal disease or disorder. Such antibodies may also be beneficial in treating or preventing associated systemic diseases or disorders, or inflammatory drug reactions which manifest systemically or in the treatment of CD1a-expressing malignancies.

### SUMMARY OF INVENTION

The invention relates to an antibody or antigen binding fragment thereof which is capable of binding to CD1a. The antibody or antigen binding fragment thereof may specifically bind to CD1a. The antibody or antigen binding fragment thereof may preferentially bind to CD1a. The antibody or antigen binding fragment thereof may induce cell death of cells expressing CD1a. The antibody or antigen binding fragment thereof may block the binding of ligands to CD1a.

In an aspect, the antibody or antigen binding fragment thereof may be a chimeric antibody comprising or consisting of:
a) a heavy chain variable region comprising:
   a CDR1 of SEQ ID NO: 33,
   a CDR2 of SEQ ID NO: 34, and
   a CDR3 of SEQ ID NO: 35,
   or sequences having at least 80%, 90%, 95%, 98%, 99% or 100% identity thereto, and/or a light chain variable region comprising:
   a CDR1 of SEQ ID NO: 36,
   a CDR2 of SEQ ID NO: 37, and
   a CDR3 of SEQ ID NO: 38,
   or sequences having at least 80%, 90%, 95%, 98%, 99% or 100% identity thereto; or
b) a heavy chain variable region comprising:
   a CDR1 of SEQ ID NO: 1,
   a CDR2 of SEQ ID NO: 2, and
   a CDR3 of SEQ ID NO: 3,
   or sequences having at least 80%, 90%, 95%, 98%, 99% or 100% identity thereto, and/or a light chain variable region comprising:
   a CDR1 of SEQ ID NO: 4,
   a CDR2 of SEQ ID NO: 5, and
   a CDR3 of SEQ ID NO: 6,
   or sequences having at least 80%, 90%, 95%, 98%, 99% or 100% identity thereto; or
c) a heavy chain variable region comprising:
   a CDR1 of SEQ ID NO: 9,
   a CDR2 of SEQ ID NO: 10, and
   a CDR3 of SEQ ID NO: 11,
   or sequences having at least 80%, 90%, 95%, 98%, 99% or 100% identity thereto, and/or
   a light chain variable region comprising:
   a CDR1 of SEQ ID NO: 12,
   a CDR2 of SEQ ID NO: 13, and
   a CDR3 of SEQ ID NO: 14,
   or sequences having at least 80%, 90%, 95%, 98%, 99% or 100% identity thereto; or
d) a heavy chain variable region comprising:
   a CDR1 of SEQ ID NO: 17,
   a CDR2 of SEQ ID NO: 18, and
   a CDR3 of SEQ ID NO: 19,
   or sequences having at least 80%, 90%, 95%, 98%, 99% or 100% identity thereto, and/or
   a light chain variable region comprising:
   a CDR1 of SEQ ID NO: 20,
   a CDR2 of SEQ ID NO: 21, and
   a CDR3 of SEQ ID NO: 22,
   or sequences having at least 80%, 90%, 95%, 98%, 99% or 100% identity thereto; or
e) a heavy chain variable region comprising:
   a CDR1 of SEQ ID NO: 25,
   a CDR2 of SEQ ID NO: 26, and
   a CDR3 of SEQ ID NO: 27,
   or sequences having at least 80%, 90%, 95%, 98%, 99% or 100% identity thereto, and/or
   a light chain variable region comprising:
   a CDR1 of SEQ ID NO: 28,
   a CDR2 of SEQ ID NO: 29, and
   a CDR3 of SEQ ID NO: 30,
   or sequences having at least 80%, 90%, 95%, 98%, 99% or 100% identity thereto; or
(f) a heavy chain variable region comprising:
   a CDR1 of SEQ ID NO: 91,
   a CDR2 of SEQ ID NO: 92, and
   a CDR3 of SEQ ID NO: 93,
   or sequences having at least 80%, 90%, 95%, 98%, 99% or 100% identity thereto; and/or
   a light chain variable region comprising:
   a CDR1 of SEQ ID NO: 94,
   a CDR2 of SEQ ID NO: 95, and
   a CDR3 of SEQ ID NO: 96
      or sequences having at least 80%, 90%, 95%, 98%, 99% or 100% identity thereto.

In any of the above, the antibody or antigen binding fragment thereof which is a chimeric antibody, any combination of the CDRs may be employed. Alternatively, the antibody or antigen binding fragment thereof which is a chimeric antibody may comprise only the recited CDR3 of the heavy and light chain variable regions above.

In another aspect, the antibody or antigen binding fragment thereof may be a chimeric antibody comprising or consisting of:
(a) a heavy chain comprising or consisting of SEQ ID NO: 211, SEQ ID NO: 212 or SEQ ID NO: 213, or a sequence having at least 80%, 90%, 95%, 98%, 99% or 100% identity thereto; and/or
   a light chain comprising or consisting of SEQ ID NO: 210,
   or a sequence having at least 80%, 90%, 95%, 98%, 99% or 100% identity thereto; or
(b) a heavy chain comprising or consisting of SEQ ID NO: 215, SEQ ID NO: 216 or SEQ ID NO: 217, or a sequence having at least 80%, 90%, 95%, 98%, 99% or 100% identity thereto; and/or
   a light chain comprising or consisting of SEQ ID NO: 214,
   or a sequence having at least 80%, 90%, 95%, 98%, 99% or 100% identity thereto; or
(c) a heavy chain comprising or consisting of SEQ ID NO: 219, SEQ ID NO: 220 or SEQ ID NO: 221, or a sequence having at least 80%, 90%, 95%, 98%, 99% or 100% identity thereto; and/or
   a light chain comprising or consisting of SEQ ID NO: 218,
   or a sequence having at least 80%, 90%, 95%, 98%, 99% or 100% identity thereto; or
(d) a heavy chain comprising or consisting of SEQ ID NO: 254, SEQ ID NO: 255 or SEQ ID NO: 256, or a sequence having at least 80%, 90%, 95%, 98%, 99% or 100% identity thereto; and/or
   a light chain comprising or consisting of SEQ ID NO: 253,
   or a sequence having at least 80%, 90%, 95%, 98%, 99% or 100% identity thereto.

The antibody or antigen binding fragment thereof may be a chimeric antibody comprising or consisting of:
a) a heavy chain comprising or consisting of SEQ ID NO: 211, SEQ ID NO: 212 or SEQ ID NO: 213; and
b) a light chain comprising or consisting of SEQ ID NO: 210.

The antibody or antigen binding fragment thereof may be a chimeric antibody comprising or consisting of:
a) a heavy chain comprising or consisting of SEQ ID NO: 215, SEQ ID NO: 216 or SEQ ID NO: 217; and
b) a light chain comprising or consisting of SEQ ID NO: 214.

The antibody or antigen binding fragment thereof may be a chimeric antibody comprising or consisting of:
a) a heavy chain comprising or consisting of SEQ ID NO: 219, SEQ ID NO: 220 or SEQ ID NO: 221; and
b) a light chain comprising or consisting of SEQ ID NO: 218.

The antibody or antigen binding fragment thereof may be a humanised antibody comprising or consisting of:
a) a heavy chain variable region comprising:
   a CDR1 of SEQ ID NO: 9,
   a CDR2 of SEQ ID NO: 10, and
   a CDR3 of SEQ ID NO: 11,
      or sequences having at least 80%, 90%, 95%, 98%, 99% or 100% identity thereto; and/or
b) a light chain variable region comprising:
   a CDR1 of SEQ ID NO: 12,
   a CDR2 of SEQ ID NO: 13, and
   a CDR3 of SEQ ID NO: 14, SEQ ID NO: 107, SEQ ID NO: 108 or SEQ ID NO: 109,
      or sequences having at least 80%, 90%, 95%, 98%, 99% or 100% identity thereto.

The antibody or antigen binding fragment thereof may be a humanised antibody comprising or consisting of:
a) a heavy chain variable region comprising:
   a CDR1 of SEQ ID NO: 17,
   a CDR2 of SEQ ID NO: 18, SEQ ID NO: 132, SEQ ID NO: 133 or SEQ ID NO: 134, and
   a CDR3 of SEQ ID NO: 19 or SEQ ID NO: 110,
      or sequences having at least 80%, 90%, 95%, 98%, 99% or 100% identity thereto; and/or
b) a light chain variable region comprising:
   a CDR1 of SEQ ID NO: 20,
   a CDR2 of SEQ ID NO: 21, and
   a CDR3 of SEQ ID NO: 22, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118 or SEQ ID NO: 119,
      or sequences having at least 80%, 90%, 95%, 98%, 99% or 100% identity thereto.

The antibody or antigen binding fragment thereof may be a humanised antibody comprising or consisting of:
a) a heavy chain variable region comprising:
   a CDR1 of SEQ ID NO: 25,
   a CDR2 of SEQ ID NO: 26, and
   a CDR3 of SEQ ID NO: 27,
      or sequences having at least 80%, 90%, 95%, 98%, 99% or 100% identity thereto; and/or
b) a light chain variable region comprising:
   a CDR1 of SEQ ID NO: 28,
   a CDR2 of SEQ ID NO: 29, and
   a CDR3 of SEQ ID NO: 30, SEQ ID NO: 120, SEQ ID NO: 121 or SEQ ID NO: 122,
      or sequences having at least 80%, 90%, 95%, 98%, 99% or 100% identity thereto.

The antibody or antigen binding fragment thereof may be a humanised antibody comprising or consisting of:
a) a heavy chain variable region comprising:
   a CDR1 of SEQ ID NO: 33,
   a CDR2 of SEQ ID NO: 34, and
   a CDR3 of SEQ ID NO: 35,
      or sequences having at least 80%, 90%, 95%, 98%, 99% or 100% identity thereto; and/or
b) a light chain variable region comprising:
   a CDR1 of SEQ ID NO: 36, SEQ ID NO: 135, SEQ ID NO: 136, SEQ ID NO: 137, SEQ ID NO: 13 or, SEQ ID NO: 139,
   a CDR2 of SEQ ID NO: 37, and
   a CDR3 of SEQ ID NO: 38, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130 or SEQ ID NO: 131,
      or sequences having at least 80%, 90%, 95%, 98%, 99% or 100% identity thereto.

The antibody or antigen binding fragment thereof may be a humanised antibody comprising or consisting of:
a) a heavy chain variable region comprising:
   a CDR1 of SEQ ID NO: 1,
   a CDR2 of SEQ ID NO: 2, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, or SEQ ID NO: 251, and
   a CDR3 of SEQ ID NO: 3,
      or sequences having at least 80%, 90%, 95%, 98%, 99% or 100% identity thereto; and/or
b) a light chain variable region comprising:
   a CDR1 of SEQ ID NO: 4,
   a CDR2 of SEQ ID NO: 5, SEQ ID NO: 140 or SEQ ID NO: 141, and
   a CDR3 of SEQ ID NO: 6,
      or sequences having at least 80%, 90%, 95%, 98%, 99% or 100% identity thereto.

The antibody or antigen binding fragment thereof may be a humanised antibody comprising or consisting of:
a) a heavy chain variable region comprising:
   a CDR1 of SEQ ID NO: 91,
   a CDR2 of SEQ ID NO: 92, SEQ ID NO: 257, SEQ ID NO: 258, or SEQ ID NO: 259 and
   a CDR3 of SEQ ID NO: 93,
   or sequences having at least 80%, 90%, 95%, 98%, 99% or 100% identity thereto; and/or
b) a light chain variable region comprising:
   a CDR1 of SEQ ID NO: 94,
   a CDR2 of SEQ ID NO: 95, and
   a CDR3 of SEQ ID NO: 96
      or sequences having at least 80%, 90%, 95%, 98%, 99% or 100% identity thereto.

The CDRs of any antibody or antigen binding fragment disclosed herein may be associated with any framework region. Preferably, the framework region is of human origin.

The antibody or antigen binding fragment thereof may be a humanised antibody comprising or consisting of:
a) a heavy chain variable region comprising or consisting of SEQ ID NO: 7, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208 or SEQ ID NO: 209, or a sequence having at least 80%, 90%, 95%, 98%, 99% or 100% identity thereto; and/or
b) a light chain variable region comprising or consisting of SEQ ID NO: 8, SEQ ID NO: 195, SEQ ID NO: 196 or SEQ ID NO: 197,
or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 97, SEQ ID NO: 261, SEQ ID NO: 262, SEQ ID NO: 263 or SEQ ID NO: 264, or a sequence having at least 80%, 90%, 95%, 98%, 99% or 100% identity thereto; and/or
b) a light chain variable region comprising or consisting of SEQ ID NO: 98 or SEQ ID NO: 260, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 15 or SEQ ID NO: 157; and
b) a light chain variable region comprising or consisting of SEQ ID NO: 16, SEQ ID NO: 153, SEQ ID NO: 154, SEQ ID NO: 155 or SEQ ID NO: 156.

The antibody or antigen binding fragment thereof may be a humanised antibody comprising or consisting of:
a) a heavy chain variable region comprising or consisting of SEQ ID NO: 23, SEQ ID NO: 168, SEQ ID NO: 169, SEQ ID NO: 170, SEQ ID NO: 171, SEQ ID NO: 172 or SEQ ID NO: 173, or a sequence having at least 80%, 90%, 95%, 98%, 99% or 100% identity thereto; and
b) a light chain variable region comprising or consisting of SEQ ID NO: 24, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166 or SEQ ID NO: 167, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 31 or SEQ ID NO: 178, or a sequence having at least 80%, 90%, 95%, 98%, 99% or 100% identity thereto; and
b) a light chain variable region comprising or consisting of SEQ ID NO: 32, SEQ ID NO: 174, SEQ ID NO: 175, SEQ ID NO: 176 or SEQ ID NO: 177, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 39 or SEQ ID NO: 194, or a sequence having at least 80%, 90%, 95%, 98%, 99% or 100% identity thereto; and
b) a light chain variable region comprising or consisting of SEQ ID NO: 40, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192 or SEQ ID NO: 193, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 7, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208 or SEQ ID NO: 209, or a sequence having at least 80%, 90%, 95%, 98%, 99% or 100% identity thereto; and
b) a light chain variable region comprising or consisting of SEQ ID NO: 8, SEQ ID NO: 195, SEQ ID NO: 196 or SEQ ID NO: 197, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 97, SEQ ID NO: 261, SEQ ID NO: 262, SEQ ID NO: 263 or SEQ ID NO: 264, or a sequence having at least 80%, 90%, 95%, 98%, 99% or 100% identity thereto; and
b) a light chain variable region comprising or consisting of SEQ ID NO: 98 or SEQ ID NO: 260, or a sequence having at least 80%, 90%, 95% 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 179, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 180, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 181, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 182, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 183, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 184, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 185, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 186, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 187, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 188, or a sequence having at least 80%, 9%, 95% 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 189, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 190, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 191, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 192, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 194, or a sequence having at least 80%, 90%, 95%, 98%, 99% or 100% identity thereto; and
b) a light chain variable region comprising or consisting of SEQ ID NO: 193, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 198, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 198, or a sequence having at least 80%, 90%, 95%, 98%, 99% or 100% identity thereto; and
b) a light chain variable region comprising or consisting of SEQ ID NO: 196, or a sequence having at least 80%, 9%, 95% 98%, 99% or 100% identity thereto.

The antibody or antigen binding fragment thereof may be a humanised antibody comprising or consisting of:
a) a heavy chain variable region comprising or consisting of SEQ ID NO: 198, or a sequence having at least 80%, 90%, 95%, 98%, 99% or 100% identity thereto; and
b) a light chain variable region comprising or consisting of SEQ ID NO: 197, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 199, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 199, or a sequence having at least 80%, 90%, 95%, 98%, 99% or 100% identity thereto; and
b) a light chain variable region comprising or consisting of SEQ ID NO: 196, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 199, or a sequence having at least 80%, 90%, 95%, 98%, 99% or 100% identity thereto; and
b) a light chain variable region comprising or consisting of SEQ ID NO: 197, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 200, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 200, or a sequence having at least 80%, 90%, 95%, 98%, 99% or 100% identity thereto; and
b) a light chain variable region comprising or consisting of SEQ ID NO: 196, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 200, or a sequence having at least 80%, 90%, 95%, 98%, 99% or 100% identity thereto; and
b) a light chain variable region comprising or consisting of SEQ ID NO: 197, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 201, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 201, or a sequence having at least 80%, 90%, 95%, 98%, 99% or 100% identity thereto; and
b) a light chain variable region comprising or consisting of SEQ ID NO: 196, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 201, or a sequence having at least 80%, 90%, 95%, 98%, 99% or 100% identity thereto; and
b) a light chain variable region comprising or consisting of SEQ ID NO: 197, or a sequence having at least 80%, 9%, 95% 98%, 99% or 100% identity thereto.

The antibody or antigen binding fragment thereof may be a humanised antibody comprising or consisting of:
a) a heavy chain variable region comprising or consisting of SEQ ID NO: 202, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 202, or a sequence having at least 80%, 90%, 95%, 98%, 99% or 100% identity thereto; and
b) a light chain variable region comprising or consisting of SEQ ID NO: 196, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 202, or a sequence having at least 80%, 90%, 95%, 98%, 99% or 100% identity thereto; and
b) a light chain variable region comprising or consisting of SEQ ID NO: 197, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 203, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 203, or a sequence having at least 80%, 90%, 95%, 98%, 99% or 100% identity thereto; and
b) a light chain variable region comprising or consisting of SEQ ID NO: 196, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 203, or a sequence having at least 80%, 90%, 95%, 98%, 99% or 100% identity thereto; and
b) a light chain variable region comprising or consisting of SEQ ID NO: 197, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 204, or a sequence having at least 80%, 90%, 95%, 98%, 99% or 100% identity thereto; and
b) a light chain variable region comprising or consisting of SEQ ID NO: 195, or a sequence having at least 80%, 9%, 95% 98%, 99% or 100% identity thereto.

The antibody or antigen binding fragment thereof may be a humanised antibody comprising or consisting of:
a) a heavy chain variable region comprising or consisting of SEQ ID NO: 204, or a sequence having at least 80%, 90%, 95%, 98%, 99% or 100% identity thereto; and
b) a light chain variable region comprising or consisting of SEQ ID NO: 196, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 204, or a sequence having at least 80%, 90%, 95%, 98%, 99% or 100% identity thereto; and
b) a light chain variable region comprising or consisting of SEQ ID NO: 197, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 205, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 205, or a sequence having at least 80%, 90%, 95%, 98%, 99% or 100% identity thereto; and
b) a light chain variable region comprising or consisting of SEQ ID NO: 196, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 205, or a sequence having at least 80%, 90%, 95%, 98%, 99% or 100% identity thereto; and
b) a light chain variable region comprising or consisting of SEQ ID NO: 197, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 206, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 206, or a sequence having at least 80%, 90%, 95%, 98%, 99% or 100% identity thereto; and
b) a light chain variable region comprising or consisting of SEQ ID NO: 196, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 206, or a sequence having at least 80%, 90%, 95%, 98%, 99% or 100% identity thereto; and
b) a light chain variable region comprising or consisting of SEQ ID NO: 197, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 207, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 207, or a sequence having at least 80%, 90%, 95%, 98%, 99% or 100% identity thereto; and
b) a light chain variable region comprising or consisting of SEQ ID NO: 196, or a sequence having at least 80%, 9%, 95% 98%, 99% or 100% identity thereto.

The antibody or antigen binding fragment thereof may be a humanised antibody comprising or consisting of:
a) a heavy chain variable region comprising or consisting of SEQ ID NO: 207, or a sequence having at least 80%, 90%, 95%, 98%, 99% or 100% identity thereto; and
b) a light chain variable region comprising or consisting of SEQ ID NO: 197, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 208, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 208, or a sequence having at least 80%, 90%, 95%, 98%, 99% or 100% identity thereto; and
b) a light chain variable region comprising or consisting of SEQ ID NO: 196, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 208, or a sequence having at least 80%, 90%, 95%, 98%, 99% or 100% identity thereto; and
b) a light chain variable region comprising or consisting of SEQ ID NO: 197, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 209, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 209, or a sequence having at least 80%, 90%, 95%, 98%, 99% or 100% identity thereto; and
b) a light chain variable region comprising or consisting of SEQ ID NO: 196, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 209, or a sequence having at least 80%, 90%, 95%, 98%, 99% or 100% identity thereto; and
b) a light chain variable region comprising or consisting of SEQ ID NO: 197, or a sequence having at least 80%, 9%, 95% 98%, 99% or 100% identity thereto.

The antibody or antigen binding fragment thereof may be a humanised antibody comprising or consisting of:
a) a heavy chain variable region comprising or consisting of SEQ ID NO: 210, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 210, or a sequence having at least 80%, 90%, 95%, 98%, 99% or 100% identity thereto; and
b) a light chain variable region comprising or consisting of SEQ ID NO: 196, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 210, or a sequence having at least 80%, 90%, 95%, 98%, 99% or 100% identity thereto; and
b) a light chain variable region comprising or consisting of SEQ ID NO: 197, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 211, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 211, or a sequence having at least 80%, 90%, 95%, 98%, 99% or 100% identity thereto; and
b) a light chain variable region comprising or consisting of SEQ ID NO: 196, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 211, or a sequence having at least 80%, 90%, 95%, 98%, 99% or 100% identity thereto; and
b) a light chain variable region comprising or consisting of SEQ ID NO: 197, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 212, or a sequence having at least 80%, 90%, 95%, 98%, 99% or 100% identity thereto; and
b) a light chain variable region comprising or consisting of SEQ ID NO: 195, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 212, or a sequence having at least 80%, 90%, 95%, 98%, 99% or 100% identity thereto; and
b) a light chain variable region comprising or consisting of SEQ ID NO: 196, or a sequence having at least 80%, 90%, 95%, 98%, 99% or 100% identity thereto.

The antibody or antigen binding fragment thereof may be a humanised antibody comprising or consisting of:
a) a heavy chain variable region comprising or consisting of SEQ ID NO: 212, or a sequence having at least 80%, 90%, 95%, 98%, 99% or 100% identity thereto; and
b) a light chain variable region comprising or consisting of SEQ ID NO: 197, or a sequence having at least 80%, 90%, 95%, 98%, 99% or 100% identity thereto.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 266, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 267, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 268, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 269, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 270, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 271, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 272, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 273, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 274, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 275, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 276, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 277, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 279, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 265 and a light chain portion of SEQ ID NO: 280, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 281 and a light chain portion of SEQ ID NO: 282, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 281 and a light chain portion of SEQ ID NO: 283, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 281 and a light chain portion of SEQ ID NO: 284, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 285 and a light chain portion of SEQ ID NO: 282, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 285 and a light chain portion of SEQ ID NO: 283, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 285 and a light chain portion of SEQ ID NO: 284, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 286 and a light chain portion of SEQ ID NO: 282, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 286 and a light chain portion of SEQ ID NO: 283, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 286 and a light chain portion of SEQ ID NO: 284, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 287 and a light chain portion of SEQ ID NO: 282, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 287 and a light chain portion of SEQ ID NO: 283, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 287 and a light chain portion of SEQ ID NO: 284, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 288 and a light chain portion of SEQ ID NO: 282, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 288 and a light chain portion of SEQ ID NO: 283, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 288 and a light chain portion of SEQ ID NO: 284, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 289 and a light chain portion of SEQ ID NO: 282, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 289 and a light chain portion of SEQ ID NO: 283, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 289 and a light chain portion of SEQ ID NO: 284, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 290 and a light chain portion of SEQ ID NO: 282, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 290 and a light chain portion of SEQ ID NO: 283, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 290 and a light chain portion of SEQ ID NO: 284, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 291 and a light chain portion of SEQ ID NO: 282, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 291 and a light chain portion of SEQ ID NO: 283, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 291 and a light chain portion of SEQ ID NO: 284, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 292 and a light chain portion of SEQ ID NO: 282, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 292 and a light chain portion of SEQ ID NO: 283, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 292 and a light chain portion of SEQ ID NO: 284, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 293 and a light chain portion of SEQ ID NO: 282, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 292 and a light chain portion of SEQ ID NO: 283, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 293 and a light chain portion of SEQ ID NO: 284, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 294 and a light chain portion of SEQ ID NO: 282, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 294 and a light chain portion of SEQ ID NO: 283, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 294 and a light chain portion of SEQ ID NO: 284, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 295 and a light chain portion of SEQ ID NO: 282, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 295 and a light chain portion of SEQ ID NO: 283, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 295 and a light chain portion of SEQ ID NO: 284, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 296 and a light chain portion of SEQ ID NO: 282, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 296 and a light chain portion of SEQ ID NO: 283, or sequences having at least 80%, 90%, 95%, 98%, 99% or 100% identity thereto; and
b) an Fc region.

The antibody or antigen binding fragment thereof may be a humanised antibody comprising or consisting of:
a) a Fab region comprising or consisting of a heavy chain portion of SEQ ID NO: 296 and a light chain portion of SEQ ID NO: 284, or sequences having at least 80%, 90%, 95%, 98%, 99% or 100% identity thereto; and
b) an Fc region.

The antibodies may comprise a human Fc region. More particular the antibody or antigen-binding fragment thereof may be a full-length antibody. More particularly the antibody may be of the IgG isotype. More particularly the antibody may be an IgG1 or IgG4. Most preferably, the Fc region comprises or consists of SEQ ID NO: 297.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 266 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 267 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 268 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 269 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and 270 b) a light chain comprising or consisting of SEQ ID NO: YY or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 271 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 272 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 273 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 274 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 275 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 276 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 277 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 278 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 279 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 300, or a sequence having at least 80%, 90%, 95%, 98%, 99% or 100% identity thereto; and
b) a light chain comprising or consisting of SEQ ID NO: 280 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 301, or a sequence having at least 80%, 90%, 95%, 98%, 99% or 100% identity thereto; and
b) a light chain comprising or consisting of SEQ ID NO: 282 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 301, or a sequence having at least 80%, 90%, 95%, 98%, 99% or 100% identity thereto; and
b) a light chain comprising or consisting of SEQ ID NO: 283 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 301, or a sequence having at least 80%, 90%, 95%, 98%, 99% or 100% identity thereto; and
b) a light chain comprising or consisting of SEQ ID NO: 284 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 302, or a sequence having at least 80%, 90%, 95%, 98%, 99% or 100% identity thereto; and
b) a light chain comprising or consisting of SEQ ID NO: 282 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 302, or a sequence having at least 80%, 90%, 95%, 98%, 99% or 100% identity thereto; and
b) a light chain comprising or consisting of SEQ ID NO: 283 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 302, or a sequence having at least 80%, 90%, 95%, 98%, 99% or 100% identity thereto; and
b) a light chain comprising or consisting of SEQ ID NO: 284 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 303, or a sequence having at least 80%, 90%, 95%, 98%, 99% or 100% identity thereto; and
b) a light chain comprising or consisting of SEQ ID NO: 282 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 303, or a sequence having at least 80%, 90%, 95%, 98%, 99% or 100% identity thereto; and
b) a light chain comprising or consisting of SEQ ID NO: 283 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 303, or a sequence having at least 80%, 90%, 95%, 98%, 99% or 100% identity thereto; and
b) a light chain comprising or consisting of SEQ ID NO: 284 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 304, or a sequence having at least 80%, 90%, 95%, 98%, 99% or 100% identity thereto; and
b) a light chain comprising or consisting of SEQ ID NO: 282 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 304, or a sequence having at least 80%, 90%, 95%, 98%, 99% or 100% identity thereto; and
b) a light chain comprising or consisting of SEQ ID NO: 283 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 304, or a sequence having at least 80%, 90%, 95%, 98%, 99% or 100% identity thereto; and
b) a light chain comprising or consisting of SEQ ID NO: 284 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 305, or a sequence having at least 80%, 90%, 95%, 98%, 99% or 100% identity thereto; and
b) a light chain comprising or consisting of SEQ ID NO: 282 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 305, or a sequence having at least 80%, 90%, 95%, 98%, 99% or 100% identity thereto; and
b) a light chain comprising or consisting of SEQ ID NO: 283 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 305, or a sequence having at least 80%, 90%, 95%, 98%, 99% or 100% identity thereto; and
b) a light chain comprising or consisting of SEQ ID NO: 284 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 306, or a sequence having at least 80%, 90%, 95%, 98%, 99% or 100% identity thereto; and
b) a light chain comprising or consisting of SEQ ID NO: 282 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 306, or a sequence having at least 80%, 90%, 95%, 98%, 99% or 100% identity thereto; and
b) a light chain comprising or consisting of SEQ ID NO: 283 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 306, or a sequence having at least 80%, 90%, 95%, 98%, 99% or 100% identity thereto; and
b) a light chain comprising or consisting of SEQ ID NO: 284 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 307, or a sequence having at least 80%, 90%, 95%, 98%, 99% or 100% identity thereto; and
b) a light chain comprising or consisting of SEQ ID NO: 282 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 307, or a sequence having at least 80%, 90%, 95%, 98%, 99% or 100% identity thereto; and
b) a light chain comprising or consisting of SEQ ID NO: 283 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 307, or a sequence having at least 80%, 90%, 95%, 98%, 99% or 100% identity thereto; and
b) a light chain comprising or consisting of SEQ ID NO: 284 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 308, or a sequence having at least 80%, 90%, 95%, 98%, 99% or 100% identity thereto; and
b) a light chain comprising or consisting of SEQ ID NO: 282 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 308, or a sequence having at least 80%, 90%, 95%, 98%, 99% or 100% identity thereto; and
b) a light chain comprising or consisting of SEQ ID NO: 283 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 308, or a sequence having at least 80%, 90%, 95%, 98%, 99% or 100% identity thereto; and
b) a light chain comprising or consisting of SEQ ID NO: 284 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 309, or a sequence having at least 80%, 90%, 95%, 98%, 99% or 100% identity thereto; and
b) a light chain comprising or consisting of SEQ ID NO: 282 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 309, or a sequence having at least 80%, 90%, 95%, 98%, 99% or 100% identity thereto; and
b) a light chain comprising or consisting of SEQ ID NO: 283 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 309, or a sequence having at least 80%, 90%, 95%, 98%, 99% or 100% identity thereto; and
b) a light chain comprising or consisting of SEQ ID NO: 284 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 310, or a sequence having at least 80%, 90%, 95%, 98%, 99% or 100% identity thereto; and
b) a light chain comprising or consisting of SEQ ID NO: 282 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 310, or a sequence having at least 80%, 90%, 95%, 98%, 99% or 100% identity thereto; and
b) a light chain comprising or consisting of SEQ ID NO: 283 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 310, or a sequence having at least 80%, 90%, 95%, 98%, 99% or 100% identity thereto; and
b) a light chain comprising or consisting of SEQ ID NO: 284 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 311, or a sequence having at least 80%, 90%, 95%, 98%, 99% or 100% identity thereto; and
b) a light chain comprising or consisting of SEQ ID NO: 282 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 311, or a sequence having at least 80%, 90%, 95%, 98%, 99% or 100% identity thereto; and
b) a light chain comprising or consisting of SEQ ID NO: 283 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 311, or a sequence having at least 80%, 90%, 95%, 98%, 99% or 100% identity thereto; and
b) a light chain comprising or consisting of SEQ ID NO: 284 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 312, or a sequence having at least 80%, 90%, 95%, 98%, 99% or 100% identity thereto; and
b) a light chain comprising or consisting of SEQ ID NO: 282 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 312, or a sequence having at least 80%, 90%, 95%, 98%, 99% or 100% identity thereto; and
b) a light chain comprising or consisting of SEQ ID NO: 283 or a sequence having at least 80%, 90%, 95%, 98%, 99% or 100% identity thereto.

The antibody or antigen binding fragment thereof may be a humanised antibody comprising or consisting of:
a) a heavy chain comprising or consisting of SEQ ID NO: 312, or a sequence having at least 80%, 90%, 95%, 98%, 99% or 100% identity thereto; and
b) a light chain comprising or consisting of SEQ ID NO: 284 or a sequence having at least 80%, 90%, 95%, 98%, 99% or 100% identity thereto.

The constant region domains of the antibody, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used. It will also be known to the person skilled in the art that antibodies may undergo a variety of posttranslational modifications. The type and extent of these modifications often depends on the host cell line used to express the antibody as well as the cell culture conditions. Such modifications may include variations in glycosylation, methionine oxidation, diketopiperazine formation, aspartate isomerization and asparagine deamidation.

In some embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

The antibody or antigen binding fragment thereof may comprise or consist of:
An ScFv comprising or consisting of SEQ ID NO: 101, or a sequence having at least 80%, 90%, 95%, 98%, 99% or 100% identity thereto.

The antibody or antigen binding fragment thereof may comprise or consist of:
An ScFv comprising or consisting of SEQ ID NO: 102, or a sequence having at least 80%, 90%, 95%, 98%, 99% or 100% identity thereto.

The antibody or antigen binding fragment thereof may comprise or consist of:
An ScFv comprising or consisting of SEQ ID NO: 103, or a sequence having at least 80%, 90%, 95%, 98%, 99% or 100% identity thereto.

The antibody or antigen binding fragment thereof may comprise or consist of:
An ScFv comprising or consisting of SEQ ID NO: 104, or a sequence having at least 80%, 90%, 95%, 98%, 99% or 100% identity thereto.

The antibody or antigen binding fragment thereof may comprise or consist of:
An ScFv comprising or consisting of SEQ ID NO: 105, or a sequence having at least 80%, 90%, 95%, 98%, 99% or 100% identity thereto.

The antibody or antigen binding fragment thereof may comprise or consist of:
An ScFv comprising or consisting of SEQ ID NO: 106, or a sequence having at least 80%, 90%, 95%, 98%, 99% or 100% identity thereto.

In another aspect, there is provided an antibody or antigen binding fragment thereof which binds to an epitope on CD1a comprising or consisting of residues Arg 83, Tyr 84, His 86, Glu 87, Gln 89, Phe 90, Glu 91, Asn 139, Met 140, Lys 142, His 143, Lys 146, Val 147 and Gln 150 of CD1a, and wherein the residue numbering is according to SEQ ID NO: 252.

In another aspect, there is provided an antibody or antigen binding fragment thereof which binds to an epitope on CD1a comprising or consisting of residues Glu 62, Glu 65, Leu 66, Thr 68, Leu 69, Ile 72, Asn 151, His 153, Glu 154, Ile 157, Asn 160, Asp 164, Thr165 and Arg 168 of CD1a, and wherein the residue numbering is according to SEQ ID NO: 252.

In another aspect, there is provided an antibody or antigen binding fragment thereof which binds to an epitope on CD1a comprising or consisting of residues Glu 79, Arg 82, Arg 83, His 86, Glu 87, Gln 89, Phe 90, Glu 91, Tyr 92, Val 147 and Asn 150 of CD1a, and wherein the residue numbering is according to SEQ ID NO: 252.

More specifically an antibody or antigen-binding fragment thereof binds to one or more above-disclosed residues, more particular to 5 or more residues.

The epitope can be identified by any suitable epitope mapping method known in the art in combination with any one of the antibodies provided herein. Examples of such methods include screening peptides of varying lengths derived from full length target protein for binding to the antibody or fragment thereof of the present disclosure and identify the smallest fragment that can specifically bind to the antibody containing the sequence of the epitope recognized by the antibody. Target peptides may be produced synthetically. Peptides that bind the antibody can be identified by, for example, mass spectrometric analysis. In another example, NMR spectroscopy or X-ray crystallography can be used to identify the epitope bound by an antibody of the present invention. Typically, when the epitope determination is performed by X-ray crystallography, amino acid residues of the antigen within 4 Å from CDRs are considered to be amino acid residues part of the epitope. Once identified, the epitope may serve for preparing fragments which bind an antibody of the present invention and, if required, used as an immunogen to obtain additional antibodies which bind the same epitope.

The epitope may be determined using a number of techniques available and known to the skilled person, such as X-ray crystallography.

One can easily determine whether an antibody binds to the same epitope as, or competes for binding with, a reference antibody by using routine methods known in the art. For example, to determine if a test antibody binds to the same epitope as a reference antibody, the reference antibody is allowed to bind to a protein or peptide under saturating conditions. Next, the ability of a test antibody to bind to the protein or peptide is assessed. If the test antibody is able to bind to the protein or peptide following saturation binding with the reference antibody, it can be concluded that the test antibody binds to a different epitope than the reference antibody. On the other hand, if the test antibody is not able to bind to protein or peptide following saturation binding with the reference antibody, then the test antibody may bind to the same epitope as the epitope bound by the reference antibody of the invention.

In an embodiment, there is provided an antibody or antigen-binding fragment thereof which competes with an antibody or antigen-binding fragment thereof for binding to CD1a.

The term “antibody or antigen-binding fragment thereof which competes with” a reference antibody or antigen-binding fragment thereof refers to an antibody or antigen-binding fragment thereof that blocks binding of the reference antibody to its antigen in a competition assay by 50% or more, and conversely, the reference antibody blocks binding of the antibody to its antigen in a competition assay by 50% or more.

To determine if an antibody competes for binding with a reference antibody, the above-described binding methodology is performed in two orientations. In a first orientation, the reference antibody is allowed to bind to a protein/peptide under saturating conditions followed by assessment of binding of the test antibody to the protein/peptide molecule. In a second orientation, the test antibody is allowed to bind to the protein/peptide under saturating conditions followed by assessment of binding of the reference antibody to the protein/peptide. If, in both orientations, only the first (saturating) antibody is capable of binding to the protein/peptide, then it is concluded that the test antibody and the reference antibody compete for binding to the protein/peptide. As will be appreciated by the skilled person, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50%, 75%, 90% or even 99% as measured in a competitive binding assay (see, e.g., Junghans et al., Cancer Res, 1990:50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

An antibody or antigen binding fragment thereof of the invention may be isolated.

In any aspect, “an antibody or antigen binding fragment thereof” may refer to one more, such as two of the recited antibodies or antigen binding fragments thereof. For example, in any aspect, two antibodies or antigen binding fragments thereof may be envisioned, for example which bind to CD1a at different binding sites.

For example, in any therapeutic application disclosed herein, and/or in any method of monitoring disclosed herein, any combination of antibodies or antigen-binding fragments may be utilised. For example, Ab 116 and 16 may be used in combination. Alternatively, Ab 16 and 110 may be used in combination.

In another embodiment, Ab 116 may be used in any therapeutic application disclosed herein, and Ab 16 may be used in monitoring of the same subject. Alternatively, Ab 16 may be used in any therapeutic application disclosed herein, and Ab 116 may be used in monitoring of the same subject.

Any reference to an antibody or antigen binding fragment thereof by its internal designation, for example Ab 116 or Ab 16, also includes to any chimeric or humanised version disclosed herein.

The term “antibody” as referred to herein refers to a glycoprotein comprising at least two heavy (H) chains and two light (L) chains inter-connected by disulfide bonds. Each heavy chain is comprised of a heavy chain variable region (V_{H}) and a heavy chain constant region. Each light chain is comprised of a light chain variable region (V_{L}) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g effector cells) and the first component (Clq) of the classical complement system.

The term “antigen-binding fragment thereof” of an antibody refers to one or more fragments of an antibody that retain the ability to selectively bind to an antigen. Antigen-binding fragments thereof may be, but are not limited to Fab, modified Fab, Fab′, modified Fab′, F(ab′)₂, Fv, single domain antibodies (e.g. VH or VL or VHH), scFv, bi, tri or tetra-valent antibodies, Bis-scFv, diabodies, triabodies, tetrabodies and epitope-binding fragments of any of the above (Holliger and Hudson, 2005, Nature Biotech. 23(9): 1126-1136; Adair and Lawson, 2005, Drug Design Reviews—Online 2(3), 209-217). The methods for creating and manufacturing these antigen-binding fragments are well known in the art (see for example Verma et al., 1998, Journal of Immunological Methods, 216, 165-181).

The term “chimeric” antibody refers to an antibody in which the variable domain (or at least a portion thereof) of the heavy and/or light chain is derived from a particular source or species, while the remainder of the heavy and/or light chain (i.e. the constant domains) is derived from a different source or species. (Morrison; PNAS 81, 6851 (1984)). Chimeric antibodies can for instance comprise non-human variable domains and human constant domains. Chimeric antibodies are typically produced using recombinant DNA methods. A subcategory of “chimeric antibodies” is “humanized antibodies”.

The term “humanized” antibody or antigen-binding fragment thereof refers to an antibody or antigen-binding fragment thereof comprising amino acid residues from non-human HVRs and amino acid residues from human FRs. Typically the heavy and/or light chain contains one or more CDRs (including, if desired, one or more modified CDRs) from a donor antibody (e.g. a non-human antibody such as a murine or rabbit monoclonal antibody) and is grafted into a heavy and/or light chain variable region framework of an acceptor antibody (a human antibody)(see e.g. Vaughan et al, Nature Biotechnology, 16, 535-539, 1998). The advantage of such humanized antibodies is to reduce immunogenicity to humans, while retaining the specificity and affinity of the parental non-human antibody. Rather than the entire CDR being transferred, only one or more of the specificity determining residues from any one of the CDRs described herein above can be transferred to the human antibody framework (see e.g., Kashmiri et al., 2005, Methods, 36, 25-34). A “humanized” antibody refers to a chimeric antibody comprising amino acid residues from non-human HVRs and amino acid residues from human FRs. A “humanized form” of an antibody, e.g., a non-human antibody, refers to an antibody that has undergone humanization.

The term “Framework” or “FR” refers to variable domain residues other than hypervariable region residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The antibody or antigen binding fragment thereof may be a monoclonal antibody, full-length antibody, bispecific antibody, multi-specific antibody, ScFv or other single chain or modified format, Fab, (Fab′)2, Fv, dAb, Fd, nanobody, camelid antibody or a diabody. Preferably, the antibody or antigen binding fragment thereof is a monoclonal antibody.

A bispecific antibody may comprise a CD1a targeting moiety which comprises an antibody or antigen binding fragment thereof of the invention, and a T-cell engaging moiety. The T-cell engaging moiety may be a CD3-targeting moiety, such as antibody UCHT1 (SEQ ID NO: 298). The CD1a targeting moiety may comprise a light chain variable region of SEQ ID NO: 314, and a heavy chain variable region of SEQ ID NO: 314, or sequences having at least 80%, 90%, 95%, 98%, 99% or 100%, identity thereto. The CD1a targeting moiety may comprise a light chain of SEQ ID NO: 210, and a heavy chain of SEQ ID NO: 299, or sequences having at least 80%, 90%, 95%, 98%, 99% or 100%, identity thereto.

The term “full length antibody” is used herein to refer to an antibody having a structure substantially similar to a native antibody structure or having heavy chains that contain an Fc region as defined herein. Each light chain is comprised of a light chain variable region (abbreviated herein as VL) and a light chain constant region (CL). Each heavy chain is comprised of a heavy variable region (abbreviated herein as VH) and a heavy chain constant region (CH) constituted of three constant domains CH1, CH2 and CH3, or four constant domains CH1, CH2, CH3 and CH4, depending on the Ig class. The constant regions of the antibodies may mediate the binding of the immunoglobulin to host tissues or factors, including various cells of the immune system (e.g., effector cells) and the first component (Clq) of the classical complement system. IgG antibodies are examples of full-length antibodies, such as IgG1 or IgG4 antibodies.

The inventors have targeted CD1a and its potential role in inflammatory skin and mucosal diseases and disorders, or associated systemic diseases or disorders, or inflammatory drug reactions which manifest systemically, by generating effective monoclonal antibodies. As CD1a is highly expressed in the skin and mucosae, use of such antibodies provides an opportunity to selectively treat inflammatory skin and mucosal diseases and disorders whilst minimising off target effects. CD1a is not expressed by mice but is expressed by other mammals. Human CD1a (UniProtKB/Swiss-Prot: P06126-CD1A_HUMAN) is expressed from a dominant allele worldwide, with a variant that is present in some Chinese ethnic groups (18). Targeting CD1a antigen presentation also intercepts the inflammatory pathway upstream of other cytokine-directed antibody therapies such as anti-IL17 therapies, or other immune therapies, and therefore provides a powerful means to modulate proinflammatory disorders early in the immune cascade. Furthermore, utilising the specificity of CD1a to the skin may provide the means to direct additional therapies to the skin, for example by use of bi-specific, or multi-specific or conjugate antibody technology, to specifically target small molecule, drug, nucleic acid, peptide, antibody, or cell conjugate therapies. Further still, as CD1a is relatively non-polymorphic, the invention provides universal potential in the prevention and/or treatment of inflammatory skin and mucosal diseases such as atopic dermatitis and psoriasis, where the frequency of CD1a-expressing dendritic cell subsets is increased, and migratory patterns of LCs are altered (13-15), or CD1a-expressing malignancies.

By modifying the number and function of CD1a-expressing cells, the antibodies will have effects beyond lipid reactivity and influence all roles of CD1a-expressing cells, including antigen presentation to peptide-specific T cells and innate pathways (for example neutrophils). The antibodies of the invention are able to reduce Langerhans cells despite their murine IgG1 nature. Such reduction offers a means of controlling broad inflammatory pathways in the absence of complement/ADCC-associated inflammation, which may offer therapeutic benefit. This is shown in the imiquimod model described herein, where antibodies according the invention for example reduce inflammation including to levels significantly below the wild-type mouse, demonstrating a profound anti-inflammatory effect on pathways beyond CD1a-expressing cells, including innate pathways such as neutrophils and eosinophils. The antibodies of the invention also inhibit the production of diverse cytokines including IFN-gamma and IL-22 which are relevant to a broad range of clinical diseases.

In another aspect, the invention provides a nucleic acid encoding an antibody or antigen binding fragment thereof of the invention. Such nucleic acids may be provided by any of SEQ ID Nos: 51-90. The skilled person will understand that due to codon redundancy, a number of DNA sequences may be used to encode an antibody or antigen binding fragment thereof of the invention. Alternatively, codon optimization of the nucleotide sequence can be used to improve the efficiency of translation in expression systems for the production of an antibody or antigen binding fragment thereof of the invention.

In another aspect, the invention provides a vector comprising a nucleic acid of the invention. Suitable vectors can be chosen or constructed, containing appropriate regulatory sequences, including promoter sequences, terminator sequences, polyadenylation sequences, enhancer sequences, marker genes and other sequences as appropriate. Vectors may be for example plasmids or viral. For further details see, for example, (Sambrook, J., E. F. Fritsch, and T. Maniatis. (1989), Molecular cloning: a laboratory manual, 2nd ed. Cold Spring Harbor Laboratory, Cold Spring Harbor, New York). Many known techniques and protocols for manipulation of nucleic acid, for example in preparation of nucleic acid constructs, mutagenesis, sequencing, introduction of DNA into cells and gene expression, and analysis of proteins, are described in detail in (Ausubel et al., Current protocols in molecular biology. New York: Greene Publishing Association; Wiley-Interscience, 1992). The vector may be an expression vector. The vector or expression vector may be a plasmid.

A nucleic acid molecule or vector of the invention may be expressed using any suitable expression system, for example in a suitable host cell or in a cell-free system.

In another aspect, the invention provides a host cell comprising an antibody or antigen binding fragment thereof, nucleic acid, and/or vector of the invention. The host cell may be selected from bacterial host cells (prokaryotic systems) such as E. coli, or eukaryotic cells such as those of yeasts, fungi, insect cells or mammalian cells. Preferably a host cell of the invention is capable of producing the antibody or antigen binding fragment thereof of the invention. The produced antibody or antigen binding fragment thereof may be enriched by means of selection and/or isolation.

An antibody or antigen binding fragment thereof of the invention may also be produced by chemical synthesis. The obtained antibody or antigen binding fragment thereof may be enriched by means of selection and/or isolation.

According to a further aspect, the invention provides a pharmaceutical composition comprising an antibody or antigen binding fragment thereof, nucleic acid, vector and/or host cell of the invention, optionally together with one or more pharmaceutically acceptable excipients or diluents.

Antibodies or antigen binding fragments thereof, nucleic acids, vectors or host cells of the invention can be formulated into pharmaceutical compositions using established methods of preparation (Gennaro, A. L. and Gennaro, A. R. (2000) Remington: The Science and Practice of Pharmacy, 20th Ed., Lippincott Williams & Wilkins, Philadelphia, PA). To prepare the pharmaceutical compositions, pharmaceutically inert inorganic or organic excipients can be used. To prepare for example pills, powders, gelatin capsules or suppositories, lactose, talc, stearic acid and its salts, fats, waxes, solid or liquid polyols, natural and hardened oils are examples of pharmaceutically acceptable excipients which can be used. Suitable excipients for the production of solutions, suspensions, emulsions, aerosol mixtures or powders for reconstitution into solutions or aerosol mixtures prior to use include water, alcohols, glycerol, polyols, and suitable mixtures thereof as well as vegetable oils.

A pharmaceutical composition of the invention may be administered via any parenteral or non-parenteral (enteral) route that is therapeutically effective. Parenteral application methods include, for example, intracutaneous, subcutaneous, intramuscular, intratracheal, intranasal, intravitreal or intravenous injection and infusion techniques, e.g. in the form of injection solutions, infusion solutions or mixtures, as well as aerosol installation and inhalation, e.g. in the form of aerosol mixtures, sprays or powders. A pharmaceutical composition of the invention can be administered systemically or topically in formulations containing conventional non-toxic pharmaceutically acceptable excipients or carriers, additives and vehicles as desired. A combination of intravenous and subcutaneous infusion and/or injection might be most convenient in case of compounds with a relatively short or long serum half-life or needing rapid onset of action. Preferably, the pharmaceutical composition is administered subcutaneously or intravenously. The pharmaceutical composition may be an aqueous solution, an oil-in water emulsion or a water-in-oil emulsion.

For intravenous injection, or injection at the site of affliction, or other site of administration, the active ingredient will be in the form of a parenterally acceptable aqueous solution which is pyrogen-free and has suitable pH, isotonicity and stability. Those of relevant skill in the art are well able to prepare suitable solutions using for example, isotonic vehicles such as Sodium Chloride Injection, Ringer's Injection, Lactated Ringer's Injection. Preservatives, stabilisers, buffers, antioxidants and/or other additives may be included, as required.

The compositions are preferably administered to an individual in a “therapeutically effective amount”, this being sufficient to show benefit to the individual. The optimal dosage will depend on the biodistribution of the antibody or antigen binding fragment thereof, the mode of administration, the severity of the disease/disorder being treated as well as the medical condition of the patient. If desired, the antibody or antigen binding fragment thereof may be given in a sustained release formulation, for example liposomal dispersions or hydrogel-based polymer microspheres, like PolyActive™ or OctoDEX™ (cf. Bos et al., Business Briefing: Pharmatech 2003: 1-6). Other sustained release formulations available are for example PLGA based polymers (PR pharmaceuticals), PLA-PEG based hydrogels (Medincell) and PEA based polymers (Medivas). Prescription of treatment, e.g., decisions on dosage etc, is within the responsibility of a medical practitioner, and typically takes account of the disorder to be treated, the condition of the individual patient, the site of delivery, the method of administration and other factors known to practitioners.

The pharmaceutical composition may also contain additives, such as, for example, fillers, binders, wetting agents, glidants, stabilizers, preservatives, emulsifiers, and furthermore solvents or solubilizers or agents for achieving a depot effect. The latter is that fusion proteins may be incorporated into slow or sustained release or targeted delivery systems, such as liposomes and microcapsules.

In another aspect, an antibody or antigen binding fragment thereof, nucleic acid, vector, host cell or pharmaceutical composition of the invention may be for use in the treatment or prevention of one or more disease or disorder in a subject.

In an aspect, there is provided a method of treating or preventing one or more disease or disorder in a subject, comprising administering to the subject an effective amount of an antibody or antigen binding fragment thereof, nucleic acid, vector, host cell or composition of the invention.

In an aspect, there is provided the use of an antibody or antigen binding fragment thereof, nucleic acid, vector, host cell or pharmaceutical composition of the invention in the manufacture of a medicament for the treatment or prevention of one or more diseases or disorders in a subject.

In any aspect, the subject may be a mammal. The mammal may express a CD1a orthologue. Preferably, the subject is a human.

The one or more disease or disorder may be one or more inflammatory skin or mucosal disorder, or disease or one or more associated systemic disease or disorder, or one or more inflammatory drug reaction which manifests systemically, or a CD1a-expressing malignancy.

An inflammatory skin or mucosal disease or disorder may be selected from:
a) a predominantly neutrophilic skin disease, such as acne, generalized pustular psoriasis, plaque psoriasis, guttate psoriasis, palmoplantar pustulosis, SAPHO syndrome, acute febrile neutrophilic dermatosis (Sweet syndrome), histiocytoid neutrophilic dermatitis, neutrophilic dermatosis of the dorsal hands, pyoderma gangrenosum, neutrophilic eccrine hidradenitis, hidradenitis suppurativa, erythema elevatum diutinum, Behcet disease, bowel-associated dermatitis-arthritis syndrome, other infection-associated inflammation, neutrophilic urticarial dermatosis, palisading neutrophilic granulomatous dermatitis, erythema gyratum repens, neutrophilic annular erythema, acute generalised exanthematous pustulosis (AGEP), vasculitis, and others;
b) an autoimmune disorder, such as connective tissue disease (eg lupus, dermatomyositis, scleroderma/systemic sclerosis, Churg Strauss syndrome), panniculitis, vasculitides, autoimmune blistering conditions (eg bullous pemphigoid, pemphigus, linear IgA disease), dermatitis herpetiformis, coeliac disease, some auto-inflammatory disease, vitiligo, alopecia areata, alopecia universalis, alopecia totalis, panniculitis, lichen planus, erythema multiforme, lichen sclerosis, other lichenoid and erythema multiforme-like diseases, vesiculation psoriatic arthritis, rheumatoid arthritis, inflammatory bowel disease, multiple sclerosis, Guillain-Barre syndrome, thyroiditis, transverse myelitis, neurodegeneration and others;
c) mast cell disorders and eosinophilic disorders, such as Muckle Wells syndrome, eosinophilia and systemic symptoms syndrome, urticaria, angioedema, keratoconjunctivitis, food allergy, other allergy or atopy including atopic dermatitis, rhinitis, conjunctivitis, asthma, eosinophilic oesophagitis and other eosinophilic mucosal diseases, contact dermatitis, chronic obstructive airways disease and others.
d) adverse drug reactions which manifest as an inflammatory skin or mucosal disease or disorder, such as Stevens Johnsons syndrome, toxic epidermal necrolysis, drug reaction with eosinophilia and systemic symptoms syndrome (DRESS) and acute generalised exanthematous pustulosis (AGEP), erythema multiforme, bullous, fixed drug eruption, checkpoint inhibitor-associated skin and other inflammation and others.
e) Graft vs host disease
f) Pruritus and pruritic conditions including nodular prurigo.

A CD1a-expressing malignancy as referred to herein may be any malignancy where CD1a expression can be detected. Such malignancies may include Langerhans cell histiocytosis, Langerhans cell sarcoma, subsets of T cell lymphomas, subsets of thymomas or rarely-occurring instances of other malignancies, such as subsets of mastocytosis. Preferably, the CD1a-expressing malignancy is subsets of T cell lymphomas.

Preferably the one or more disease or disorder comprises or consists of psoriasis, dermatitis, lupus erythematosus, neutrophilic dermatoses, an associated systemic disease or disorder, and/or or an inflammatory drug reaction which manifests systemically, or a CD1a-expressing malignancy.

An associated systemic disease or disorder as used herein may refer to any non-cutaneous site involvement that may be associated with an inflammatory skin or mucosal disease or disorder as defined herein. This may include non-cutaneous lupus erythematosus.

An inflammatory drug reaction which manifests systemically, may be at a non-cutaneous site such as the spleen. An associated systemic disease or disorder, or inflammatory drug reaction which manifests systemically, may be as a result of an inflammatory response. The inflammatory response may be for example to a drug such as Aldara (5% imiquimod cream). The inflammatory response may result in increased numbers or activity of CD4 T-cells, CD8 T-cells, neutrophils or eosinophils, and/or increased levels of IL-23, IL-12, IL-10 and/or MCP-1, and/or decreased IL-10 and/or IL-27.

Furthermore, an antibody or antigen binding fragment thereof, nucleic acid, vector, host cell or pharmaceutical composition of the invention may be administered alone or in combination with one or more other therapeutic agent, either simultaneously, sequentially or separately, dependent upon the condition to be treated. The one or more other therapeutic agent may be selected from the group comprising cytotoxic agents, immune activation agents such as checkpoint inhibitors or TLR agonists, anti-inflammatory agents such as steroids, CAR-T cells such as regulatory or cytolytic CAR-T cells, or other cells expressing or presenting one or more antibody or antigen binding fragment of the invention.

In another aspect, there is provided a method of monitoring treatment efficacy or disease status in a subject diagnosed with a CD1a-expressing malignancy, comprising:
i. providing a biological sample obtained from the subject;
ii. determining the level of binding of one or more antibodies or antigen binding fragments of the invention to CD1a-expressing cells in the sample obtained from the subject before treatment, or at intervals between treatments, or at time intervals in the absence of treatment;
iii. determining that the treatment is effective, or that the disease status is improving, if the tumour volume, or level of binding of one or more antibodies or antigen binding fragments of the invention to CD1a-expressing cells, is reduced after treatment or between treatment intervals or at time intervals in the absence of treatment.

A biological sample as referred to herein may be a blood or serum sample, tissue biopsy, cerebrospinal fluid, saliva, or urine sample. Preferably, the biological sample may be a blood or serum sample.

The level of binding of one or more antibodies or antigen binding fragments of the invention to CD1a-expressing cells in the sample may be determined using any method known to the skilled person. One such method is for example using flow cytometry or any other technique utilising a detectable label, to be able to determine the number of CD1a expressing cells in the sample.

Tumour volume may be determined by any suitable technique known to the skilled person.

The reduction in tumour volume or level of binding of one or more antibodies or antigen binding fragments of the invention to CD1a-expressing cells may be by 10% or more, such as 25% or more, 50% or more, 75% or more, or 90% or more.

The treatment intervals or time intervals in the absence of treatment may be two weeks or more, such as four weeks or more, 8 weeks or more, 12 weeks or more, six months or more, or 12 months or more.

In another aspect, there is provided a method of diagnosing a subject with an inflammatory skin and mucosal disease or disorder, or associated systemic disease or disorder, or inflammatory drug reaction which manifests systemically, or CD1a-expressing malignancy, comprising:
i. providing a biological sample obtained from the subject;
ii. using one or more antibody or antigen-binding fragment thereof of the invention to determine the level of expression of CD1a in the sample obtained from the subject;
iii. comparing the level of expression of CD1a in the sample obtained from the subject with the level of expression of CD1a in a positive or negative reference sample;
iv. determining that the subject has an inflammatory skin and mucosal disease or disorder, or associated systemic disease or disorder, or inflammatory drug reaction which manifests systemically, or CD1a-expressing malignancy, if the level of expression of CD1a in the sample obtained from the subject is higher than the level of expression of CD1a in the negative reference sample, or equal to or higher than the level of expression of CD1a positive reference sample.

Alternatively, in step iv, the subject may be determined to not have has an inflammatory skin and mucosal disease or disorder, or associated systemic disease or disorder, or inflammatory drug reaction which manifests systemically, or CD1a-expressing malignancy, if the level of expression of CD1a in the sample obtained from the subject is equal to or lower than the level of expression of CD1a in the negative reference sample, or lower than the level of expression of CD1a the positive reference sample.

A negative reference sample may refer to a biological sample taken from a healthy subject, known not to have an inflammatory skin and mucosal disease or disorder, or associated systemic disease or disorder, or inflammatory drug reaction which manifests systemically, or CD1a-expressing malignancy. A positive reference sample may refer to a biological sample taken from a subject already diagnosed with an inflammatory skin and mucosal disease or disorder, or associated systemic disease or disorder, or inflammatory drug reaction which manifests systemically, or CD1a-expressing malignancy.

The level of expression of CD1a in the method of diagnosing may refer to the level of CD1a molecules expressed on a given cell or cells in a population, or the percentage of cells in a population or sample which are determined to express CD1a.

Techniques for the production of antibodies and antigen binding fragments thereof are well known in the art. The term “antibody” also includes immunoglobulins (Ig's) of different classes (i.e. IgA, IgG, IgM, IgD and IgE) and subclasses (such as IgG1, IgG2 etc.). Illustrative examples of an antibodies or antigen binding fragments thereof include Fab fragments, F(ab′)2, Fv fragments, single-chain Fv fragments (scFv), diabodies, domain antibodies or bispecific antibodies (Holt L J et al., Trends Biotechnol. 21(11), 2003, 484-490). Examples also include a dAB fragment which consists of a single CH domain or VL domain which alone is capable of binding an antigen. An antibody or antigen binding fragment thereof may be chimeric, a nanobody, single chain and/or humanized. The antibody or antigen binding fragment thereof may be a human IgG1 isotype or a human IgG4 isotype or other natural or modified isotype. Antibodies may be monoclonal (mAb) or polyclonal.

The antibody or antigen binding fragment thereof may be modified to change in vivo stability and/or half-life. The modification for example may be PEGylation.

The antibody or antigen binding fragment thereof may be an antibody-like molecule which includes the use of CDRs separately or in combination in synthetic molecules such as SMIPs and small antibody mimetics.

The percent identity of two amino acid sequences or of two nucleic acid sequences is generally determined by aligning the sequences for optimal comparison purposes (e.g., gaps can be introduced in the first sequence for best alignment with the second sequence) and comparing the amino acid residues or nucleotides at corresponding positions. The “best alignment” is an alignment of two sequences that results in the highest percent identity. The percent identity is determined by comparing the number of identical amino acid residues or nucleotides within the sequences (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul, 1990, PNAS, 87(6):2264-8, modified as in Karlin and Altschul, 1993, PNAS, 90(12):5873-5877 The NBLAST and XBLAST programs of Altschul et al., 1990, J. Mol. Biol., 215:403-10 have incorporated such an algorithm. BLAST nucleotide searches can be performed with the NBLAST program, score=100, word length=12 to obtain nucleotide sequences homologous to a nucleic acid molecules of the invention. BLAST protein searches can be performed with the XBLAST program, score=50, word length=3 to obtain amino acid sequences homologous to a protein molecules of the invention. To obtain gapped alignments for comparison purposes, Gapped BLAST can be utilized as described in Altschul et al. (1997). Alternatively, PSI-Blast can be used to perform an iterated search that detects distant relationships between molecules (Id.). When utilizing BLAST, GappedBLAST, and PSI-Blast programs, the default parameters of the respective programs (e.g., XBLAST and NBLAST) can be used. See http://www.ncbi.nlm.nih.gov. Another example of a mathematical algorithm utilized for the comparison of sequences is the algorithm of Myers and Miller. The ALIGN program (version 2.0) which is part of the GCG sequence alignment software package has incorporated such an algorithm. Other algorithms for sequence analysis known in the art include ADVANCE and ADAM as described in Torellis and Robotti (1994); and FASTA described in Pearson and Lipman (1988). Within FASTA, ktup is a control option that sets the sensitivity and speed of the search.

An antibody or antigen binding fragment thereof of the invention may comprise one or more mutated amino acid residues. The terms “mutated”, “mutant” and “mutation” in reference to a nucleic acid or an antibody or antigen binding fragment thereof of the invention refers to the substitution, deletion, or insertion of one or more nucleotides or amino acids, respectively, compared to the “naturally” occurring nucleic acid or polypeptide, i.e. to a reference sequence that can be taken to define the wild-type.

The amino acid variations in the CDR sequences may be conservative amino acid substitutions.

A mutation may be a substitution wherein the substitution is a conservative substitution. Conservative substitutions are generally the following substitutions, listed according to the amino acid to be mutated, each followed by one or more replacement(s) that can be taken to be conservative: Ala→Gly, Ser, Val; Arg→Lys; Asn→Gln, His; Asp→Glu; Cys→Ser; Gln→Asn; Glu→Asp; Gly→Ala; His→Arg, Asn, Gln; Ile→Leu, Val; Leu→Ile, Val; Lys→Arg, Gln, Glu; Met→Leu, Tyr, He; Phe→Met, Leu, Tyr; Ser→Thr; Thr→Ser; Trp→Tyr; Tyr→Trp, Phe; Val→He, Leu. Other substitutions are also permissible and can be determined empirically or in accord with other known conservative or non-conservative substitutions.

1, 2 or 3 conservative substitutions may be made in the CDRs of the antibody or antigen binding fragment thereof of the invention.

Methods of making an antibody or antigen binding fragment thereof are well known in the art. The skilled person may use hybridoma technology for example, or may use recombinant DNA technology to clone the respective antibody sequence into a vector, such as an expression vector. Methods of making a bispecific antibody molecule are known in the art, e.g. recombinant DNA technology, chemical conjugation of two different monoclonal antibodies or for example, also chemical conjugation of two antibody fragments, for example, of two Fab fragments. Alternatively, bispecific antibody molecules are made by quadroma technology, which is by fusion of the hybridomas producing the parental antibodies. Because of the random assortment of H and L chains, a potential mixture of ten different antibody structures are produced of which only one has the desired binding specificity. A bispecific antibody molecule of the invention can act as a monoclonal antibody (mAb) with respect to each target. The antibody or antigen binding fragment thereof may be chimeric, humanized or fully human. The antibody or antigen binding fragment thereof may be a human IgG1 isotype or a human IgG4 isotype or other natural or modified isotype. A bispecific antibody molecule or multi-specific antibody may for example be a bispecific tandem single chain Fv, a bispecific Fab2, or a bispecific diabody.

Reference to “OX16”, “OX116”, “OX110”, “OX111”, “OX77a” or “OX25” refers to antibodies 16, 116, 110, 111, 77a or 25, respectively (as defined in Table 11).

All of the features disclosed in this specification may be combined in any combination, including with any aspect or any embodiment.

### BRIEF DESCRIPTION OF THE FIGURES

FIG. 1—shows the inhibition of polyclonal T cell responses by a panel of anti-CD1a antibodies. FIG. 1A. Dose titration curve of polyclonal T cell IFNγ response with increasing concentration of anti-CD1a antibody (0.01-10 μg/ml) (n=6 donors). FIG. 1B. IC50 values calculated for the panel of newly generated anti-CD1a antibodies and commercial antibodies (OKT6, HI149 and SK9, n=6 donors)

FIG. 2—demonstrates the inhibition of CD1a-restricted enriched T cell line responses by a panel of anti-CD1a antibodies. FIG. 2A-2B. Cytokine secretion response of CD1a-restricted enriched T cell lines induced by empty vector (EV) or CD1a transfected K562 presenting endogenous ligands. Inhibition of IFNγ (FIG. 2A.) or IL-22 (FIG. 2B.) was assessed for the panel of newly generated anti-CD1a antibodies by flow cytometry. FIG. 2C. IFNγ secretion response of CD1a-restricted enriched T cell lines induced by CD1a coated beads presenting endogenous ligands. Inhibition was assessed for the panel of newly generated anti-CD1a antibodies by flow cytometry. Inhibition was assessed for the panel of newly generated anti-CD1a antibodies by flow cytometry. (N=4-19 enriched T cell lines, 2-way-ANOVA with Tukey's test, *, P<0.05; **, P<0.01; ***, P<0.001; ****, P<0.0001 where * indicates significance on comparison to “CD1a”.

FIG. 3—demonstrates the characterisation of CD1a transgenic mouse. FIG. 3A. Representative flow cytometry plots and FIG. 3B. graphical summary of CD1a protein expression by cells of wild-type (WT) and CD1a transgenic (CD1a) mice. CD1a protein expression evaluated on (left-right) total live ear skin cells, CD45+ skin cells, dermal dendritic cells (dDCs, CD45+/CD11c+/langerin−) and Langerhans cells (LCs, CD45+/CD11c+/langerin+). FIG. 3C. CD1a protein expression within ear skin of wild-type (WT) and CD1a transgenic (CD1a) mice, visualised by immunofluorescence. Cryosections were stained with DAPI (blue) and anti-CD1a AF-594 (OKT6, red), scale bars left to right 50 μm, 50 μm and 10 μm. FIG. 3D. Exemplar PCR genotyping of CD1a transgenic mouse line litter (lanes A-F) using CD1a forward and reverse primers and tail genomic DNA. Expected CD1a band at 655 bp. Lane G: positive control genomic DNA from founder mouse. Lane H: negative control lacking DNA template. FIG. 3E Representative flow cytometry plots of thymic CD1a protein expression by wild-type (WT) and CD1a transgenic (CD1a) mice.

FIG. 4—Characterisation of anti-CD1a antibodies in vivo. FIG. 4A. Schematic of imiquimod-induced skin inflammation and anti-CD1a preventative administration. FIG. 4B. Daily measurement of ear swelling induced by imiquimod treatment of wild-type (WT) and CD1a transgenic mice (CD1a) injected i.p. with mouse IgG1 isotype control and CD1a transgenic injected with the refined panel of anti-CD1a antibodies as in the schematic panel A. (N=6, 2-way-ANOVA with Dunnett's test, **, P<0.01; ****, P<0.0001 indicates significance on comparison to “CD1a” at day 6 or as shown).

FIG. 5—demonstrates the effect of anti-CD1a on the imiquimod-induced cutaneous immune response. FIG. 5A-5C. Flow cytometric analysis of ear skin of mouse IgG1 isotype treated wildtype (WT) and CD1a transgenic (CD1a) and CD1a transgenic injected with the refined panel of anti-CD1a antibodies following the preventative model of administration. Skin T cells were enumerated (FIG. 5A.) and assessed for cell surface CD69 expression (FIG. 5B.) and skin neutrophil (FIG. 5C.) and eosinophil (FIG. 5D.) frequency was determined. (N=4, 1-way-ANOVA with Dunnett's test, *, P<0.05; **, P<0.01; ***, P<0.001).

FIG. 6—demonstrates the effect of anti-CD1a on the imiquimod-induced cellular Langerhans cell skin and lymph node response. Flow cytometric analysis of ear skin (FIGS. 6A-6B.) and draining cervical lymph node (FIGS. 6C-6D.) of mouse IgG1 isotype treated wildtype (WT) and CD1a transgenic (CD1a) and CD1a transgenic injected with the refined panel of anti-CD1a antibodies following the preventative model of administration. Skin LCs were enumerated (FIG. 6A.) and assessed for cell surface CD1a expression (FIG. 6B.). Lymph node LCs were enumerated (FIG. 6C.) and assessed for cell surface CD1a expression (FIG. 6D.). (N=4, 1-way-ANOVA with Dunnett's test, *, P<0.05; **, P<0.01; ***, P<0.001; ****, P<0.0001).

FIG. 7—demonstrates antibody dependent depletion (phenotypic change). FIG. 7A. Flow cytometric analysis of antibody induced CD1a dependent cell reduction (such as death). Anti-CD1a antibodies or mouse IgG1 isotype control (iso, 5 μg/ml) were incubated with EV or CD1a-K562 as indicated for 48 hours and percentage of antibody induced reduction was calculated in relation to a reference population of untreated K562 and was normalised to EV control cells. FIG. 7B. Dose titration curve of antibody induced CD1a-K562 cell reduction with increasing concentration of anti-CD1a antibody (0.625-5 μg/ml). FIGS. 7C-7D. Anti-CD1a antibodies or mouse IgG1 isotype control (iso, 5 μg/ml) were incubated with MoDCs (upper panel) and MoLCs (lower panel) as indicated for 5 days with antibodies and cytokines added on day 0 or day 2 and percentage of antibody induced reduction was calculated in relation the isotype control as measured by percentage confluence using Incucyte live cell imaging (N=4, 2-way-ANOVA with Tukey's test.) (FIG. 7C.) and representative images of MoLCs (FIG. 7D.). FIG. 7E. K562-CD1a or K562-EV (empty vector) were incubated with anti-CD1a antibodies for 24 hours and stained for Annexin V and analysed by flow cytometry. (N=3-4, 1-way-ANOVA with Tukey's test.) FIG. 7F. Flow cytometric analysis of complement-dependent cytotoxicity (CDC). K562-CD1a cells were incubated with 10% normal human serum for 3-hours at 37° C. in the presence of either 5 μg/ml isotype control antibody or indicated antibodies. Percentage cytotoxicity was calculated in relation to a reference population of untreated K562 and was normalised to isotype control treated cells. (N=6, 1-way-ANOVA with Tukey's test.) FIG. 7G. Flow cytometric analysis of antibody-dependent cell-mediated cytotoxicity (ADCC). K562-CD1a cells were co-cultured with PBMC at 1:50 ratio for 5-hour at 37° C. in the presence of either 5 μg/ml isotype control antibody or indicated antibodies. Percentage cytotoxicity was calculated in relation to a reference population of untreated K562 and was normalised to isotype control treated cells. (N=4-6, 1-way-ANOVA with Tukey's test.) FIG. 7H. NSG mice were subcutaneously injected with 0.25 million CD1a-K562 cells in the flank and tumours were allowed to develop for 18 days. Mice were treated with 100 μg isotype control antibody or indicated antibodies on days 6, 10, and 14 intraperitoneally. Measurement of tumour volume over time. (N=6-15, 2-way-ANOVA with Tukey's test, asterisks indicate significance on comparison to “CD1a-iso” at day 18). *, P<0.05; **, P<0.01; ***, P<0.001; ****, P<0.0001.

FIG. 8 (FIG. 8A)—is a heatmap from CD1a epitope analysis. Matrix heatmap representation of CD1a antibody binding by flow cytometry as measured by CD1a-AF647 mean fluorescence intensity (MFI). Before staining of CD1a-K652 with anti-CD1a antibodies conjugated to fluorophore AF647, the relevant purified antibodies were incubated with the cells to assess interference in CD1a binding of the AF647-conjugated antibodies. Grayscale shows degree of interference with the tone in the top row (−) indicating no interference. (FIG. 8B)—demonstrates in vivo CD1a antibody epitope competition assay results. A. Flow cytometry plots of CD1a expression as measured by staining with anti-CD1a antibodies SK9 (left panels) or HI149 (right panels). Anti-CD1a antibody 116 (100 μg i.p.) was administered on days 0, 2 and 4 and ear skin tissue collected, processed and stained for CD1a on day 5.

FIG. 9—demonstrates the effectiveness of application of anti-CD1a antibodies in the treatment of imiquimod-induced inflammation. FIG. 9A. Schematic of imiquimod-induced inflammation model with therapeutic anti-CD1a administration. FIG. 9B. Daily measurement of ear swelling and FIG. 9C. representative images of inflammation (day 8) induced by imiquimod treatment of wild-type (WT) and CD1a transgenic mice (CD1a) followed by the treatment i.p. with mouse IgG1 isotype control or CD1a transgenic injected with the refined panel of anti-CD1a antibodies as in the schematic panel A (at day 3 arrowpoint) (N=2-10, 2-way-ANOVA with Dunnett's test, **, P<0.01; ****, P<0.0001 indicates significance on comparison to “CD1a” at day 8 or as shown). FIG. 9D. Ear and epidermal thickness and CD1a protein expression within ear skin of wild-type (WT) and CD1a transgenic (CD1a) mice treated with imiquimod (Imiq) or untreated (U) visualised by immunofluorescence. Cryosections were stained with DAPI (blue) and anti-CD1a AF-594 (OKT6, red), scale bars 10 μm upper panels and 100 μm lower panels. FIGS. 9E-9G. Flow cytometric analysis of ear skin of mouse IgG1 isotype treated wild-type (WT) and CD1a transgenic (CD1a) and CD1a transgenic injected with the refined panel of anti-CD1a antibodies following the treatment model of administration. Skin T cells were enumerated and assessed for cell surface CD11a expression (FIG. 9E.) and neutrophil (FIG. 9F.) and eosinophil (FIG. 9G.) frequency was determined. (N=7-9, 1-way-ANOVA with Dunnett's test, *, P<0.05; **, P<0.01; ***, P<0.001).

FIG. 10—demonstrates the CD1a dependency of the systemic effects of imiquimod application. FIG. 10A. Spleen weight (mg) measurements and representative images on day 8 by imiquimod treatment of wild-type (WT) and CD1a transgenic mice (CD1a) followed by treatment i.p. with mouse IgG1 isotype control or CD1a transgenic injected with the refined panel of anti-CD1a antibodies as in the schematic (FIG. 9A). B-E. Flow cytometric analysis of spleen of mouse IgG1 isotype treated wild-type (WT) and CD1a transgenic (CD1a); and CD1a transgenic injected with the refined panel of anti-CD1a antibodies following the treatment model of administration. Splenic CD4 (FIG. 10B.) and CD8 (FIG. 10C.) T cell CD69 expression was assessed and neutrophils (FIG. 10D.) and eosinophils (E.) were enumerated. (N=7-9, 1-way-ANOVA with Dunnett's test, *, P<0.05; **, P<0.01; ***, P<0.001; ****, P<0.0001). FIG. 10F. Plasma cytokine levels of the blood of mouse IgG1 isotype treated wild-type (WT) and CD1a transgenic (CD1a); and CD1a transgenic injected with anti-CD1a antibodies following the treatment model of administration (N=7-9, 1-way-ANOVA with Dunnett's test, *, P<0.05; **, P<0.01; ***, P<0.001; ****, P<0.0001).

FIG. 11—demonstrates CD1a dependency of the systemic effects of imiquimod application. FIGS. 11A-11E. Blood cellular analysis of the blood of mouse IgG1 isotype treated wild-type (WT) and CD1a transgenic (CD1a); and CD1a transgenic injected with the refined panel of anti-CD1a antibodies following the treatment model of administration. Circulating T cells (FIG. 11A.), CD4+(FIG. 11B.) and CD8+(FIG. 11C.), neutrophils (FIG. 11D.) and eosinophils (FIG. 11E.) were enumerated. (N=5-7, 1-way-ANOVA with Dunnett's test, *, P<0.05; **, P<0.01; ***, P<0.001; ****, P<0.0001).

FIG. 12—shows that imiquimod does not constitute a CD1a ligand. Isoelectric point dependent migration of mock and imiquimod “loaded” CD1a protein on isoelectric focusing (IEF) gel pH3-7. Mock: vehicle control TBS 2% CHAPS 7% DMSO.

FIG. 13—effectiveness of application of anti-CD1a antibodies in sustained control of imiquimod-induced inflammation. FIG. 13A. Schematic of imiquimod re-challenge model without later anti-CD1a administration. FIG. 13B. Daily measurement of ear swelling induced by imiquimod treatment of wild-type (WT) and CD1a transgenic mice (CD1a) injected i.p. with mouse IgG1 isotype control and CD1a transgenic injected with the refined panel of anti-CD1a antibodies as in the schematic panel 13A (2-way-ANOVA with Dunnett's test, *, P<0.05; **, P<0.01 indicates significance on comparison to “CD1a” isotype at day 7 of imiquimod re-application).

FIG. 14—effectiveness of application of anti-CD1a antibodies in treatment of imiquimod-induced inflammation, compared to a standard of care. Daily measurement of ear swelling induced by imiquimod treatment of wild-type (WT) and CD1a transgenic mice (CD1a) followed by the treatment i.p. with mouse IgG1 isotype control (CD1a) or CD1a transgenic injected with the refined panel of anti-CD1a antibodies and anti-IL-17A as in the schematic panel FIG. 9A. dx=day of model that significance was reached compared to CD1a transgenic ear thickness.

FIG. 15—comparator analysis of the effectiveness of application of anti-CD1a antibodies in the treatment of imiquimod/MC903-induced inflammation. FIG. 15A. Schematic of imiquimod-induced inflammation with therapeutic anti-CD1a administration. FIG. 15B. Daily measurement of ear swelling induced by imiquimod treatment of wild-type (WT) and CD1a transgenic mice (CD1a) followed by the treatment i.p. with mouse IgG1 isotype control or CD1a transgenic injected with the refined panel of anti-CD1a antibodies or CR2113 as in the schematic panel A. N=2-7, 2-way-ANOVA with Dunnett's test, *, P<0.05, **, P<0.01; ****, P<0.0001 indicates significance on comparison to “CD1a” at day 8 or OX116 vs CR2113 at day 8. FIG. 15C. Data and comparisons presented in (B), corrected for WT. D. Schematic of MC903-induced inflammation with preventative MC903-induced inflammation. E. Daily measurement of ear swelling induced by MC903 treatment of wild-type (WT) and CD1a transgenic mice (CD1a) after the treatment i.p. with mouse IgG1 isotype control or CD1a transgenic injected with 16, 110 or 116 anti-CD1a antibody or CR2113 as in the schematic panel FIG. 15D. Corrected for WT. N=3-4, 2-way-ANOVA with Dunnett's test, *, P<0.05, indicates significance on comparison to “CD1a” at day 7. F. Skin T cell percentage and eosinophil count measured by flow cytometry. N=3-4, 2-way-ANOVA with Dunnett's test, *, P<0.05; **, P<0.01; ***, P<0.001.

FIG. 16—comparator analysis of the effect of anti-CD1a antibodies in skin and systemic immune responses with imiquimod-induced inflammation. Ear skin, draining cervical lymph node and plasma samples were analysed from mouse IgG1 isotype treated wildtype (WT) and CD1a transgenic (CD1a) and CD1a transgenic injected with the refined panel of anti-CD1a antibodies following the treatment model of administration as shown in schematic FIG. 15A. FIG. 16A. Skin T cell IL-17A expression was analysed using intracellular cytokine expression detected by flow cytometry directly ex vivo (left panel), and cervical lymph node eosinophils were enumerated (right panel). FIGS. 16B-16C. Plasma (FIG. 16B) and skin digest (FIG. 16C) cytokine levels were measured by ELISA (N=2-7, 1-way-ANOVA with Dunnett's test, *, P<0.05; **, P<0.01; ***, P<0.001; ****, P<0.0001).

FIG. 17—crystal structure analysis of OX16, OX110, OX116 Overview of the crystal structures corresponding to human CD1a bound to three different antibody fragments. LEFT PANELS: Heavy chain of CD1a is shown in grey, 02-microglobulin in blue and the corresponding scFv molecules are depicted in green for OX16, yellow for OX110 and pink for OX116. The segment of each scFv fragment corresponding to VH domain is shown in darker colour and V_{L} in a lighter tone. RIGHT PANELS: Surface representation of the region of CD1a (grey) recognised by each antibody fragment. The residues of CD1a contacting each scFv molecule are shown in colour as depicted. Contact residues are considered to be within 3.5 Å distance from the interacting chain.

FIG. 18—Effects of lipid in binding of OX16 and OX116 to CD1a Interaction between OX16 or OX116 scFv and CD1a carrying different lipid ligands was measured using surface plasmon resonance (SPR). Binding curves were calculated by fitting the response units measured upon injection of CD1a-endogenous lipids (red), CD1a-sphingomyelin (blue), CD1a-lysophosphatidylcholine (green) or CD1a-GD3 ganglioside (brown) over a flow cell containing OX16-scFv (top) or OX116-scFv (bottom). The units of Bmax values are relative response units and K_{D} values are provided in nM.

FIG. 19—Effects of blocking of polyclonal and clonal T cell function by anti-CD1a antibodies

FIGS. 19A-19B. Determination of the capacity anti-CD1a antibody variants to inhibit the CD1a dependent activation of polyclonal T cell IFNγ (FIG. 19A.) and IL-22 (FIG. 19B.) production. T cells were isolated from donor PBMCs by CD3 microbead separation. T cells were cocultured overnight with CD1a-K562 or EV-K562 and IFNγ or IL-22 production was detected by ELISpot in the presence of 10 μg/ml anti-CD1a antibodies. Antibody formats were compared to the mouse IgG1 isotype control (top row statistics) or against the respective isotype (bottom row statistics). % blockade was calculated upon comparison of the antibody treated and isotype control following subtraction of the EV background level of cytokine spots. (N=4 donors, 1-way-ANOVA with Sidak's test, *, P<0.05; **, P<0.01; ***, P<0.001; ****, P<0.0001, mean±SD). FIG. 19C. Cytokine secretion response of CD1a-restricted T cell clones induced by empty vector (EV) or CD1a transfected K562 presenting endogenous ligands. Inhibition of IFNγ was assessed for the anti-CD1a antibodies by flow cytometry, and normalised to the isotype response (N=4-8 T cell clones, 1-way-ANOVA with Tukey's test, *, P<0.05; **, P<0.01; ***, P<0.001; ****, P<0.0001

FIG. 20—Complement-dependent cytotoxicity (CDC) and antibody-dependent cytotoxicity (ADCC) of anti-CD1a antibodies

FIG. 20A. Flow cytometric analysis of complement-dependent cytotoxicity (CDC). K562-CD1a cells were incubated with 10% normal human serum for 3-hours at 37° C. in the presence of either 5 μg/ml isotype control antibody or indicated antibodies. Percentage cytotoxicity was calculated in relation to a reference population of untreated K562 and was normalised to isotype control treated cells.

FIG. 20B. Flow cytometric analysis of antibody-dependent cytotoxicity (ADCC). K562-CD1a cells were incubated with PBMCs at an effector/target ratio of 50:1 for 5-hours at 37° C. in the presence of either 5 μg/ml isotype control antibody or indicated antibodies and 0.5% FCS and 100 U/ml IL-2. Percentage cytotoxicity was calculated in relation to a reference population of untreated K562 and was normalised to isotype control treated cells.

(N=4-6, 1-way-ANOVA with Tukey's test.) *, P<0.05; **, P<0.01; ***, P<0.001, ****, P<0.0001.

FIG. 21—inhibition of TCR binding to CD1a by OX116

FIG. 21A. Schematic of experimental set-up to determine ability of OX116 to inhibit TCR binding to CD1a. FIG. 21B. Binding curves showing the interaction of CD1a restricted TCRs (CO3 γδTCR—red; BK6 αβTCR—blue; CO22 γδTCR—green) with CD1a bound to OX116 Ab fragment.

FIG. 22—clone OX25 CD1a binding characteristics

FIG. 22A. CD1a transfected cells and recombinant protein (and controls) were investigated for binding by antibody OX25 by flow cytometry (Fluorescence Intensity Geomean) and by ELISA (optical density). Target cells and protein included the major and minor variants of CD1a as well as Cynomolgus CD1a, and cells naturally expressing CD1a (MOLT4). FIG. 22B. ELISA (optical density) of full length CD1a compared to chimeric CD1a with human alpha 1 and alpha 2 domains fused to murine alpha 3 domain of CD1d using size exclusion pool B. FIG. 22C. Surface plasmon resonance of OX25 interaction with CD1a. FIG. 22D. Heavy and light chain CDR3 regions of OX25 (SEQ ID NOs 315 and 316 respectively).

FIG. 23—Humanised antibodies can deplete CD1a-expressing transfectants. Modified human IgG1 anti-CD1a antibodies or isotype control (5 g/ml) were incubated with K562-CD1a as indicated for 48 hours. Percentage of antibody induced reduction was calculated in relation the isotype control as measured by percentage confluence using Incucyte live cell imaging (N=3, 2-way-ANOVA with Dunnett's test. **, P<0.01; ****, P<0.0001 where * indicates significance on comparison to “CD1a”.)

FIG. 24—Humanised antibodies can inhibit CD1a-autoreactive T cells. Cytokine secretion response of CD1a-restricted enriched T cell lines induced by empty vector (EV) or CD1a transfected K562 presenting endogenous ligands. Inhibition of IFNγ was assessed for the panel of modified human IgG1 anti-CD1a antibodies and isotype control (5 g/ml) by flow cytometry. (N=5-6 enriched T cell lines, 1-way-ANOVA with Dunnett's test, *, P<0.05; **, P<0.01; ***, P<0.001; ****, P<0.0001 where * indicates significance on comparison to “CD1a”.)

FIG. 25—OX25 binds alpha-3 domain of CD1a. Characterisation of anti-CD1a antibody OX25. FIG. 25A. ELISpot analysis of OX25 and commercially available comparators (SK9—non-blocking, OKT6 and HI148—blocking antibodies). Polyclonal T cell IFNγ response to overnight coculture with CD1a-transfected (CD1a) or empty vector (EV) K562 model antigen presenting cells. The impact of anti-CD1a antibodies (10 μg/ml) upon T cell activation was measured by IFNγ (IFNγ spots) ELISpot (n=8 T cell donors). FIG. 25B. Matrix heatmap representation of CD1a antibody binding by flow cytometry as measured by CD1a-AF647 mean fluorescence intensity (MFI). Before staining of CD1a-K652 or empty vector (EV) K562 with anti-CD1a antibodies conjugated to fluorophore AF647, the relevant purified antibodies were incubated with the K562 cells to assess interference in CD1a binding of the AF647-conjugated antibodies. Grayscale shows degree of interference with the tone in the top row (−) indicating no interference, 100% binding.

FIG. 26—Demonstrates effectiveness of OX116 in the treatment of CD1a and checkpoint inhibition dependent skin inflammation. FIG. 26A. Schematic of imiquimod-induced skin inflammation and anti-CD1a (clone OX116) and anti-PD1 (clone J43) or isotype control administration. FIG. 26B. Daily measurement of ear swelling induced by imiquimod treatment of wild-type (WT) and CD1a transgenic mice (CD1a) injected i.p. with mouse IgG1 isotype control, anti-PD1 (J43) or anti-CD1a OX116 as in the schematic panel A. (N=4, 2-way-ANOVA with Dunnett's test, *, P<0.05, **, P<0.01; indicates significance at day 6 as shown). C. Intracellular flow cytometric analysis of wild-type (WT) and CD1a transgenic (CD1a) ear skin treated with or without imiquimod and injected i.p. with mouse IgG1 isotype control, anti-PD1 (J43) or anti-CD1a (OX116). Skin T cells were identified by CD3+ surface staining and IL-17 production was analysed ex vivo by intracellular staining with anti-IL17 antibody.

FIG. 27—FIG. 27A. Pruritus and pruritogenic cytokines are significantly reduced following administration of anti-CD1a antibodies. The ear tissue of hCD1a and WT mice were topically treated with 1 nmol MC903/EtOH on days 0, 2 and 5. 100 ug/100 ul of OX116, OX16 and isotype control were administered intraperitoneally on days −2, 0, 2 and 4. (A) Itching frequency was evaluated at endpoint. Each data plot represents an individual ear. Plots show mean±SEM. Statistics were calculated using one-way ANOVA with Tukey post-test. *P<0.05, *** P<0.001, NS=nonsignificant. FIG. 27B. Epidermal cytokines are reduced in skin tissue following administration of anti-CD1a antibodies. The ear tissue of hCD1a and WT mice were topically treated with 1 nmol MC903/EtOH on days 0, 2 and 5. 100 ug/100 ul of OX116, OX16 and isotype control were administered intraperitoneally on days −2, 0, 2 and 4. TSLP (left panel) and IL-33 (right panel) (pg/ml/per ear) levels were assessed at endpoint by Legendplex™. Each data plot represents an individual ear. Plots show mean±SEM. Statistics were calculated using one-way ANOVA with Tukey post-test. *P<0.05, **P<0.01, NS=nonsignificant.

FIG. 28—Shows that a bispecific CD1a Ab controls CD1a-expressing target cells in vitro and in vivo. FIG. 28A. Left panel: K562 cells expressing CD1a were co-cultured in vitro with Jurkat cells (CD1a-KO) expressing an activation reporter gene (NFAT-GFP) (50,000 each at effector:target 1:1). WIMM-3 bispecific antibody was added and incubated at 37° C. 5% CO2 for 18 hours. Cells were stained with a viability dye and an anti-CD1a antibody in order to identify the live CD1a negative Jurkat T cells. Right panel: K562-CD1a-GFP, and K562-empty vector-mCherry cells were mixed at equal numbers (25,000 each) and co-cultured with 125,000 human CD8+ T cells that had been rested for 12 days, following isolation from PBMC and stimulation with anti-CD3 and anti-CD28 beads. The co-culture was incubated over 48 hours. FIG. 28B. Anaesthetized NSG mice (n=7/group) were injected subcutaneously on the lateral flank with 0.5 million K562 cells expressing CD1a. Four days later mice were injected intravenously with Human CD8+ T cells that had been isolated from PBMC and stimulated with anti-CD3/anti-CD28 beads 14 days previously. Mice were also injected with 100 ul of bispecific antibody WIMM-3 (0.5 mg/Kg) or PBS. Mice were treated a second time with human CD8+ T cells and WIMM-3 or PBS 14 days after the first injection. Left panel shows average tumour size, right panel shows data for individual mice, bottom panel shows survival. Student t-test was used at each time point. P values <0.0001****, <0.001***, <0.005**, <0.05*.

### MATERIALS AND METHODS

### Mice

All mice were bred in a specific pathogen-free facility. In individual experiments, mice were matched for age, sex and background strain with wild-type litter mates used as matched controls. All experiments undertaken in this study were done so with the approval of the UK Home Office.

### CD1a Transgenic Mouse Generation

Mice were generated by the Wellcome Trust Centre for Human Genetics, Oxford. A 5.7 kb genomic fragment encompassing the entire CD1A gene, including 0.8 kb of upstream sequence and 0.8 kb of downstream sequence, was amplified from human genomic DNA by PCR using primers 5′-ATGGTACCAAGAGGAATGTAAATGTGTCCGGC-3′ and 5′-AAGCGGCCGCGATCATGTTAACCAAGGTCAGGAA-3′ and subcloned into the Litmus28 vector (NEB) via the KpnI and NotI sites incorporated into these PCR primers. After sequence verification of the coding exons, the fragment transgene was excised from the vector backbone, purified and resuspended at 2 ng/ul in microinjection buffer (10 mM Tris-HCl, pH 7.4, 0.25 mM EDTA) and microinjected into a pronucleus of fertilized zygotes prepared from C57BL/6J mice. After overnight culture, the resulting 2-cell embryos were surgically implanted into the oviduct of pseudopregnant CD1 foster mother and carried to term. Transgenic offspring were identified by PCR using transgene specific primers and bred as individual lines with wild-type C57BL/6J mice.

### CD1a Genotyping

Crude genomic DNA preparation was performed on ear notch samples from CD1a transgenic mice. 100 μl of DirectPCR ear lysis buffer (Viagen) supplemented with 0.4 mg/ml proteinase K (Sigma) was added to ear notches and incubated at 55° C. overnight. Enzymes were then heat inactivated at 85° C. for 1 hour. The samples were centrifuged to pellet debris and the lysate was transferred to a clean tube. 1 μl of lysate was used as a template for genotyping. The below PCR reaction was used for genotyping. PCR products were loaded on to a 1% TAE agarose gel with SyberSafe, electrophoresis run and the gel imaged under UV. If the expected band at 655 bp was detected, mice were considered positive for the CD1a transgene.

| | | |
|---|---|---|
| DNA template | 1 | ul |
| MyTaqRed Mix (BioLine) 2x | 25 | ul |
| CD1a forward primer | 0.4 | μM |
| CD1a reverse primer | 0.4 | μM |

| | |
|---|---|
| dd-H20 | Up to 50 ul reaction volume |

| | |
|---|---|
| 1. Initial denaturation | 95° C., 2 min |
| Cycle: Steps 2-4 | X34 |
| 2. Denaturation | 95° C., 20 sec |
| 3. Annealing | 60° C., 15 sec |
| 4. Extension | 72° C., 20 sec |
| 5. Final extension | 72° C., 2 mins |
| 6. Hold | 4° C. |

### Cell Lines

Empty vector-transfected K562 (EV-K562) and CD1a-transfected K562 (CD1a-K562) cells (a gift from B. Moody, Brigham and Women's Hospital, Harvard Medical School, Boston, MA) were maintained in RPMI 1640 medium supplemented with 10% FCS, 100 IU/ml penicillin, 100 g/ml streptomycin (Sigma-Aldrich), 2 mM L-glutamine (Gibco), 1× nonessential amino acids (NEAAs) (Gibco), 1 mM sodium pyruvate (Gibco), 10 mM HEPES (Gibco), 500 μM 2-mercaptoethanol (Gibco), and 200 g/ml G418 antibiotic (Thermo Fisher Scientific).

### ELISpot Analysis

ELISpot assay (IFNγ ELISpot kit, Mabtech, AB) was used to detect activation-induced cytokine secretion from polyclonal T cells upon coculture with model CD1a expressing antigen presenting cells. PBMCs from healthy donor blood were isolated by density gradient (Lymphoprep) and T cells purified using anti-CD3 magnetic bead sorting following the manufacturer's protocol (MACS, Miltenyi). All study participants gave fully informed written consent [National Health Service (NHS) National Research Ethics Service (NRES) research ethics committee 14/SC/0106. T cells were then cultured for 3 days with IL-2 (200 U/ml) to expand in number prior to overnight co-culture with unpulsed/endogenous lipid bound CD1a-transfected K562 (CD1a-K562) or control empty-vector transfected K562 (EV-K562). To assess the functionality of the anti-CD1a antibodies, K562 were incubated with 10 μg/ml anti-CD1a antibodies 1 hour prior to and during co-culture with polyclonal T cells in an anti-IFNγ or anti-IL-22 capture antibody coated ELISpot plate (Millipore Corp., MA). IFNγ and IL-22 secretion was detected with a biotinylated anti-cytokine detection antibody and visualised with streptavidin-alkaline phosphatase development. Resulting spots were indicative of cytokine producing T cells and were enumerated using an automated ELISpot reader (Autimmun Diagnostika gmbh ELISpot Reader Classic), and the % blockade was calculated upon comparison of the antibody treated and untreated groups following subtraction of the EV background level of cytokine production spots. The EV-K562 contribution was subtracted from the CD1a IFNγ/IL-22 spot number. The adjusted CD1a-K562 antibody-treated group spot number was then divided by the CD1a without antibody group and used to calculate % blockade.

### CD1a-Reactive T Cell Generation and Activation Analysis:

CD1a-restricted T cells were isolated by fluorescence activated cell sorting. T cells were co-cultured with EV-K562 of CD1a-K562 and cytokine producing responder T cells were detected using Miltenyi MACS Cytokine Secretion assays following the manufacturer's instructions. Briefly T cells were coated with anti-cytokine (IL-22 or IFNγ) antibody after a 6-hour culture with CD1a-K562 to detect CD1a dependent autocrine cytokine production. The live responder cells were then sorted into a culture plate. CD1a-restricted T cells were expanded with mixed lymphocyte reaction, and purity and CD1a-responsiveness were assessed with the above FACS-based cytokine secretion assay method using an analysing flow cytometer. The activation of CD1a-restricted T cells was analysed as follows. 2×10⁵ K562 cells were co-cultured with 1-5×10⁵ CD1a-autoreactive T cell clones for 4 hr. Helper cytokines were added to the co-culture to support CD1a-dependent cytokine production. IFNγ-producing T cell culture was supplied with IL-12 (1 ng/mL, BioLegend), IL-18 (1 ng/mL, BioLegend), and IL-2 (25 U/mL, BioLegend); Activation of T cells was assessed by cytokine production of T cells using a cytokine secretion assay (Miltenyi Biotec) following the manufacturer's instructions.

### Murine Imiquimod Administration

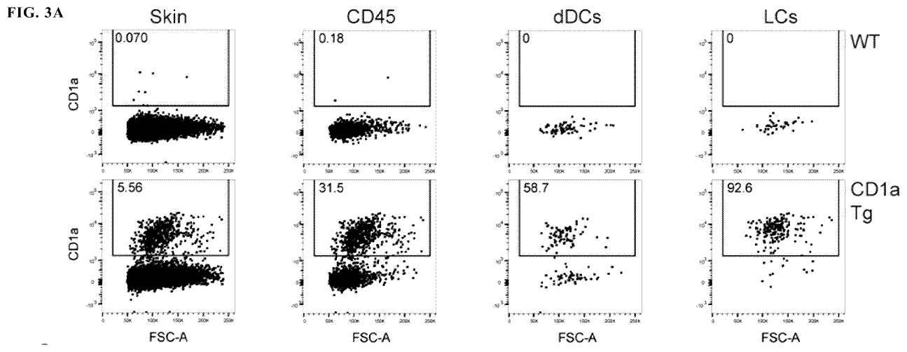

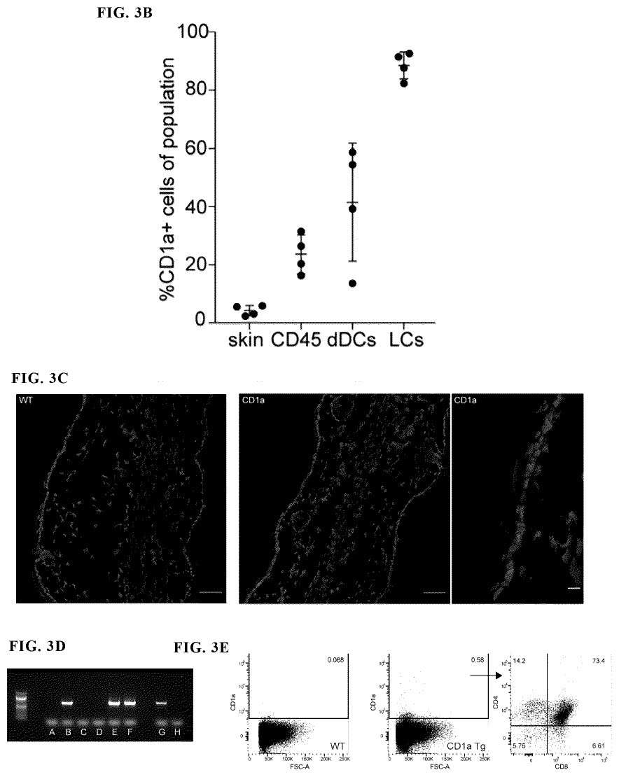

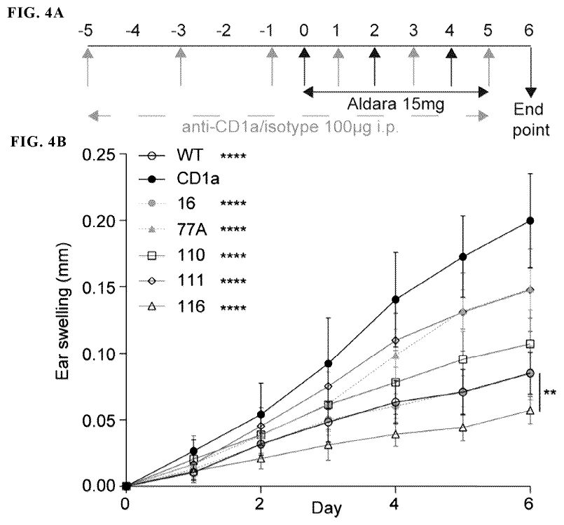

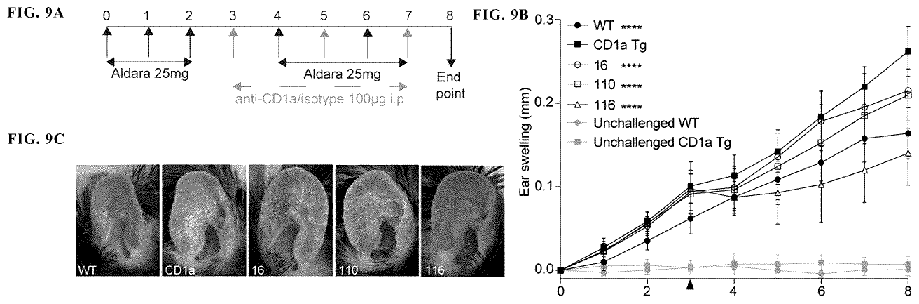

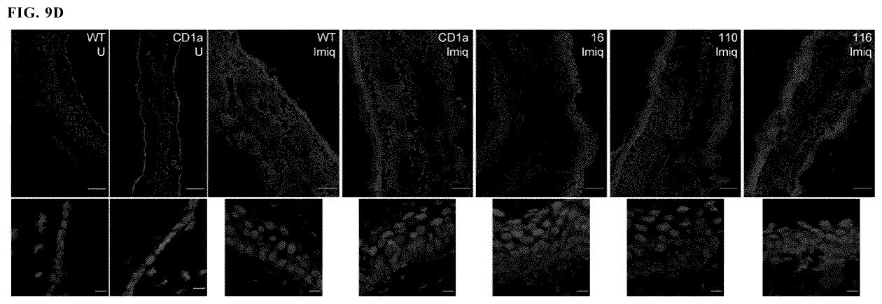

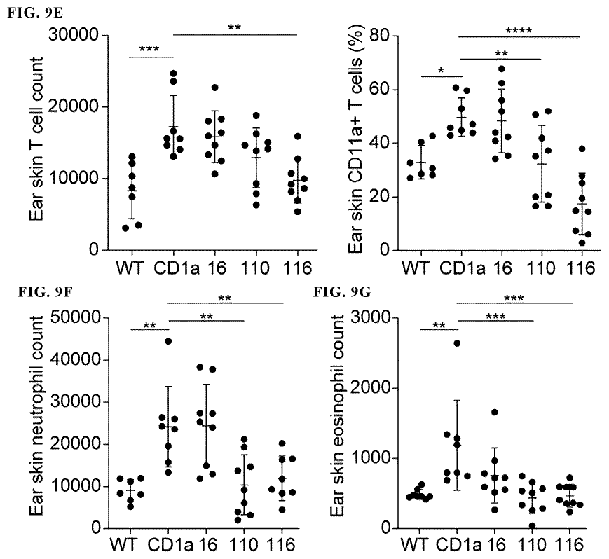

Mice were lightly anaesthetised with isoflurane and 15 mg Aldara cream containing 5% imiquimod was applied to the dorsal and ventral sides of the ear pinnae on days 0, 1, 2, 3, 4, 5 in the prevention model (FIG. 4A) or 0, 1, 2 and 4, 5, 6, 7 in the treatment model (FIG. 9A). 100 μg anti-CD1a antibodies or mouse IgG1 isotype control were administered intraperitoneally on days −5, −3, −1, 1, 3, 5 in the prevention model (FIG. 4A) or 3, 5, 7 in the treatment model (FIG. 9A). Ear thickness measurements were taken daily throughout the duration of Aldara application days 0-6 in the prevention model (FIG. 4A) or 0-8 in the treatment model (FIG. 9A) using a micrometer (Mitutoyo). Mice were sacrificed and tissues taken 24 h after challenge.

### Murine MC903 Administration

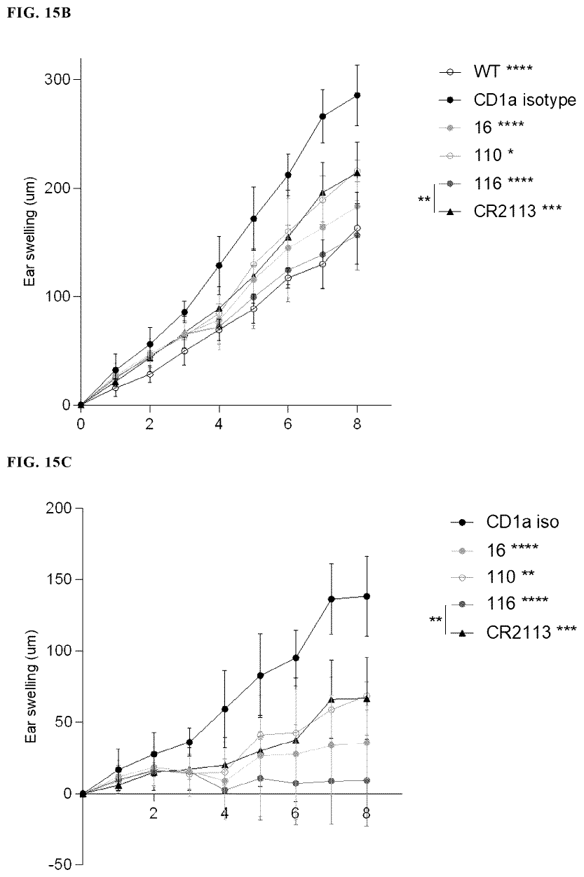

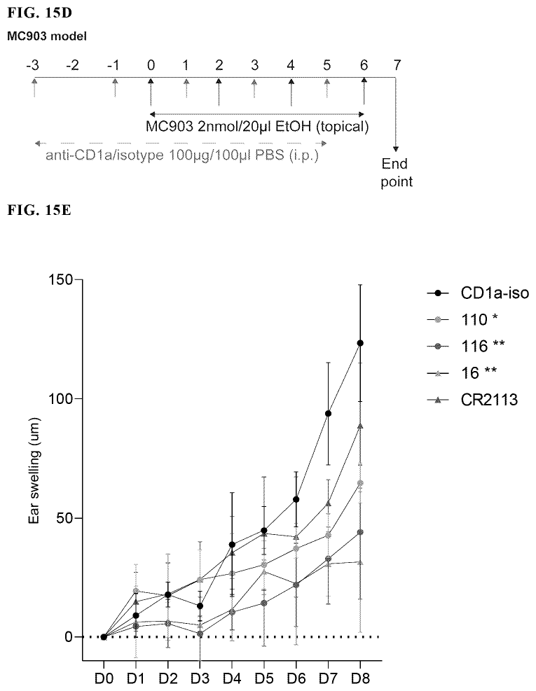

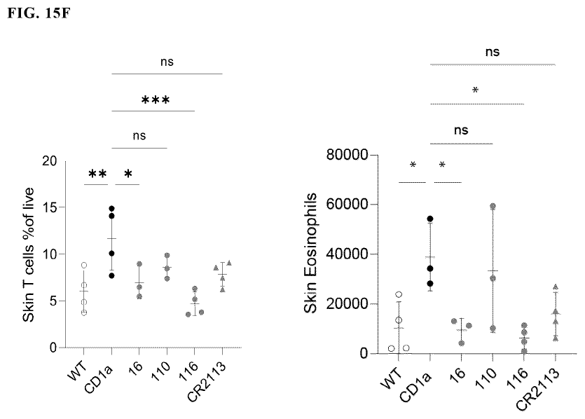

Mice were lightly anaesthetised with isoflurane and 2 nmol per dose of MC903 daily for 7 days applied to ventral and dorsal side of ear (10 microlitres each side of the ear). 100 μg anti-CD1a antibodies or mouse IgG1 isotype control were administered intraperitoneally as indicated in FIG. 15D. Ear thickness measurements were taken daily using a micrometer (Mitutoyo).

### Tissue Processing

Mice were sacrificed and tissues taken 24 h after final imiquimod challenge. Ears, cervical lymph nodes (cLN) and spleen were collected for immunophenotyping or imaging. Cell suspensions of spleen and cLN, were obtained by passing the tissues through a 70 m strainer and washed with RPMI containing 10% FCS. Spleen cell suspension red blood cells were removed by incubation with RBC lysis solution (eBioscience).

Ear skin tissue was washed in HBSS to remove excess imiquimod, split ventrally, diced into <0.5 mm pieces and digested with 1 mg/mL collagenase P (Roche) and 0.1 mg/mL DNaseI (Sigma-Aldrich) DMEM for 3×30 mins with agitation, dispase 5 mg/mL was added to the final 30 min digest step. A single cell suspension wash obtained upon washing with DMEM containing 10% FCS through a 70 m strainer prior to analysis by flow cytometry.

### Flow Cytometry

For FACS surface staining the cells were labelled with the following anti-mouse antibodies (Biolegend sourced unless otherwise stated): CD3 (500A2, BUV495: 741064 BD Pharmingen), CD11b (M1/70, BUV395: 563553 BD Pharmingen), CD11c (N418, BV711: 117349), CD8 (53-6.7, BUV805: 612898 BD Pharmingen), CD4 (GK1.5, AF700: 100430), CD45 (2D1, FITC: 368507), CD11a (121/7, PECy7: 153108), CD69 (H1.2F3, BV650: 104541), Langerin (4C7, PE: 144204), Ly6C (RB6-8C5, BV605: 108440), Ly6G (1A8, PETxRed: 127648), MHCII (M5/114.15.2, BV785: 107645), SiglecF (S17007L, BV421: 155509), IL-17A (TC11-18H10.1, PECy7: 506922) Live/Dead Aqua (Invitrogen), and anti-human CD1a (APC or purified SK9, HI149, OKT6, NA1/34).

### Flow Cytometry: Epitope Competition Assay

CD1a-K562 cells were incubated with purified primary newly generated and commercially available anti-CD1a antibodies on ice for 30 minutes (25 μg/ml), the unbound antibody was then washed away and Alexa-Fluor-647 conjugated forms of the different antibodies were then incubated with the cells on ice for 30 minutes (10 μg/ml) in the matrix arrangement. Mean fluorescent intensity (MFI) was used to assess the degree of binding of the fluorophore conjugated antibody.

### Confocal Imaging

Murine ear skin was frozen in optimal cutting temperature embedding compound and stored at −80° C. 10 μm cryosections were cut using a Leica cryostat and collected onto Superfrost Plus slides to air-dry for 30 min before being stored at −80° C. Slides were rehydrated in PBS for 10 min before staining. The endogenous peroxidase activity of the sample was quenched by adding 0.15% hydrogen peroxide solution for 5 minutes at room temperature. Endogenous biotin was blocked with Avidin/Biotin Blocking Kit (Vector Laboratories Ltd), and 10% goat serum was used to reduce nonspecific binding of antibodies. Anti-CD1a antibody was used for confocal microscopy (1:100, OKT6; in-house production and conjugated to Biotin). Alexa Fluor 594 Tyramide SuperBoost kit (streptavidin; Thermo Fisher Scientific) was used to enhance the signal following manufacturer's instructions. Briefly, slides were incubated at 4° C. with primary antibodies overnight. After washing, HRP-conjugated streptavidin was added to the sections and incubated at 4° C. overnight. Excess streptavidin-HRP was washed away, the tissues were incubated with tyramide working solution for 8 min at room temperature, and the reaction was stopped with Reaction Stop Reagent. After staining, slides were mounted using antifade mounting medium with DAPI (Vector Laboratories Ltd), coverslips were applied, and slides were refrigerated in the dark until analyzed by confocal microscopy (Zeiss LSM 780 Confocal Microscope-Inverted Microscope; 25×/0.8 Imm Korr DIC M27; room temperature; Axiocam camera; Zen software), and Fiji was used for image processing.

### Cell Phenotype and Cytotoxicity Assays:

Anti-CD1a antibodies (5 μg/ml) and/or commercially available comparator NA1/34 (5 μg/ml) were incubated with CD1a expressing K562 or EV control K562 for 48 hours and cell reduction assessed by flow cytometry. To measure direct antibody induced cell reduction, K562 were fluorescently labelled with CellTraceViolet prior to incubation with anti-CD1a antibodies for 48 hours. Prior to assessment of reduction by flow cytometry, a reference population of untreated CFSE labelled K562 was added to the antibody-treated K562 in a 1:1 ratio. The percentage of induced reduction was then calculated with the following equation by comparing the frequency of live cells of the different populations analysed, antibody treated and untreated reference CD1a+ and EV K562. % reduction=100−((% live cells of antibody-treated CD1a-K562/% live cells of reference CFSE labelled K562)/(% live cells of untreated CD1a-K562/% live cells of reference CFSE labelled K562)×100). To examine effects of anti-CD1a antibodies on apoptosis of CD1a-expressing cells, K562-CD1a or K562-EV were incubated with either isotype control or anti-CD1a antibodies (5 μg/ml) and stained for Annexin-V (Biolegend) 24 hours after incubation.

### Complement-Mediated Lysis (CDC) and Antibody-Dependent Cytotoxicity ADCC Assays:

For CDC assays, K562-CD1a cells (5×10⁴ cells per well) were pre-treated with either 5 μg/ml isotype control antibody or indicated antibodies for 30 minutes and incubated with 10% normal human serum for 3-hours at 37° C. in 5% CO₂. For ADCC assays, PBMCs were used. K562-CD1a cells (5×10³ cells per well) were co-cultured with PBMCs (2.5×10⁵ cells per well) for 5 h at 37° C. in 5% CO₂ with IL-2 (100 U/ml) in combination of either 5 μg/ml isotype control antibody or indicated antibodies (an effector/target ratio of 50:1). Cytotoxicity was determined by calculating the percentage of survived target K562-CD1a using the following equation: % cytotoxicity=100−((% live cells of CD1a-antibody-treated CD1a-K562/% live reference K562)/(% live cells of isotype-antibody-treated CD1a-K562/% live reference K562)×100).

### In Vivo CD1a+ Cell Depletion:

“NSG” (NOD-scid IL2Rgamma^{null}) mice were subcutaneously injected with 0.25 million CD1a-K562 cells in ECM gel (Merck) suspension (vol=100 l) to the flank and tumours were allowed to develop for 18 days. Mice were treated with 100 μg isotype control antibody or indicated antibodies on days 6, 10, and 14 intraperitoneally, and tumour size was measured.

### Isoelectric Focusing Assay (IEF):

Lipid loading was assessed by incubating 10 g of CD1a with a 100× molar excess of imiquimod (Invivogen) solubilized in Tris Buffer saline and 2% CHAPS 7% DMSO or vehicle alone (mock) for 2 h at 37° C. and overnight at room temperature. CD1a samples were separated by isoelectric focusing (IEF). Briefly, CD1a-imiquimod and CD1a-mock proteins were loaded on an IEF pH 3-7 gel (Novex) that was then run for 1 hour at 100V, 1 hour and 200V and finally 30 mins at 500V. The gel was then fixed with 12% TCA and stained with SimplyBlue SafeStain for 7 minutes and destained in DI water overnight.

### Statistical Analysis:

The one and two-way ANOVA tests were performed using GraphPad Prism version 6.00 (GraphPad Software). Error bars represent standard deviation as indicated.

### Generation and Selection of Therapeutic Anti-CD1a Antibodies 77A (VR11851), 110 (VR12112), 111 (VR12113), 116 (VR12117) and 16 (VR11834)

A number of animals across different species (including mice and rabbits) were immunized. Mice were immunized with NIH3T3 cells transfected with human CD1a and mouse B2M. Rabbits were immunized with Rab9 cells transfected with human CD1a and rabbit B2M. Following 3-5 shots, the animals were sacrificed and PBMC, spleen, bone marrow and lymph nodes harvested. Sera was monitored for binding to HEK-293 cells expressing human CD1a and human B2M via flow cytometry.

Memory B cell cultures (relevant for 77A (VR11851), 110 (VR12112), 111 (VR12113) and 116 (VR12117)) were set up and supernatants were first screened for their ability to bind HEK-293 cells transiently transfected with human CD1a in a bead-based assay on the TTP Labtech Mirrorball system. This was a multiplex assay using HEK-293 cells expressing human CD1a and human B2M stained with a cellular dye and counter-screened against counter-stained HEK-293 cells expressing CD1b, CD1c or CD1d with human B2M, using a goat anti-species Fc-FITC conjugate as a reveal agent.

Approx. 3500 CD1a-specific positive hits were identified in the primary Mirrorball screens from a total of 10×200-plate B culture experiments. Positive supernatants from this assay were then progressed for further characterization by:
ELISA, to confirm binding to human CD1a protein (details below)
ELISA, to confirm binding to the CD1a lipid binding domain on chimeric CD1a protein (human lipid binding domain of CD1a, mouse Ig domain of CD1d) (details below)
Flow cytometry, to confirm binding to human CD1a expressed on HEK-293 cells (co-expressed with human 02M) (details below)

Wells demonstrating binding in the above assays were progressed for V region recovery using the fluorescent foci method. Plasma cells from bone marrow were also directly screened for their ability to bind human CD1a using the fluorescent foci method (relevant for 16 (VR11834)). Here, B cells secreting CD1a-specific antibodies were picked on biotinylated human CD1a immobilised on streptavidin beads using a goat anti-species Fc-FITC conjugate reveal reagent. Approx. 300 direct foci were picked.

Following reverse transcription (RT) and PCR of the picked cells, ‘transcriptionally active PCR’ (TAP) products encoding the antibodies' V regions were generated and used to transiently transfect HEK-293 cells. The resultant TAP supernatants, containing recombinant antibody, were further characterized by;
ELISA, to confirm binding to human CD1a protein and chimeric CD1a protein (human lipid binding domain of CD1a, mouse Ig domain of CD1d) (details below)
Flow cytometry, to confirm binding to human CD1a expressed on HEK-293 cells (co-expressed with human 02M) and counter-screen for cross-binding to relevant similar proteins: CD1b, CD1c or CD1d expressed on HEK-293 cells (co-expressed with human 02M). (details below)

Heavy and light chain variable region gene pairs from interesting TAP products were then cloned as either rabbit or mouse full length antibodies and re-expressed in a HEK-293 transient expression system. In total 119 V regions were cloned and registered. Recombinant cloned antibodies were then further characterized by:
Repeats of the above flow cytometry and ELISA assays.
Flow cytometry, to assess binding to CD1a expressed in multiple cell lines. This gave an initial indication that binding was lipid independent. Supernatants were screened for binding to:
   Stably transduced C1R cells expressing CD1a or empty vector (co-expressed with human β2M). These are relevant for 110 (VR12112), 111 (VR12113) and 116 (VR12117). (details below)
   MOLT4 cells endogenously expressing CD1a, CD1b, CD1c, CD1d and 02M. These are relevant for 110 (VR12112), 111 (VR12113) and 116 (VR12117). (details below)
Profiling in BIAcore to estimate off-rate and affinity (details below)

Antibodies demonstrating binding in the above assays and <100 nM affinity were selected for purification. Cell culture supernatants were purified using Protein A affinity purification. Purified samples were buffer exchanged in to 10 mM PBS pH 7.4 and analysed for its recovery and purity using UV spectroscopy, analytical size exclusion chromatography, SDS Page electrophoresis and LAL endotoxin assay respectively. Where required samples were subject to second round of purification to increase the monomer levels. Final samples were sterile filtered and stored in 10 mM PBS pH 7.4

Following purification, all 5 antibodies were then further characterized by:
Repeats of the above flow cytometry, ELISA and BIAcore assays
ELISA, to assess binding to Cynomolgus monkey CD1a protein and the variant of human CD1a protein common in China (18) (details below)
Flow cytometry, to assess binding to HEK-293 cells transiently transfected with: (details below)
   Cynomolgus monkey CD1a co-transfected with Cynomolgus monkey β2M
   The variant of human CD1a common in China co-transfected with human β2M

77A (VR11851), 110 (VR12112), 111 (VR12113), 116 (VR12117) and 16 (VR11834) demonstrated the capacity to bind to all tested forms of recombinant and cell expressed CD1a proteins at the respective stages of antibody discovery (Tables 1-9). The only exception was 116 (VR12117) which showed no binding to recombinant or cell expressed Cynomolgus CD1a (Table 4 and 9). Inclusion of antibody 116 in the subsequent in vitro and in vivo analyses was not considered obvious but was nevertheless a deliberate step in order to focus on epitope binding regions where the lipid-binding domain differs from human and cynomolgus with potentially different functional effects. None of the antibodies demonstrated binding to CD1b, CD1c or CD1d expressed on HEK-293 cells (Table 5), indicating these antibodies are CD1a-specific. CD1a, CD1b, CD1c and CD1d expression in HEK-293 cells was confirmed with commercially available antibodies, supporting this conclusion (data not shown). Binding to CD1a expressed on multiple cell types (HEK, C1R and MOLT4) gave an initial indication that antibody binding may be lipid-independent as CD1a is likely loaded from a different pool of lipids in each cell line.

Following antibody discovery, the antibodies were assessed for in vitro function in T cell assays as below.

DNA encoding the heavy and light chain V-regions of 77A (VR11851), 110 (VR12112), 111 (VR12113) and 116 (VR12117) on a mouse IgG1 backbone was synthesized at ATUM and expressed in a HEK-293 transient expression system in house. The antibodies then underwent purification and endotoxin removal and were tested in in vivo assays, as below.

### Affinity of 77A (VR11851), 110 (VR12112), 111 (VR12113), 116 (VR12117) and 16 (VR11834) for Human CD1a

The affinity of the purified antibodies to human CD1a was assessed using a Biacore T200 instrument (GE Healthcare) by capturing the antibody to an immobilized anti-species IgG F(ab′)2 followed by titration of human CD1a. Affinipure Goat anti-species IgG-F(ab′)2 fragment specific (Jackson ImmunoResearch) was immobilized on a CM5 Sensor Chip (GE Healthcare) via amine coupling chemistry to a capture level of ˜5000 response units (RUs). HBS-EP+ buffer (10 mM HEPES pH 7.4, 0.15 M NaCl, 3 mM EDTA, 0.05% Surfactant P20, GE Healthcare) was used as the running buffer with a flow rate of 10 μL/min. A 10 μL injection of test antibody at 0.5 μg/mL was used for capture by the immobilized Goat Anti-species Fab. Human CD1a was titrated over the captured antibodies (at 0 nM, 0.6 nM, 1.8 nM, 5.5 nM, 16.6 nM and 50 nM, diluted in running buffer) at a flow rate of 30 μL/min to assess affinity.

The surface was regenerated between cycles by injection of 2×10 μL of 40 mM HCl, interspersed by a 10 μL injection of 5 mM NaOH at flowrate of 10 μL/min. Background subtraction binding curves were analyzed using the Biacore T200 evaluation software following standard procedures. Kinetic parameters were determined from the fitting algorithm. This assay was performed at the clone supernatant and purified antibody stage. The kinetic parameters of antibody binding to human CD1a are shown in Table 10.

### Binding of 77A (VR11851), 110 (VR12112), 111 (VR12113), 116 (VR12117) and 16 (VR11834) Assessed by ELISA

CD1a-specific antibodies were identified by ELISA. ELISA plates were coated with 2 μg/mL protein of interest (human CD1a pool B, chimeric CD1a pool B [human lipid binding domain and mouse CD1d Ig domain], Chinese variant CD1a or Cynomolgus CD1a) (20 μL/well) at 4° C. overnight and then washed with wash buffer (0.2% (v/v) Tween-20 in PBS (pH7.4). Plates were then blocked with 80 μl/well block buffer (1% (w/v) bovine serum albumin) for 1 hour at room temperature and then washed in wash buffer. 20 μL antibody sample (B cell culture supernatant, TAP supernatant, clone supernatant, purified antibody solution) dilutions was transferred to the ELISA plates and incubated at room temperature for 1 hour, followed by washing with wash buffer. 20 μl/well of peroxidase-conjugated goat anti-species IgG Fc-specific F(ab′)2 fragment (Jackson ImmunoResearch), diluted 1:5000 in block buffer was added and incubated at room temperature for 1 hour, followed by washing with wash buffer. TMB substrate (EMD Millipore) was added (20 μL/well) to visualize binding, and the reaction incubated at room temperature for 5 minutes before measuring the optical density at 630 nM using a microplate reader. This assay was performed at the B-cell supernatant stage (human CD1a pool B), TAP supernatant stage (human CD1a pool B, chimeric CD1a pool B), clone supernatant stage (human CD1a pool B, chimeric CD1a pool B) and purified antibody stage (human CD1a pool B, chimeric CD1a pool B, Chinese variant CD1a, Cynomolgus CD1a). Data for purified antibodies shown in Tables 1-4.

### Binding of 77A (VR11851), 110 (VR12112), 111 (VR12113), 116 (VR12117) and 16 (VR11834) Assessed by Flow Cytometry

CD1a-specific antibodies were identified by flow cytometry. Binding to proteins expressed on HEK, CIR and MOLT4 cell lines was assessed. HEK-293 cells were transfected with a protein of interest (CD1a, CD1b, CD1c, CD1d, Chinese variant CD1a or Cynomolgus CD1a) and the species-specific 02M (as indicated above). The transfections were performed using the Expifectamine 293 kit (Gibco) and incubated overnight. The C1R-CD1a, C1R-empty vector and MOLT4 cell lines were washed in 1×PBS on the day required. All cell lines were counted and resuspended in 1×PBS and then stained for 30 minutes at 37° C. using the DiI or DiO cellular stains (Invitrogen). Cells were washed with flow cytometry buffer (1% bovine serum albumin, 2 mM EDTA and 0.1% sodium azide in PBS) before mixing 2 DiI-stained and DiO-stained populations together. The cells (20 μl/well) were then added to dilutions of antibody sample (B cell culture supernatant, TAP supernatant, clone supernatant, purified antibody solution) (20 μl/well) and incubated for 1 hour at 4° C. in a flow cytometry assay plate, before being washed with flow cytometry buffer. 10 μl/well of Alexafluor647-conjugated goat anti-species IgG Fc-specific F(ab′)2 fragment (Jackson ImmunoResearch), diluted 1:2500 in flow cytometry buffer, was added and incubated at 4° C. for 30 minutes, followed by washing with wash buffer. The fluorescence intensity was then measured on an iQue screener PLUS. This assay was performed at the B-cell supernatant stage (HEK-293 cells expressing human CD1a), TAP supernatant stage (HEK-293 cells expressing human CD1a, CD1b, CD1c or CD1d), clone supernatant stage (HEK-293 cells expressing human CD1a, CD1b, CD1c or CD1d; C1R cells expressing human CD1a or empty vector; MOLT4 cell line) and purified antibody stage (HEK-293 cells expressing human CD1a, CD1b, CD1c, CD1d, Chinese variant CD1a or Cynomolgus CD1a; C1R cells expressing human CD1a or empty vector; MOLT4 cells). Data for purified antibodies is shown in Tables 5-9.

TABLE 1
Antibody binding to human CD1a pool B protein. 77A (VR11851), 110 (VR12112),
111 (VR12113), 116 (VR12117) and 16 (VR11834) were tested for their ability
to bind human CD1a protein in an ELISA. The antibodies were titrated through
a dilution series and compared to a control rabbit IgG antibody. All 5 antibodies
bound to human CD1a pool B protein. Data shown for purified antibodies.

| | Optical Density (OD) | | | | | | | |
|---|---|---|---|---|---|---|---|---|
| Antibody | 100 | 31.6 | 10 | 3.16 | 1 | 0.316 | 0.1 | 0.0316 |
| Concentration | μg/ml | μg/ml | μg/ml | μg/ml | μg/ml | μg/ml | μg/ml | μg/ml |
| 77A (VR11851) | 1.39 | 1.28 | 1.34 | 1.33 | 1.34 | 1.39 | 1.36 | 1.25 |
| 110 (VR12112) | 1.23 | 1.24 | 1.28 | 1.27 | 1.19 | 1.18 | 1.12 | 0.81 |
| 111 (VR12113) | 1.12 | 1.07 | 1.12 | 1.13 | 1.14 | 1.04 | 1.06 | 1.10 |
| 116 (VR12117) | 1.15 | 1.16 | 1.18 | 1.15 | 1.15 | 1.15 | 1.07 | 0.95 |
| 16 (VR11834) | NA | NA | NA | NA | NA | NA | 0.59 | NA |
| Control IgG | 0.07 | 0.06 | 0.07 | 0.06 | 0.07 | 0.07 | 0.06 | 0.06 |

TABLE 2
Antibody binding to chimeric CD1a pool B protein. 77A (VR11851), 110 (VR12112),
111 (VR12113), 116 (VR12117) and 16 (VR11834) were tested for their ability
to bind chimeric CD1a [human CD1a lipid binding domain, mouse CD1d Ig domain]
protein in an ELISA. The antibodies were titrated through a dilution series
and compared to a control rabbit IgG antibody. All 5 antibodies bound to chimeric
CD1a pool B protein. Data shown for purified antibodies.

| | Optical Density (OD) | | | | | | | |
|---|---|---|---|---|---|---|---|---|
| Antibody | 100 | 31.6 | 10 | 3.16 | 1 | 0.316 | 0.1 | 0.0316 |
| Conc. (μg/ml | μg/ml | μg/ml | μg/ml | μg/ml | μg/ml | μg/ml | μg/ml | μg/ml |
| 77A (VR11851) | 1.27 | 1.33 | 1.31 | 1.28 | 1.26 | 1.28 | 1.25 | 1.09 |
| 110 (VR12112) | 1.40 | 1.40 | 1.46 | 1.53 | 1.45 | 1.24 | 1.02 | 0.73 |
| 111 (VR12113) | 1.27 | 1.35 | 1.40 | 1.41 | 1.36 | 1.32 | 1.25 | 1.19 |
| 116 (VR12117) | 1.37 | 1.35 | 1.40 | 1.38 | 1.42 | 1.32 | 1.25 | 1.13 |
| 16 (VR11834) | NA | NA | NA | NA | NA | NA | 0.70 | NA |
| Control IgG | 0.08 | 0.08 | 0.08 | 0.10 | 0.09 | 0.09 | 0.08 | 0.09 |

TABLE 3
Antibody binding to Chinese variant CD1a protein. 77A (VR11851),
110 (VR12112), 111 (VR12113), 116 (VR12117) and 16 (VR11834)
were tested for their ability to bind Chinese variant CD1a
protein in an ELISA. The antibodies were titrated through
a dilution series and compared to a control rabbit IgG
antibody. All 5 antibodies bound to Chinese variant CD1a
protein. Data shown for purified antibodies.

| Antibody | Optical Density (OD) | | | |
|---|---|---|---|---|
| Concentration | 10 μg/ml | 1 μg/ml | 0.1 μg/ml | 0.01 μg/ml |
| 77A | 0.99 | 0.78 | 1.05 | 0.79 |
| (VR11851) | | | | |
| 110 | 1.75 | 1.79 | 1.57 | 1.40 |
| (VR12112) | | | | |
| 111 | 1.41 | 1.44 | 1.52 | 1.33 |
| (VR12113) | | | | |
| 116 | 1.51 | 1.51 | 1.53 | 1.47 |
| (VR12117) | | | | |
| 16 | 1.44 | 1.35 | 1.30 | 1.01 |
| (VR11834) | | | | |
| Control IgG | 0.14 | 0.08 | 0.08 | 0.08 |

TABLE 4
Antibody binding to Cynomolgus monkey CD1a protein. 77A
(VR11851), 110 (VR12112), 111 (VR12113), 116 (VR12117)
and 16 (VR11834) were tested for their ability to bind
Cynomolgus CD1a protein in an ELISA. The antibodies
were titrated through a dilution series and compared
to a control rabbit IgG antibody. All 5 antibodies,
except 116 (VR12117), bound to Cynomolgus monkey CD1a
protein. Data shown for purified antibodies.

| Antibody | Optical Density (OD) | | | |
|---|---|---|---|---|
| Concentration | 10 μg/ml | 1 μg/ml | 0.1 μg/ml | 0.01 μg/ml |
| 77A | 0.52 | 0.43 | 0.24 | 0.10 |
| (VR11851) | | | | |
| 110 | 0.86 | 0.94 | 0.53 | 0.22 |
| (VR12112) | | | | |
| 111 | 0.79 | 0.69 | 0.66 | 0.48 |
| (VR12113) | | | | |
| 116 | 0.16 | 0.14 | 0.07 | 0.07 |
| (VR12117) | | | | |
| 16 | 0.49 | 0.58 | 0.57 | 0.46 |
| (VR11834) | | | | |
| Control IgG | 0.09 | 0.07 | 0.07 | 0.06 |

TABLE 5
Antibody binding to human CD1a, CD1b, CD1c or CD1d expressed on HEK-293
cells. HEK-293 cells were transiently transfected with human CD1a, CD1b,
CD1c or CD1d and co-transfected with human β2M. 77A (VR11851), 110 (VR12112),
111 (VR12113), 116 (VR12117) and 16 (VR11834) were titrated through a dilution
series and tested for binding to the transfected proteins. Binding was
quantified as fold change in fluorescence intensity geomean over background
assessed by flow cytometry. All 5 antibodies bound to human CD1a expressed
on HEK-293 cells. No binding to CD1b, CD1c or CD1d expressed on HEK-293
cells was observed. Data shown for purified antibodies.

| | | Fluorescence Intensity Geomean (normalized to background) | | | | | | | |
|---|---|---|---|---|---|---|---|---|---|
| Target | Antibody | 100 | 31.6 | 10 | 3.16 | 1 | 0.316 | 0.1 | 0.0316 |
| cell | Concentration | μg/ml | μg/ml | μg/ml | μg/ml | μg/ml | μg/ml | μg/ml | μg/ml |
| HEK - | 77A (VR11851) | 4.1 | 8.2 | 13.7 | 23.3 | 41.9 | 58.6 | 43.8 | 24.9 |
| CD1a | 110 (VR12112) | 5.9 | 10.9 | 15.7 | 31.5 | 49.2 | 38.3 | 20.2 | 8.9 |
| | 111 (VR12113) | 4.3 | 6.3 | 11.1 | 24.9 | 42.0 | 30.8 | 23.1 | 11.1 |
| | 116 (VR12117) | 3.9 | 5.6 | 12.6 | 21.9 | 48.2 | 36.5 | 26.7 | 15.7 |
| | 16 (VR11834) | NA | NA | 10.2 | NA | 19.6 | NA | 12.9 | NA |
| HEK - | 77A (VR11851) | 1.0 | 1.0 | 1.1 | 0.9 | 1.1 | 1.1 | 1.0 | 0.9 |
| CD1b | 110 (VR12112) | 1.1 | 1.1 | 1.1 | 1.1 | 1.1 | 1.0 | 1.0 | 1.0 |
| | 111 (VR12113) | 1.1 | 1.0 | 1.1 | 1.1 | 1.0 | 1.0 | 1.0 | 1.0 |
| | 116 (VR12117) | 1.1 | 1.0 | 1.0 | 1.0 | 1.1 | 1.0 | 1.0 | 1.0 |
| | 16 (VR11834) | NA | NA | NA | NA | NA | NA | NA | NA |
| HEK - | 77A (VR11851) | 0.9 | 0.9 | 0.9 | 0.9 | 1.0 | 1.0 | 0.9 | 1.0 |
| CD1c | 110 (VR12112) | 1.0 | 1.0 | 1.0 | 1.1 | 1.1 | 0.9 | 1.0 | 1.0 |
| | 111 (VR12113) | 1.1 | 1.0 | 1.0 | 1.1 | 1.1 | 1.0 | 1.0 | 1.0 |
| | 116 (VR12117) | 1.0 | 1.0 | 1.1 | 1.1 | 1.1 | 1.1 | 1.0 | 1.0 |
| | 16 (VR11834) | NA | NA | NA | NA | NA | NA | NA | NA |
| HEK - | 77A (VR11851) | 1.0 | 1.0 | 1.0 | 0.9 | 1.0 | 1.0 | 0.9 | 0.9 |
| CD1d | 110 (VR12112) | 1.1 | 1.1 | 1.0 | 1.0 | 1.1 | 1.0 | 1.0 | 1.0 |
| | 111 (VR12113) | 1.1 | 1.0 | 1.1 | 1.1 | 1.1 | 0.9 | 1.0 | 1.0 |
| | 116 (VR12117) | 1.0 | 1.0 | 1.1 | 1.1 | 1.0 | 1.0 | 1.1 | 1.0 |
| | 16 (VR11834) | NA | NA | NA | NA | NA | NA | NA | NA |

TABLE 6
Antibody binding to human CD1a, CD1b, CD1c or CD1d expressed on C1R cells.
77A (VR11851), 110 (VR12112), 111 (VR12113), 116 (VR12117) and 16 (VR11834)
were titrated through a dilution series and tested for binding to C1R
cells stably transduced with human CD1a or empty vector and human β2M.
Binding was quantified as fold change in fluorescence intensity geomean
over background assessed by flow cytometry. All 5 antibodies bound to
human CD1a expressed on C1R cells. No binding to C1R cells expressing
empty vector was observed. Data shown for purified antibodies.

| | | Fluorescence Intensity Geomean (normalized to background) | | | | | | | |
|---|---|---|---|---|---|---|---|---|---|
| Target | Antibody | 100 | 31.6 | 10 | 3.16 | 1 | 0.316 | 0.1 | 0.0316 |
| cell | Concentration | μg/ml | μg/ml | μg/ml | μg/ml | μg/ml | μg/ml | μg/ml | μg/ml |
| C1R - | 77A (VR11851) | 0.9 | 1.3 | 2.3 | 4.2 | 10.3 | 19.0 | 16.4 | 17.0 |
| CD1a | 110 (VR12112) | 4.8 | 6.2 | 8.3 | 15.2 | 28.2 | 29.7 | 25.8 | 17.1 |
| | 111 (VR12113) | 7.3 | 7.0 | 11.7 | 15.6 | 28.0 | 28.8 | 25.4 | 26.0 |
| | 116 (VR12117) | 11.9 | 13.7 | 16.9 | 24.0 | 28.7 | 27.5 | 27.0 | 24.1 |
| | 16 (VR11834) | NA | NA | 7.0 | NA | 10.1 | NA | 10.2 | NA |
| C1R - | 77A (VR11851) | 1.0 | 1.0 | 1.0 | 1.0 | 1.0 | 1.0 | 1.0 | 0.9 |
| Empty | 110 (VR12112) | 1.0 | 0.8 | 0.9 | 1.0 | 0.9 | 0.8 | 0.9 | 0.4 |
| Vector | 111 (VR12113) | 1.1 | 1.0 | 1.0 | 1.0 | 0.9 | 0.9 | 1.0 | 1.2 |
| | 116 (VR12117) | 1.0 | 1.0 | 1.2 | 1.4 | 1.3 | 1.0 | 1.0 | 0.9 |
| | 16 (VR11834) | NA | NA | 0.9 | NA | 0.9 | NA | 0.9 | NA |

TABLE 7
Antibody binding to MOLT4 cells. 77A (VR11851), 110 (VR12112), 111
(VR12113), 116 (VR12117) and 16 (VR11834) were titrated through a
dilution series and tested for binding to MOLT4 cells which endogenously
express CD1a, CD1b, CD1c, CD1d and β2M. Binding was quantified
as fold change in fluorescence intensity geomean over background assessed
by flow cytometry. All 5 antibodies bound to MOLT4 cell surface proteins,
most likely CD1a. Data shown for purified antibodies.

| | Fluorescence Intensity Geomean (normalized to background) | | | | | | | |
|---|---|---|---|---|---|---|---|---|
| Antibody | 100 | 31.6 | 10 | 3.16 | 1 | 0.316 | 0.1 | 0.0316 |
| Concentration | μg/ml | μg/ml | μg/ml | μg/ml | μg/ml | μg/ml | μg/ml | μg/ml |
| 77A (VR11851) | 0.8 | 2.2 | 4.1 | 7.4 | 13.5 | 15.2 | 12.4 | 8.7 |
| 110 (VR12112) | 5.2 | 7.5 | 14.7 | 22.6 | 33.0 | 27.1 | 16.8 | 1.6 |
| 111 (VR12113) | 3.9 | 5.4 | 9.3 | 18.9 | 31.2 | 27.3 | 20.6 | 12.4 |
| 116 (VR12117) | 3.7 | 5.6 | 11.7 | 23.9 | 30.6 | 29.6 | 22.6 | 14.9 |
| 16 (VR11834) | NA | NA | 7.5 | NA | 15.0 | NA | 14.4 | NA |

TABLE 8
Antibody binding to a common Chinese variant CD1a expressed
on HEK-293 cells. 77A (VR11851), 110 (VR12112), 111 (VR12113),
116 (VR12117) and 16 (VR11834) were titrated through a dilution
series and tested for binding to HEK-293 cells transiently
transfected with a common Chinese variant CD1a (18) and human
β2M. Binding was quantified as fold change in fluorescence
intensity geomean over background assessed by flow cytometry.
All 5 antibodies bound to Chinese variant CD1a expressed on
HEK-293 cells. Data shown for purified antibodies.

| | Fluorescence Intensity Geomean | | | |
|---|---|---|---|---|
| Antibody | (normalized to background) | | | |
| Concentration | 10 μg/ml | 1 μg/ml | 0.1 μg/ml | 0.01 μg/ml |
| 77A | 6.9 | 47.2 | 99.3 | 76.4 |
| (VR11851) | | | | |
| 110 | 48.3 | 111.5 | 119.1 | 29.1 |
| (VR12112) | | | | |
| 111 | 35.4 | 39.5 | 96.4 | 33.8 |
| (VR12113) | | | | |
| 116 | 48.9 | 12.8 | 100.0 | 34.5 |
| (VR12117) | | | | |
| 16 (VR11834) | 11.4 | 11.2 | 11.2 | 6.0 |

TABLE 9
Antibody binding to Cynomolgus monkey CD1a expressed on HEK-293
cells. 77A (VR11851), 110 (VR12112), 111 (VR12113), 116 (VR12117)
and 16 (VR11834) were titrated through a dilution series and
tested for binding to HEK-293 cells transiently transfected
with Cynomolgus monkey CD1a and Cynomolgus monkey β2M. Binding
was quantified as fold change in fluorescence intensity geomean
over background assessed by flow cytometry. All 5 antibodies,
except 116 (VR12117), bound to Cynomolgus monkey CD1a expressed
on HEK-293 cells. Data shown for purified antibodies.

| | Fluorescence Intensity Geomean | | | |
|---|---|---|---|---|
| Antibody | (normalized to background) | | | |
| Concentration | 10 μg/ml | 1 μg/ml | 0.1 μg/ml | 0.01 μg/ml |
| 77A | 1.4 | 10.6 | 5.7 | 1.7 |
| (VR11851) | | | | |
| 110 | 13.6 | 11.2 | 3.3 | 1.2 |
| (VR12112) | | | | |
| 111 | 16.3 | 12.9 | 25.8 | 8.9 |
| (VR12113) | | | | |
| 116 | 1.7 | 0.8 | 1.0 | 1.0 |
| (VR12117) | | | | |
| 16 (VR11834) | 8.8 | 7.6 | 8.5 | 6.9 |

TABLE 10
Antibody affinity for human CD1a. The affinity of 77A (VR11851), 110
(VR12112), 111 (VR12113), 116 (VR12117) and 16 (VR11834) for human
CD1a was assessed using biacore. The 1:1 binding model was used to
fit the data in all cases, except 16 (VR11834) which required the heterogenous
ligand binding model. Affinity was required to be <100 nM to be
considered for progression. Data shown for purified antibodies.

| Ligand | ka (1/Ms) | ka 2 (1/Ms) | kd (1/s) | kd 2 (1/s) | KD (M) |
|---|---|---|---|---|---|
| 77A (VR11851) | 4.69E+05 | | 1.13E−04 | | 2.40E−10 |
| 110 (VR12112) | 4.43E+05 | | 8.74E−04 | | 1.97E−09 |
| 111 (VR12113) | 3.21E+05 | | 1.47E−08 | | 3.12E−11 |
| 116 (VR12117) | 4.83E+05 | | 1.40E−03 | | 2.89E−09 |
| 16 (VR11834) | 1.86E+06 | 5.14E+05 | 7.29E−04 | 6.58E−03 | 3.92E−10 |

| | KD2 | Chi² | Ligand Level | | Rmax |
|---|---|---|---|---|---|
| Ligand | (M) | (RU²) | (RU) | Model | (RU) |
| 77A | | 0.306 | 177.2 | 1:1 Binding | 118.5 |
| (VR11851) | | | | | |
| 110 | | 3.94E−02 | 128.1 | 1:1 Binding | 24.2 |
| (VR12112) | | | | | |
| 111 | | 2.48E−02 | 68.5 | 1:1 Binding | Local Fit |
| (VR12113) | | | | | |
| 116 | | 1.19E−01 | 120.3 | 1:1 Binding | 49.0 |
| (VR12117) | | | | | |
| 16 | 1.28E−08 | NA | 78.1 | Heterogeneous | NA |
| (VR11834) | | | | Ligand | |

### Antibody Production

For the surface plasmon resonance and crystallisation studies, single chain variable regions (ScFv) were generated for OX16, OX110 and OX116 using a flexible glycine-serine linker between the heavy and light chains. For the functional assays in vitro, a number of constructs were generated as previously described, including the existing mouse or rabbit variable regions on human IgG1 Fc region as: wild-type; Leu234Ala, Leu235Ala and Gly237Ala “LALAGA”; afucosylated; as well as Fab versions. These were established for OX16, OX110, OX116 as well as comparator antibodies CR2113 and mAB571 (U.S. Ser. No. 10/844,118 and WO/2022/077021). All were expressed in a HEK-293 transient expression system in-house and the antibodies then underwent purification and endotoxin removal.

### Crystallisation

Human CD1a/β2m heterodimer carrying fos-jun zippers was expressed in HEK293S cells and purified by nickel affinity and size exclusion chromatography steps. CD1a was deglycosylated using EndoH (NEB) and fos-jun zippers, BirA tag and His-tag were cleaved off using thrombin overnight at room temperature. In the case of OX scFvs BirA and His-tag were cleaved off using 3C protease overnight at 4 C. CD1a protein used in crystallisation trials contained a heterogenous mixture of lipids derived from the expression system (CD1a-endo). Monoclonal antibody fragments were expressed as scFv constructs in suspension HEK293F cells and were purified by nickel and size exclusion chromatography. CD1a and antibody fragments were mixed at 1:1 molar ratio and incubated overnight at 4 C. Sitting drop crystallisation trials were performed at Monash Macromolecular Crystallisation Facility and the sample concentration used in each case was within 5-10 mg/ml range. Initial hits were further optimised in hand trays by hanging drop method. Crystals of OX16-CD1a appeared in 0.2M sodium malonate, 20% PEG3350 and diffracted up to 3.2 Å. Crystals of OX110-CD1a were obtained in 0.1M MES pH 6, 20% PEG 8000, 0.2M sodium acetate and diffracted up to 3.4 Å. Crystals of OX116-CD1a grew in 1.5M Ammonium sulfate, 0.1M Bis-Tris pH 6 and diffracted up to 2.7 Å. In each case the structures were solved by molecular replacement using CD1a binary structure (PDB: 6NUX) and an Alphafold-generated model of the corresponding antibody fragment. The structures were then refined by cycles of manual refinement in Coot followed by automated refinement in Phenix.

### Surface Plasmon Resonance

SPR experiments were performed on Biacore 3000 using Streptavidin coated chips (Cytiva). Antibody fragments were expressed with a biotinylation tag on their C-termini and were biotinylated overnight using BirA ligase. Depending on the experiment biotinylated scFv molecules were coupled onto the chip surface until a total of 150 or 1000 response units per flow cell were achieved. To assess the effect of lipid antigen headgroup on binding to OX16 and OX116 antibodies increasing concentration of deglycosylated CD1a-endo or CD1a loaded with a specific lipid were injected over each flow cell. Lipid loading of CD1a was performed as previously described (Cotton et al., J Exp Med. 5; 218:e20202699 (2021)). Briefly, lipids used were sphingomyelin (Avanti 860593), lyso-phosphatidylcholine (Avanti 845875), GD3 ganglioside (Avanti 860060), egg PG (Avanti 841138), sulfatide (Avanti 131305), phosphatidylcholine (Avanti 850375). Each lipid was solubilised up to 5-10 mM in 20 mM Tris pH8, 150 mM NaCl and 0.5% CHAPS. CD1a-endo was incubated at room temperature overnight with 15-40× molar excess of lipid. The mixture was subsequently purified by anion exchange chromatography using a MonoQ column (GE Healthcare). Fractions corresponding to lipid-loaded CD1a were pooled together and up-concentrated to 5 μM. Serial dilutions of CD1a up to a maximum concentration of 1 μM were injected for 60 s at 25 C in 20 mM Tris pH8, 150 mM NaCl buffer. The dissociation time between the injections was between 5 minutes up to 1 h. For binding of TCRs to CD1a-Ab complexes, 1000 response units of biotinylated OX116 were coupled onto SA Chip. Each injection cycle consisted of a 60 s injection of 1 μM CD1a alone immediately followed by increasing concentrations of TCRs (0 to 50 μM) supplemented with 100 nM CD1a to prevent further dissociation of CD1a from the coupled Ab fragment. The runs were performed at 25 C in 20 mM Tris pH8, 150 mM NaCl, 0.5% BSA buffer. In all SPR experiments relative binding response was calculated by subtracting the non-specific response on a reference cell, where an unrelated protein was coupled. The binding curves were obtained by fitting the measured response to a 1:1 specific binding model in GraphPad.

### Antibody Binding Detected by ELISA

ELISA plates were coated with 2 μg/mL protein of interest (human CD1a pool B, chimeric CD1a pool B [human lipid binding alpha 1/2 domains and mouse CD1d alpha 3 Ig domain], minor variant CD1a or Cynomolgus CD1a) (20 μL/well) at 4° C. overnight and then washed with wash buffer (0.2% (v/v) Tween-20 in PBS (pH7.4). Plates were then blocked with 80 μl/well block buffer (1% (w/v) bovine serum albumin) for 1 hour at room temperature and then washed in wash buffer. 20 μL antibody sample (B cell culture supernatant, TAP supernatant, clone supernatant, purified antibody solution) dilutions was transferred to the ELISA plates and incubated at room temperature for 1 hour, followed by washing with wash buffer. 20 μl/well of peroxidase-conjugated goat anti-species IgG Fc-specific F(ab′)2 fragment (Jackson ImmunoResearch), diluted 1:5000 in block buffer was added and incubated at room temperature for 1 hour, followed by washing with wash buffer. TMB substrate (EMD Millipore) was added (20 μL/well) to visualize binding, and the reaction incubated at room temperature for 5 minutes before measuring the optical density at 630 nM using a microplate reader.

### Antibody Binding Detected by Flow Cytometry

CD1a-specific antibodies were identified by flow cytometry. Binding to proteins expressed on HEK, CIR and MOLT4 cell lines was assessed. HEK-293 cells were transfected with a protein of interest (CD1a, CD1b, CD1c, CD1d, Chinese variant CD1a or Cynomolgus CD1a) and the species-specific 02M (as indicated above). The transfections were performed using the Expifectamine 293 kit (Gibco) and incubated overnight. The C1R-CD1a, C1R-empty vector and MOLT4 cell lines were washed in 1×PBS on the day required. All cell lines were counted and resuspended in 1×PBS and then stained for 30 minutes at 37° C. using the DiI or DiO cellular stains (Invitrogen). Cells were washed with flow cytometry buffer (1% bovine serum albumin, 2 mM EDTA and 0.1% sodium azide in PBS) before mixing 2 DiI-stained and DiO-stained populations together. The cells (20 μl/well) were then added to dilutions of antibody sample (B cell culture supernatant, TAP supernatant, clone supernatant, purified antibody solution) (20 μl/well) and incubated for 1 hour at 4° C. in a flow cytometry assay plate, before being washed with flow cytometry buffer. 10 μl/well of Alexafluor647-conjugated goat anti-species IgG Fc-specific F(ab′)2 fragment (Jackson ImmunoResearch), diluted 1:2500 in flow cytometry buffer, was added and incubated at 4° C. for 30 minutes, followed by washing with wash buffer. The fluorescence intensity was then measured on an iQue screener PLUS. This assay was performed at the B-cell supernatant stage (HEK-293 cells expressing human CD1a), TAP supernatant stage (HEK-293 cells expressing human CD1a, CD1b, CD1c or CD1d), clone supernatant stage (HEK-293 cells expressing human CD1a, CD1b, CD1c or CD1d; C1R cells expressing human CD1a or empty vector; MOLT4 cell line) and purified antibody stage (HEK-293 cells expressing human CD1a, CD1b, CD1c, CD1d, Chinese variant CD1a or Cynomolgus CD1a; C1R cells expressing human CD1a or empty vector; MOLT4 cells).

### Antibody Humanisation Methods for Antibody 77A (VR11851), 110 (VR12112), 111 (VR12113), 116 (VR12117) and 16 (VR11834)

Antibodies were humanised by grafting the CDRs from the rabbit and mouse antibody V-regions onto human germline antibody V-region frameworks. In order to recover the activity of the antibody, a number of framework residues from the rabbit and mouse V-regions were also retained in the humanised sequences. These residues were selected using the protocol outlined by Adair et al. (1991) (Humanised antibodies. WO91/09967). The CDRs grafted from the donor to the acceptor sequence are as defined by Kabat (Kabat et al., 1987), with the exception of CDRH1 where the combined Chothia/Kabat definition is used (see Adair et al., 1991 Humanised antibodies. WO91/09967). Commonly the VH genes of rabbit antibodies are shorter than the selected human VH acceptor genes. When aligned with the human acceptor sequences, framework 1 of the VH regions of rabbit antibodies typically lack the N-terminal residue, which is retained in the humanised antibody. Framework 3 of the rabbit antibody VH regions also typically lack one or two residues (75, or 75 and 76) in the loop between beta sheet strands D and E: in the humanised antibodies the gap is filled with the corresponding residues from the selected human acceptor sequence.

### Antibody 77A

Human V-region IGKV1-5 plus IGKJ4 J-region (IMGT, http://www.imgt.org/) was chosen as an acceptor for antibody 11851 light chain CDRs. In addition to the CDRs, 0, 1, 2, 3 or 4 of the following framework residues from the 11851 VK gene (donor residues) may be retained at positions 1, 2, 3 and 71 (Kabat numbering): Alanine (A1), Valine (V2), Glutamic acid (E3) and Tyrosine (Y71), respectively. In some cases, CDRL3 may be mutated to remove an un-paired Cysteine residue at position 90 (Kabat numbering) (C90, CDRL3 variants, SEQ ID NOs: X-Y).

Human V-region IGHV3-23 plus IGHJ5 J-region (IMGT, http://www.imgt.org/) was chosen as an acceptor for the heavy chain CDRs of antibody 11851. In addition to the CDRs, 0, 1, 2, 3, 4, 5, 6 or 7 of the following framework residues from the 11851 VH gene (donor residues) may be retained at positions 24, 48, 49, 71, 73, 78 and 94 (Kabat numbering): Valine (V24), Isoleucine (I48), Glycine (G49), Lysine (K71), Serine (S73), Valine (V78) and Arginine (R94), respectively.

### Antibody 110

Human V-region IGKV1-D13 plus IGKJ4 J-region (IMGT, http://www.imgt.org/) was chosen as an acceptor for antibody 12112 light chain CDRs. In addition to the CDRs, 0, 1, 2 or 3 of the following framework residues from the 12112 VK gene (donor residues) may be retained at positions 2, 3 and 70 (Kabat numbering): Glutamine (Q2), Valine (V3) and Glutamine (Q70), respectively. In some cases, CDRL3 may be mutated to remove a disulphide bond between Cysteine residues at positions 94 and 95d (Kabat numbering) (C94 and C95d, CDRL3 variants, SEQ ID NOs: X-Y).

Human V-region IGHV3-48 plus IGHJ2 J-region (IMGT, http://www.imgt.org/) was chosen as an acceptor for the heavy chain CDRs of antibody 12112. In addition to the CDRs, 0, 1, 2, 3, 4, 5 or 6 of the following framework residues from the 12112 VH gene (donor residues) may be retained at positions 24, 48, 49, 71, 73 and 78 (Kabat numbering): Valine (V24), Isoleucine (148), Glycine (G49), Lysine (K71), Serine (S73) and Valine (V78), respectively. In some cases, CDRH2 may be mutated to remove a potential N-linked glycosylation site (CDRH2 variants, SEQ ID NOs: X-Y). In some cases, CDRH3 may be mutated to modify a potential Aspartic Acid-Proline hydrolysis site (CDRH3 variants, SEQ ID NOs: X-Y).

### Antibody 111

Human V-region IGKV1-5 plus IGKJ4 J-region (IMGT, http://www.imgt.org/) was chosen as an acceptor for antibody 12113 light chain CDRs. In addition to the CDRs, 0, 1, 2, 3 or 4 of the following framework residues from the 12113 VK gene (donor residues) may be retained at positions 1, 2, 3 and 71 (Kabat numbering): Alanine (A1), Valine (V2), Glutamic acid (E3) and Tyrosine (Y71), respectively. In some cases, CDRL3 may be mutated to remove an un-paired Cysteine residue at position 90 (Kabat numbering) (C90, CDRL3 variants, SEQ ID NOs: X-Y).

Human V-region IGHV3-23 plus IGHJ2 J-region (IMGT, http://www.imgt.org/) was chosen as an acceptor for the heavy chain CDRs of antibody 12113. In addition to the CDRs, 0, 1, 2, 3, 4, 5 or 6 of the following framework residues from the 12113 VH gene (donor residues) may be retained at positions 48, 49, 71, 73, 78 and 94 (Kabat numbering): Isoleucine (148), Glycine (G49), Lysine (K71), Serine (S73), Valine (V78) and Arginine (R94), respectively.

### Antibody 116

Human V-region IGKV1-D13 plus IGKJ4 J-region (IMGT, http://www.imgt.org/) was chosen as an acceptor for antibody 12117 light chain CDRs. In addition to the CDRs, 0, 1, 2 or 3 of the following framework residues from the 12117 VK gene (donor residues) may be retained at positions 2, 3 and 70 (Kabat numbering): Glutamine (Q2), Valine (V3) and Glutamine (Q70), respectively. In some cases, CDRL1 may be mutated to modify a potential deamidation site (CDRL1 variants, SEQ ID NOs: X-Y). In some cases, CDRL3 may be mutated to remove a disulphide bond between Cysteine residues at positions 94 and 95d (Kabat numbering) (C94 and C95d, CDRL3 variants, SEQ ID NOs: X-Y).

Human V-region IGHV3-66 plus IGHJ4 J-region (IMGT, http://www.imgt.org/) was chosen as an acceptor for the heavy chain CDRs of antibody 12117. In addition to the CDRs, 0, 1, 2, 3, 4, 5 or 6 of the following framework residues from the 12117 VH gene (donor residues) may be retained at positions 24, 48, 49, 71, 73 and 78 (Kabat numbering): Valine (V24), Isoleucine (148), Glycine (G49), Lysine (K71), Serine (S73) and Valine (V78), respectively.

### Antibody 16

Human V-region IGKV1-39 plus IGKJ1 J-region (IMGT, http://www.imgt.org/) was chosen as an acceptor for antibody 11834 light chain CDRs. In addition to the CDRs, 0, 1, 2, 3 or 4 of the following framework residues from the 11834 VK gene (donor residues) may be retained at positions 48, 70, 71 and 85 (Kabat numbering): Valine (V48), Glutamine (Q70), Tyrosine (Y71) and Arginine (R85), respectively. In some cases, CDRL2 may be mutated to remove a potential Aspartic acid isomerisation site (CDRL 2 variants, SEQ ID NOs: X).

Human V-region IGHV3-23 plus IGHJ4 J-region (IMGT, http://www.imgt.org/) was chosen as an acceptor for the heavy chain CDRs of antibody 11834. In addition to the CDRs, 0, 1, 2 or 3 of the following framework residues from the 11834 VH gene (donor residues) may be retained at positions 44, 49 and 94 (Kabat numbering): Arginine (R44), Alanine (A49) and Arginine (R94), respectively. In some cases, CDRH2 may be mutated to modify two potential Asparagine deamidation sites (CDRH2 variants, SEQ ID NOs: X-Y).

### EXAMPLES

### Example 1—Anti-CD1a Panel Refinement: Functional Assessment of Anti-CD1a Antibodies

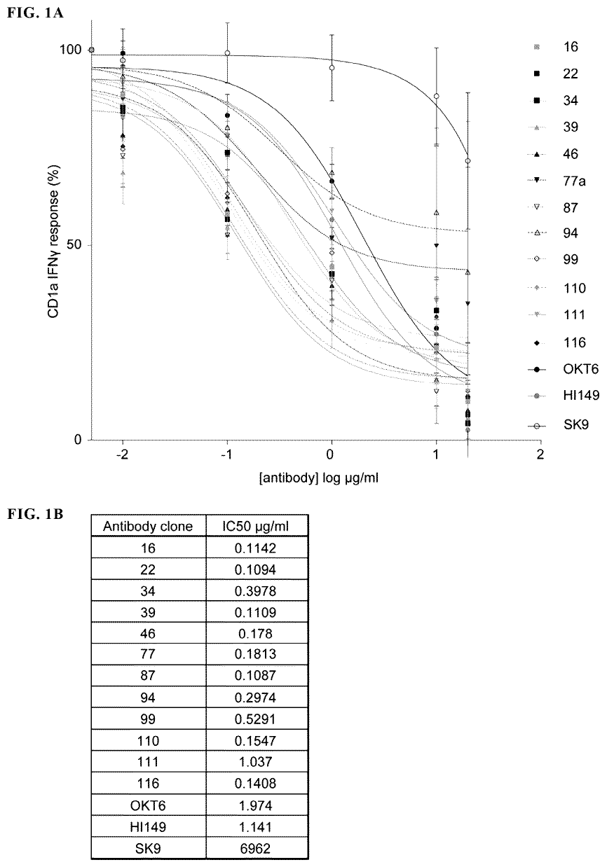

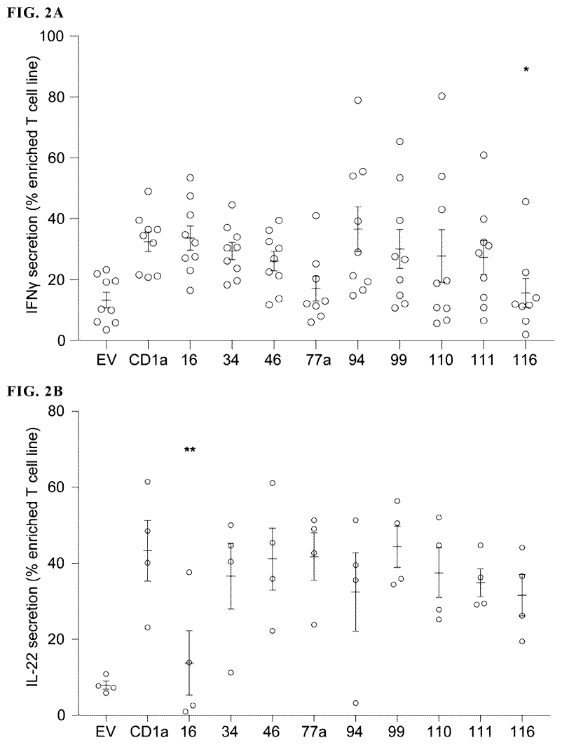

Following CD1a binding assessment a large panel of anti-CD1a antibodies generated for inhibitory function were screened. T cell cytokine production was measured in an in vitro antigen presentation model by EliSpot. A summary of these data is presented in FIG. 1. It was determined that a number of the newly generated antibodies were more potent in the inhibition of CD1a T cell responses than commercial anti-CD1a antibodies OKT6, HI149 and SK9. Of note, antibodies 16, 22, 39, 46, 77, 87, 110, 116 all had at least a log lower IC50 than OKT6 (FIG. 1B) which is an improvement over antibodies described in the prior art, despite the use of polyclonal T cells which would be expected to be less sensitive than transduced clonal immortal T-cells.

### Example 2—Anti-CD1a Panel Refinement: Inhibition of CD1a-Restricted Enriched T Cell Lines Responses

To aid the short listing of antibody candidates for in vivo analyses, a different approach was taken to assess CD1a T cell responses; CD1a-restricted enriched T cell lines were isolated and expanded to analyse the CD1a response in isolation, rather than in a mixed polyclonal T cell background where the low signal to noise ratio can partially mask the potential of the inhibitory antibodies.

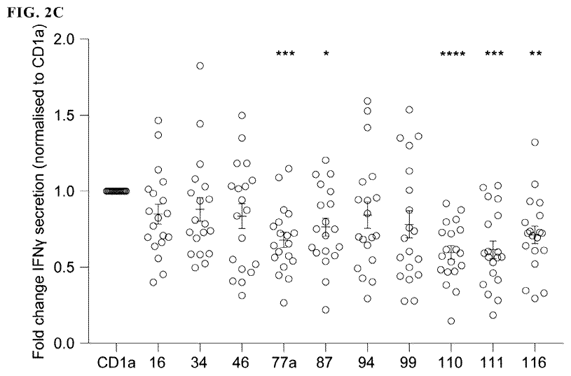

In these assays antibodies 116 and 16 stood out as potent inhibitory antibodies, with 16 uniquely inhibiting the autoreactive/endogenous production of IL-22 (FIGS. 2A and 2B). This improvement shows the possibility of using the antibodies in conditions on which IL-22 plays a pathogenic role, in addition to conditions which have a role for IFNγ. It was surprising to see differential effects on different cytokines. Further, an APC-free system was used to assess antibody dependent inhibition of CD1a-restricted T cell activation. CD1a-coated beads were used as a surrogate for the APC, and resulting T cell IFNγ production was measured by flow cytometry. This assay revealed significant inhibition of the CD1a-dependent cytokine response with all antibodies, but particularly 77a, 87, 110, 111 and 116 (FIG. 2C).

### Example 3—In Vivo Assessment of Inhibitory Antibodies in Skin Inflammation

The aim of this study has been to produce antibodies that would be of clinical use in treating human diseases and disorders, thus it was essential to ascertain efficacy in a complex immune system akin to human disease. A highly refined panel of the best of the newly generated antibodies were chosen from analysis of the above data (antibodies 16, 77a, 110, 111 and 116), and it was sought to determine their potential in an in vivo model of psoriasis, dermatitis, lupus and as a model of drug reactions which manifest as an inflammatory skin or mucosal disease or disorder, or associated systemic disease or disorder, or one or more inflammatory drug reaction which manifests systemically. Experimental psoriasis and dermatitis have been shown to be exacerbated in the CD1a transgenic mouse as compared to WT, and the CD1a-dependent inflammation can be ameliorated with administration of anti-CD1a antibody (Kim et al 2016). It is also of note that some individuals develop a skin/mucosal inflammatory drug reaction to imiquimod, used topically for a number of skin disorders; such drug reactions include psoriatic reactions, dermatitis reactions, bullous disease, alopecia, vesiculation, lichenoid reactions, neutrophilic diseases, lupus erythematosus, erythema multiforme, oral erosions and severe drug reactions such as DRESS, AGEP, Stevens-Johnson syndrome and toxic epidermal necrolysis (19-29).

### Generation of CD1a Transgenic Mice

To assess a possible role for CD1a in skin and associated systemic inflammation the inventors generated a CD1a transgenic mouse. CD1a is absent from the mouse genome, and so the human CD1a gene locus with 0.8 kb 5′ and 0.8 kb 3′ flanking region that includes the promoter element, was cloned and the transgene inserted by microinjection, akin to the published CD1a transgenic model, but requiring additional transgene fragment stitching (Illing et al., Nature 486, 554-558 (2012)). The genotype positive founder mice were bred and lines screened for CD1a transgene expression. The inventors went on to phenotype the mice and determine whether CD1a protein expression followed the expected profile and was representative of human CD1a cellular expression. Ear skin of wild-type and CD1a transgenic (CD1aTg) mice was collected and enzymatically processed to allow analysis of the cutaneous cellular environment by flow cytometry (FIG. 3A). CD1a expression was detected in the skin constituting 4.2% (+/−1.79) of total skin cells and 23.6% (+/−6.68) of CD45+ cells. To assess the cellular regulation of expression, dermal DCs (dDCs) and Langerhans cells (LCs) were assessed for CD1a protein. Dermal DC subsets have been reported to express CD1a and Langerhans cells are characteristically constitutive CD1a^{high}. CD1a was found to be expressed by 41.5% (+/−20.38) of dDCs and 88% (+/−4.606) of LCs (FIG. 3A-3B). CD1a protein expression was further characterised in the skin by immunofluorescence revealing characteristic epidermal location and cells with dendrites typical of LCs (FIG. 3C). CD1a genotype was confirmed (FIG. 3D), and CD1a expression within the thymus was observed, predominantly by a proportion of CD4+CD8+ double positive thymocytes (FIG. 3E). CD1aTg mice showed no aberrant skin inflammation at steady state. In summary, the inventors generated a CD1a transgenic mouse that displays CD1a expression in a manner phenotypically analogous to human tissue expression.

This model was used to test the anti-CD1a antibodies for prevention of inflammatory skin diseases and disorders (FIG. 4A). Application of Aldara cream, containing 5% imiquimod a TLR7/8 agonist, is an established model which induces psoriasis-like, dermatitis-like, lupus-like skin inflammation typified by skin thickening, scaling and reddening (30, 31). It was found that inflammation of the ear of CD1a-transgenic mice was considerably higher than of WT counterparts in response to Aldara. Furthermore, all anti-CD1a antibodies administered before the imiquimod reduced subsequent ear thickening, however antibodies 116 and 16 ablated CD1a-dependent inflammation to at least the WT level (FIG. 4B). By the end point of the experiment CD1a-transgenic (-Tg) mice treated with antibodies 16 and 110 showed reduction of inflammation to the WT level of ear thickening. Strikingly and unexpectedly, antibody 116 treatment reduced the level of CD1a-Tg ear skin inflammation significantly below that of WT skin (FIG. 4B).

### Example 4—In Vivo Effects of Inhibitory Antibodies on the Skin Immune Response

It was sought to analyse the contribution of cutaneous immune populations to imiquimod-induced CD1a-dependent ear inflammation.

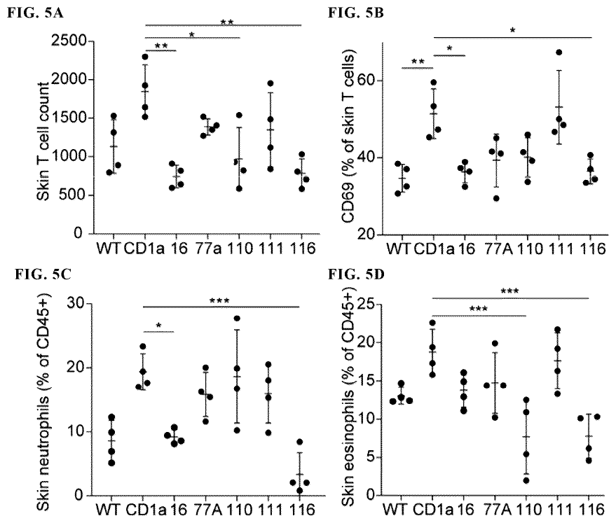

It was found that skin T cell infiltration was elevated in the CD1a transgenic mouse and the frequency of this population was reduced by the anti-CD1a antibodies, in particular antibodies 116, 16 and 110 in the prevention model (FIG. 5A). Of note, 16 and 116 were able to reduce skin T cell infiltrate to levels below wild-type suggesting an improved and profound effect on inflammation in vivo. Furthermore, activation marker CD69 was increased on the surface of skin T cells in the CD1a transgenic mouse, and was inhibited by some of the anti-CD1a antibodies, in particular 116 and 16 in the prevention model (FIG. 5B). Neutrophils are known to be important cells of a number of inflammatory disorders, including the psoriatic response and the murine imiquimod model. Here, elevated neutrophil frequency was found in the skin upon imiquimod treatment and further increase in the CD1a transgenic mouse, which was reduced to the WT level or below with anti-CD1a antibodies 116 and 16 in the prevention model (FIG. 5C). A reduction in skin eosinophils in response to the antibodies was also noted, which is of interest given the known role of eosinophils in many forms of drug reactivity (FIG. 5D). This unexpected finding represents an improvement as effects on eosinophils have not previously been observed.

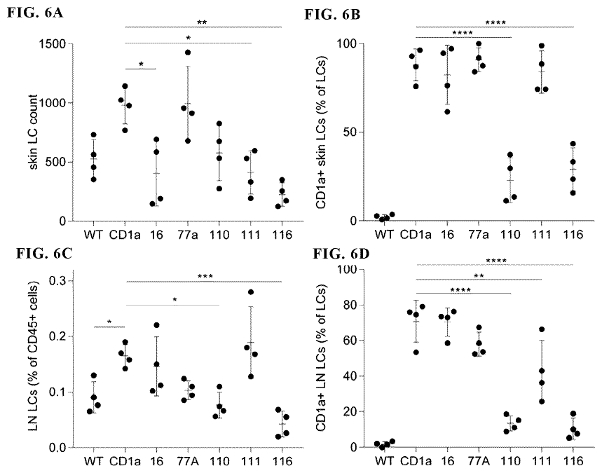

Langerhans cells, defined here as CD11c+ Langerin+, were also increased, compared to WT, in the skin upon imiquimod challenge of the CD1a transgenic mouse, as has been observed in human skin inflammatory disorders. With administration of antibodies 16, 116, 111 and non-significantly 110, skin LC count was diminished in the prevention model (FIG. 6A). Notably, antibody 116 reduced skin LC numbers below those in the wild-type skin showing an improved and surprising level of effect. As the predominant CD1a expressing population, the effect of antibodies on LC CD1a expression was assessed. It was of note that antibodies 110 and 116 had reduced staining, but this was due to interference of the 110/116 antibodies to binding by the HI149 detection antibody (FIG. 6B). This shows sustained binding of the antibodies in vivo which is a surprising effect and is associated with therapeutic advantage. The findings also raise the possibility of using the antibodies for diagnostic or prognostic purposes or monitoring CD1a-expressing cells before and during treatment. This observation was not seen with a non-competing SK9 detection antibody as presented below. The observed LC reduction could be due to antibody-dependent LC death or migration or altered phenotype. As such the cervical lymph nodes were analysed for presence of CD11c+ Langerin+ LCs. It was found that an increased number of LCs in the lymph node of CD1a transgenic mice, compared to WT, however migration to the LN did not appear to explain the reduction in skin LCs for mice treated with antibodies 110 and 116 (FIG. 6C). Notably, antibody 116 brought immunological improvements close to those in the wild-type skin showing an improved and surprising level of effect. Interestingly the level of expression of CD1a on the lymph node-derived LCs followed a similar pattern to that of the skin, in that LC had reduced staining, which was due to interference of the 110/116 antibodies to binding by the HI149 detection antibody (FIG. 6D) as discussed further below. This was not seen with a non-competing SK9 detection antibody. It is of note that the lymph node derived LCs expressed less CD1a per cell than those of the skin, this may be a control mechanism to prevent systemic inflammation. The antibodies therefore maintain effects on LC in vivo in the skin and even after migration to the lymph nodes. This is an important enhancement as the clinical effects will be more long-lasting.

### Example 5—Anti-CD1a Antibody Observed Cytotoxicity Expressed in Effects on CD1a-Expressing Cell Phenotype

Given that enhanced migration did not fully explain skin LC reduction, the potential for antibody induced alterations in phenotype of CD1a+ cells was investigated, despite the murine IgG1 nature.

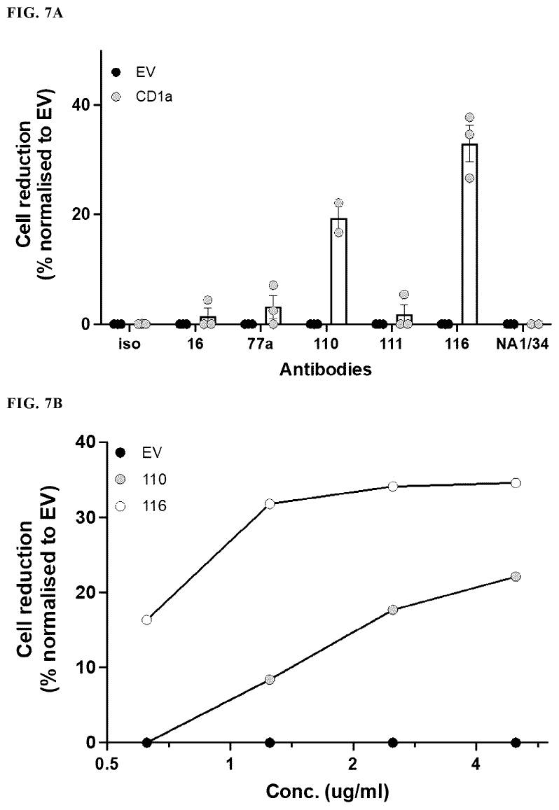

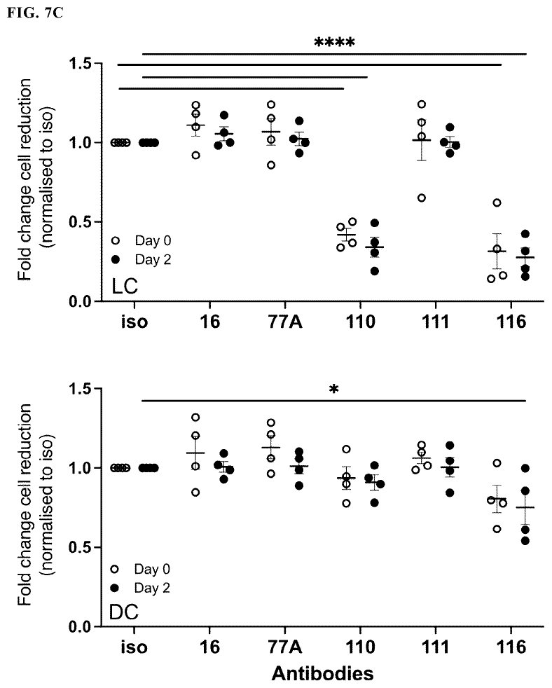

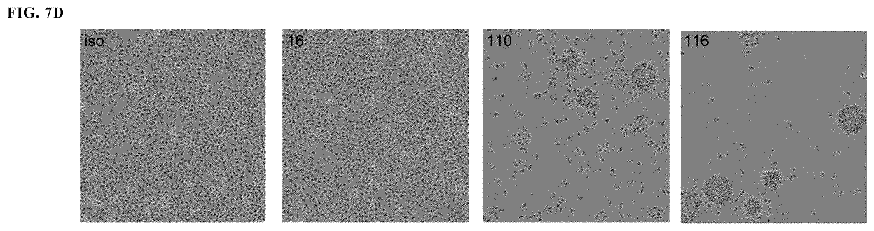

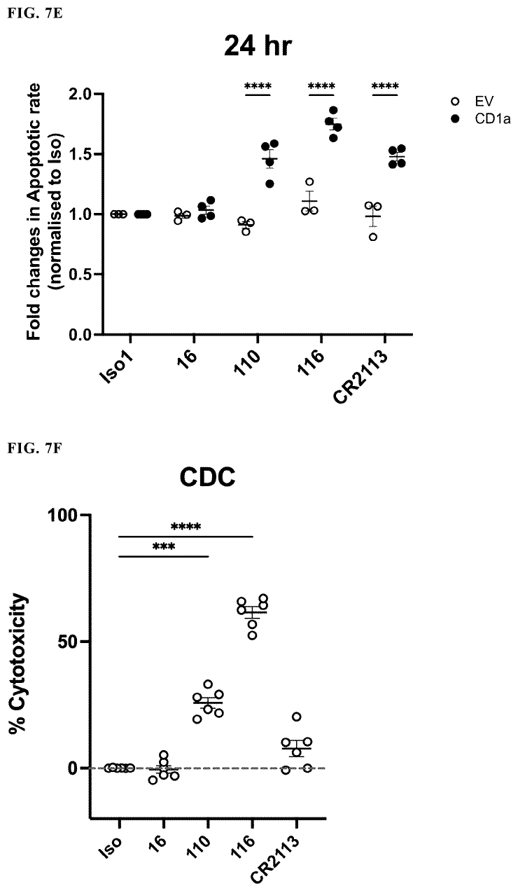

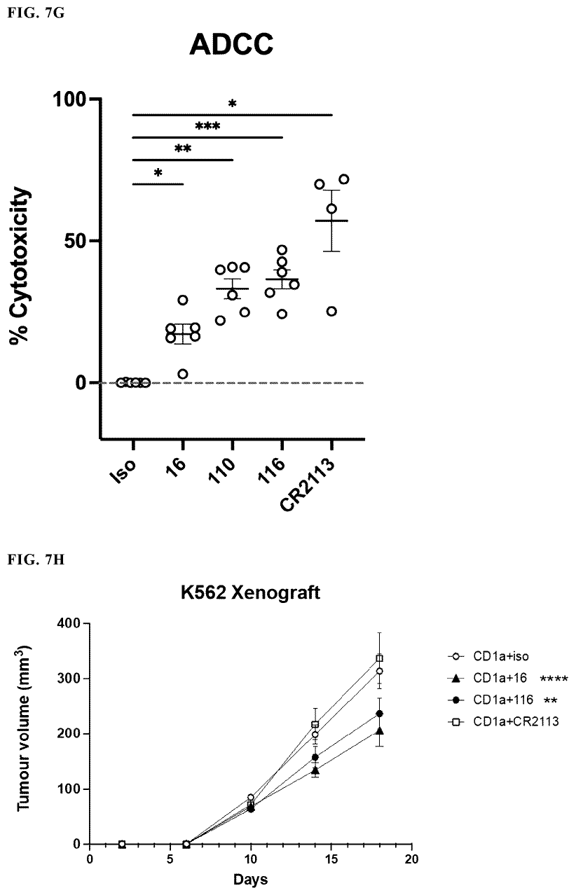

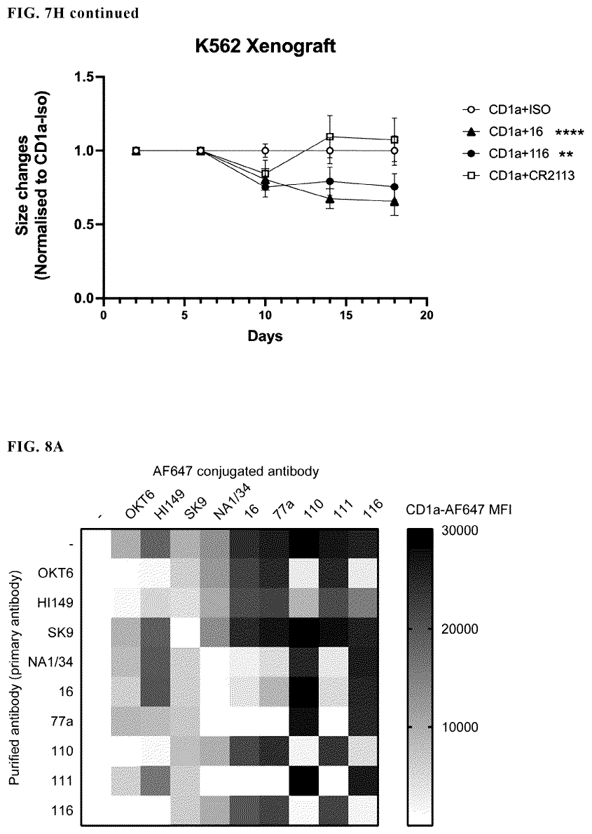

It was demonstrated that all anti-CD1a antibodies, but in particular 110 and 116, were capable of in vitro reduction in number of CD1a+ K562 cells which lack MHC class I and II and so permit comparison of responses (FIG. 7A). Antibodies 110 and 116 were tested in more detail which showed reduction in a dose dependent manner (FIG. 7B) which was an improvement and a surprise given the murine IgG1 isotype. This was apparently different to the published CR2113 antibody (16, 18) (U.S. Ser. No. 10/844,118B2 and CA 2924882 A1) which is stated to require complement and/or antibody-dependent cellular cytotoxicity. Specifically, it is stated “CR2113 does not directly induce apoptosis” (17) and it was noted that NA1/34 does not induce direct killing. However, as different Fc regions influence effector functions, the comparative effects of CR2113 on a murine IgG1 background are addressed directly below. The inventors went on to assess the capacity of the antibodies to induce direct reduction of primary human CD1a expressing cells. DC- and LC-like cells were generated through 5 day in vitro differentiation of monocytes using cytokines IL-4/GM-CSF, and IL-4/GM-CSF/TGF-β respectively with the addition of anti-CD1a antibodies on day 0 or 2 of culture. It was observed that antibodies 110 and 116 reduced LCs and to a lesser extent DCs in vitro (FIG. 7C upper and lower panel respectively). In exploration of the mechanisms underlying this reduction, the inventors found the reduction to be associated with a striking cell clustering culture phenotype morphology (FIG. 7D). The reduction in number could be partly explained by this clustering, but in addition, it was tested whether the antibodies could induce apoptosis of CD1a-expressing target cells and compared to CR2113 (on murine IgG1 background). FIG. 7E shows that 110 and 116 (but not 16) and CR2113 (on murine IgG1 background) induce annexin V expression by CD1a-expressing K562, even in absence of complement or ADCC. This suggests that 110, 116 and CR2113 antibodies can mediate K562 cell death to some extent. In order to investigate the role of complement-mediated lysis (CDC) and antibody-dependent cytotoxicity (ADCC), K562-CD1a were incubated with complement (FIG. 7F) and/or with human PBMC (FIG. 7G). Despite the murine IgG1 nature of the antibodies, there was evidence of complement-mediated lysis and ADCC. The effects of the antibodies on human IgG1 Fc regions are investigated below. To further investigate mechanisms in vivo, a new model was established using K562-CD1a subcutaneous tumours in an immunodeficient NSG model where there are broadly deficient lymphocyte responses and other effects. The data showed that all three antibodies reduced the size of the lymphoid cell tumours by day 10, with the effects sustained (to at 25% or greater reduction in CD1a-expressing tumour cell volume) for 16 and 116 by days 15-20 but lost for CR2113 (FIG. 7H). The differences in in vitro and in vivo responses may be explained by other cofactors present in vivo such as complement, numerous innate cell subsets bearing FcR with specificity for different Fc, differential target cell density, reduced antibody half-life in vivo, and altered tissue access. Such a direct alteration of phenotype of CD1a-expressing target cells may facilitate a less inflammatory response of CD1a-expressing cells. As such, the reduction of LCs in the skin of CD1a-Tg mice treated with 110 and 116 may be partly explained by direct antibody dependent change in phenotype of CD1a+ LCs and contribute to the clinical effect, for example in 116 reducing inflammation to below that of wild-type. The data also raise the possibility that the antibodies may have utility in treatment of CD1a-expressing malignancies which include Langerhans cell histiocytosis and some forms of T cell lymphoma and some forms of thymoma. However, phenotypic alteration of target cells does not explain the reduction of T cell functional responses shown in FIG. 2, as the CD1a-bead assay (FIG. 2C) would not be affected by any depletion effects.

### Example 6—Epitope Binding Analysis of CD1a Antibodies

The data presented herein demonstrates that the five newly generated anti-CD1a antibodies have a range of functionality and it was sought to determine whether the antibodies have overlapping binding sites, using a flow cytometry cross-blocking assay. Additionally, epitope overlap was assessed with commercially available antibodies OKT6, HI149, SK9 and NA1/34 (binding site known to overlap with CR2113, as above).

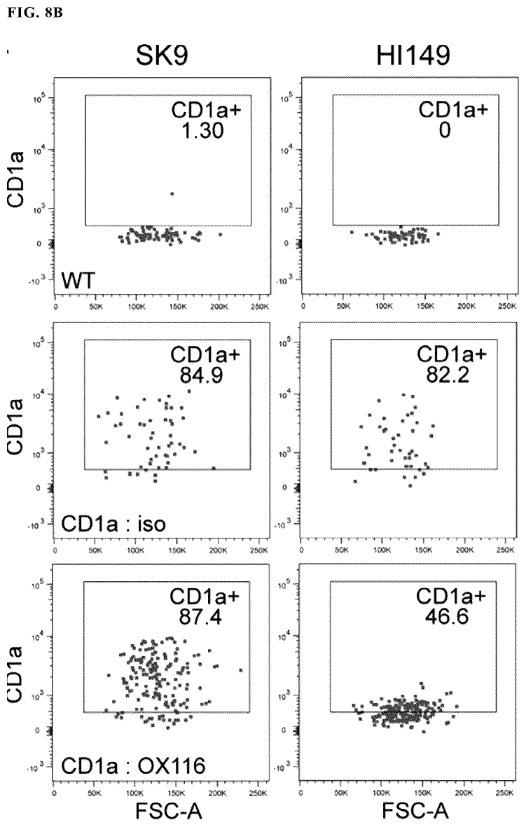

CD1a-K562 cells were incubated with purified primary anti-CD1a antibodies (Y axis FIG. 8A, 25 μg/ml), the unbound antibody was then washed away and Alexa-Fluor-647 conjugated forms of the different antibodies were then incubated with the cells in the matrix arrangement of FIG. 8A (X axis, 10 μg/ml). Mean fluorescent intensity (MFI) was used to assess the degree of binding of the fluorophore conjugated antibody and so any steric interference caused by binding of the primary purified antibody would be represented by a decrease in MFI. The results indicated that antibodies HI149, OKT6, 110 and 116 may have overlapping or closely associated epitopes and a second group containing antibodies NA1/34, 77a, 111 and 16 may have closely related binding sites. This suggests the reduction in CD1a expression observed in vivo (FIGS. 6B and 6D) was due to interference of the 110/116 antibodies to binding by the HI/149 detection antibody. Indeed, this effect was not seen with a non-competing SK9 detection antibody (FIG. 8B). Importantly and unexpectedly, the antibodies therefore maintain presence on LC in vivo in the skin and even after migration to the lymph nodes and following skin tissue enzymatic digestion. This will likely associate with a more prolonged and substantial clinical benefit. As the antibodies fall into two main groups which do not compete, FIG. 8 (FIGS. 8A and 8B) shows that combinations of antibody members selected from each group can be used together, for example as therapeutic/monitoring or combined therapeutics. One such combination would be 116 and 16.

### Example 7—Demonstration of Effectiveness of Antibodies of the Invention on Treatment of Imiquimod-Induced Inflammation and Also Systemic Associated Inflammation

Given the skin-dominant expression of CD1a, most studies have focused on skin-specific functional effects, although the presence of circulating CD1a-reactive T cells has been demonstrated (11). A role for CD1a in inflammation of tissues beyond the skin has not been extensively studied. Furthermore, CD1a is known to amplify the imiquimod skin response (16), but there have been no studies on associated systemic sequelae. The inventors generate a novel CD1a transgenic mouse and CD1a-reactive T cells, and characterize anti-CD1a antibodies for functionality in vitro and in vivo using human and mouse assays respectively. The findings confirm CD1a-dependent effects extend to systemic effects, with implications for treatment of systemic associations of skin disease including adverse inflammatory drug reactivity.

### Therapeutic Potential of Anti-CD1a Antibodies

To further evaluate the therapeutic potential of the newly generated anti-CD1a antibodies, the inventors tested the three most clinically effective antibodies 16, 110 and 116 in an imiquimod treatment model, where the anti-CD1a antibodies were introduced after the establishment of imiquimod-induced inflammation (FIG. 9A). All three antibodies improved clinical responses rapidly after initiation despite ongoing imiquimod application (FIGS. 9B-9C). The responses were most marked for 116, which reduced ear thickness (FIG. 9B). Whole skin (upper panel) and epidermal (lower panel) thickening was visualised by confocal microscopy (FIG. 9D), which confirmed the micrometer assessment (FIG. 9B). CD1a protein expression was assessed (anti-CD1a OKT6 AF-594, red) in the CD1a transgenic epidermis and noted to be reduced, through cell death and epitope competition, in 110 and 116 treated skin (FIG. 8A and FIG. 9D). Upon analysis of the cutaneous cellular immune response following the imiquimod treatment model, reduced skin T cell count and activation, reduced skin LCs, and reduced skin neutrophils after introduction of the antibodies was observed (FIGS. 9E-9G).

### CD1a is Involved in the Systemic Immune Reaction to Imiquimod

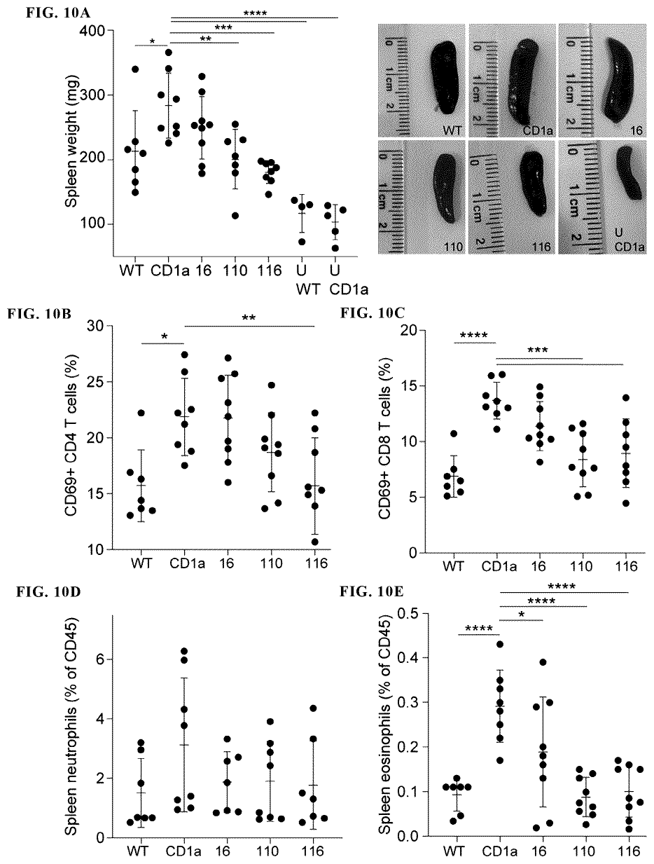

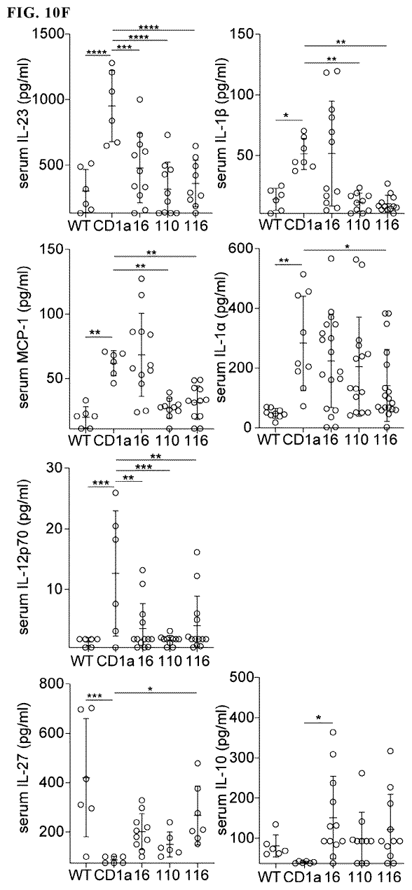

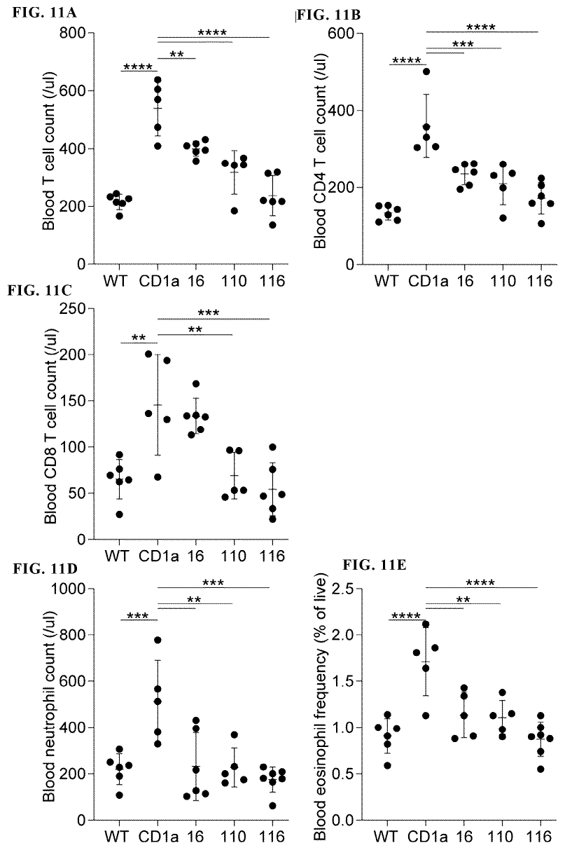

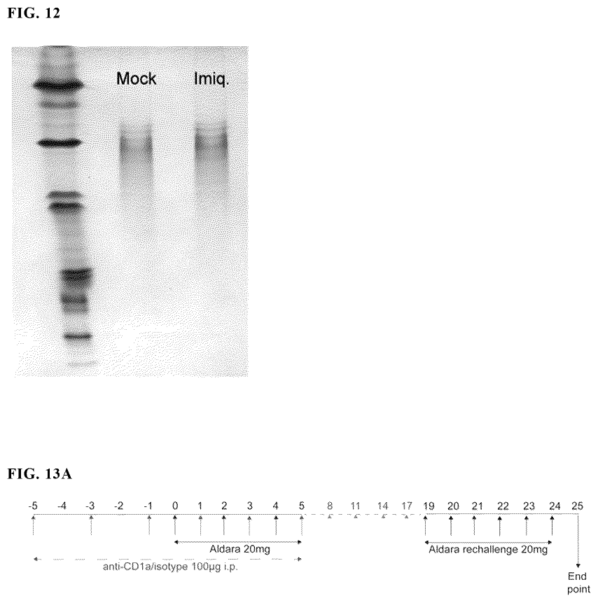

The human effects of imiquimod treatment can extend beyond the skin, and in the murine model have been shown to induce splenomegaly. The contribution of CD1a to this pathway was evaluated. Strikingly, spleen weight was increased in the imiquimod treated CD1a Tg mouse compared to wild-type and the antibodies reduced spleen size and weight, consistent with systemic effects beyond the skin (FIG. 10A). Furthermore, the antibodies reduced CD4 and CD8 T cells activation as determined by CD69 expression (116 and 110, FIGS. 10B-10C), splenic neutrophil (non-significant trend) and eosinophil frequencies (16, 110, 116) (FIGS. 10D and 10E respectively). Plasma cytokine levels were assessed at day 8. Significant increases in IL-23, IL-12p70, IL-1β, IL-1α and MCP-1 were observed in the imiquimod treated CD1a transgenic mice, and were reduced in some or all of the 16, 110 and 116 treated groups (FIG. 10F). Plasma immunoregulatory cytokines IL-10 and IL-27 were increased in the presence of the antibodies 16 and 116 respectively (and trend with the others). The impact on circulating immune cells was then ascertained. Similar to the spleen, blood CD4 and CD8 T cell counts, neutrophilia and eosinophilia were increased in the imiquimod-treated CD1a transgenic group. This increase was significantly blocked following treatment with 16, 110 or 116 (FIGS. 11A-11E). Lastly, the inventors investigated whether imiquimod itself might be a CD1a ligand and showed that this is not the case, implicating wider autoimmune and autoinflammatory effects of the CD1a pathway (FIG. 12). Therefore, it can be suggested that broad systemic inflammatory immune responses are primed or influenced by CD1a in the skin.

In order to investigate whether the anti-CD1a antibodies could produce a sustained reset of skin inflammation following imiquimod application, the model depicted in schematic FIG. 13A was undertaken where imiquimod re-challenge was used in the absence of re-administration of the anti-CD1a antibodies (FIG. 13B). Surprisingly, 16, 110 and 116 all produced sustained improvement in ear thickness in the absence of repeat antibody administration, consistent with a sustained immunological effect. The immunological response was also sustained with significant reductions in the frequency of skin T cells (110, 116), skin T cell activation (16, 110, 116), skin eosinophils (116) and skin neutrophils (16, 110, 116), lymph node T cell frequency (110, 116), lymph node T cell activation (16, 116), lymph node Langerhans cells (116), lymph node eosinophils (116) and lymph node neutrophils (116), blood T cell frequency (110, 116), blood T cell activation (116), blood eosinophils (110, 116), plasma IL-1α (116), IFNγ (16, 110, 116), IL-1β (16, 110, 116), IL-6 (16, 116), IL-17A (16, 110, 116).

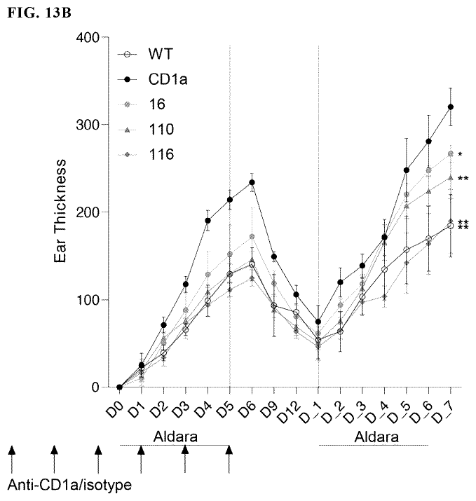

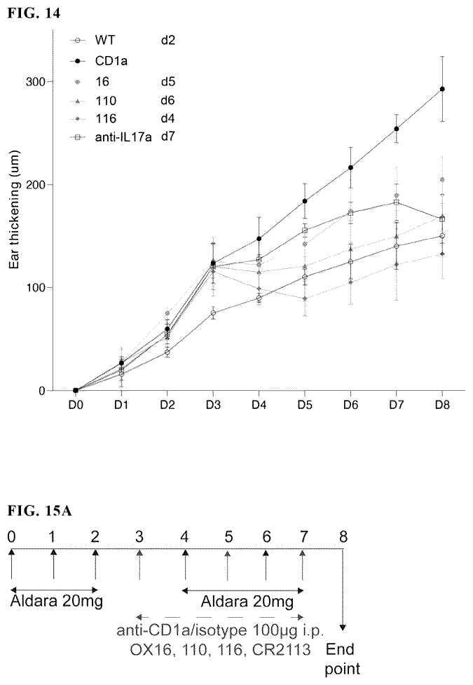

In order to compare performance of the antibodies with a current standard of care in the management of moderate-severe psoriasis, the imiquimod treatment model (FIG. 9A) was repeated alongside anti-IL-17A (IgG1 isotype) administered at the same time and dose (100 μg) as the anti-CD1a antibodies (FIG. 14). All anti-CD1a antibodies again showed significant improvement in ear thickness outcomes, with all producing significant improvements earlier than anti-IL-17A. It was noted that in contrast to the different anti-CD1a antibodies, the anti-IL-17A did not significantly reduce frequency of skin T cells, skin Langerhans cells, skin eosinophils, lymph node T cells, lymph node neutrophils, lymph node eosinophils, plasma IL-23, MCP-1, IL-6.

In order to directly compare skin and systemic inflammatory outcomes between the antibodies described herein and CR2113, the imiquimod skin treatment model was undertaken (FIG. 15A). All anti-CD1a antibodies had a beneficial effect on ear thickness, but antibody 116 was significantly improved over CR2113 (FIG. 15B-C). To extend the investigation of the improvement of the anti-CD1a antibodies 16,110 and 116 over CR2113, a comparison was made for an additional model of skin inflammation, namely MC903-induced inflammation (FIG. 15D) and a significant benefit was observed for antibodies 16, 110 and 116, but not CR2113, thus showing an improvement (FIG. 15E). It was noted that 16 and 116 showed a significant reduction in skin T cell percentage and skin eosinophil count, whereas CR2113 did not show significant reduction (FIG. 15F). Skin extract cytokines were significantly reduced where CR2113 did not show significant reduction for IL-5 (16, 110, 116), IL-6 (16, 110, 116), IL-9 (16), IL-23 (116), IL-17F (16, 110, 116).

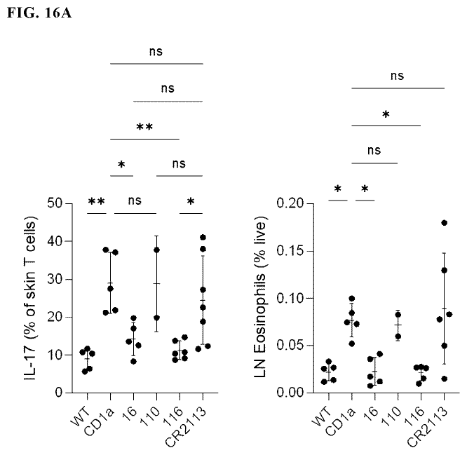

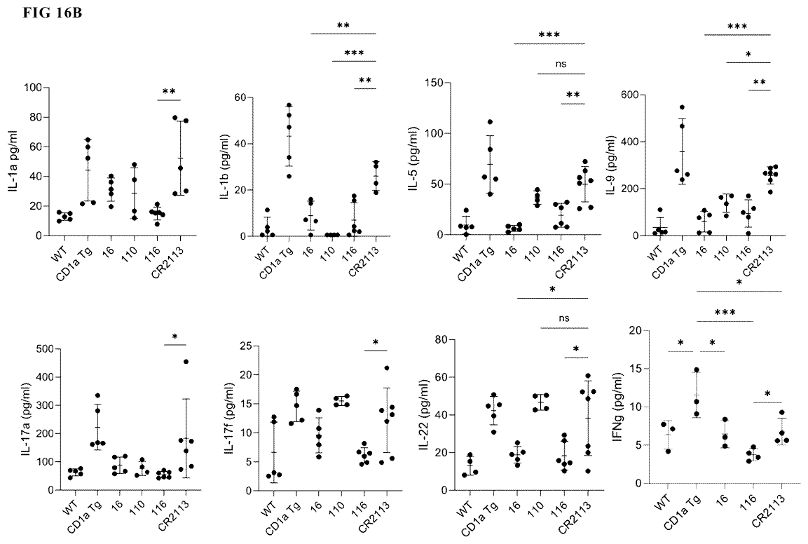

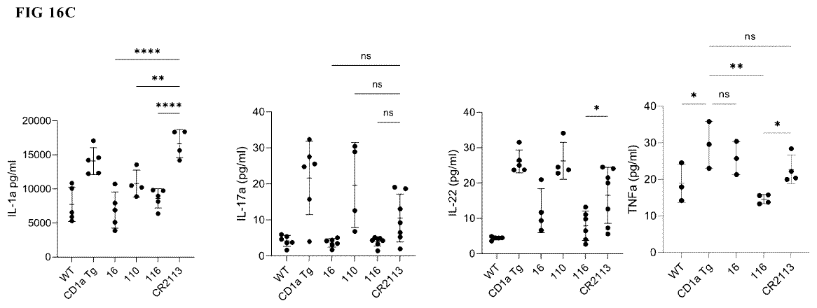

It was further observed that 116 showed consistent improvement over CR2113 in reducing skin, lymph node and plasma inflammatory responses to imiquimod (FIG. 16). For some outcomes, 16 was also significantly improved over CR2113 (FIG. 16). Specifically, antibody 116 was improved over CR2113 in reducing IL-17A expression by skin T cells, and in the frequency of draining lymph node eosinophils. 116 was also improved over CR2113 in reducing plasma IFNγ, IL-1α, IL-1β, IL-5, IL-9, IL-17A, IL-17F, IL-22 and skin digest IL-1α, IL-22 and TNFα. 16 was improved over CR2113 in reducing lymph node eosinophils, plasma IL-1β, IL-22, IL-9 and IL-5; and skin digest IL-1α and strong trends in IL-17A. Overall, the data confirm that the antibodies described herein are able to inhibit skin and systemic inflammatory responses to imiquimod and MC903.

### Example 8—Anti-CD1a Crystal Structures

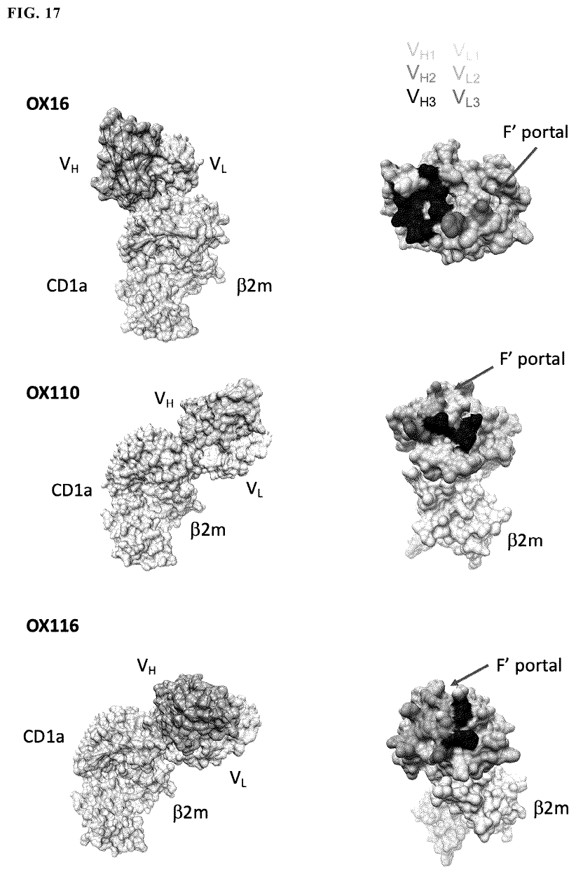

The crystal structures of CD1a bound to the single chain variable constructs of OX16, OX110 and OX116 antibodies were solved at 3.3 Å, 3.5 Å and 2.7 Å resolution respectively (FIG. 17). The electron density maps of CD1a, β2m and the scFv chains were of good quality and allowed a detailed molecular analysis of the interactions. The molecular details of the complexes are as follows:

OX16-CD1a: OX16 binds directly atop CD1a where it spans across the whole A′ roof docking on both, α 1 and α 2, helices. The total buried surface area (BSA) of the interface is 1528A (781 Å for OX16 and 747 for CD1a). CD1a residues that contribute to the interaction are: Glu 62, Glu 65, Leu 66, Thr 68, Leu 69, Ile 72 on α1 helix and Asn 151, His 153, Glu 154, Ile 157, Asn 160, Asp 164, Thr165 and Arg 168 on helix α2. Heavy chain provides 70% of the interaction and 30% corresponds to the light chain of the antibody. The variable loops of the antibody that are involved in the interactions are heavy chain: H1 (Tyr34) H3 (Arg100 to Trp106; Arg100, Tyr103 Tyr104, Tyr106) light chain: L1 (Tyr 169) L2 (Tyr 186) L3 (Tyr 229, Trp 233). CDR3 loop of the heavy chain is central for the interaction as it comprises 60% of all the buried surface area.

The blocking capacity of OX16 seems to be obvious considering its epitope greatly overlaps with the one of the autoreactive TCR BK6, the only ap TCR with a known crystal structure bound to CD1a. Most of the CD1a residues recognised by BK6 overlap with those central for the OX16-CD1a interaction (Glu 62, Glu 65, Ile 157, Asn 160, Asp 164, Thr 165, Arg 168 (Birkinshaw et al, Nature Immunology 2015) thus making binding of OX16 incompatible with BK6. Even though the Ab does not occlude the F′ portal the L1 loop is situated almost immediately above it leaving a limited amount of space for the protruding headgroups, which prompted SPR experiments to investigate whether the size and ‘bulkiness’ of the headgroup might have an impact on CD1a recognition by OX16.

OX110-CD1a. OX110 antibody binds CD1a on the edge of α1 domain of CD1a, just on the side of the F′ pocket and is reminiscent of the binding shown by the recently published γδ T cell receptor CO3 (Wegrecki et al, Nat comm 2022). In this crystal structure four CD1a-antibody complexes are observed in the asymmetric unit and surprisingly minor differences between them can be seen in terms of interacting side chains, however the overall docking mode remains nearly identical. For instance, the loop Tyr19-Trp23 of CD1a can adopt variable conformation and interacts with the antibody in two copies of the complex but not in the other two. This points towards a certain degree of flexibility in the recognition of CD1a by OX110, hence proves that the interaction is quite robust. From a functional point of view this might be important because even if CD1a suffers minor conformational changes upon binding of particular lipid ligands on the cell surface, these changes are unlikely to affect the recognition of CD1a by OX110. For clarity the analysis focused on the complex with the best electron density map within the asymmetric unit. The total buried area upon complex formation is 1404 Å of which 680 Å correspond to OX110 Å and 724 to CD1a. The residues from CD1a α1 helix that contact the antibody are: Glu 79, Arg 82, Arg 83, His 86, Glu 87, Gln 89, Phe 90, Glu 91, Tyr 92, and from the α2 domain Val 147, Asn 150. His86 seems to be central to this interaction as it establishes H-bonds and salt bridges with 3 residues from the heavy chain of OX110, which explains why a point mutant CD1a[H86A] completely disrupts the binding of OX110 to CD1a as seen in the epitope mapping experiments. Antibody contribution is 68% and 32% for heavy and light chains respectively. The variable loops interacting with CD1a are: H1 (Ser31, Ser32) H2 (Asn53, Ser54, Ser 55) H3 (Asp97, Tyr 99, Tyr 101, Tyr 103, Gly 104, Trp 105) L1 (Phe 165, Asn 166) and L3 (Glu 228, Phe 229, Ser 230, Cys 231). Most of the antibody contribution again comes from the H3 loop which provides 30% of the total buried area. Interestingly L3 contains and intra-loop disulphide bond between Cys231-Cys236, which is common in single chain antibodies where it stabilises long CDR3 loops. Here it does not play an obvious role, as L3 has only a minor contribution to the interface of the interaction.

OX116-CD1a. OX116 antibody also binds the side of CD1a laterally to the F′ pocket. The epitope partially overlaps with the one of OX110, however OX116 spans across both α1 and α2 domains of CD1a. The buried area of the assembly is 1526A (CD1a provides 793A and OX116 733 Å). CD1a residues interacting with the antibody include: Arg 83, Tyr 84, His 86, Glu 87, Gln 89, Phe 90, Glu 91 on α1 domain and Asn 139, Met 140, Lys 142, His 143, Lys 146, Val 147, Gln 150 on α2 domain. In this case, even though His 86 makes contacts with the antibody, these do not include H-bonds and only consist of weak Van der Waals contacts, which explains why CD1a[H86A] mutant did not affect the interaction in epitope binding experiments. OX116 residues involved in the complex formation belong to H1 (Ser 31, Asn 32, Ala 34), H2 (Tyr 53, Thr 54, Thr 55, Gly 56, Phe 57, Tyr 59) H3 (Ala 99, Thr 100, Tyr 101, Val 102, Pro 104) L1 (Tyr 166, Asn 167) and L3 (Glu 229, Phe 230, Ser 231, Cys 232). Like in the other two complexes, here the VH domain comprises 75% of the assembly interface and the VL provides the remaining 25%. However, H3 loop that dominates the interaction in OX16-CD1a and OX110-CD1a here provides only 25% of the total interaction area. Surprisingly 35% of the BSA comes from the germline encoded H2 loop, which had minimal contribution (16% of BSA) in OX110-CD1a and none in OX16-CD1a. Here again there is an intra-loop disulphide bond within L3. In fact, the sequence of the loop L3 is nearly identical between OXI 10(GEFSCSSTDCVTF) and OX116 (GEFSCSSVDCATF) and in each case identical residues from L3 contact the same segment of CD1a (Gln 89), however the angle of the interaction is different and the heavy chains dock on a different epitope.

Even though both OX110 and OX116 bind the F′ pocket side of CD1a their binding mode is different. Binding of OX110 around His86 induces conformational changes in that part of α1 helix, which adopts a conformation different from any other structures of CD1a. Moreover, it affects the amino-terminal part of the α1 helix in the A′ roof area and slightly changes the way α1 and α2 helices interact to form A′ roof Similar effects were not observed upon binding of OX116 to α1-α2 interface on the F′ side of the cleft. In summary, the shape of the binding cleft of CD1a does not seem to be affected by association with OX16 or OX116 however binding of OX110 impacts the α 1 helix and its association with a 2 helix.

### Example 9—Effects of Lipid in Binding of OX16 and OX116 to CD1a

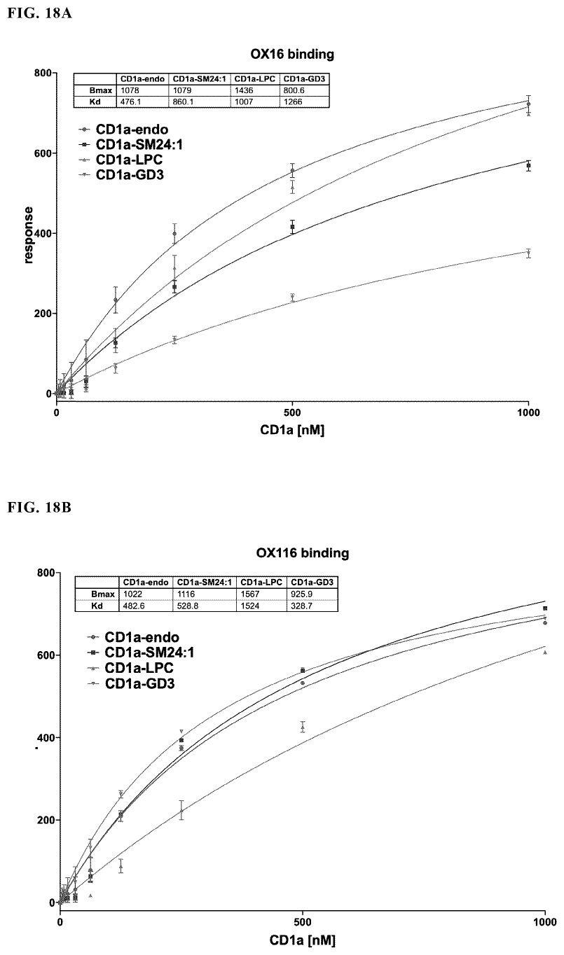

CD1a was loaded with different lipids which are known to be permissive (endogenous “endo”, lysophosphatidylcholine 18:1 (LPC)) or non-permissive (sphingomyelin 24:1 (SM24:1)) or a large head-group lipid control (ganglioside GD3). The CD1a loaded with lipids was then tested for OX16 (FIG. 18A) and OX116 (FIG. 18B) antibody binding using surface plasmon resonance. OX16 bound to CD1a containing all the lipids with some enhanced binding to permissive ligands, consistent with a degree of selectivity towards lipids which may promote an autoreactive T cell response. Lipid antigens that significantly protrude through the F′ portal (Sphingomyelin, GD3) seem to negatively impact the recognition by OX16. As described in example 8, this may be explained by an OX16 overhang to the F′ portal which could limit binding to non-permissive lipids with large protruding head-group antigens. Hence, binding of OX16 to CD1a exactly follows the molecular pattern previously described for autoreactive ap T cell receptors. OX116 showed binding to all lipids tested including endogenous lipids (“endo”), SM24:1, LPC, GD3, sulfatide and phosphatidylcholine without preferential binding to classes of known permissive or non-permissive ligands. This was unexpected given the proximity with which OX116 binds to the F′ portal. Further, it demonstrates that the recognition of CD1a by OX116 is robust and is unlikely to be affected by the identity of antigenic lipids present in the cleft of CD1a at a cellular level. Overall, these data show that OX16 and OX116 can bind CD1a loaded with a wide range of lipids.

### Example 10—Effects of Blocking of Polyclonal and Clonal T Cell Function by Anti-CD1a Antibodies

K562 cells expressing CD1a or empty-vector controls (EV) were incubated with different anti-CD1a antibodies and with polyclonal T cells isolated from healthy adult donors overnight. The number of cells expressing IFNg or IL-22 was measured using ELISpot and the percentage inhibition was compared to isotype control (FIG. 19A-B). The wild-type human IgG1 Fc showed significant reductions for IL-22 production with all antibodies, but only for antibodies OX16, OX110, OX116 for IFNg production. The afucosylated IgG1 showed significant IL-22 reductions for OX16, OX110, OX116, CR2113 and mAb571. The afucosylated IgG1 showed increases in IFNg production with OX16, OX110, CR2113 and mAb571 consistent with the known enhanced Fc effector function of afucosylated IgG1. Of note, despite the enhanced effector function of afucosylated IgG1, antibody OX116 did not show significantly increased IFNg induction. Potential mechanisms were explored below. Overall, these data show that Fab versions of the antibodies can inhibit polyclonal CD1a-dependent T cell reactivity with evidence of improvements of OX16, OX110 and OX116 over CR2113 and mAb571 which were highlighted through use of different IgG1 Fc region comparators. The ability of the antibodies to modulate the IFNg production by CD1a-reactive T cell clones was next investigated (FIG. 19C). All antibodies showed the capacity to inhibit CD1a-reactive T cell clone production of IFNg whether on wild-type human IgG1 Fc region or as a Fab variant.

### Example 11—CDC and ADCC Effects of Anti-CD1a Antibodies

Given the findings in FIG. 7 using murine Fc regions, the ability of the antibodies on different human Fc backgrounds was investigated for capacity to induce complement mediated cytotoxicity compared to CR2113 and mAb571 (FIG. 20A). OX16 and OX110 did not induce CDC but OX116 induced significant killing in the presence of complement when placed on all variations of human IgG1 backgrounds. However, no CDC was observed when using Fab version of OX116 implicating an Fc-dependent effect. When placed on different IgG1 Fc regions, the antibody OX116 showed improvements over the published antibodies CR2113 and mAb571 with relevance of use of the antibodies for particular indications, for example where cytotoxicity of CD1a expressing cells may be of patient benefit such as in the setting of CD1a-expressing malignancies. The anti-CD1a antibodies on different human Fc backgrounds were next tested for capacity to induce antibody-dependent cellular cytotoxicity (ADCC). Human IgG1 and a-fucosylated IgG1 for all anti-CD1a antibodies showed evidence of ADCC of CD1a-expressing target cells (FIG. 20B). Significant ADCC was not observed for the Fab versions of the antibodies. Of note, ADCC could not solely explain the findings in FIG. 19 because in the latter, the effector population was comprised of T cells and did not include NK cells, and a different effector:target ratio was used. Furthermore, the inhibitory responses in FIG. 19 were observed with use of the Fab forms of the antibodies. The anti-CD1a antibodies therefore show T cell blocking function as well as some Fc forms of the antibodies showing ADCC. In addition, as shown collectively above, OX116 may also induce direct killing of CD1a-expressing cells.

### Example 12—Inhibition of TCR Binding to CD1a by OX116

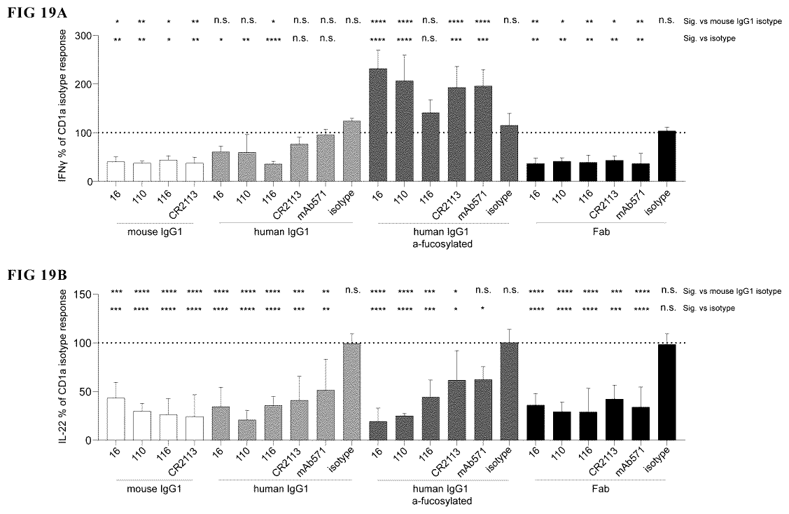

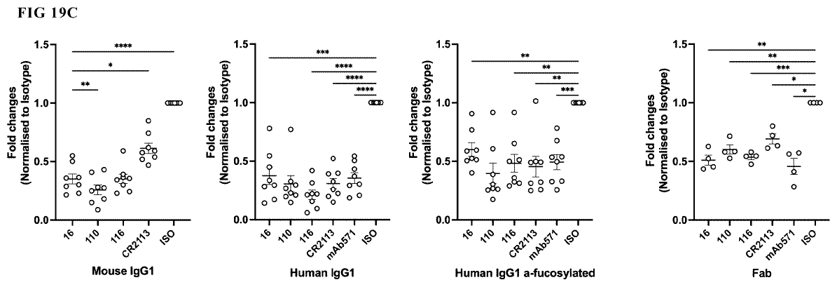

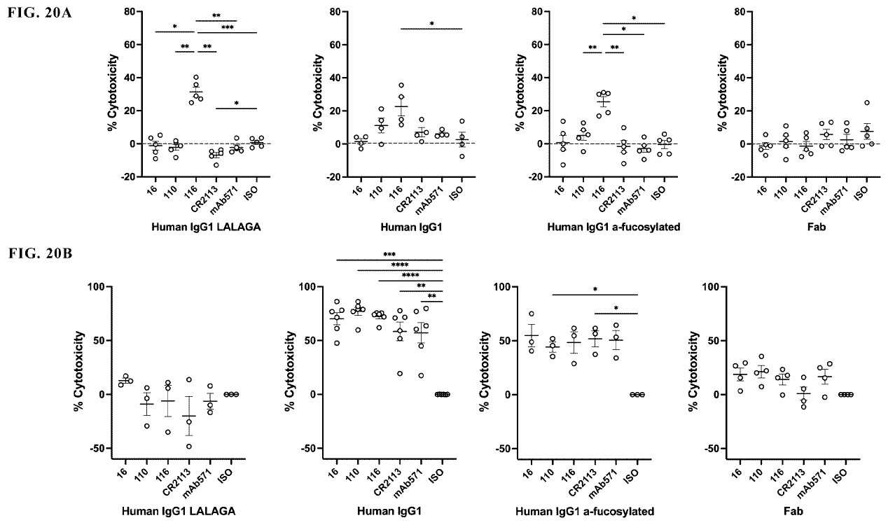

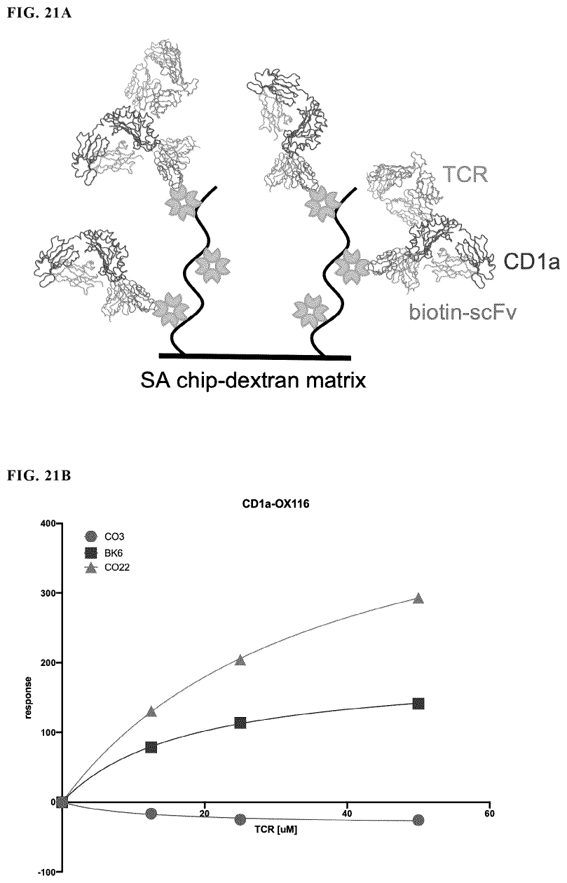

It was next investigated whether OX116 could inhibit binding of known CD1a-reactive TCR. Biotinylated ScFv of OX116 was captured on a streptavidin chip (FIG. 21A) followed by injection of CD1a and then three different TCRs, CO22, CO3 and BK6 (Birkinshaw R W et al., Nat Immunol. 16:258-66 (2015); Wegrecki M et al., Nat Commun. 13:3872 (2022)). BK6 αβ TCR binds the A′ roof of CD1a and CO3 γδ TCR recognises the α1 domain of CD1a within the region overlapping with the epitope of OX110 and OX116 antibodies. CO22 binding site is not established but is independent of the A′ roof and requires α3 domain of CD1a instead. As expected, CO22 TCR bound to CD1a-OX116 complex (FIG. 21B green curve). In order to explore whether the binding of OX116 to the side of CD1a could have an indirect distal effect on the shape of the A′ roof of CD1a and its recognition by autoreactive TCRs we used BK6 TCR. In this case however, the binding was still detectable (FIG. 21B blue curve) suggesting that OX116 did not interfere with the recognition of the A′ roof We used scFv fragments in the SPR experiments, hence it is possible that the binding of a full-size antibody would result in a more pronounced steric hindrance with an inhibitory effect on autoreactive TCRs. As expected, the interaction of CO3 TCR, which is known to bind close to the F′ portal, with CD1a was completely abolished by OX116 (FIG. 21B, red curve) confirming that, due to neighbouring epitopes on the surface of CD1a, the binding of the TCR and the antibody was mutually exclusive. Overall, these data show that OX116 can inhibit TCR engagement with CD1a.

### Example 13—Generation of OX25 Antibody Binding to the Alpha 3 Domain of CD1a

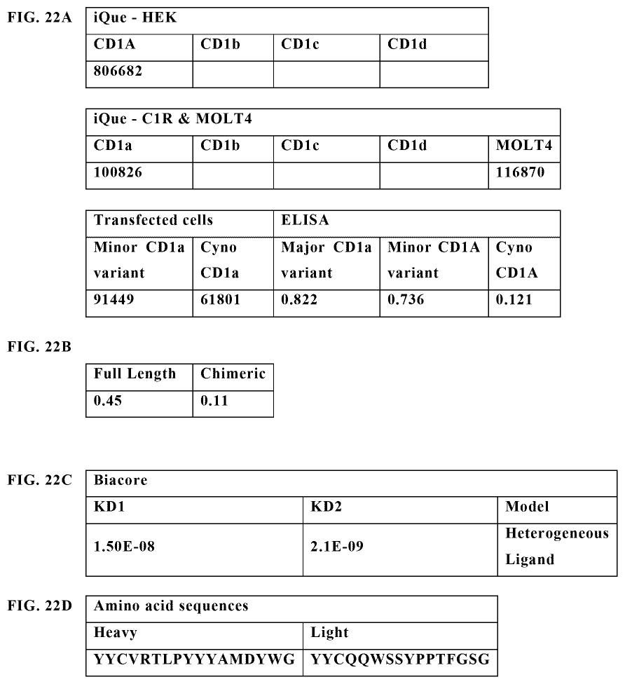

Having established the footprint of OX16, OX110 and OX116 anti-CD1a antibodies was to the alpha 1 and alpha 2 domains, it was sought to discover antibodies to the alpha 3 domain. Ab 25 was generated and selected as per the “Generation and selection of therapeutic anti-CD1a antibodies” materials and methods section (FIG. 22).

### Example 14—Humanised Antibodies can Deplete CD1a-Expressing Transfectants and Inhibit CD1a-Autoreactive T Cells

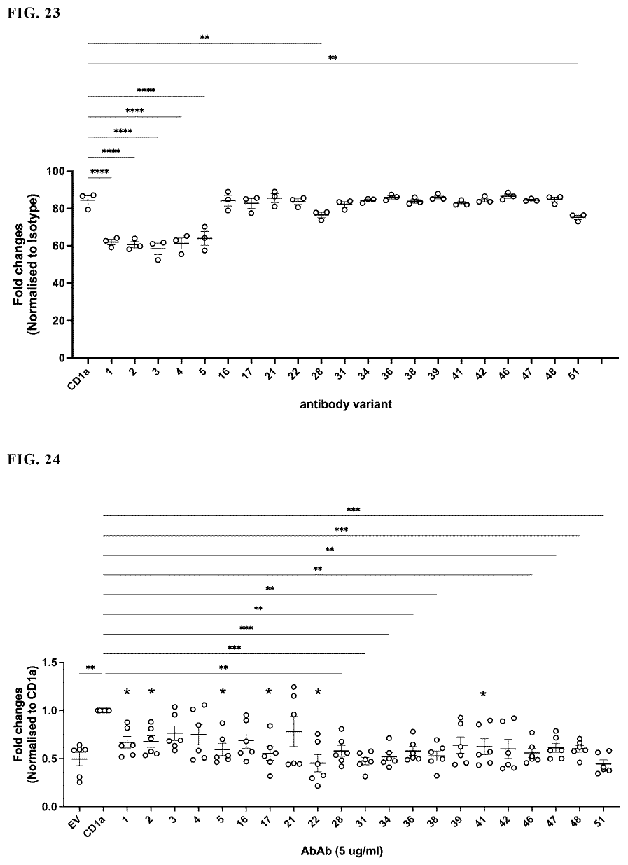

Humanised variants of the antibodies were generated (Abl-51) and tested for binding to K562-CD1a transfectants. Abl-15 are derived from OX116, Ab16-51 are derived from OX16. All 51 variant antibodies showed evidence of binding CD1a expressed by transfectants. However, it was noted that there was a range of mean fluorescence intensity (MFI) and the top antibodies were progressed towards functional analyses. FIG. 23 shows the depletion of K562-CD1a transfectants in the presence of anti-CD1a antibodies. The humanised antibodies derived from OX116 (1,2,3,4,5) all preserved depletion capacity, and of those derived from OX16 (16, 17, 21, 22, 28, 31, 34, 36, 38, 39, 41, 42, 46, 47, 48, 51) had no intrinsic depletion activity except for variants 28 and 51 which showed significant depletion capacity. The CD1a-reactive T cell clone blocking capacity of the variants was next tested (FIG. 24) and the majority showed significant ability to block IFNg production, with antibody 51 performing most strongly. The humanised antibodies may have direct diagnostic/monitoring/therapeutic utility or may be included as part of other approaches including bispecific or multispecific molecules or as part of cellular therapeutics.

### Example 15—Antibody 25 Activity

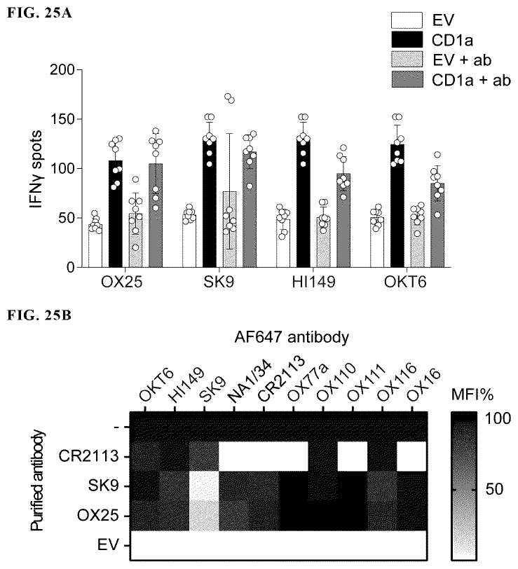

Antibody OX25 has been established to bind the alpha 3 domain of CD1a (FIG. 22) and was next tested for its ability to block CD1a-reactive T cells (FIG. 25). This confirmed that polyclonal CD1a-autoreactive T cell production of IFNg was not inhibited by OX25 as expected. Furthermore, OX25 was found to compete with CD1a binding with SK9 but not with antibodies which bind to alpha 1 and alpha 2 domains of CD1a. These data confirm the membrane proximal binding site of OX25 which may provide diagnostic, monitoring and/or therapeutic utility where a non-competing membrane-proximal domain is advantageous as observed with certain checkpoint agonists.

### Example 16—CD1a Antibodies can Treat Skin Inflammation

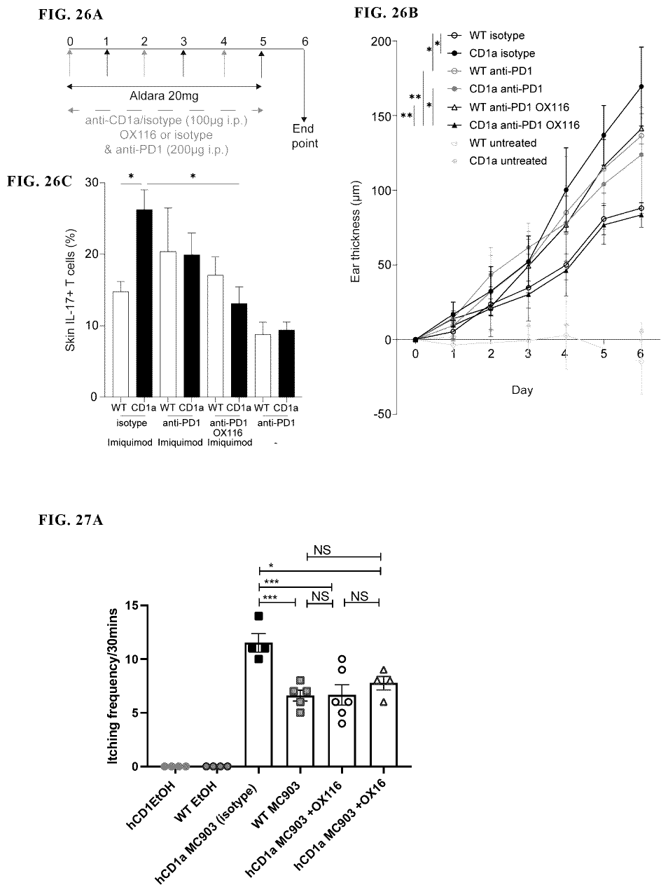

Checkpoint inhibitors are increasingly used for management of malignancy but can be limited by side effects, including various inflammatory skin reactions such as psoriasiform inflammation. This can sometimes necessitate the stopping of the checkpoint inhibitor with consequences for management of the underlying malignancy, or the use of broad immunosuppressants which also carries potential impact on the immunological control of the underlying malignancy. Approaches to treat tissue inflammation to allow ongoing use of the checkpoint inhibitor would have therapeutic utility. It was investigated whether CD1a and Langerhans cell pathways may be involved by utilising OX116 and anti-PD-1 antibodies alone or in combination in the imiquimod model of skin inflammation. FIG. 26A shows a schematic of the approach which established a significant reduction in ear thickness of PD-1 associated inflammation in the presence of OX116 (FIG. 26B) and a reduction in percentage of cutaneous IL-17A+ T cells (FIG. 26C). The extent of reduction in ear thickness was surprising and dramatic and reveals that these pathways are relevant.

### Example 17—CD1a Antibodies can Treat Pruritus

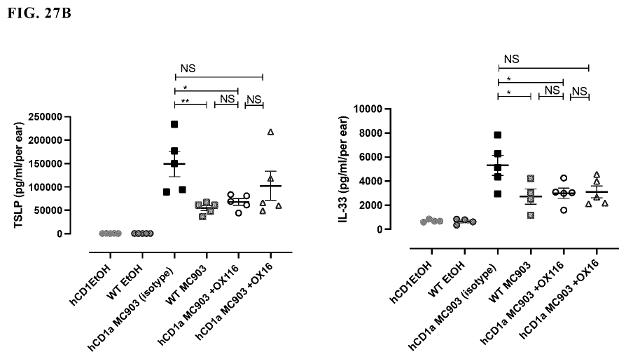

A major impact of inflammatory skin disease and other conditions on quality of life is pruritus. CD1a has not previously been linked to pruritus and so establishing the relevance of the pathways would represent a new area of biology. The role of CD1a and Langerhans cells in pruritus was next assessed using the MC903 model of skin inflammation. FIG. 27A shows that OX116 and OX16 both significantly reduce pruritus which was accompanied by reductions in alarmin cytokines known to play a role in pruritus (FIG. 27B). These data are surprising, novel and inventive as the CD1a pathway has not been implicated in pruritus previously. The findings would support the use of anti-CD1a antibodies in prevention and treatment of pruritus and associated diseases.

### Example 18—CD1a Antibodies can be Coupled to Other Antibodies to Increase Effectiveness

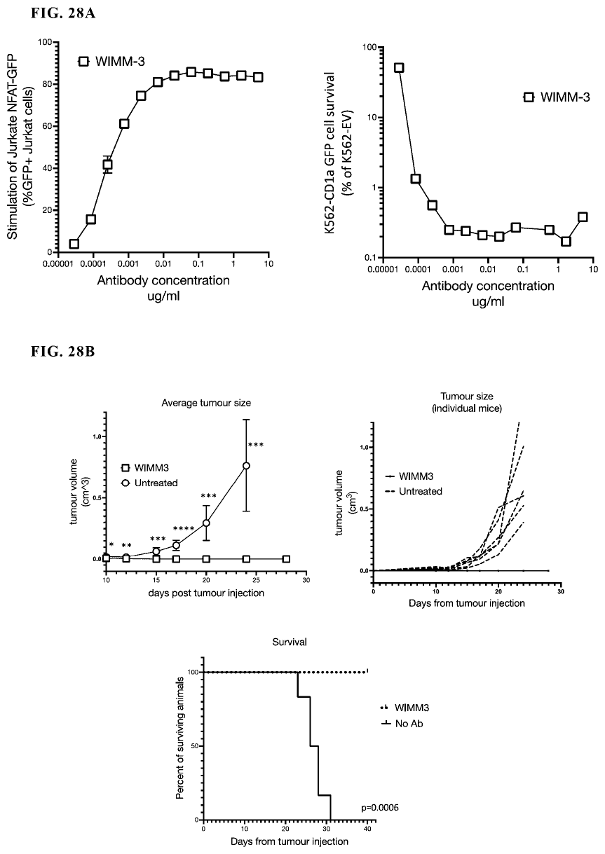

Anti-CD1a antibodies can act as an approach to target bispecific modalities, including T cell engagement. OX16 was coupled to a humanised version of UCHT-1 (anti-CD3 (Shalaby M Y, Journal Experimental Medicine 1992)) and tested for its capacity to activate reporter T cells when co-cultured with CD1a-expressing transfectants. Table B shows the sequence of WIMM-3 utilised. FIG. 28A (left panel) shows that the WIMM-3 T cell engager successfully activates reporter T cells in vitro. FIG. 28A (right panel) shows that WIMM3 promotes effective CD8+ T cell-mediated cytotoxicity of CD1a-expressing target cells. FIG. 28B shows that this is also reflected in an immunodeficient xenograft model where CD1a-expressing target cells were administered with human CD8+ T cells. The WIMM-3 CD1a-T cell engager effectively inhibited tumour growth which associated with significant improvement in survival. These data show that the anti-CD1a antibodies can effectively target bispecific molecules to CD1a-expressing cells which has many potential applications including treatment of CD1a-expressing malignancies as well as the principle of targeting other functional or binding modalities to CD1a-expressing cells.

### DISCUSSION

Skin inflammation such as dermatitis, psoriasis and lupus are common disorders with significant associated physical and psychological morbidity. Cutaneous adverse reactions to drugs are also common, ranging at 1.8-7 per 1000 hospitalised patients. Severe cutaneous adverse reactions, with widespread and systemic effects such as SJS, TEN, AGEP and DRESS are less common; for example, SJS/TEN has an incidence of approximately 1-6 cases per million individuals per year (M. Mockenhaupt, Allergol Select 1, 96-108 (2017)). Gell and Coombs defined a classification of hypersensitivities in the 1960s in which delayed type IV hypersensitivity required a role for effector T cells (R. R. A. Coombs, Gell, P. G. H., Classification of allergic reactions responsible for drug hypersensitivity reactions. In Clinical Aspects of Immunology. (Davis, Philadelphia, ed. second, 1968)). Although there is increasing recognition that the classification cannot account for all aspects of drug hypersensitivity, there has still largely been a focus on altered recognition of covalent haptens or non-covalently modified peptide/MHC molecules. However, the current models do not explain the dominance of skin and mucosal involvement of drug hypersensitivity (M. Mockenhaupt, Allergol Select 1, 96-108 (2017).

Through generation of a CD1a transgenic mouse and autoreactive human CD1a restricted enriched T cell lines, and characterisation of functional anti-CD1a antibodies, the data presented here show induction of CD1a presentation of endogenous lipid ligands. This leads to an autoreactive T cell-mediated cutaneous and systemic inflammation. The anti-CD1a antibodies had clinical and immunological effects, whether they were blocking or blocking/modulating, suggesting that CD1a lipid presentation to T cells is of importance. TLR7 can recognize single stranded RNA, and so it is of interest that reactivity to viral infections can mimic the clinical phenotype of different severe forms of cutaneous inflammation including psoriasis, dermatitis, lupus and adverse inflammatory reactions to drugs, including SJS and TEN. Such shared final common clinical manifestations might indicate that a number of precipitants can promote CD1a-autoreactivity and auto-inflammation. The model might also help explain the increased risk of autoimmunity associated with certain drug reactions, including lupus erythematosus and DRESS syndrome. Furthermore, the findings would implicate CD1a-autoreactivity in the breaking of wider T cell tolerance.

In addition to effects on the T cell response to the imiquimod-containing drug Aldara, increased neutrophil and eosinophil responses in the skin, draining lymph node and spleen were observed in the CD1a transgenic mouse. These effects were inhibited by the administration of antibodies of the invention, in particular 16, 110 and 116. This implicates a CD1a-dependent immune cascade that is wider reaching that initially anticipated. Neutrophil depletion has been shown to ameliorate the severity of imiquimod-induced inflammation (H. Sumida et al., Interplay between CXCR2 and BLT1 facilitates neutrophil infiltration and resultant keratinocyte activation in a murine model of imiquimod-induced psoriasis. J Immunol 192, 4361-4369 (2014).

Aldara/imiquimod application recapitulates key aspects of different forms of skin inflammation and associated systemic diseases and disorders, including psoriasis, dermatitis, lupus and severe cutaneous hypersensitivity reactions including T cell and neutrophil infiltration as discussed above. The data demonstrated herein shows that imiquimod-dependent eosinophil infiltration of the skin, lymph nodes and spleen was enhanced in the CD1a-transgenic mouse and reduced by administration of antibodies of the invention, in particular 16, 110 and 116.

Furthermore it has been reported that LC numbers are increased in lesional skin compared to non-lesional skin of patients with different forms of inflammatory skin diseases or disorders including psoriasis, dermatitis, lupus; and a maculopapular drug eruption, and were decreased to non-lesional levels as the eruption resolved (D. I. Dascalu, Y. Kletter, M. Baratz, S. Brenner, Acta Derm Venereol 72, 175-177 (1992)). Interestingly, psoriasis is associated with altered LC migration, suggesting that although imiquimod application is a well-studied and effective murine model of psoriasis and lupus and dermatitis, it also has applicability to include adverse drug inflammatory drug reactions. Here, the inventors show that CD1a-antibody dependent modulation of LCs was associated with reduced skin inflammation upon administration of antibodies of the invention, in particular 110 and 116, which may be of therapeutic importance to the treatment of psoriasis, dermatitis, lupus, inflammatory drug reactions and other conditions. The epitope analysis highlights the potential therapeutic importance of epitope binding site; the anti-CD1a antibodies fell into two groups based on binding site and resultant effector function. The epitope site may facilitate the clustering and change in phenotype effect seen with antibodies 110 and 116, but not 77a, 111 and 16, which were primarily blocking antibodies. The clustering may indeed lead to cross-linking/agglutination-like cell morphology, which may also explain the reduction of CD1a-transfected K562 and monocyte derived LCs as both cell types express high levels of CD1a, higher than monocyte derived DCs. The different antibody binding sites of the two groups do not compete and so there is utility for combinations selected from each of the two groups, for example in therapeutics/monitoring or in combination therapies.

The role of CD1a in the pathogenesis of skin inflammation and associated systemic disease implicates its role in many diseases, including psoriasis, dermatitis and lupus erythematosus and drug hypersensitivity. Furthermore, characterization of CD1a blocking and modulating antibodies offers a new potential route to preventative and therapeutic development for skin inflammation and CD1a-expressing malignancies.

The data shown herein define the CD1a contact points for anti-CD1a antibodies OX16, OX110 and OX116. The binding sites of OX110 and OX116 are close to the F′ portal and are different to OX16 and other published structures for anti-CD1a antibodies which bind over the A′ roof (U.S. Ser. No. 10/844,118 and WO/2022/077021). OX16 and OX116 were able to bind CD1a loaded with different lipids including permissive and non-permissive ligands. This was a surprise given the proximity of OX116 binding to the F′ portal of CD1a but suggests that OX116 may have broad utility in CD1a binding and/or CD1a blockade. Binding of OX16 was negatively affected by lipid antigens with bulky protruding headgroups, which mimics the behaviour of autoreactive T cells and gives rise to the possibility of using OX16 as a blocker of CD1a carrying only autoreactive permissive/small lipids but not lipids with properties where the co-recognition of the headgroup would be expected to trigger a desirable immune response, eg in responses to Mtb lipids. These findings were consistent with the ability of OX16, OX110 and OX116 antibodies to inhibit polyclonal CD1a-dependent reactivity with broad relevance including for IFNγ and IL-22 responses. Comparisons were made with published antibodies CR2113 and mAb571 (U.S. Ser. No. 10/844,118 and WO/2022/077021) for functional inhibition of polyclonal T cell responses. The antibodies showed broad and significant ability to reduce CD1a-dependent autoreactivity, with significant improvements of OX16, OX110 and OX116 over the published antibody comparators.

The use of different human IgG1 Fc variants altered the functional effects of OX16, OX110 and OX116 on CD1a-expressing target cells. Although all three antibodies showed clear improvements over published CR2113 and mAb571 antibodies, it was noted that OX116 produced the most CDC and loss of confluency of CD1a-expressing target cells, implicating the potential utility of OX116 in settings where depletion of CD1a-expressing cells may be advantageous, for example in CD1a-expressing malignancies. The use of Fab versions of the antibodies did not induce CDC or loss of confluency, suggesting that the Fc region and/or dimerization is required for such effector functions.

Thus collectively, a family of antibodies are described with binding sites across CD1a. As shown above in examples 2-7, having a range of binding sites has utility in detecting CD1a or modulating CD1a function in different ways in isolation or in combination.

In it is demonstrated that antibody OX25 binds the alpha 3 domain of CD1a. The effects of the antibodies on IFNg and IL-22 production were examined. These cytokines are broadly relevant to inflammatory skin disease and associated systemic disease. For example, IFNg is known to promote T cell and neutrophil responses and IgG class switching, as well as MHC class I and II induction, thereby amplifying innate and adaptive immune responses. IL-22 is known to have broad effects on epithelia and stromal cells, promoting cell proliferation, anti-microbial peptide expression and cutaneous and systemic inflammation. IL-22 has been linked to many inflammatory diseases including systemic lupus erythematosus, atopic dermatitis, rheumatoid arthritis and psoriasis (Dudakov J A et al., Ann Review Immunol 33:747-85 (2015)).

The antibodies OX16, OX110, OX116 and OX25 have different binding footprints with different associated functions. The ability to bind the alpha 1, alpha 2, or alpha 3 domains of CD1a provide opportunities to identify CD1a and modulate CD1a function either in isolation or in combination. It may be that this is through use of the anti-CD1a antibodies in a linked format or separately, or as part of other bispecific constructs (or other binding agents) or cell-based treatment approaches. The different binding sites, also offer the potential to utilise combinations in diagnosis or monitoring of treatment.

### SUMMARY

In summary, the inventors have generated a refined panel of anti-CD1a antibodies with therapeutic potential in the prevention and/or treatment of inflammatory skin and mucosal disorders. The antibodies 16, 77a, 110, 111 and 116 were shown to be potent inhibitors of in vitro human CD1a antigen presentation and showed efficacy in exemplar inflammatory skin disease prevention and treatment models which have features of psoriasis, dermatitis, lupus erythematosus and drug reactions which manifest as an inflammatory skin or mucosal disease or disorder, as well as those which are systemic (non-cutaneous), and in a xenograft tumour model. The success of the antibody discovery process in identifying improved antibodies may be attributed to combining: a) the screening of large numbers of hits (3500) with; b) the use of the novel chimeric immunogen, whereby the human CD1a lipid binding domain was fused to the host organism CD1d Ig domain, thus targeting antibody generation to the lipid binding domain where functional inhibition potential may lie with; c) a variety of polyclonal and enriched T cell analyses examining different functional outcomes.

In vitro human functional assays showed the antibodies to be more potent than commercially available antibodies, measured by IC50 assessment of inhibition of a primary polyclonal T cell response. Furthermore, using highly sensitive human CD1a-restricted T cell clonal assays, it was determined that anti-CD1a antibodies 16 and 116 were capable of blocking IL-22 production, which is a key regulator of inflammatory skin and mucosal disease. Such an activity was an improvement and surprise as this was not shown in existing publications or patents of anti-CD1a CR2113 ((16, 17), U.S. Ser. No. 10/844,118B2 and CA 2924882 A1), where IL-17 or IFNγ production was induced and inhibited in the murine system. IL-22 inhibition is an important advantage of the antibodies as IL-22 is a key regulator of skin and mucosal disease.

The parallel analyses of human and in vivo murine models provide a powerful means to assess the therapeutic benefit of the newly generated antibodies. In vivo, imiquimod was utilised to induce a psoriasis-like, dermatitis-like, lupus-like, drug-reaction-like phenotype and provide a model skin inflammation system, and may be more widely applicable to a number of inflammatory diseases and disorders as well as for associated systemic diseases or disorders and inflammatory drug reactions which manifest systemically. Here it was shown that antibodies 110, 116 and 16 significantly reduce the CD1a-dependent inflammation induced by imiquimod, with improvements over standard of care (anti-IL-17A) and a comparator anti-CD1a antibody on the same murine IgG1 background (CR2113). Importantly, and unexpectedly, antibody 116 reduced the skin inflammation below that of the WT imiquimod-treated mice, and normalised many of the skin and systemic immunological markers to that of WT, suggestive of a mechanism by which anti-CD1a 116 has effects beyond the inhibition of CD1a-TCR signalling. The skin was immunophenotyped and reduction in T cell numbers and activation was observed, as was neutrophil infiltration to the WT level with administration of antibodies 110, 116 and 16. Observation of reduced neutrophilia to the WT level is an unexpected improvement upon published anti-CD1a CR2113, highlighting the potential of antibodies 110, 116 and 16.

Importantly when the LC population within the skin was analysed, significant reduction in the CD11c+Langerin+ LCs was observed following administration of the antibodies 110 and 116. This reduction was not explained by enhanced migration to the draining lymph node. It is however possible that the antibodies 110 and to a greater extent 116 are capable of directly reducing CD1a+ cells in vivo, explaining the reduction in skin LCs in vivo and evidenced by the striking reduction of human CD1a+ cells in vitro. This is a surprising result given the mouse IgG1 isotype of the antibodies—where a murine IgG2a isotype is more likely to lead to cytotoxicity via complement-mediated lysis or antibody-dependent cellular cytotoxicity, and further patented and published anti-CD1a CR2113 has been reported not capable of direct depletion (17), although here it was shown that apoptosis of CD1a-expressing cells could also be induced by CR2113 on a murine IgG1 background. The modulation ability of these antibodies could help explain the reduction of imiquimod induced inflammation below that of WT isotype treated mice. Antibody 116 not only blocks the interaction of CD1a with the TCR but also modifies LCs reducing/resetting the inflammatory potential of the skin and normalised many of the skin and systemic immunological markers to that of WT. This may explain the ameliorating effect over and above the CD1a-dependent response to improvement beyond wild-type, which anti-CD1a CR2113 does not.

Furthermore, the data suggest that the 16, 110 and/or 116 antibodies presented here have utility in the treatment of CD1a-expressing malignancies such as Langerhans cell histiocytosis or some forms of T cell lymphoma and thymomas. This may be by direct effects or wherein an anti-CD1a antibody is coupled or associated with one or more other therapeutic agent is selected from the group comprising cytotoxic agents, anti-inflammatory agents such as steroids, and CAR-T cells such as regulatory or cytolytic CAR-T cells, or other cells expressing or presenting the antibody or antigen binding fragment.

This investigation demonstrates antibody 16 as a highly effective blocking antibody ablating CD1a dependent inflammation in vivo without inducing direct apoptosis, 110 modifies LC phenotype and function, significantly reducing CD1a dependent inflammation in vivo, and 116 is a highly effective blocking and modifying antibody which reduces inflammation below the WT level and normalised many of the skin and systemic immunological markers to that of WT. This grouping of antibodies is consistent with the basic epitope analysis where directly modifying antibodies 110 and 116 cluster and blocking antibodies 77a, 111 and 16 cluster. The epitope analysis also revealed group 77a, 111 and 16 overlapped with the epitope recognised by non-depleting NA 1/34; this is important to note as NA1/34 has been shown to cross-block binding of anti-CD1a CR2113. Antibodies 110 and 116 did not cross-block NA1/34 and therefore likely represents a different epitope region. The antibodies maintain presence on LC in vivo in the skin and even after migration to the lymph nodes. This is an important enhancement as the clinical effects will be more long-lasting.

With these data the inventors demonstrate the potential of this refined panel of improved anti-CD1a antibodies in the prevention and treatment of inflammatory skin and mucosal conditions including, but not limited to, psoriasis, dermatitis, lupus as well as for use in treating and/or preventing one or more associated systemic diseases or disorders, or one or more inflammatory drug reactions which manifest systemically. The effects on a wide cascade of inflammation including LC, T cells and neutrophils, particularly of antibodies 110, 116 and 16, would have wide reaching effects in inflammatory skin and mucosal disorders including psoriasis, dermatitis, lupus and drug reactions which manifest as an inflammatory skin or mucosal disease or disorder, or CD1a-expressing malignancies.

Here the structural basis for the antibodies binding to CD1a is defined and the lipid-dependency of binding is examined, as well as the functional effects of different human IgG1 variants on function in vitro. It is shown that OX16 and OX116 bind at different sites and that OX116 binding is fully lipid antigen independent despite proximity to the F′ portal explaining the broad effect on CD1a blockade. However, the interaction of CD1a with OX16 is moderately affected by the headgroup of the protruding lipids, which suggest a possible mechanism of selective autoreactive recognition of CD1a molecules carrying only certain species of smaller permissive auto-lipids, but not those with larger headgroups which might be required for immunity. The anti-CD1a antibody OX25 is produced, which has a binding site on the alpha 3 domain of CD1a; the data collectively present a range of antibodies with CD1a binding sites with different associated functions. Improvements of the antibodies over other published antibodies CR2113 and mAb571 are also shown, consistent with a role for the antibodies in diagnosis, monitoring, prevention and treatment of CD1a-dependent disease.

In conclusion the inventors demonstrate improved anti-CD1a antibodies 16, 77a, 110, 111 and 116 as a method for preventing and treating inflammatory skin and mucosal diseases or disorders, or as associated systemic diseases or disorders, or inflammatory drug reactions which manifest systemically, or CD1a-expressing malignancies through blocking of CD1a and/or modifying the phenotype/function of CD1a+ cells.

### REFERENCES

1. G. F. Murphy, A. K. Bhan, S. Sato, M. C. Mihm, Jr., T. J. Harrist, A new immunologic marker for human Langerhans cells. N Engl J Med 304, 791-792 (1981).
2. Y. L. Chen et al., Re-evaluation of human BDCA-2+DC during acute sterile skin inflammation. J Exp Med 217, (2020).
3. S. G. Turville et al., Diversity of receptors binding HIV on dendritic cell subsets. Nat Immunol 3, 975-983 (2002).
4. M. Alcantara-Hernandez et al., High-Dimensional Phenotypic Mapping of Human Dendritic Cells Reveals Interindividual Variation and Tissue Specialization. Immunity 47, 1037-1050 e1036 (2017).
5. C. S. Hardman et al., CD1a presentation of endogenous antigens by group 2 innate lymphoid cells. Sci Immunol 2, (2017).
6. E. L. Reinherz, P. C. Kung, G. Goldstein, R. H. Levey, S. F. Schlossman, Discrete stages of human intrathymic differentiation: analysis of normal thymocytes and leukemic lymphoblasts of T-cell lineage. Proc Natl Acad Sci USA 77, 1588-1592 (1980).
7. D. B. Moody et al., Structural requirements for glycolipid antigen recognition by CD1b-restricted T cells. Science 278, 283-286 (1997).
8. L. Gapin, D. I. Godfrey, J. Rossjohn, Natural Killer T cell obsession with self-antigens. Curr Opin Immunol 25, 168-173 (2013).
9. T. Mallevaey et al., A molecular basis for NKT cell recognition of CD1d-self-antigen. Immunity 34, 315-326 (2011).
10. K. S. Wun et al., T cell autoreactivity directed toward CD1c itself rather than toward carried self lipids. Nat Immunol 19, 397-406 (2018).
11. A. de Jong et al., CD1a-autoreactive T cells recognize natural skin oils that function as headless antigens. Nat Immunol 15, 177-185 (2014).
12. E. Layre, A. de Jong, D. B. Moody, Human T cells use CD1 and MR1 to recognize lipids and small molecules. Curr Opin Chem Biol 23, 31-38 (2014).
13. H. He et al., Tape strips detect distinct immune and barrier profiles in atopic dermatitis and psoriasis. J Allergy Clin Immunol 147, 199-212 (2021).
14. E. G. Langeveld-Wildschut et al., Clinical and immunologic variables in skin of patients with atopic eczema and either positive or negative atopy patch test reactions. J Allergy Clin Immunol 105, 1008-1016 (2000).
15. A. Wollenberg, S. Kraft, D. Hanau, T. Bieber, Immunomorphological and ultrastructural characterization of Langerhans cells and a novel, inflammatory dendritic epidermal cell (IDEC) population in lesional skin of atopic eczema. J Invest Dermatol 106, 446-453 (1996).
16. J. H. Kim et al., CD1a on Langerhans cells controls inflammatory skin disease. Nat Immunol 17, 1159-1166 (2016).
17. G. I. Bechan et al., Phage display generation of a novel human anti-CD1A monoclonal antibody with potent cytolytic activity. Br J Haematol 159, 299-310 (2012).
18. Oteo M, Parra J F, Mirones I, Giménez L I, Setién F, Martinez-Naves E. Single strand conformational polymorphism analysis of human CD1 genes in different ethnic groups. Tissue Antigens 53, 545-50. (1999).
19. Fanti P A, Dika E, Vaccari S, Miscial C, Varotti C. Generalized psoriasis induced by topical treatment of actinic keratosis with imiquimod. Int J Dermatol 45, 1464-5 (2006).
20. Rajan N, Langtry J A. Generalized exacerbation of psoriasis associated with imiquimod cream treatment of superficial basal cell carcinomas. Clin Exp Dermatol 31, 140-1 (2006).
21. Patel U, Mark N M, Machler B C, Levine V J. Imiquimod 5% cream induced psoriasis: a case report, summary of the literature and mechanism. Br J Dermatol. 164, 670-2 (2011).
22. Tedman A, Malla U, Vasanthakumar L, Buzacott K, Banney L. Stevens-Johnson syndrome due to topical imiquimod 5%. Aust J Gen Pract. 49, 662-664. (2020).
23. Yanes D A, Kaffenberger J A, Carr D R. Erythema multiforme as a reaction to imiquimod 5% cream. Dermatol Online J. 23, 13030 (2017).
24. Tandon Y, Brodell R T. Local reactions to imiquimod in the treatment of basal cell carcinoma. Dermatol Online J. 18, 1 (2012).
25. Giraud S, Leducq S, Kervarrec T, Barbarot S, Laghmari O, Samimi M. Spectrum of imiquimod-induced lupus-like reactions: Report of two cases. Dermatol Ther. 33, e13148 (2020).
26. Maxfield L, Gaston D, Peck A, Hansen K. Topical Imiquimod and Subsequent Erythema Multiforme. J Am Osteopath Assoc. doi: 10.7556/jaoa.2020.010. (2019)
27. Hammerl V, Parlar B, Navarini A, Gantenbein L, Vath H, Mueller S M. Mucosal side effects in patients treated with topical imiquimod-A scoping review of the literature. Dermatol Ther. 34, e14355 (2021).
28. Furuoka K, Fukumoto T, Nagai H, Nishigori C. Topical imiquimod-induced lichenoid drug reaction successfully treated with tacrolimus ointment. Dermatol Ther. 33, e14480 (2020).
29. Maguiness S M, Farsani T T, Zedek D C, Berger T G. Imiquimod-induced subacute cutaneous lupus erythematosus-like changes. Cutis. 95, 349-51 (2015).
30. Yokogawa M, Takaishi M, Nakajima K, Kamijima R, Fujimoto C, Kataoka S, Terada Y, Sano S. Epicutaneous application of toll-like receptor 7 agonists leads to systemic autoimmunity in wild-type mice: a new model of systemic Lupus erythematosus. Arthritis Rheumatol. 66, 694-706 (2014).
31. Stockenhuber K, Hegazy A N, West N R, Ilott N E, Stockenhuber A, Bullers S J, Thornton E E, Arnold I C, Tucci A, Waldmann H, Ogg G S, Powrie F. Foxp3+ T reg cells control psoriasiform inflammation by restraining an IFN-I-driven CD8+ T cell response. J Exp Med. 215, 1987-1998 (2018).

All references cited herein, including patents, patent applications, papers, textbooks and the like, and the references cited therein, to the extent that they are not already, are hereby incorporated herein by reference in their entirety.

TABLE 11
Sequence IDs

| SEQ | | |
|---|---|---|
| ID NO | Feature | Sequence |
| 1 | 16 CDR1 H | GFTFSNYAMS |
| 2 | 16 CDR2 H | AINSNGGSAY YPDTVKD |
| 3 | 16 CDR3 H | RFYYDYGWFA Y |
| 4 | 16 CDR1 L | RASENIDSYL A |
| 5 | 16 CDR2 L | AATLLAD |
| 6 | 16 CDR3 L | QHYYSSPWT |
| 7 | 16-Hv full | EVQLVESGGG LVKPGGSLKL SCAASGFTFS NYAMSWVRQT |
| | | PEKRLEWVAA INSNGGSAYY PDTVKDRFTI SRDNAKNTLY |
| | | LQMSSLRSED TALYYCARRF YYDYGWFAYW GQGTLVTVSS |
| 8 | 16-Lv full | DIVLTQSPAS LSASVGETVT ITCRASENID SYLAWYQQKQ |
| | | GKSPQLLVYA ATLLADGVPS RFSGSGSGTQ YSLKINSLQS |
| | | EDVARYYCQH YYSSPWTFGG GTKLEIK |
| 9 | 77a CDR1 H | GFSLSSYAMS |
| 10 | 77a CDR2 H | IISSSGTTYY ASWAKG |
| 11 | 77a CDR3 H | VDYYSSGWGG L |
| 12 | 77a CDR1 L | QASEDIYSNL A |
| 13 | 77a CDR2 L | GASTLAS |
| 14 | 77a CDR3 L | QCTYDTSSYG NT |
| 15 | 77a-Hv full | QSVEESGGRL VTPGTPLTLT CTVSGFSLSS YAMSWVRQAP |
| | | GKGLEWIGII SSSGTTYYAS WAKGRFTISK TSTTVDLKIT |
| | | SPTTEDTATY FCARVDYYSS GWGGLWGPGT LVTVSS |
| 16 | 77a -Lv full | AVEMTQTPAS MSAAVGGTVT IKCQASEDIY SNLAWYQQKP |
| | | GQPPKLLIYG ASTLASGVPS RFKGSGSGTE YTLTISGVQC |
| | | DDAATYYCQC TYDTSSYGNT FGGGTEMVVE |
| 17 | 110 CDR1 H | GFSLSSYAMI |
| 18 | 110 CDR2 H | IINSSDNTHY ATWAKG |
| 19 | 110 CDR3 H | DPYDYGYGWY FDL |
| 20 | 110 CDR1 L | QASQSVENNK NLA |
| 21 | 110 CDR2 L | KASTLAS |
| 22 | 110 CDR3 L | QGEFSCSSTD CVT |
| 23 | 110-Hv full | QSVEESGGRL VTPGTPLTLT CTVSGFSLSS YAMIWVRQAP |
| | | GKGLEWIGII NSSDNTHYAT WAKGRFTISK TSTTVDLKIT |
| | | SPTTEDTATY FCARDPYDYG YGWYFDLWGP GTLVTVSS |
| 24 | 110 -Lv full | AQVLTQTPSP VSAAVGGTVT INCQASQSVF NNKNLAWYQQ |
| | | KPGQPPKLLI YKASTLASGV SSRFKGSGSG TQFALTISGV |
| | | QCDDAATYYC QGEFSCSSTD CVTFGGGTEV VVK |
| 25 | 111 CDR1 H | GFSLSTYAMS |
| 26 | 111 CDR2 H | IISSSGSTYY ASWAKG |
| 27 | 111 CDR3 H | ETWYWLDL |
| 28 | 111 CDR1 L | QASEDIYSNL A |
| 29 | 111 CDR2 L | GASTLAS |
| 30 | 111 CDR3 L | QCAYDSSSYG TP |
| 31 | 111-Hv full | QSVEESGGRL VTPGTPLTLT CTASGFSLST YAMSWVRQAP |
| | | GKGLEWIGII SSSGSTYYAS WAKGRFTISK TSTTVDLKIT |
| | | SPTTEDTATY FCARETWYWL DLWGQGTLVT VSS |
| 32 | 111-Lv full | AVEMTQTPAS VSAAVGGTVT INCQASEDIY SNLAWYQQKP |
| | | GQPPKLLIYG ASTLASGVPS RFKGSGSGTE YTLTISGVQC |
| | | DDAATYYCQC AYDSSSYGTP FGGGTEVVVK |
| 33 | 116 CDR1 H | GFSLSNYAMS |
| 34 | 116 CDR2 H | IIYTTGFTYY ASWVKG |
| 35 | 116 CDR3 H | GLATYVSPPT RLDL |
| 36 | 116 CDR1 L | QASQSIYNSK NLA |
| 37 | 116 CDR2 L | SASTLAS |
| 38 | 116 CDR3 L | QGEFSCSSVD CAT |
| 39 | 116-Hv full | QSVEESGGRL VTPGTPLTLT CTVSGFSLSN YAMSWVRQAP |
| | | GKGLEWIGII YTTGFTYYAS WVKGRFTISK TSTTVDLKIT |
| | | SPTTEDTATY FCARGLATYV SPPTRLDLWG QGTLVTVSS |
| 40 | 116-Lv full | AQVLTQTPSP VSAAVGGTVT INCQASQSIY NSKNLAWYQQ |
| | | KPGQPPKLLI YSASTLASGV PSRFKGSGSG TQFTLTISDL |
| | | ECDDAATYYC QGEFSCSSVD CATFGGGTEV VVK |
| 41 | 16 H full | EVQLVESGGG LVKPGGSLKL SCAASGFTFS NYAMSWVRQT |
| | | PEKRLEWVAA INSNGGSAYY PDTVKDRFTI SRDNAKNTLY |
| | | LQMSSLRSED TALYYCARRF YYDYGWFAYW GQGTLVTVSS |
| | | AKTTPPSVYP LAPGSAAQTN SMVTLGCLVK GYFPEPVTVT |
| | | WNSGSLSSGV HTFPAVLQSD LYTLSSSVTV PSSTWPSETV |
| | | TCNVAHPASS TKVDKKIVPR DCGCKPCICT VPEVSSVFIF |
| | | PPKPKDVLTI TLTPKVTCVV VDISKDDPEV QFSWFVDDVE |
| | | VHTAQTQPRE EQFNSTFRSV SELPIMHQDW LNGKEFKCRV |
| | | NSAAFPAPIE KTISKTKGRP KAPQVYTIPP PKEQMAKDKV |
| | | SLTCMITDFF PEDITVEWQW NGQPAENYKN TQPIMDTDGS |
| | | YFVYSKLNVQ KSNWEAGNTF TCSVLHEGLH NHHTEKSLSH |
| | | SPGK |
| 42 | 16 L full | DIVLTQSPAS LSASVGETVT ITCRASENID SYLAWYQQKQ |
| | | GKSPQLLVYA ATLLADGVPS RFSGSGSGTQ YSLKINSLQS |
| | | EDVARYYCQH YYSSPWTFGG GTKLEIKRTD AAPTVSIFPP |
| | | SSEQLTSGGA SVVCFLNNFY PKDINVKWKI DGSERQNGVL |
| | | NSWTDQDSKD STYSMSSTLT LTKDEYERHN SYTCEATHKT |
| | | STSPIVKSEN RNEC |
| 43 | 77a H full | QSVEESGGRL VTPGTPLTLT CTVSGFSLSS YAMSWVRQAP |
| | | GKGLEWIGII SSSGTTYYAS WAKGRFTISK TSTTVDLKIT |
| | | SPTTEDTATY FCARVDYYSS GWGGLWGPGT LVTVSSAKTT |
| | | PPSVYPLAPG SAAQTNSMVT LGCLVKGYFP EPVTVTWNSG |
| | | SLSSGVHTFP AVLQSDLYTL SSSVTVPSST WPSETVTCNV |
| | | AHPASSTKVD KKIVPRDCGC KPCICTVPEV SSVFIFPPKP |
| | | KDVLTITLTP KVTCVVVDIS KDDPEVQFSW FVDDVEVHTA |
| | | QTQPREEQFN STFRSVSELP IMHQDWLNGK EFKCRVNSAA |
| | | FPAPIEKTIS KTKGRPKAPQ VYTIPPPKEQ MAKDKVSLTC |
| | | MITDFFPEDI TVEWQWNGQP AENYKNTQPI MDTDGSYFVY |
| | | SKLNVQKSNW EAGNTFTCSV LHEGLHNHHT EKSLSHSPGK |
| 44 | 77a L full | AVEMTQTPAS MSAAVGGTVT IKCQASEDIY SNLAWYQQKP |
| | | GQPPKLLIYG ASTLASGVPS RFKGSGSGTE YTLTISGVQC |
| | | DDAATYYCQC TYDTSSYGNT FGGGTEMVVE RTDAAPTVSI |
| | | FPPSSEQLTS GGASVVCFLN NFYPKDINVK WKIDGSERQN |
| | | GVLNSWTDQD SKDCTYSMSS TLTLTKDEYE RHNSYTCEAT |
| | | HKTSTSPIVK SFNRNEC |
| 45 | 110 H full | QSVEESGGRL VTPGTPLTLT CTVSGFSLSS YAMIWVRQAP |
| | | GKGLEWIGII NSSDNTHYAT WAKGRFTISK TSTTVDLKIT |
| | | SPTTEDTATY FCARDPYDYG YGWYFDLWGP GTLVTVSSAK |
| | | TTPPSVYPLA PGSAAQTNSM VTLGCLVKGY FPEPVTVTWN |
| | | SGSLSSGVHT FPAVLQSDLY TLSSSVTVPS STWPSETVTC |
| | | NVAHPASSTK VDKKIVPRDC GCKPCICTVP EVSSVFIFPP |
| | | KPKDVLTITL TPKVTCVVVD ISKDDPEVQF SWFVDDVEVH |
| | | TAQTQPREEQ FNSTFRSVSE LPIMHQDWLN GKEFKCRVNS |
| | | AAFPAPIEKT ISKTKGRPKA PQVYTIPPPK EQMAKDKVSL |
| | | TCMITDFFPE DITVEWQWNG QPAENYKNTQ PIMDTDGSYF |
| | | VYSKLNVQKS NWEAGNTFTC SVLHEGLHNH HTEKSLSHSP |
| | | GK |
| 46 | 110 L full | AQVLTQTPSP VSAAVGGTVT INCQASQSVF NNKNLAWYQQ |
| | | KPGQPPKLLI YKASTLASGV SSRFKGSGSG TQFALTISGV |
| | | QCDDAATYYC QGEFSCSSTD CVTFGGGTEV VVKRTDAAPT |
| | | VSIFPPSSEQ LTSGGASVVC FLNNFYPKDI NVKWKIDGSE |
| | | RQNGVLNSWT DQDSKDCTYS MSSTLTLTKD EYERHNSYTC |
| | | EATHKTSTSP IVKSFNRNEC |
| 47 | 111 H full | QSVEESGGRL VTPGTPLTLT CTASGFSLST YAMSWVRQAP |
| | | GKGLEWIGII SSSGSTYYAS WAKGRFTISK TSTTVDLKIT |
| | | SPTTEDTATY FCARETWYWL DLWGQGTLVT VSSAKTTPPS |
| | | VYPLAPGSAA QTNSMVTLGC LVKGYFPEPV TVTWNSGSLS |
| | | SGVHTFPAVL QSDLYTLSSS VTVPSSTWPS ETVTCNVAHP |
| | | ASSTKVDKKI VPRDCGCKPC ICTVPEVSSV FIFPPKPKDV |
| | | LTITLTPKVT CVVVDISKDD PEVQFSWFVD DVEVHTAQTQ |
| | | PREEQFNSTF RSVSELPIMH QDWLNGKEFK CRVNSAAFPA |
| | | PIEKTISKTK GRPKAPQVYT IPPPKEQMAK DKVSLTCMIT |
| | | DFFPEDITVE WQWNGQPAEN YKNTQPIMDT DGSYFVYSKL |
| | | NVQKSNWEAG NTFTCSVLHE GLHNHHTEKS LSHSPGK |
| 48 | 111 L full | AVEMTQTPAS VSAAVGGTVT INCQASEDIY SNLAWYQQKP |
| | | GQPPKLLIYG ASTLASGVPS RFKGSGSGTE YTLTISGVQC |
| | | DDAATYYCQC AYDSSSYGTP FGGGTEVVVK RTDAAPTVSI |
| | | FPPSSEQLTS GGASVVCFLN NFYPKDINVK WKIDGSERQN |
| | | GVLNSWTDQD SKDCTYSMSS TLTLTKDEYE RHNSYTCEAT |
| | | HKTSTSPIVK SFNRNEC |
| 49 | 116 H full | QSVEESGGRL VTPGTPLTLT CTVSGFSLSN YAMSWVRQAP |
| | | GKGLEWIGII YTTGFTYYAS WVKGRFTISK TSTTVDLKIT |
| | | SPTTEDTATY FCARGLATYV SPPTRLDLWG QGTLVTVSSA |
| | | KTTPPSVYPL APGSAAQTNS MVTLGCLVKG YFPEPVTVTW |
| | | NSGSLSSGVH TFPAVLQSDL YTLSSSVTVP SSTWPSETVT |
| | | CNVAHPASST KVDKKIVPRD CGCKPCICTV PEVSSVFIFP |
| | | PKPKDVLTIT LTPKVTCVVV DISKDDPEVQ FSWFVDDVEV |
| | | HTAQTQPREE QFNSTERSVS ELPIMHQDWL NGKEFKCRVN |
| | | SAAFPAPIEK TISKTKGRPK APQVYTIPPP KEQMAKDKVS |
| | | LTCMITDFFP EDITVEWQWN GQPAENYKNT QPIMDTDGSY |
| | | FVYSKLNVQK SNWEAGNTFT CSVLHEGLHN HHTEKSLSHS |
| | | PGK |
| 50 | 116 L full | AQVLTQTPSP VSAAVGGTVT INCQASQSIY NSKNLAWYQQ |
| | | KPGQPPKLLI YSASTLASGV PSRFKGSGSG TQFTLTISDL |
| | | ECDDAATYYC QGEFSCSSVD CATFGGGTEV VVKRTDAAPT |
| | | VSIFPPSSEQ LTSGGASVVC FLNNFYPKDI NVKWKIDGSE |
| | | RQNGVLNSWT DQDSKDCTYS MSSTLTLTKD EYERHNSYTC |
| | | EATHKTSTSP IVKSFNRNEC |
| 51 | 16 H DNA | AAGCTTGCCA CCATGGAATG GAGCTGGGTC TTTCTCTTCT |
| | | TCCTGTCAGT AACTACAGGA GTCCATTCTG AGGTGCAGCT |
| | | GGTGGAGTCT GGGGGAGGCT TAGTGAAGCC TGGAGGGTCC |
| | | CTGAAACTCT CCTGTGCAGC CTCTGGATTC ACTTTCAGTA |
| | | ACTATGCCAT GTCTTGGGTT CGCCAGACTC CAGAGAAGAG |
| | | GCTGGAGTGG GTCGCAGCCA TTAATAGTAA TGGTGGTAGC |
| | | GCCTACTATC CAGACACTGT GAAGGACCGA TTCACCATCT |
| | | CCAGAGACAA TGCCAAGAAC ACCCTGTACC TGCAAATGAG |
| | | CAGTCTGAGG TCTGAGGACA CAGCCTTGTA TTACTGTGCA |
| | | AGACGCTTCT ACTATGATTA CGGCTGGTTT GCTTACTGGG |
| | | GCCAAGGGAC TCTGGTCACA GTCTCGAGC |
| 52 | 16 L DNA | AAGCTTGCCA CCATGTCTGT CCCCACCCAA GTCCTCGGAC |
| | | TCCTGCTACT CTGGCTTACA GATGCCAGAT GCGACATTGT |
| | | GCTGACCCAA TCTCCAGCTT CCCTGTCTGC ATCTGTGGGA |
| | | GAAACTGTCA CCATCACATG TCGAGCAAGT GAGAATATTG |
| | | ACAGTTATTT AGCATGGTAT CAGCAGAAAC AGGGAAAATC |
| | | TCCTCAGCTC CTGGTCTATG CTGCAACACT CTTAGCAGAT |
| | | GGTGTGCCAT CAAGGTTCAG TGGCAGTGGA TCAGGCACAC |
| | | AGTATTCTCT CAAGATCAAC AGCCTGCAGT CTGAAGATGT |
| | | TGCGAGATAT TACTGTCAAC ATTATTATAG TTCTCCGTGG |
| | | ACGTTCGGTG GAGGCACCAA GCTGGAAATA AAACGTACG |
| 53 | 77a H DNA | AAGCTTGCCA CCATGGAATG GAGCTGGGTC TTTCTCTTCT |
| | | TCCTGTCAGT AACTACAGGA GTCCATTCTC AGTCGGTGGA |
| | | GGAGTCCGGG GGTCGCCTGG TCACGCCTGG GACACCCCTG |
| | | ACACTCACAT GCACAGTCTC TGGATTCTCC CTCAGTAGCT |
| | | ATGCGATGAG CTGGGTCCGC CAGGCTCCAG GGAAGGGGCT |
| | | GGAATGGATC GGAATCATTA GTAGCAGTGG TACCACATAC |
| | | TACGCGAGCT GGGCGAAAGG CCGATTCACC ATTTCCAAAA |
| | | CCTCGACCAC GGTGGATCTG AAAATCACCA GTCCGACAAC |
| | | CGAGGACACG GCCACCTATT TCTGTGCCAG AGTCGATTAC |
| | | TATAGTAGTG GCTGGGGTGG CTTGTGGGGC CCAGGCACCC |
| | | TGGTCACCGT CTCGAGC |
| 54 | 77a H DNA | AAGCTTCGAA GCCACCATGG ACACGAGGGC CCCCACTCAG |
| | | CTGCTGGGGC TCCTGCTGCT CTGGCTCCCA GGTGCCACAT |
| | | TTGCCGTTGA AATGACCCAG ACTCCAGCCT CGATGTCTGC |
| | | CGCTGTGGGA GGCACAGTCA CCATCAAGTG CCAGGCCAGT |
| | | GAGGACATTT ATAGCAATTT GGCCTGGTAT CAGCAGAAAC |
| | | CAGGGCAGCC TCCCAAGCTC CTGATCTATG GTGCATCCAC |
| | | TCTGGCTTCT GGGGTCCCAT CGCGGTTCAA AGGCAGTGGA |
| | | TCTGGGACAG AGTACACTCT CACCATCAGC GGTGTGCAGT |
| | | GTGACGATGC TGCCACTTAC TATTGTCAAT GCACTTATGA |
| | | TACTAGTAGT TATGGTAATA CTTTCGGCGG AGGGACCGAG |
| | | ATGGTAGTCG AACGTACG |
| 55 | 110 H DNA | AAGCTTGCCA CCATGGAATG GAGCTGGGTC TTTCTCTTCT |
| | | TCCTGTCAGT AACTACAGGA GTCCATTCTC AGTCGGTGGA |
| | | GGAGTCCGGG GGTCGCCTGG TCACGCCTGG GACACCCCTG |
| | | ACACTCACCT GCACAGTCTC TGGATTCTCC CTCAGTAGCT |
| | | ATGCAATGAT CTGGGTCCGC CAGGCTCCAG GGAAGGGGCT |
| | | GGAATGGATC GGAATCATTA ATAGTAGTGA TAACACACAC |
| | | TACGCGACCT GGGCGAAAGG CCGATTCACC ATCTCCAAAA |
| | | CCTCGACCAC GGTGGATCTA AAAATCACCA GTCCGACAAC |
| | | CGAGGACACG GCCACCTATT TCTGTGCCAG AGATCCCTAC |
| | | GACTATGGTT ATGGTTGGTA CTTTGACTTG TGGGGCCCAG |
| | | GCACCCTGGT CACCGTCTCG AGC |
| 56 | 110 L DNA | AAGCTTCGAA GCCACCATGG ACACGAGGGC CCCCACTCAG |
| | | CTGCTGGGGC TCCTGCTGCT CTGGCTCCCA GGTGCCACAT |
| | | TTGCCCAAGT GCTGACCCAG ACTCCATCCC CTGTGTCTGC |
| | | AGCTGTGGGA GGCACAGTCA CCATCAACTG CCAGGCCAGT |
| | | CAGAGTGTTT TTAATAACAA AAATTTAGCC TGGTATCAGC |
| | | AGAAACCAGG GCAGCCTCCC AAGCTCCTGA TCTACAAGGC |
| | | ATCCACTCTG GCATCTGGCG TCTCATCGCG GTTCAAAGGC |
| | | AGTGGATCTG GGACACAGTT CGCTCTCACC ATCAGCGGCG |
| | | TGCAGTGTGA CGATGCTGCC ACTTACTACT GTCAAGGCGA |
| | | ATTTAGTTGT AGTAGTACTG ATTGCGTGAC TTTCGGCGGA |
| | | GGGACCGAGG TGGTGGTCAA ACGTACG |
| 57 | 111 H DNA | AAGCTTGCCA CCATGGAATG GAGCTGGGTC TTTCTCTTCT |
| | | TCCTGTCAGT AACTACAGGA GTCCATTCTC AGTCGGTGGA |
| | | GGAGTCCGGG GGTCGCCTGG TCACGCCTGG GACACCCCTG |
| | | ACACTCACCT GCACAGCCTC TGGATTCTCC CTCAGTACCT |
| | | ATGCAATGAG TTGGGTCCGC CAGGCTCCAG GGAAGGGGCT |
| | | GGAATGGATC GGAATCATTA GTAGTAGTGG TAGCACATAC |
| | | TACGCGAGCT GGGCGAAAGG CCGATTCACC ATCTCCAAAA |
| | | CCTCGACCAC GGTGGATCTG AAAATCACCA GTCCGACAAC |
| | | CGAGGACACG GCCACCTATT TCTGTGCCAG AGAGACTTGG |
| | | TACTGGTTGG ATCTCTGGGG CCAGGGCACC CTGGTCACCG |
| | | TCTCGAGC |
| 58 | 111 L DNA | AAGCTTCGAA GCCACCATGG ACATGAGGGC CCCCACTCAG |
| | | CTGCTGGGGC TCCTGCTGCT CTGGCTCCCA GGTGCCACAT |
| | | TTGCCGTTGA AATGACCCAG ACTCCAGCCT CGGTGTCTGC |
| | | CGCTGTGGGA GGCACAGTCA CCATCAATTG CCAGGCCAGT |
| | | GAGGACATTT ATAGCAATTT GGCCTGGTAT CAGCAGAAAC |
| | | CAGGGCAGCC TCCCAAGCTC CTGATCTATG GTGCATCCAC |
| | | TCTGGCATCT GGGGTCCCAT CGCGGTTCAA AGGCAGTGGA |
| | | TCTGGGACAG AGTACACTCT CACCATCAGC GGTGTGCAGT |
| | | GTGACGATGC TGCCACTTAC TACTGTCAAT GCGCTTATGA |
| | | TAGTAGTAGT TATGGTACCC CTTTCGGCGG AGGGACCGAG |
| | | GTGGTGGTCA AACGTACG |
| 59 | 116 H DNA | AAGCTTGCCA CCATGGAATG GAGCTGGGTC TTTCTCTTCT |
| | | TCCTGTCAGT AACTACAGGA GTCCATTCTC AGTCGGTGGA |
| | | GGAGTCCGGG GGTCGCCTGG TCACGCCTGG GACACCCCTG |
| | | ACACTCACCT GCACAGTCTC TGGATTCTCC CTCAGTAACT |
| | | ATGCAATGAG CTGGGTCCGC CAGGCTCCAG GGAAGGGGCT |
| | | GGAATGGATC GGAATCATTT ATACTACTGG TTTCACATAC |
| | | TACGCGAGCT GGGTGAAAGG CCGATTCACC ATCTCCAAAA |
| | | CCTCGACCAC GGTGGACCTG AAAATCACCA GTCCGACAAC |
| | | CGAGGACACG GCCACCTATT TCTGTGCCAG AGGGCTGGCT |
| | | ACTTATGTTA GTCCCCCGAC TCGGTTGGAT CTCTGGGGCC |
| | | AGGGCACCCT GGTCACCGTC TCGAGC |
| 60 | 116 L DNA | AAGCTTCGAA GCCACCATGA ACATGAGGGC CCCCACTCAG |
| | | CTGCTGGGGC TCCTGCTGCT CTGGCTCCCA GGTGCCACAT |
| | | TTGCCCAAGT GCTGACCCAG ACTCCATCCC CTGTGTCTGC |
| | | AGCTGTGGGA GGCACAGTCA CCATCAACTG CCAGGCCAGT |
| | | CAGAGTATTT ATAATAGCAA AAATTTAGCC TGGTATCAGC |
| | | AGAAACCAGG GCAGCCTCCC AAGCTCCTGA TCTATTCTGC |
| | | ATCCACTCTG GCATCTGGGG TCCCATCGCG GTTCAAAGGC |
| | | AGTGGATCTG GGACACAGTT CACTCTCACC ATCAGCGACC |
| | | TGGAGTGTGA CGATGCTGCC ACTTACTACT GTCAAGGCGA |
| | | ATTTAGTTGT AGTAGTGTTG ATTGCGCCAC TTTCGGCGGA |
| | | GGGACCGAGG TGGTGGTCAA ACGTACG |
| 61 | 16 CDR1 H | GGATTCACTT TCAGTAACTA TGCCATGTCT |
| 62 | 16 CDR2 H | GCCATTAATA GTAATGGTGG TAGCGCCTAC TATCCAGACA |
| | | CTGTGAAGGA C |
| 63 | 16 CDR3 H | CGCTTCTACT ATGATTACGG CTGGTTTGCT TAC |
| 64 | 16 CDR1 L | CGAGCAAGTG AGAATATTGA CAGTTATTTA GCA |
| 65 | 16 CDR2 L | GCTGCAACAC TCTTAGCAGA T |
| 66 | 16 CDR3 L | CAACATTATT ATAGTTCTCC GTGGACG |
| 67 | 77a CDR1 H | GGATTCTCCC TCAGTAGCTA TGCGATGAGC |
| 68 | 77a CDR2 H | ATCATTAGTA GCAGTGGTAC CACATACTAC GCGAGCTGGG |
| | | CGAAAGGC |
| 69 | 77a CDR3 H | GTCGATTACT ATAGTAGTGG CTGGGGTGGC TTG |
| 70 | 77a CDR1 L | CAGGCCAGTG AGGACATTTA TAGCAATTTG GCC |
| 71 | 77a CDR2 L | GGTGCATCCA CTCTGGCTTC T |
| 72 | 77a CDR3 L | CAATGCACTT ATGATACTAG TAGTTATGGT AATACT |
| 73 | 110 CDR1 H | GGATTCTCCC TCAGTAGCTA TGCAATGATC |
| 74 | 110 CDR2 H | ATCATTAATA GTAGTGATAA CACACACTAC GCGACCTGGG |
| | | CGAAAGGC |
| 75 | 110 CDR3 H | GATCCCTACG ACTATGGTTA TGGTTGGTAC TTTGACTTG |
| 76 | 110 CDR1 L | CAGGCCAGTC AGAGTGTTTT TAATAACAAA AATTTAGCC |
| 77 | 110 CDR2 L | AAGGCATCCA CTCTGGCATC T |
| 78 | 110 CDR3 L | CAAGGCGAAT TTAGTTGTAG TAGTACTGAT TGCGTGACT |
| 79 | 111 CDR1 H | GGATTCTCCC TCAGTACCTA TGCAATGAGT |
| 80 | 111 CDR2 H | ATCATTAGTA GTAGTGGTAG CACATACTAC GCGAGCTGGG |
| | | CGAAAGGC |
| 81 | 111 CDR3 H | GAGACTTGGT ACTGGTTGGA TCTC |
| 82 | 111 CDR1 L | CAGGCCAGTG AGGACATTTA TAGCAATTTG GCC |
| 83 | 111 CDR2 L | GGTGCATCCA CTCTGGCATC T |
| 84 | 111 CDR3 L | CAATGCGCTT ATGATAGTAG TAGTTATGGT ACCCCT |
| 85 | 116 CDR1 H | GGATTCTCCC TCAGTAACTA TGCAATGAGC |
| 86 | 116 CDR2 H | ATCATTTATA CTACTGGTTT CACATACTAC GCGAGCTGGG |
| | | TGAAAGGC |
| 87 | 116 CDR3 H | GGGCTGGCTA CTTATGTTAG TCCCCCGACT CGGTTGGATC |
| | | TC |
| 88 | 116 CDR1 L | CAGGCCAGTC AGAGTATTTA TAATAGCAAA AATTTAGCC |
| 89 | 116 CDR2 L | TCTGCATCCA CTCTGGCATC T |
| 90 | 116 CDR3 L | CAAGGCGAAT TTAGTTGTAG TAGTGTTGAT TGCGCCACT |
| 91 | 25 CDR1 H | GFSLTTYEIN |
| 92 | 25 CDR2 H | VIWTGGSTNYNSALIS |
| 93 | 25 CDR3 H | TLPYYYAMDY |
| 94 | 25 CDR1 L | SVSSSISSSNLH |
| 95 | 25 CDR2 L | GTSNLAS |
| 96 | 25 CDR3 L | QQWSSYPPT |
| 97 | 25-Hv full | QVQLQQSGPGLVAPSQSLFITCTVSGFSLTTYEINWVRQPPGKGLEWL |
| | | GVIWTGGSTNYNSALISRLSISKDNSKSLVFLKMNSLQTDDTAIYYCV |
| | | RTLPYYYAMDYWGQGTSVTVSS |
| 98 | 25-Lv full | EIVLTQSPALMAASPGEKVTITCSVSSSISSSNLHWYQQKSGTSPKPW |
| | | IYGTSNLASGVPVRFSGSGSGTSYSLTISSMEAEDAATYYCQQWSSYP |
| | | PTFGSGTKLEIK |
| 99 | 25 H full | EIVLTQSPALMAASPGEKVTITCSVSSSISSSNLHWYQQKSGTSPKPW |
| | | IYGTSNLASGVPVRFSGSGSGTSYSLTISSMEAEDAATYYCQQWSSYP |
| | | PTFGSGTKLEIKRTDAAPTVSIFPPSSEQLTSGGASVVCFLNNFYPKD |
| | | INVKWKIDGSERQNGVLNSWTDQDSKDSTYSMSSTLTLTKDEYERHNS |
| | | YTCEATHKTSTSPIVKSFNRNEC |
| 100 | 25 L full | QVQLQQSGPGLVAPSQSLFITCTVSGFSLTTYEINWVRQPPGKGLEWL |
| | | GVIWTGGSTNYNSALISRLSISKDNSKSLVFLKMNSLOTDDTAIYYCV |
| | | RTLPYYYAMDYWGQGTSVTVSSAKTTPPSVYPLAPGSAAQTNSMVTLG |
| | | CLVKGYFPEPVTVTWNSGSLSSGVHTFPAVLQSDLYTLSSSVTVPSST |
| | | WPSETVTCNVAHPASSTKVDKKIVPRDCGCKPCICTVPEVSSVFIFPP |
| | | KPKDVLTITLTPKVTCVVVDISKDDPEVQFSWFVDDVEVHTAQTQPRE |
| | | EQFNSTFRSVSELPIMHQDWLNGKEFKCRVNSAAFPAPIEKTISKTKG |
| | | RPKAPQVYTIPPPKEQMAKDKVSLTCMITDFFPEDITVEWQWNGQPAE |
| | | NYKNTQPIMDTDGSYFVYSKLNVQKSNWEAGNTFTCSVLHEGLHNHHT |
| | | EKSLSHSPGK |
| 101 | 116 ScFv full | METDTLLLWVLLLWVPGSTGDAQSVEESGGRLVTPGTPLTLTCTVSGF |
| | | SLSNYAMSWVRQAPGKGLEWIGIIYTTGFTYYASWVKGRFTISKTSTT |
| | | VDLKITSPTTEDTATYFCARGLATYVSPPTRLDLWGQGTLVTVSSGGG |
| | | GSGGGGSGGGGSAQVLTQTPSPVSAAVGGTVTINCQASQSIYNSKNLA |
| | | WYQQKPGQPPKLLIYSASTLASGVPSRFKGSGSGTQFTLTISDLESDD |
| | | AATYYCQGEFSCSSVDCATFGGGTEVVVKSGLEVLFQGPGSGLNDIFE |
| | | AQKIEWHEHHHHHH |
| 102 | 16 ScFv full | METDTLLLWVLLLWVPGSTGDAEVQLVESGGGLVKPGGSLKLSCAASG |
| | | FTFSNYAMSWVRQTPEKRLEWVAAINSNGGSAYYPDTVKDRFTISRDN |
| | | AKNTLYLQMSSLRSEDTALYYCARRFYYDYGWFAYWGQGTLVTVSSGG |
| | | GGSGGGGSGGGGSDIVLTQSPASLSASVGETVTITCRASENIDSYLAW |
| | | YQQKQGKSPQLLVYAATLLADGVPSRFSGSGSGTQYSLKINSLQSEDV |
| | | ARYYCQHYYSSPWTFGGGTKLEIKSGLEVLFQGPGSGLNDIFEAQKIE |
| | | WHEHHHHHH |
| 103 | 110 ScFv full | METDTLLLWVLLLWVPGSTGDAQSVEESGGRLVTPGTPLTLTCTVSGF |
| | | SLSSYAMIWVRQAPGKGLEWIGIINSSDNTHYATWAKGRFTISKTSTT |
| | | VDLKITSPTTEDTATYFCARDPYDYGYGWYFDLWGPGTLVTVSSGGGG |
| | | SGGGGSGGGGSAQVLTQTPSPVSAAVGGTVTINCQASQSVENNKNLAW |
| | | YQQKPGQPPKLLIYKASTLASGVSSRFKGSGSGTQFALTISGVQSDDA |
| | | ATYYCQGEFSCSSTDCVTFGGGTEVVVKSGLEVLFQGPGSGLNDIFEA |
| | | QKIEWHEHHHHHH |
| 104 | 116 ScFv | QSVEESGGRLVTPGTPLTLTCTVSGFSLSNYAMSWVRQAPGKGLEWIG |
| | full - no tags | IIYTTGFTYYASWVKGRFTISKTSTTVDLKITSPTTEDTATYFCARGL |
| | | ATYVSPPTRLDLWGQGTLVTVSSGGGGSGGGGSGGGGSAQVLTQTPSP |
| | | VSAAVGGTVTINCQASQSIYNSKNLAWYQQKPGQPPKLLIYSASTLAS |
| | | GVPSRFKGSGSGTQFTLTISDLESDDAATYYCQGEFSCSSVDCATFGG |
| | | GTEVVVK |
| 105 | 16 ScFv full - | QSVEESGGRLVTPGTPLTLTCTVSGFSLSSYAMIWVRQAPGKGLEWIG |
| | no tags | IINSSDNTHYATWAKGRFTISKTSTTVDLKITSPTTEDTATYFCARDP |
| | | YDYGYGWYFDLWGPGTLVTVSSGGGGSGGGGSGGGGSAQVLTQTPSPV |
| | | SAAVGGTVTINCQASQSVENNKNLAWYQQKPGQPPKLLIYKASTLASG |
| | | VSSRFKGSGSGTQFALTISGVQSDDAATYYCQGEFSCSSTDCVTFGGG |
| | | TEVVVK |
| 106 | 110 ScFv | EVQLVESGGGLVKPGGSLKLSCAASGFTFSNYAMSWVRQTPEKRLEWV |
| | full - no tags | AAINSNGGSAYYPDTVKDRFTISRDNAKNTLYLQMSSLRSEDTALYYC |
| | | ARRFYYDYGWFAYWGQGTLVTVSSGGGGSGGGGGSGGGSDIVLTQSPA |
| | | SLSASVGETVTITCRASENIDSYLAWYQQKOGKSPQLLVYAATLLADG |
| | | VPSRFSGSGSGTQYSLKINSLQSEDVARYYCQHYYSSPWTFGGGTKLE |
| | | IK |
| 107 | 77A CDR3 L | QSTYDTSSYGNT |
| | humanised | |
| | variant 1 | |
| 108 | 77A CDR3 L | QATYDTSSYGNT |
| | humanised | |
| | variant 2 | |
| 109 | 77A CDR3 L | QVTYDTSSYGNT |
| | humanised | |
| | variant 3 | |
| 110 | 110 CDR3 H | EPYDYGYGWYFDL |
| | humanised | |
| | variant 1 | |
| 111 | 110 CDR3 L | QGEFSSSSTDSVT |
| | humanised | |
| | variant 1 | |
| 112 | 110 CDR3 L | QGEFSSSSTDAVT |
| | humanised | |
| | variant 2 | |
| 113 | 110 CDR3 L | QGEFSSSSTDVVT |
| | humanised | |
| | variant 3 | |
| 114 | 110 CDR3 L | QGEFSASSTDSVT |
| | humanised | |
| | variant 4 | |
| 115 | 110 CDR3 L | QGEFSASSTDAVT |
| | humanised | |
| | variant 5 | |
| 116 | 110 CDR3 L | QGEFSASSTDVVT |
| | humanised | |
| | variant 6 | |
| 117 | 110 CDR3 L | QGEFSVSSTDSVT |
| | humanised | |
| | variant 7 | |
| 118 | 110 CDR3 L | QGEFSVSSTDAVT |
| | humanised | |
| | variant 8 | |
| 119 | 110 CDR3 L | QGEFSVSSTDVVT |
| | humanised | |
| | variant 9 | |
| 120 | 111 CDR3 L | QSAYDSSSYGTP |
| | humanised | |
| | variant 1 | |
| 121 | 111 CDR3 L | QAAYDSSSYGTP |
| | humanised | |
| | variant 2 | |
| 122 | 111 CDR3 L | QVAYDSSSYGTP |
| | humanised | |
| | variant 3 | |
| 123 | 116 CDR3 L | QGEFSSSSVDSAT |
| | humanised | |
| | variant 1 | |
| 124 | 116 CDR3 L | QGEFSSSSVDAAT |
| | humanised | |
| | variant 2 | |
| 125 | 116 CDR3 L | QGEFSSSSVDVAT |
| | humanised | |
| | variant 3 | |
| 126 | 116 CDR3 L | QGEFSASSVDSAT |
| | humanised | |
| | variant 4 | |
| 127 | 116 CDR3 L | QGEFSASSVDAAT |
| | humanised | |
| | variant 5 | |
| 128 | 116 CDR3 L | QGEFSASSVDVAT |
| | humanised | |
| | variant 6 | |
| 129 | 116 CDR3 L | QGEFSVSSVDSAT |
| | humanised | |
| | variant 7 | |
| 130 | 116 CDR3 L | QGEFSVSSVDAAT |
| | humanised | |
| | variant 8 | |
| 131 | 116 CDR3 L | QGEFSVSSVDVAT |
| | humanised | |
| | variant 9 | |
| 132 | 110 CDR2 H | IIQSSDNTHYATWAKG |
| | humanised | |
| | variant 1 | |
| 133 | 110 CDR2 H | IIDSSDNTHYATWAKG |
| | humanised | |
| | variant 1 | |
| 134 | 110 CDR2 H | IISSSDNTHYATWAKG |
| | humanised | |
| | variant 1 | |
| 135 | 116 CDR1 L | QASQSIYNTKNLA |
| | humanised | |
| | variant 1 | |
| 136 | 116 CDR1 L | QASQSIYNAKNLA |
| | humanised | |
| | variant 2 | |
| 137 | 116 CDR1 L | QASQSIYNNKNLA |
| | humanised | |
| | variant 3 | |
| 138 | 116 CDR1 L | QASQSIYQSKNLA |
| | humanised | |
| | variant 4 | |
| 139 | 116 CDR1 L | QASQSIYDSKNLA |
| | humanised | |
| | variant 5 | |
| 140 | 16 CDR2 L | AATLLAE |
| | humanised | |
| | variant 1 | |
| 141 | 16 CDR2 L | AATLLAS |
| | humanised | |
| | variant 2 | |
| 142 | 16 CDR2 H | AINSNSGSAYYPDTVKD |
| | humanised | |
| | variant 1 | |
| 143 | 16 CDR2 H | AINSNAGSAYYPDTVKD |
| | humanised | |
| | variant 2 | |
| 144 | 16 CDR2 H | AINSNVGSAYYPDTVKD |
| | humanised | |
| | variant 3 | |
| 145 | 16 CDR2 H | AINSQGGSAYYPDTVKD |
| | humanised | |
| | variant 4 | |
| 146 | 16 CDR2 H | AINSDGGSAYYPDTVKD |
| | humanised | |
| | variant 5 | |
| 147 | 16 CDR2 H | AINANGGSAYYPDTVKD |
| | humanised | |
| | variant 6 | |
| 148 | 16 CDR2 H | AINTNGGSAYYPDTVKD |
| | humanised | |
| | variant 7 | |
| 149 | 16 CDR2 H | AIQSNGGSAYYPDTVKD |
| | humanised | |
| | variant 8 | |
| 150 | 16 CDR2 H | AIDSNGGSAYYPDTVKD |
| | humanised | |
| | variant 9 | |
| 151 | 16 CDR2 H | AITSNGGSAYYPDTVKD |
| | humanised | |
| | variant 10 | |
| 152 | 16 CDR2 H | AISSNGGSAYYPDTVKD |
| | humanised | |
| | variant 11 | |
| 153 | 77a Lv | AVEMTQSPSTLSASVGDRVTITCQASEDIYSNLAWYQQKPGKAPKLLI |
| | humanised | YGASTLASGVPSRFSGSGSGTEYTLTISSLQPDDFATYYCQCTYDTSS |
| | variant 1 | YGNTFGGGTKVEIK |
| 154 | 77a Lv | AVEMTQSPSTLSASVGDRVTITCQASEDIYSNLAWYQQKPGKAPKLLI |
| | humanised | YGASTLASGVPSRFSGSGSGTEYTLTISSLQPDDFATYYCQSTYDTSS |
| | variant 2 | YGNTFGGGTKVEIK |
| 155 | 77a Lv | AVEMTQSPSTLSASVGDRVTITCQASEDIYSNLAWYQQKPGKAPKLLI |
| | humanised | YGASTLASGVPSRFSGSGSGTEYTLTISSLQPDDFATYYCQATYDTSS |
| | variant 3 | YGNTFGGGTKVEIK |
| 156 | 77a Lv | AVEMTQSPSTLSASVGDRVTITCQASEDIYSNLAWYQQKPGKAPKLLI |
| | humanised | YGASTLASGVPSRFSGSGSGTEYTLTISSLQPDDFATYYCQVTYDTSS |
| | variant 4 | YGNTFGGGTKVEIK |
| 157 | 77a Hv | EVQLLESGGGLVQPGGSLRLSCAVSGFSLSSYAMSWVRQAPGKGLEWI |
| | humanised | GIISSSGTTYYASWAKGRFTISKDSSKNTVYLQMNSLRAEDTAVYYCA |
| | variant 1 | RVDYYSSGWGGLWGQGTLVTVSS |
| 158 | 110 Lv | AQVLTQSPSSLSASVGDRVTITCQASQSVENNKNLAWYQQKPGKAPKL |
| | humanised | LIYKASTLASGVPSRFSGSGSGTQFTLTISSLQPEDFATYYCQGEFSC |
| | variant 1 | SSTDCVTFGGGTKVEIK |
| 159 | 110 Lv | AQVLTQSPSSLSASVGDRVTITCQASQSVENNKNLAWYQQKPGKAPKL |
| | humanised | LIYKASTLASGVPSRFSGSGSGTQFTLTISSLQPEDFATYYCQGEFSS |
| | variant 2 | SSTDSVTFGGGTKVEIK |
| 160 | 110 Lv | AQVLTQSPSSLSASVGDRVTITCQASQSVENNKNLAWYQQKPGKAPKL |
| | humanised | LIYKASTLASGVPSRFSGSGSGTQFTLTISSLQPEDFATYYCQGEFSS |
| | variant 3 | SSTDAVTFGGGTKVEIK |
| 161 | 110 Lv | AQVLTQSPSSLSASVGDRVTITCQASQSVENNKNLAWYQQKPGKAPKL |
| | humanised | LIYKASTLASGVPSRFSGSGSGTQFTLTISSLQPEDFATYYCQGEFSS |
| | variant 4 | SSTDVVTFGGGTKVEIK |
| 162 | 110 Lv | AQVLTQSPSSLSASVGDRVTITCQASQSVENNKNLAWYQQKPGKAPKL |
| | humanised | LIYKASTLASGVPSRFSGSGSGTOFTLTISSLQPEDFATYYCQGEFSA |
| | variant 5 | SSTDSVTFGGGTKVEIK |
| 163 | 110 Lv | AQVLTQSPSSLSASVGDRVTITCQASQSVENNKNLAWYQQKPGKAPKL |
| | humanised | LIYKASTLASGVPSRFSGSGSGTQFTLTISSLQPEDFATYYCQGEFSA |
| | variant 6 | SSTDAVTFGGGTKVEIK |
| 164 | 110 Lv | AQVLTQSPSSLSASVGDRVTITCQASQSVFNNKNLAWYQQKPGKAPKL |
| | humanised | LIYKASTLASGVPSRFSGSGSGTQFTLTISSLQPEDFATYYCQGEFSA |
| | variant 7 | SSTDVVTFGGGTKVEIK |
| 165 | 110 Lv | AQVLTQSPSSLSASVGDRVTITCQASQSVENNKNLAWYQQKPGKAPKL |
| | humanised | LIYKASTLASGVPSRFSGSGSGTQFTLTISSLQPEDFATYYCQGEFSV |
| | variant 8 | SSTDSVTFGGGTKVEIK |
| 166 | 110 Lv | AQVLTQSPSSLSASVGDRVTITCQASQSVFNNKNLAWYQQKPGKAPKL |
| | humanised | LIYKASTLASGVPSRFSGSGSGTQFTLTISSLQPEDFATYYCQGEFSV |
| | variant 9 | SSTDAVTFGGGTKVEIK |
| 167 | 110 Lv | AQVLTQSPSSLSASVGDRVTITCQASQSVENNKNLAWYQQKPGKAPKL |
| | humanised | LIYKASTLASGVPSRFSGSGSGTQFTLTISSLQPEDFATYYCQGEFSV |
| | variant 10 | SSTDVVTFGGGTKVEIK |
| 168 | 110 Hv | EVQLVESGGGLVQPGGSLRLSCAVSGFSLSSYAMIWVRQAPGKGLEWI |
| | humanised | GIINSSDNTHYATWAKGRFTISKDSAKNSVYLOMNSLRDEDTAVYYCA |
| | variant 1 | RDPYDYGYGWYFDLWGRGTLVTVSS |
| 169 | 110 Hv | EVQLVESGGGLVQPGGSLRLSCAVSGFSLSSYAMIWVRQAPGKGLEWI |
| | humanised | GIIQSSDNTHYATWAKGRFTISKDSAKNSVYLQMNSLRDEDTAVYYCA |
| | variant 2 | RDPYDYGYGWYFDLWGRGTLVTVSS |
| 170 | 110 Hv | EVQLVESGGGLVQPGGSLRLSCAVSGFSLSSYAMIWVRQAPGKGLEWI |
| | humanised | GIIDSSDNTHYATWAKGRFTISKDSAKNSVYLOMNSLRDEDTAVYYCA |
| | variant 3 | RDPYDYGYGWYFDLWGRGTLVTVSS |
| 171 | 110 Hv | EVQLVESGGGLVQPGGSLRLSCAVSGFSLSSYAMIWVRQAPGKGLEWI |
| | humanised | GIITSSDNTHYATWAKGRFTISKDSAKNSVYLQMNSLRDEDTAVYYCA |
| | variant 4 | RDPYDYGYGWYFDLWGRGTLVTVSS |
| 172 | 110 Hv | EVQLVESGGGLVQPGGSLRLSCAVSGFSLSSYAMIWVRQAPGKGLEWI |
| | humanised | GIISSSDNTHYATWAKGRFTISKDSAKNSVYLQMNSLRDEDTAVYYCA |
| | variant 5 | RDPYDYGYGWYFDLWGRGTLVTVSS |
| 173 | 110 Hv | EVQLVESGGGLVQPGGSLRLSCAVSGFSLSSYAMIWVRQAPGKGLEWI |
| | humanised | GIINSSDNTHYATWAKGRFTISKDSAKNSVYLQMNSLRDEDTAVYYCA |
| | variant 6 | REPYDYGYGWYFDLWGRGTLVTVSS |
| 174 | 111 Lv | AVEMTQSPSTLSASVGDRVTITCQASEDIYSNLAWYQQKPGKAPKLLI |
| | humanised | YGASTLASGVPSRFSGSGSGTEYTLTISSLQPDDFATYYCQCAYDSSS |
| | variant 1 | YGTPFGGGTKVEIK |
| 175 | 111 Lv | AVEMTQSPSTLSASVGDRVTITCQASEDIYSNLAWYQQKPGKAPKLLI |
| | humanised | YGASTLASGVPSRFSGSGSGTEYTLTISSLQPDDFATYYCQSAYDSSS |
| | variant 2 | YGTPFGGGTKVEIK |
| 176 | 111 Lv | AVEMTQSPSTLSASVGDRVTITCQASEDIYSNLAWYQQKPGKAPKLLI |
| | humanised | YGASTLASGVPSRFSGSGSGTEYTLTISSLQPDDFATYYCQAAYDSSS |
| | variant 3 | YGTPFGGGTKVEIK |
| 177 | 111 Lv | AVEMTQSPSTLSASVGDRVTITCQASEDIYSNLAWYQQKPGKAPKLLI |
| | humanised | YGASTLASGVPSRFSGSGSGTEYTLTISSLQPDDFATYYCQVAYDSSS |
| | variant 4 | YGTPFGGGTKVEIK |
| 178 | 111 Hv | EVQLLESGGGLVQPGGSLRLSCAASGFSLSTYAMSWVRQAPGKGLEWI |
| | humanised | GIISSSGSTYYASWAKGRFTISKDSSKNTVYLQMNSLRAEDTAVYYCA |
| | variant 1 | RETWYWLDLWGRGTLVTVSS |
| 179 | 116 Lv | AQVLTQSPSSLSASVGDRVTITCQASQSIYNSKNLAWYQQKPGKAPKL |
| | humanised | LIYSASTLASGVPSRFSGSGSGTQFTLTISSLQPEDFATYYCQGEFSC |
| | variant 1 | SSVDCATFGGGTKVEIK |
| 180 | 116 Lv | AQVLTQSPSSLSASVGDRVTITCQASQSIYNTKNLAWYQQKPGKAPKL |
| | humanised | LIYSASTLASGVPSRFSGSGSGTQFTLTISSLQPEDFATYYCQGEFSC |
| | variant 2 | SSVDCATFGGGTKVEIK |
| 181 | 116 Lv | AQVLTQSPSSLSASVGDRVTITCQASQSIYNAKNLAWYQQKPGKAPKL |
| | humanised | LIYSASTLASGVPSRFSGSGSGTQFTLTISSLQPEDFATYYCQGEFSC |
| | variant 3 | SSVDCATFGGGTKVEIK |
| 182 | 116 Lv | AQVLTQSPSSLSASVGDRVTITCQASQSIYNNKNLAWYQQKPGKAPKL |
| | humanised | LIYSASTLASGVPSRFSGSGSGTOFTLTISSLQPEDFATYYCQGEFSC |
| | variant 4 | SSVDCATFGGGTKVEIK |
| 183 | 116 Lv | AQVLTQSPSSLSASVGDRVTITCQASQSIYQSKNLAWYQQKPGKAPKL |
| | humanised | LIYSASTLASGVPSRFSGSGSGTQFTLTISSLQPEDFATYYCQGEFSC |
| | variant 5 | SSVDCATFGGGTKVEIK |
| 184 | 116 Lv | AQVLTQSPSSLSASVGDRVTITCQASQSIYDSKNLAWYQQKPGKAPKL |
| | humanised | LIYSASTLASGVPSRFSGSGSGTQFTLTISSLQPEDFATYYCQGEFSC |
| | variant 6 | SSVDCATFGGGTKVEIK |
| 185 | 116 Lv | AQVLTQSPSSLSASVGDRVTITCQASQSIYNSKNLAWYQQKPGKAPKL |
| | humanised | LIYSASTLASGVPSRFSGSGSGTQFTLTISSLQPEDFATYYCQGEFSS |
| | variant 7 | SSVDSATFGGGTKVEIK |
| 186 | 116 Lv | AQVLTQSPSSLSASVGDRVTITCQASQSIYNSKNLAWYQQKPGKAPKL |
| | humanised | LIYSASTLASGVPSRFSGSGSGTOFTLTISSLQPEDFATYYCQGEFSS |
| | variant 8 | SSVDAATFGGGTKVEIK |
| 187 | 116 Lv | AQVLTQSPSSLSASVGDRVTITCQASQSIYNSKNLAWYQQKPGKAPKL |
| | humanised | LIYSASTLASGVPSRFSGSGSGTQFTLTISSLQPEDFATYYCQGEFSS |
| | variant 9 | SSVDVATFGGGTKVEIK |
| 188 | 116 Lv | AQVLTQSPSSLSASVGDRVTITCQASQSIYNSKNLAWYQQKPGKAPKL |
| | humanised | LIYSASTLASGVPSRFSGSGSGTQFTLTISSLQPEDFATYYCQGEFSA |
| | variant 10 | SSVDSATFGGGTKVEIK |
| 189 | 116 Lv | AQVLTQSPSSLSASVGDRVTITCQASQSIYNSKNLAWYQQKPGKAPKL |
| | humanised | LIYSASTLASGVPSRFSGSGSGTQFTLTISSLQPEDFATYYCQGEFSA |
| | variant 11 | SSVDAATFGGGTKVEIK |
| 190 | 116 Lv | AQVLTQSPSSLSASVGDRVTITCQASQSIYNSKNLAWYQQKPGKAPKL |
| | humanised | LIYSASTLASGVPSRFSGSGSGTQFTLTISSLQPEDFATYYCQGEFSA |
| | variant 12 | SSVDVATFGGGTKVEIK |
| 191 | 116 Lv | AQVLTQSPSSLSASVGDRVTITCQASQSIYNSKNLAWYQQKPGKAPKL |
| | humanised | LIYSASTLASGVPSRFSGSGSGTQFTLTISSLQPEDFATYYCQGEFSV |
| | variant 13 | SSVDSATFGGGTKVEIK |
| 192 | 116 Lv | AQVLTQSPSSLSASVGDRVTITCQASQSIYNSKNLAWYQQKPGKAPKL |
| | humanised | LIYSASTLASGVPSRFSGSGSGTQFTLTISSLQPEDFATYYCQGEFSV |
| | variant 14 | SSVDAATFGGGTKVEIK |
| 193 | 116 Lv | AQVLTQSPSSLSASVGDRVTITCQASQSIYNSKNLAWYQQKPGKAPKL |
| | humanised | LIYSASTLASGVPSRFSGSGSGTQFTLTISSLQPEDFATYYCQGEFSV |
| | variant 15 | SSVDVATFGGGTKVEIK |
| 194 | 116 Hv | EVQLVESGGGLVQPGGSLRLSCAVSGFSLSNYAMSWVRQAPGKGLEWI |
| | humanised | GIIYTTGFTYYASWVKGRFTISKDSSKNTVYLQMNSLRAEDTAVYYCA |
| | variant 1 | RGLATYVSPPTRLDLWGQGTLVTVSS |
| 195 | 16 Lv | DIQMTQSPSSLSASVGDRVTITCRASENIDSYLAWYQQKPGKAPKLLV |
| | humanised | YAATLLADGVPSRFSGSGSGTQYTLTISSLQPEDFARYYCQHYYSSPW |
| | variant 1 | TFGQGTKVEIK |
| 196 | 16 Lv | DIQMTQSPSSLSASVGDRVTITCRASENIDSYLAWYQQKPGKAPKLLV |
| | humanised | YAATLLAEGVPSRFSGSGSGTQYTLTISSLQPEDFARYYCQHYYSSPW |
| | variant 2 | TFGQGTKVEIK |
| 197 | 16 Lv | DIQMTQSPSSLSASVGDRVTITCRASENIDSYLAWYQQKPGKAPKLLV |
| | humanised | YAATLLASGVPSRFSGSGSGTQYTLTISSLQPEDFARYYCQHYYSSPW |
| | variant 3 | TFGQGTKVEIK |
| 198 | 16 Hv | EVQLLESGGGLVQPGGSLRLSCAASGFTFSNYAMSWVRQAPGKRLEWV |
| | humanised | AAINSNGGSAYYPDTVKDRFTISRDNSKNTLYLQMNSLRAEDTAVYYC |
| | variant 1 | ARRFYYDYGWFAYWGQGTLVTVSS |
| 199 | 16 Hv | EVQLLESGGGLVQPGGSLRLSCAASGFTFSNYAMSWVRQAPGKRLEWV |
| | humanised | AAINSNSGSAYYPDTVKDRFTISRDNSKNTLYLQMNSLRAEDTAVYYC |
| | variant 2 | ARRFYYDYGWFAYWGQGTLVTVSS |
| 200 | 16 Hv | EVQLLESGGGLVQPGGSLRLSCAASGFTFSNYAMSWVRQAPGKRLEWV |
| | humanised | AAINSNAGSAYYPDTVKDRFTISRDNSKNTLYLQMNSLRAEDTAVYYC |
| | variant 3 | ARRFYYDYGWFAYWGQGTLVTVSS |
| 201 | 16 Hv | EVQLLESGGGLVQPGGSLRLSCAASGFTFSNYAMSWVRQAPGKRLEWV |
| | humanised | AAINSNVGSAYYPDTVKDRFTISRDNSKNTLYLQMNSLRAEDTAVYYC |
| | variant 4 | ARRFYYDYGWFAYWGQGTLVTVSS |
| 202 | 16 Hv | EVQLLESGGGLVQPGGSLRLSCAASGFTFSNYAMSWVRQAPGKRLEWV |
| | humanised | AAINSQGGSAYYPDTVKDRFTISRDNSKNTLYLQMNSLRAEDTAVYYC |
| | variant 5 | ARRFYYDYGWFAYWGQGTLVTVSS |
| 203 | 16 Hv | EVQLLESGGGLVQPGGSLRLSCAASGFTFSNYAMSWVRQAPGKRLEWV |
| | humanised | AAINSDGGSAYYPDTVKDRFTISRDNSKNTLYLOMNSLRAEDTAVYYC |
| | variant 6 | ARRFYYDYGWFAYWGQGTLVTVSS |
| 204 | 16 Hv | EVQLLESGGGLVQPGGSLRLSCAASGFTFSNYAMSWVRQAPGKRLEWV |
| | humanised | AAINANGGSAYYPDTVKDRFTISRDNSKNTLYLQMNSLRAEDTAVYYC |
| | variant 7 | ARRFYYDYGWFAYWGQGTLVTVSS |
| 205 | 16 Hv | EVQLLESGGGLVQPGGSLRLSCAASGFTFSNYAMSWVRQAPGKRLEWV |
| | humanised | AAINTNGGSAYYPDTVKDRFTISRDNSKNTLYLQMNSLRAEDTAVYYC |
| | variant 8 | ARRFYYDYGWFAYWGQGTLVTVSS |
| 206 | 16 Hv | EVQLLESGGGLVQPGGSLRLSCAASGFTFSNYAMSWVRQAPGKRLEWV |
| | humanised | AAIQSNGGSAYYPDTVKDRFTISRDNSKNTLYLQMNSLRAEDTAVYYC |
| | variant 9 | ARRFYYDYGWFAYWGQGTLVTVSS |
| 207 | 16 Hv | EVQLLESGGGLVQPGGSLRLSCAASGFTFSNYAMSWVRQAPGKRLEWV |
| | humanised | AAIDSNGGSAYYPDTVKDRFTISRDNSKNTLYLQMNSLRAEDTAVYYC |
| | variant 10 | ARRFYYDYGWFAYWGQGTLVTVSS |
| 208 | 16 Hv | EVQLLESGGGLVQPGGSLRLSCAASGFTFSNYAMSWVRQAPGKRLEWV |
| | humanised | AAITSNGGSAYYPDTVKDRFTISRDNSKNTLYLQMNSLRAEDTAVYYC |
| | variant 11 | ARRFYYDYGWFAYWGQGTLVTVSS |
| 209 | 16 Hv | EVQLLESGGGLVQPGGSLRLSCAASGFTFSNYAMSWVRQAPGKRLEWV |
| | humanised | AAISSNGGSAYYPDTVKDRFTISRDNSKNTLYLQMNSLRAEDTAVYYC |
| | variant 12 | ARRFYYDYGWFAYWGQGTLVTVSS |
| 210 | 16 light chain | DIVLTQSPASLSASVGETVTITCRASENIDSYLAWYQQKOGKSPQLLV |
| | (human | YAATLLADGVPSRFSGSGSGTQYSLKINSLQSEDVARYYCQHYYSSPW |
| | kappa) | TFGGGTKLEIKRTVAAPSVFIFPPSDEQLKSGTASVVCLLNNFYPREA |
| | | KVQWKVDNALQSGNSQESVTEQDSKDSTYSLSSTLTLSKADYEKHKVY |
| | | ACEVTHQGLSSPVTKSFNRGEC |
| 211 | 16 heavy | EVQLVESGGGLVKPGGSLKLSCAASGFTFSNYAMSWVRQTPEKRLEWV |
| | chain (human | AAINSNGGSAYYPDTVKDRFTISRDNAKNTLYLQMSSLRSEDTALYYC |
| | IgG1 Fab) | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 212 | 16 heavy | EVQLVESGGGLVKPGGSLKLSCAASGFTFSNYAMSWVRQTPEKRLEWV |
| | chain (human | AAINSNGGSAYYPDTVKDRFTISRDNAKNTLYLQMSSLRSEDTALYYC |
| | IgG1) | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 213 | 16 heavy | EVQLVESGGGLVKPGGSLKLSCAASGFTFSNYAMSWVRQTPEKRLEWV |
| | chain (human | AAINSNGGSAYYPDTVKDRFTISRDNAKNTLYLQMSSLRSEDTALYYC |
| | IgG1 L234A | ARRFYYDYGWFAYWGQGTLVTVSSASTKGPSVFPLAPSSKSTSGGTAA |
| | L235A | LGCLVKDYFPEPVTVSWNSGALTSGVHTFPAVLQSSGLYSLSSVVTVP |
| | G237A) | SSSLGTQTYICNVNHKPSNTKVDKKVEPKSCDKTHTCPPCPAPEAAGA |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 214 | 110 light | AQVLTQTPSPVSAAVGGTVTINCQASQSVENNKNLAWYQQKPGQPPKL |
| | chain (human | LIYKASTLASGVSSRFKGSGSGTQFALTISGVOCDDAATYYCQGEFSC |
| | kappa S171C) | SSTDCVTFGGGTEVVVKRTVAAPSVFIFPPSDEQLKSGTASVVCLLNN |
| | | FYPREAKVQWKVDNALQSGNSQESVTEQDSKDCTYSLSSTLTLSKADY |
| | | EKHKVYACEVTHQGLSSPVTKSFNRGEC |
| 215 | 110 heavy | QSVEESGGRLVTPGTPLTLTCTVSGFSLSSYAMIWVRQAPGKGLEWIG |
| | chain (human | IINSSDNTHYATWAKGRFTISKTSTTVDLKITSPTTEDTATYFCARDP |
| | IgG1 Fab) | YDYGYGWYFDLWGPGTLVTVSSASTKGPSVFPLAPSSKSTSGGTAALG |
| | | CLVKDYFPEPVTVSWNSGALTSGVHTFPAVLQSSGLYSLSSVVTVPSS |
| | | SLGTQTYICNVNHKPSNTKVDKKVEPKSC |
| 216 | 110 heavy | QSVEESGGRLVTPGTPLTLTCTVSGFSLSSYAMIWVRQAPGKGLEWIG |
| | chain (human | IINSSDNTHYATWAKGRFTISKTSTTVDLKITSPTTEDTATYFCARDP |
| | IgG1) | YDYGYGWYFDLWGPGTLVTVSSASTKGPSVFPLAPSSKSTSGGTAALG |
| | | CLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEA |
| | | LHNHYTQKSLSLSPGK |
| 217 | 110 heavy | QSVEESGGRLVTPGTPLTLTCTVSGFSLSSYAMIWVRQAPGKGLEWIG |
| | chain (human | IINSSDNTHYATWAKGRFTISKTSTTVDLKITSPTTEDTATYFCARDP |
| | IgG1 L234A | YDYGYGWYFDLWGPGTLVTVSSASTKGPSVFPLAPSSKSTSGGTAALG |
| | L235A | CLVKDYFPEPVTVSWNSGALTSGVHTFPAVLQSSGLYSLSSVVTVPSS |
| | G237A) | SLGTQTYICNVNHKPSNTKVDKKVEPKSCDKTHTCPPCPAPEAAGAPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPSREEMTKNQVSLTCLVKGFYPSDIAVEWES |
| | | NGQPENNYKTTPPVLDSDGSFFLYSKLTVDKSRWQQGNVFSCSVMHEA |
| | | LHNHYTQKSLSLSPGK |
| 218 | 116 light | AQVLTQTPSPVSAAVGGTVTINCQASQSIYNSKNLAWYQQKPGQPPKL |
| | chain (human | LIYSASTLASGVPSRFKGSGSGTQFTLTISDLECDDAATYYCOGEFSC |
| | kappa S171C) | SSVDCATFGGGTEVVVKRTVAAPSVFIFPPSDEQLKSGTASVVCLLNN |
| | | FYPREAKVQWKVDNALQSGNSQESVTEQDSKDCTYSLSSTLTLSKADY |
| | | EKHKVYACEVTHQGLSSPVTKSFNRGEC |
| 219 | 116 heavy | QSVEESGGRLVTPGTPLTLTCTVSGFSLSNYAMSWVRQAPGKGLEWIG |
| | chain (human | IIYTTGFTYYASWVKGRFTISKTSTTVDLKITSPTTEDTATYFCARGL |
| | IgG1 Fab) | ATYVSPPTRLDLWGQGTLVTVSSASTKGPSVFPLAPSSKSTSGGTAAL |
| | | GCLVKDYFPEPVTVSWNSGALTSGVHTFPAVLQSSGLYSLSSVVTVPS |
| | | SSLGTQTYICNVNHKPSNTKVDKKVEPKSC |
| 220 | 116 heavy | QSVEESGGRLVTPGTPLTLTCTVSGFSLSNYAMSWVRQAPGKGLEWIG |
| | chain (human | IIYTTGFTYYASWVKGRFTISKTSTTVDLKITSPTTEDTATYFCARGL |
| | IgG1) | ATYVSPPTRLDLWGQGTLVTVSSASTKGPSVFPLAPSSKSTSGGTAAL |
| | | GCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVMHE |
| | | ALHNHYTQKSLSLSPGK |
| 221 | 116 heavy | QSVEESGGRLVTPGTPLTLTCTVSGFSLSNYAMSWVRQAPGKGLEWIG |
| | chain (human | IIYTTGFTYYASWVKGRFTISKTSTTVDLKITSPTTEDTATYFCARGL |
| | IgG1 L234A | ATYVSPPTRLDLWGQGTLVTVSSASTKGPSVFPLAPSSKSTSGGTAAL |
| | L235A | GCLVKDYFPEPVTVSWNSGALTSGVHTFPAVLQSSGLYSLSSVVTVPS |
| | G237A) | SSLGTQTYICNVNHKPSNTKVDKKVEPKSCDKTHTCPPCPAPEAAGAP |
| | | SVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHE |
| | | ALHNHYTQKSLSLSPGK |
| 222 | 16 CDR2 H | AINANSGSAYYPDTVKD |
| | humanised | |
| | variant 12 | |
| 223 | 16 CDR2 H | AINTNSGSAYYPDTVKD |
| | humanised | |
| | variant 13 | |
| 224 | 16 CDR2 H | AIQSNSGSAYYPDTVKD |
| | humanised | |
| | variant 14 | |
| 225 | 16 CDR2 H | AIDSNSGSAYYPDTVKD |
| | humanised | |
| | variant 15 | |
| 226 | 16 CDR2 H | AITSNSGSAYYPDTVKD |
| | humanised | |
| | variant 16 | |
| 227 | 16 CDR2 H | AISSNSGSAYYPDTVKD |
| | humanised | |
| | variant 17 | |
| 228 | 16 CDR2 H | AINANAGSAYYPDTVKD |
| | humanised | |
| | variant 18 | |
| 229 | 16 CDR2 H | AINTNAGSAYYPDTVKD |
| | humanised | |
| | variant 19 | |
| 230 | 16 CDR2 H | AIQSNAGSAYYPDTVKD |
| | humanised | |
| | variant 20 | |
| 231 | 16 CDR2 H | AIDSNAGSAYYPDTVKD |
| | humanised | |
| | variant 21 | |
| 232 | 16 CDR2 H | AITSNAGSAYYPDTVKD |
| | humanised | |
| | variant 22 | |
| 233 | 16 CDR2 H | AISSNAGSAYYPDTVKD |
| | humanised | |
| | variant 23 | |
| 234 | 16 CDR2 H | AINANVGSAYYPDTVKD |
| | humanised | |
| | variant 24 | |
| 235 | 16 CDR2 H | AINTNVGSAYYPDTVKD |
| | humanised | |
| | variant 25 | |
| 236 | 16 CDR2 H | AIQSNVGSAYYPDTVKD |
| | humanised | |
| | variant 26 | |
| 237 | 16 CDR2 H | AIDSNVGSAYYPDTVKD |
| | humanised | |
| | variant 27 | |
| 238 | 16 CDR2 H | AITSNVGSAYYPDTVKD |
| | humanised | |
| | variant 28 | |
| 239 | 16 CDR2 H | AISSNVGSAYYPDTVKD |
| | humanised | |
| | variant 29 | |
| 240 | 16 CDR2 H | AINAQGGSAYYPDTVKD |
| | humanised | |
| | variant 30 | |
| 241 | 16 CDR2 H | AINTQGGSAYYPDTVKD |
| | humanised | |
| | variant 31 | |
| 242 | 16 CDR2 H | AIQSQGGSAYYPDTVKD |
| | humanised | |
| | variant 32 | |
| 243 | 16 CDR2 H | AIDSQGGSAYYPDTVKD |
| | humanised | |
| | variant 33 | |
| 244 | 16 CDR2 H | AITSQGGSAYYPDTVKD |
| | humanised | |
| | variant 34 | |
| 245 | 16 CDR2 H | AISSQGGSAYYPDTVKD |
| | humanised | |
| | variant 35 | |
| 246 | 16 CDR2 H | AINADGGSAYYPDTVKD |
| | humanised | |
| | variant 36 | |
| 247 | 16 CDR2 H | AINTDGGSAYYPDTVKD |
| | humanised | |
| | variant 37 | |
| 248 | 16 CDR2 H | AIQSDGGSAYYPDTVKD |
| | humanised | |
| | variant 38 | |
| 249 | 16 CDR2 H | AIDSDGGSAYYPDTVKD |
| | humanised | |
| | variant 39 | |
| 250 | 16 CDR2 H | AITSDGGSAYYPDTVKD |
| | humanised | |
| | variant 40 | |
| 251 | 16 CDR2 H | AISSDGGSAYYPDTVKD |
| | humanised | |
| | variant 41 | |
| 252 | CD1a | ATGLKEPLSFHVIWIASFYNHSWKQNLVSGWLSDLQTHTWDSNSSTIV |
| | | FLWPWSRGNFSNEEWKELETLFRIRTIRSFEGIRRYAHELQFEYPFEI |
| | | QVTGGCELHSGKVSGSFLQLAYQGSDFVSFQNNSWLPYPVAGNMAKHF |
| | | CKVLNQNQHENDITHNLLSDTCPRFILGLLDAGKAHLQRQVKPEAWLS |
| | | HGPSPGPGHLQLVCHVSGFYPKPVWVMWMRGEQEQQGTORGDILPSAD |
| | | GTWYLRATLEVAAGEAADLSCRVKHSSLEGQDIVLYWEGSLVPRG |
| 253 | 25 light chain | EIVLTQSPALMAASPGEKVTITCSVSSSISSSNLHWYQQKSGTSPKPW |
| | (human | IYGTSNLASGVPVRFSGSGSGTSYSLTISSMEAEDAATYYCQQWSSYP |
| | kappa) | PTFGSGTKLEIKRTVAAPSVFIFPPSDEQLKSGTASVVCLLNNFYPRE |
| | | AKVQWKVDNALQSGNSQESVTEQDSKDSTYSLSSTLTLSKADYEKHKV |
| | | YACEVTHQGLSSPVTKSFNRGEC |
| 254 | 25 heavy | QVQLQQSGPGLVAPSQSLFITCTVSGFSLTTYEINWVRQPPGKGLEWL |
| | chain (human | GVIWTGGSTNYNSALISRLSISKDNSKSLVFLKMNSLQTDDTAIYYCV |
| | IgG1 Fab) | RTLPYYYAMDYWGQGTSVTVSSASTKGPSVFPLAPSSKSTSGGTAALG |
| | | CLVKDYFPEPVTVSWNSGALTSGVHTFPAVLQSSGLYSLSSVVTVPSS |
| | | SLGTQTYICNVNHKPSNTKVDKKVEPKSC |
| 255 | 25 heavy | QVQLQQSGPGLVAPSQSLFITCTVSGFSLTTYEINWVRQPPGKGLEWL |
| | chain (human | GVIWTGGSTNYNSALISRLSISKDNSKSLVFLKMNSLQTDDTAIYYCV |
| | IgG1) | RTLPYYYAMDYWGQGTSVTVSSASTKGPSVFPLAPSSKSTSGGTAALG |
| | | CLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEA |
| | | LHNHYTQKSLSLSPGK |
| 256 | 25 heavy | QVQLQQSGPGLVAPSQSLFITCTVSGFSLTTYEINWVRQPPGKGLEWL |
| | chain (human | GVIWTGGSTNYNSALISRLSISKDNSKSLVFLKMNSLQTDDTAIYYCV |
| | IgG1 L234A | RTLPYYYAMDYWGQGTSVTVSSASTKGPSVFPLAPSSKSTSGGTAALG |
| | L235A | CLVKDYFPEPVTVSWNSGALTSGVHTFPAVLQSSGLYSLSSVVTVPSS |
| | G237A) | SLGTQTYICNVNHKPSNTKVDKKVEPKSCDKTHTCPPCPAPEAAGAPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPSREEMTKNQVSLTCLVKGFYPSDIAVEWES |
| | | NGQPENNYKTTPPVLDSDGSFFLYSKLTVDKSRWQQGNVFSCSVMHEA |
| | | LHNHYTQKSLSLSPGK |
| 257 | 25 CDR2 H | VIWTGGSTNYNAALIS |
| | humanised | |
| | variant 1 | |
| 258 | 25 CDR2 H | VIWTGGSTNYNTALIS |
| | humanised | |
| | variant 2 | |
| 259 | 25 CDR2 H | VIWTGGSTNYQSALIS |
| | humanised | |
| | variant 3 | |
| 260 | 25 Lv | EIVLTQSPDFQSVTPKEKVTITCSVSSSISSSNLHWYQQKPDQSPKPW |
| | humanised | IYGTSNLASGVPVRFSGSGSGTSYTLTINSLEAEDAATYYCQQWSSYP |
| | variant 1 | PTFGQGTKLEIK |
| 261 | 25 Hv | EVQLQESGPGLVKPSETLSLTCTVSGFSLTTYEINWVRQPPGKGLEWL |
| | humanised | GVIWTGGSTNYNSALISRVTISKDNSKSQVSLKLSSVTAADTAVYYCV |
| | variant 1 | RTLPYYYAMDYWGQGTTVTVSS |
| 262 | 25 Hv | EVQLQESGPGLVKPSETLSLTCTVSGFSLTTYEINWVRQPPGKGLEWL |
| | humanised | GVIWTGGSTNYNAALISRVTISKDNSKSQVSLKLSSVTAADTAVYYCV |
| | variant 2 | RTLPYYYAMDYWGQGTTVTVSS |
| 263 | 25 Hv | EVQLQESGPGLVKPSETLSLTCTVSGFSLTTYEINWVRQPPGKGLEWL |
| | humanised | GVIWTGGSTNYNTALISRVTISKDNSKSQVSLKLSSVTAADTAVYYCV |
| | variant 3 | RTLPYYYAMDYWGQGTTVTVSS |
| 264 | 25 Hv | EVQLQESGPGLVKPSETLSLTCTVSGFSLTTYEINWVRQPPGKGLEWL |
| | humanised | GVIWTGGSTNYQSALISRVTISKDNSKSQVSLKLSSVTAADTAVYYCV |
| | variant 4 | RTLPYYYAMDYWGQGTTVTVSS |
| 265 | AH1 Fab | EVQLVESGGGLVQPGGSLRLSCAVSGFSLSNYAMSWVRQAPGKGLEWI |
| | heavy | GIIYTTGFTYYASWVKGRFTISKDSSKNTVYLQMNSLRAEDTAVYYCA |
| | | RGLATYVSPPTRLDLWGQGTLVTVSSASTKGPSVFPLAPSSKSTSGGT |
| | | AALGCLVKDYFPEPVTVSWNSGALTSGVHTFPAVLQSSGLYSLSSVVT |
| | | VPSSSLGTQTYICNVNHKPSNTKVDKKVEPKSC |
| 266 | AL1 light | AQVLTQSPSSLSASVGDRVTITCQASQSIYNSKNLAWYQQKPGKAPKL |
| | | LIYSASTLASGVPSRFSGSGSGTOFTLTISSLQPEDFATYYCQGEFSC |
| | | SSVDCATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSFNRGEC |
| 267 | AL2 | AQVLTQSPSSLSASVGDRVTITCQASQSIYNTKNLAWYQQKPGKAPKL |
| | | LIYSASTLASGVPSRFSGSGSGTOFTLTISSLQPEDFATYYCQGEFSC |
| | | SSVDCATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSFNRGEC |
| 268 | AL3 | AQVLTQSPSSLSASVGDRVTITCQASQSIYNAKNLAWYQQKPGKAPKL |
| | | LIYSASTLASGVPSRFSGSGSGTQFTLTISSLQPEDFATYYCQGEFSC |
| | | SSVDCATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSFNRGEC |
| 269 | AL4 | AQVLTQSPSSLSASVGDRVTITCQASQSIYNNKNLAWYQQKPGKAPKL |
| | | LIYSASTLASGVPSRFSGSGSGTOFTLTISSLQPEDFATYYCOGEFSC |
| | | SSVDCATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSFNRGEC |
| 270 | AL5 | AQVLTQSPSSLSASVGDRVTITCQASQSIYQSKNLAWYQQKPGKAPKL |
| | | LIYSASTLASGVPSRFSGSGSGTQFTLTISSLQPEDFATYYCOGEFSC |
| | | SSVDCATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSENRGEC |
| 271 | AL6 | AQVLTQSPSSLSASVGDRVTITCQASQSIYDSKNLAWYQQKPGKAPKL |
| | | LIYSASTLASGVPSRFSGSGSGTQFTLTISSLQPEDFATYYCQGEFSC |
| | | SSVDCATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSFNRGEC |
| 272 | AL7 | AQVLTQSPSSLSASVGDRVTITCQASQSIYNSKNLAWYQQKPGKAPKL |
| | | LIYSASTLASGVPSRFSGSGSGTQFTLTISSLQPEDFATYYCQGEFSS |
| | | SSVDSATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSENRGEC |
| 273 | AL8 | AQVLTQSPSSLSASVGDRVTITCQASQSIYNSKNLAWYQQKPGKAPKL |
| | | LIYSASTLASGVPSRFSGSGSGTOFTLTISSLQPEDFATYYCQGEFSS |
| | | SSVDAATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSFNRGEC |
| 274 | AL9 | AQVLTQSPSSLSASVGDRVTITCQASQSIYNSKNLAWYQQKPGKAPKL |
| | | LIYSASTLASGVPSRFSGSGSGTQFTLTISSLQPEDFATYYCQGEFSS |
| | | SSVDVATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSFNRGEC |
| 275 | AL10 | AQVLTQSPSSLSASVGDRVTITCQASQSIYNSKNLAWYQQKPGKAPKL |
| | | LIYSASTLASGVPSRFSGSGSGTQFTLTISSLQPEDFATYYCQGEFSA |
| | | SSVDSATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSFNRGEC |
| 276 | AL11 | AQVLTQSPSSLSASVGDRVTITCQASQSIYNSKNLAWYQQKPGKAPKL |
| | | LIYSASTLASGVPSRFSGSGSGTOFTLTISSLQPEDFATYYCQGEFSA |
| | | SSVDAATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSENRGEC |
| 277 | AL12 | AQVLTQSPSSLSASVGDRVTITCQASQSIYNSKNLAWYQQKPGKAPKL |
| | | LIYSASTLASGVPSRFSGSGSGTQFTLTISSLQPEDFATYYCQGEFSA |
| | | SSVDVATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSFNRGEC |
| 278 | AL13 | AQVLTQSPSSLSASVGDRVTITCQASQSIYNSKNLAWYQQKPGKAPKL |
| | | LIYSASTLASGVPSRFSGSGSGTQFTLTISSLQPEDFATYYCQGEFSV |
| | | SSVDSATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSFNRGEC |
| 279 | AL14 | AQVLTQSPSSLSASVGDRVTITCQASQSIYNSKNLAWYQQKPGKAPKL |
| | | LIYSASTLASGVPSRFSGSGSGTQFTLTISSLQPEDFATYYCQGEFSV |
| | | SSVDAATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSENRGEC |
| 280 | AL15 | AQVLTQSPSSLSASVGDRVTITCQASQSIYNSKNLAWYQQKPGKAPKL |
| | | LIYSASTLASGVPSRFSGSGSGTQFTLTISSLQPEDFATYYCQGEFSV |
| | | SSVDVATFGGGTKVEIKRTVAAPSVFIFPPSDEQLKSGTASVVCLLNN |
| | | FYPREAKVQWKVDNALQSGNSQESVTEQDSKDSTYSLSSTLTLSKADY |
| | | EKHKVYACEVTHQGLSSPVTKSFNRGEC |
| 281 | BH1 Fab | EVQLLESGGGLVQPGGSLRLSCAASGFTFSNYAMSWVRQAPGKRLEWV |
| | heavy | AAINSNGGSAYYPDTVKDRFTISRDNSKNTLYLOMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 282 | BL1 | DIQMTQSPSSLSASVGDRVTITCRASENIDSYLAWYQQKPGKAPKLLV |
| | | YAATLLADGVPSRFSGSGSGTQYTLTISSLQPEDFARYYCQHYYSSPW |
| | | TFGQGTKVEIKRTVAAPSVFIFPPSDEQLKSGTASVVCLLNNFYPREA |
| | | KVQWKVDNALQSGNSQESVTEQDSKDSTYSLSSTLTLSKADYEKHKVY |
| | | ACEVTHQGLSSPVTKSENRGEC |
| 283 | BL2 | DIQMTQSPSSLSASVGDRVTITCRASENIDSYLAWYQQKPGKAPKLLV |
| | | YAATLLAEGVPSRFSGSGSGTQYTLTISSLQPEDFARYYCQHYYSSPW |
| | | TFGQGTKVEIKRTVAAPSVFIFPPSDEQLKSGTASVVCLLNNFYPREA |
| | | KVQWKVDNALQSGNSQESVTEQDSKDSTYSLSSTLTLSKADYEKHKVY |
| | | ACEVTHQGLSSPVTKSENRGEC |
| 284 | BL3 | DIQMTQSPSSLSASVGDRVTITCRASENIDSYLAWYQQKPGKAPKLLV |
| | | YAATLLASGVPSRFSGSGSGTQYTLTISSLQPEDFARYYCQHYYSSPW |
| | | TFGQGTKVEIKRTVAAPSVFIFPPSDEQLKSGTASVVCLLNNFYPREA |
| | | KVQWKVDNALQSGNSQESVTEQDSKDSTYSLSSTLTLSKADYEKHKVY |
| | | ACEVTHQGLSSPVTKSENRGEC |
| 285 | BH2 Fab | EVQLLESGGGLVQPGGSLRLSCAASGFTFSNYAMSWVRQAPGKRLEWV |
| | heavy | AAINSNSGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 286 | BH3 Fab | EVQLLESGGGLVQPGGSLRLSCAASGFTFSNYAMSWVRQAPGKRLEWV |
| | heavy | AAINSNAGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 287 | BH4 Fab | EVQLLESGGGLVQPGGSLRLSCAASGFTFSNYAMSWVRQAPGKRLEWV |
| | heavy | AAINSNVGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 288 | BH5 Fab | EVQLLESGGGLVQPGGSLRLSCAASGFTFSNYAMSWVRQAPGKRLEWV |
| | heavy | AAINSQGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 289 | BH6 Fab | EVQLLESGGGLVQPGGSLRLSCAASGFTFSNYAMSWVRQAPGKRLEWV |
| | heavy | AAINSDGGSAYYPDTVKDRFTISRDNSKNTLYLOMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 290 | BH7 Fab | EVQLLESGGGLVQPGGSLRLSCAASGFTFSNYAMSWVRQAPGKRLEWV |
| | heavy | AAINANGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 291 | BH8 Fab | EVQLLESGGGLVQPGGSLRLSCAASGFTFSNYAMSWVRQAPGKRLEWV |
| | heavy | AAINTNGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 292 | BH9 Fab | EVQLLESGGGLVQPGGSLRLSCAASGFTFSNYAMSWVRQAPGKRLEWV |
| | heavy | AAIQSNGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 293 | BH9 Fab | EVQLLESGGGLVQPGGSLRLSCAASGFTFSNYAMSWVRQAPGKRLEWV |
| | heavy | AAIDSNGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 294 | BH10 Fab | EVQLLESGGGLVQPGGSLRLSCAASGFTFSNYAMSWVRQAPGKRLEWV |
| | heavy | AAIDSNGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 295 | BH11 Fab | EVQLLESGGGLVQPGGSLRLSCAASGFTFSNYAMSWVRQAPGKRLEWV |
| | heavy | AAITSNGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 296 | BH12 Fab | EVQLLESGGGLVQPGGSLRLSCAASGFTFSNYAMSWVRQAPGKRLEWV |
| | heavy | AAISSNGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSC |
| 297 | Fc region | DKTHTCPPCPAPELLGGPSVFLFPPKPKDTLMISRTPEVTCVVVDVSH |
| | | EDPEVKFNWYVDGVEVHNAKTKPREEQYNSTYRVVSVLTVLHQDWLNG |
| | | KEYKCKVSNKALPAPIEKTISKAKGQPREPQVYTLPPSRDELTKNQVS |
| | | LTCLVKGFYPSDIAVEWESNGQPENNYKTTPPVLDSDGSFFLYSKLTV |
| | | DKSRWQQGNVFSCSVMHEALHNHYTQKSLSLSPGK |
| 298 | UCHT1 | EVQLVESGGGLVQPGGSLRLSCAASGYSFTGYTMNWVRQAPGKGLEWV |
| | | ALINPYKGVTTYADSVKGRFTISVDKSKNTAYLQMNSLRAEDTAVYYC |
| | | ARSGYYGDSDWYFDVWGQGTLVTVSSggggsggggggggsDIQMTQS |
| | | PSSLSASVGDRVTITCRASQDIRNYLNWYQQKPGKAPKLLIYYTSRLE |
| | | SGVPSRFSGSGSGTDYTLTISSLQPEDFATYYCQQGNTLPWTFGQGTK |
| | | VEIKGGGGSDKTHTCPPCPAPEAAGGPSVFLFPPKPKDTLMISRTPEV |
| | | TCVVVDVSHEDPEVKFNWYVDGVEVHNAKTKPREEQYNSTYRVVSVLT |
| | | VLHQDWLNGKEYKCKVSNKALPAPIEKTISKAKGQPREPQVCTLPPSR |
| | | DELTKNQVSLSCAVKGFYPSDIAVEWESNGQPENNYKTTPPVLDSDGS |
| | | FFLVSKLTVDKSRWQQGNVFSCSVMHEALHNHYTQKSLSLSPGK |
| 299 | WIMM3 | EVQLVESGGGLVKPGGSLKLSCAASGFTFSNYAMSWVRQTPEKRLEWV |
| | heavy chain | AAINSNGGSAYYPDTVKDRFTISRDNAKNTLYLQMSSLRSEDTALYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPEAAGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPCRDELTKNQVSLWCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 300 | AH1 + Fc | EVQLVESGGGLVQPGGSLRLSCAVSGFSLSNYAMSWVRQAPGKGLEWI |
| | | GIIYTTGFTYYASWVKGRFTISKDSSKNTVYLQMNSLRAEDTAVYYCA |
| | | RGLATYVSPPTRLDLWGQGTLVTVSSASTKGPSVFPLAPSSKSTSGGT |
| | | AALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSV |
| | | MHEALHNHYTQKSLSLSPGK |
| 301 | BH1 + Fc | EVQLLESGGGLVQPGGSLRLSCAASGFTFSNYAMSWVRQAPGKRLEWV |
| | | AAINSNGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLOSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 302 | BH2 + Fc | EVQLLESGGGLVQPGGSLRLSCAASGFTFSNYAMSWVRQAPGKRLEWV |
| | | AAINSNSGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 303 | BH3 + Fc | EVQLLESGGGLVQPGGSLRLSCAASGFTFSNYAMSWVRQAPGKRLEWV |
| | | AAINSNAGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 304 | BH4 + Fc | EVQLLESGGGLVQPGGSLRLSCAASGFTFSNYAMSWVRQAPGKRLEWV |
| | | AAINSNVGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 305 | BH5 + Fc | EVQLLESGGGLVQPGGSLRLSCAASGFTFSNYAMSWVRQAPGKRLEWV |
| | | AAINSQGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKENWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 306 | BH6 + Fc | EVQLLESGGGLVQPGGSLRLSCAASGFTFSNYAMSWVRQAPGKRLEWV |
| | | AAINSDGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 307 | BH7 + Fc | EVQLLESGGGLVQPGGSLRLSCAASGFTFSNYAMSWVRQAPGKRLEWV |
| | | AAINANGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 308 | BH8 + Fc | EVQLLESGGGLVQPGGSLRLSCAASGFTFSNYAMSWVRQAPGKRLEWV |
| | | AAINTNGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 309 | BH9 + Fc | EVQLLESGGGLVQPGGSLRLSCAASGFTFSNYAMSWVRQAPGKRLEWV |
| | | AAIQSNGGSAYYPDTVKDRFTISRDNSKNTLYLOMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 310 | BH10 + Fc | EVQLLESGGGLVQPGGSLRLSCAASGFTFSNYAMSWVRQAPGKRLEWV |
| | | AAIDSNGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 311 | BH11 + Fc | EVQLLESGGGLVQPGGSLRLSCAASGFTFSNYAMSWVRQAPGKRLEWV |
| | | AAITSNGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 312 | BH12 + Fc | EVQLLESGGGLVQPGGSLRLSCAASGFTFSNYAMSWVRQAPGKRLEWV |
| | | AAISSNGGSAYYPDTVKDRFTISRDNSKNTLYLQMNSLRAEDTAVYYC |
| | | ARRFYYDYGWFAYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 313 | WIMM3 | EVQLVESGGGLVKPGGSLKLSCAASGFTFSNYAMSWVRQTPEKRLEWV |
| | heavy chain | AAINSNGGSAYYPDTVKDRFTISRDNAKNTLYLQMSSLRSEDTALYYC |
| | variable | ARRFYYDYGWFAYWGQGTLVTVSS |
| 314 | WIMM3 light | DIVLTQSPASLSASVGETVTITCRASENIDSYLAWYQQKOGKSPQLLV |
| | chain variable | YAATLLADGVPSRFSGSGSGTQYSLKINSLQSEDVARYYCQHYYSSPW |
| | | TFGGGTKLEIK |

Underlined portions of any DNA sequence above denote a signal sequence.

1. An antibody or antigen binding fragment thereof that binds to cluster of differentiation 1a (CD1a), wherein the antibody or antigen binding fragment thereof is chimeric, and wherein the antibody or antigen binding fragment thereof comprises:
a) a heavy chain variable region comprising:
a CDR1 of SEQ ID NO: 33,
a CDR2 of SEQ ID NO: 34, and
a CDR3 of SEQ ID NO: 35,
or sequences having at least 80%, 90%, 95%, 98%, 99% or 100% identity thereto, and/or
a light chain variable region comprising:
a CDR1 of SEQ ID NO: 36,
a CDR2 of SEQ ID NO: 37, and
a CDR3 of SEQ ID NO: 38,
or sequences having at least 80%, 90%, 95%, 98% 99% or 100%, identity thereto; or
b) a heavy chain variable region comprising:
a CDR1 of SEQ ID NO: 1,
a CDR2 of SEQ ID NO: 2, and
a CDR3 of SEQ ID NO: 3,
or sequences having at least 80%, 90%, 95%, 98%, 99% or 100% identity thereto, and/or
a light chain variable region comprising:
a CDR1 of SEQ ID NO: 4,
a CDR2 of SEQ ID NO: 5, and
a CDR3 of SEQ ID NO: 6,
or sequences having at least 80%, 90%, 95%, 98% 99% or 100% identity thereto; or
c) a heavy chain variable region comprising:
a CDR1 of SEQ ID NO: 9,
a CDR2 of SEQ ID NO: 10, and
a CDR3 of SEQ ID NO: 11,
or sequences having at least 80%, 90%, 95%, 98%, 99% or 100% identity thereto, and/or
a light chain variable region comprising:
a CDR1 of SEQ ID NO: 12,
a CDR2 of SEQ ID NO: 13, and
a CDR3 of SEQ ID NO: 14,
or sequences having at least 80%, 90%, 95%, 98%, 99% or 100% identity thereto; or
d) a heavy chain variable region comprising:
a CDR1 of SEQ ID NO: 17,
a CDR2 of SEQ ID NO: 18, and
a CDR3 of SEQ ID NO: 19,
or sequences having at least 80%, 90%, 95%, 98% 99% or 100% identity thereto, and/or
a light chain variable region comprising:
a CDR1 of SEQ ID NO: 20,
a CDR2 of SEQ ID NO: 21, and
a CDR3 of SEQ ID NO: 22,
or sequences having at least 80%, 90%, 95%, 98%, 99% or 100% identity thereto; or
e) a heavy chain variable region comprising:
a CDR1 of SEQ ID NO: 25,
a CDR2 of SEQ ID NO: 26, and
a CDR3 of SEQ ID NO: 27,
or sequences having at least 80%, 90%, 95%, 98%, 99% or 100% identity thereto, and/or
a light chain variable region comprising:
a CDR1 of SEQ ID NO: 28,
a CDR2 of SEQ ID NO: 29, and
a CDR3 of SEQ ID NO: 30,
or sequences having at least 80%, 90%, 95%, 98%, 99% or 100% identity thereto; or
f) a heavy chain variable region comprising:
a CDR1 of SEQ ID NO: 91,
a CDR2 of SEQ ID NO: 92, and
a CDR3 of SEQ ID NO: 93,
or sequences having at least 80%, 90%, 95%, 98%, 99% or 100% identity thereto; and/or
a light chain variable region comprising:
a CDR1 of SEQ ID NO: 94,
a CDR2 of SEQ ID NO: 95, and
a CDR3 of SEQ ID NO: 96
or sequences having at least 80%, 90%, 95%, 98%, 99% or 100% identity thereto.

2. The antibody or antigen binding fragment of claim 1, wherein the antibody or antigen binding fragment comprises:
(a) a heavy chain comprising or consisting of SEQ ID NO: 219, SEQ ID NO: 220 or SEQ ID NO: 221, or a sequence having at least 80%, 90%, 95%, 98%, 99% or 100% identity thereto; and/or
a light chain comprising or consisting of SEQ ID NO: 218,
or a sequence having at least 80%, 90%, 95%, 98%, 99% or 100% identity thereto; or
(b) a heavy chain comprising or consisting of SEQ ID NO: 215, SEQ ID NO: 216 or SEQ ID NO: 217, or a sequence having at least 80%, 90%, 95%, 98%, 99% or 100% identity thereto; and/or
a light chain comprising or consisting of SEQ ID NO: 214,
or a sequence having at least 80%, 90%, 95%, 98%, 99% or 100% identity thereto; or
(c) a heavy chain comprising or consisting of SEQ ID NO: 211, SEQ ID NO: 212 or SEQ ID NO: 213, or a sequence having at least 80%, 90%, 95%, 98%, 99% or 100% identity thereto; and/or
a light chain comprising or consisting of SEQ ID NO: 210,
or a sequence having at least 80%, 90%, 95%, 98%, 99% or 100% identity thereto; or
(d) a heavy chain comprising or consisting of SEQ ID NO: 254, SEQ ID NO: 255 or SEQ ID NO: 256, or a sequence having at least 80%, 90%, 95%, 98%, 99% or 100% identity thereto; and/or
a light chain comprising or consisting of SEQ ID NO: 253,
or a sequence having at least 80%, 90%, 95%, 98%, 99% or 100% identity thereto.

3. An antibody or antigen binding fragment thereof that binds to CD1a, wherein the antibody or antigen binding fragment thereof is humanized, and wherein the antibody or antigen binding fragment thereof comprises:
(a) a heavy chain variable region comprising:
a CDR1 of SEQ ID NO: 33,
a CDR2 of SEQ ID NO: 34, and
a CDR3 of SEQ ID NO: 35,
or sequences having at least 80%, 90%, 95%, 98%, 99% or 100% identity thereto; and/or
a light chain variable region comprising:
a CDR1 of SEQ ID NO: 36, SEQ ID NO: 135, SEQ ID NO: 136, SEQ ID NO: 137, SEQ ID NO: 13 or, SEQ ID NO: 139,
a CDR2 of SEQ ID NO: 37, and
a CDR3 of SEQ ID NO: 38, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130 or SEQ ID NO: 131,
or sequences having at least 80%, 90%, 95%, 98%, 99% or 100% identity thereto; or
(b) a heavy chain variable region comprising:
a CDR1 of SEQ ID NO: 17,
a CDR2 of SEQ ID NO: 18, SEQ ID NO: 132, SEQ ID NO: 133 or SEQ ID NO: 134, and
a CDR3 of SEQ ID NO: 19 or SEQ ID NO: 110,
or sequences having at least 80%, 90%, 95%, 98%, 99% or 100% identity thereto; and/or
a light chain variable region comprising:
a CDR1 of SEQ ID NO: 20,
a CDR2 of SEQ ID NO: 21, and
a CDR3 of SEQ ID NO: 22, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118 or SEQ ID NO: 119,
or sequences having at least 80%, 90%, 95%, 98%, 99% or 100% identity thereto; or
(c) a heavy chain variable region comprising:
a CDR1 of SEQ ID NO: 25,
a CDR2 of SEQ ID NO: 26, and
a CDR3 of SEQ ID NO: 27,
or sequences having at least 80%, 90%, 95%, 98%, 99% or 100% identity thereto; and/or
a light chain variable region comprising:
a CDR1 of SEQ ID NO: 28,
a CDR2 of SEQ ID NO: 29, and
a CDR3 of SEQ ID NO: 30, SEQ ID NO: 120, SEQ ID NO: 121 or SEQ ID NO: 122,
or sequences having at least 80%, 90%, 95%, 98%, 99% or 100% identity thereto; or
(d) a heavy chain variable region comprising:
a CDR1 of SEQ ID NO: 9,
a CDR2 of SEQ ID NO: 10, and
a CDR3 of SEQ ID NO: 11,
or sequences having at least 80%, 90%, 95%, 98%, 99% or 100% identity thereto; and/or
a light chain variable region comprising:
a CDR1 of SEQ ID NO: 12,
a CDR2 of SEQ ID NO: 13, and
a CDR3 of SEQ ID NO: 14, SEQ ID NO: 107, SEQ ID NO: 108 or SEQ ID NO: 109,
or sequences having at least 80%, 90%, 95%, 98%, 99% or 100% identity thereto; or
(e) a heavy chain variable region comprising:
a CDR1 of SEQ ID NO: 1,
a CDR2 of SEQ ID NO: 2, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, or SEQ ID NO: 251, and
a CDR3 of SEQ ID NO: 3,
or sequences having at least 80%, 90%, 95%, 98%, 99% or 100% identity thereto; and/or
a light chain variable region comprising:
a CDR1 of SEQ ID NO: 4,
a CDR2 of SEQ ID NO: 5, SEQ ID NO: 140 or SEQ ID NO: 141, and
a CDR3 of SEQ ID NO: 6,
or sequences having at least 80%, 90%, 95%, 98%, 99% or 100% identity thereto; or
(f) a heavy chain variable region comprising:
a CDR1 of SEQ ID NO: 91,
a CDR2 of SEQ ID NO: 92, SEQ ID NO: 257, SEQ ID NO: 258, or SEQ ID NO: 259 and
a CDR3 of SEQ ID NO: 93,
or sequences having at least 80%, 90%, 95%, 98%, 99% or 100% identity thereto; and/or
(g) a light chain variable region comprising:
a CDR1 of SEQ ID NO: 94,
a CDR2 of SEQ ID NO: 95, and
a CDR3 of SEQ ID NO: 96
or sequences having at least 80%, 90%, 95%, 98%, 99% or 100% identity thereto.

4. The antibody or antigen binding fragment thereof of claim 3, wherein the antibody or antigen binding fragment thereof comprises:
(a) a heavy chain variable region comprising or consisting of SEQ ID NO: 39 or SEQ ID NO: 194, or a sequence having at least 80%, 90%, 95%, 98%, 99% or 100% identity thereto; and/or
a light chain variable region comprising or consisting of SEQ ID NO: 40, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192 or SEQ ID NO:193, or a sequence having at least 80%, 90%, 95%, 98%, 99% or 100% identity thereto; or
(b) a heavy chain variable region comprising or consisting of SEQ ID NO: 23, SEQ ID NO: 168, SEQ ID NO: 169, SEQ ID NO: 170, SEQ ID NO: 171, SEQ ID NO: 172 or SEQ ID NO: 173, or a sequence having at least 80%, 90%, 95%, 98%, 99% or 100% identity thereto; and/or
a light chain variable region comprising or consisting of SEQ ID NO: 24, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166 or SEQ ID NO: 167, or a sequence having at least 80%, 90%, 95%, 98%, 99% or 100% identity thereto; or
(c) a heavy chain variable region comprising or consisting of SEQ ID NO: 31 or SEQ ID NO: 178 or a sequence having at least 80%, 90%, 95%, 98%, 99% or 100% identity thereto; and/or
a light chain variable region comprising or consisting of SEQ ID NO: 32, SEQ ID NO: 174, SEQ ID NO: 175, SEQ ID NO: 176 or SEQ ID NO: 177, or a sequence having at least 80%, 90%, 95%, 98%, 99% or 100% identity thereto; or
(d) a heavy chain variable region comprising or consisting of SEQ ID NO: 15 or SEQ ID NO: 157, or a sequence having at least 80%, 90%, 95%, 98%, 99% or 100% identity thereto; and/or
a light chain variable region comprising or consisting of SEQ ID NO: 16, SEQ ID NO: 153, SEQ ID NO: 154, SEQ ID NO: 155 or SEQ ID NO: 156, or a sequence having at least 80%, 90%, 95%, 98%, 99% or 100% identity thereto; or
(e) a heavy chain variable region comprising or consisting of SEQ ID NO: 7, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208 or SEQ ID NO: 209, or a sequence having at least 80%, 90%, 95%, 98%, 99% or 100% identity thereto; and/or
a light chain variable region comprising or consisting of SEQ ID NO: 8, SEQ ID NO: 195, SEQ ID NO: 196 or SEQ ID NO: 197, or a sequence having at least 80%, 90%, 95%, 98%, 99% or 100% identity thereto; or
(f) a heavy chain variable region comprising or consisting of SEQ ID NO: 97, SEQ ID NO: 261, SEQ ID NO: 262, SEQ ID NO: 263 or SEQ ID NO: 264, or a sequence having at least 80%, 90%, 95%, 98%, 99% or 100% identity thereto; and/or
a light chain variable region comprising or consisting of SEQ ID NO: 98 or SEQ ID NO: 260, or a sequence having at least 80%, 90%, 95%, 98%, 99% or 100% identity thereto.

5. The antibody or antigen binding fragment thereof of claim 3, wherein the antibody is a full-length antibody.

6. The antibody of claim 5 wherein the antibody is an IgG1 antibody or an IgG1 antibody having one or more substitutions in the constant region.

7. An anti body or antigen binding fragment thereof that binds to CD1a, comprising or consisting of:
(a) an ScFv comprising or consisting of SEQ ID NO: 104, or a sequence having at least 80%, 90%, 95%, 98%, 99% or 100% identity thereto; or
(b) an ScFv comprising or consisting of SEQ ID NO: 105, or a sequence having at least 80%, 90%, 95%, 98%, 99% or 100% identity thereto; or
(c) an ScFv comprising or consisting of SEQ ID NO: 106, or a sequence having at least 80%, 90%, 95%, 98%, 99% or 100% identity thereto.

8. The antibody or antigen binding fragment thereof of claim 3, wherein the antibody or antigen binding fragment thereof binds to an epitope on CD1a, wherein the epitope of CD1a comprises or consists of
(a) residues Arg 83, Tyr 84, His 86, Glu 87, Gln 89, Phe 90, Glu 91, Asn 139, Met 140, Lys 142, His 143, Lys 146, Val 147 and Gln 150 of CD1a, and wherein the residue numbering is according to SEQ ID NO: 252; or
(b) residues Glu 62, Glu 65, Leu 66, Thr 68, Leu 69, Ile 72, Asn 151, His 153, Glu 154, Ile 157, Asn 160, Asp 164, Thr165 and Arg 168 of CD1a, and wherein the residue numbering is according to SEQ ID NO: 252; or
(c) residues Glu 79, Arg 82, Arg 83, His 86, Glu 87, Gln 89, Phe 90, Glu 91, Tyr 92, Val 147 and Asn 150 of CD1, and wherein the residue numbering is according to SEQ ID NO: 252.

9. A nucleic acid encoding an antibody or antigen binding fragment thereof of claim 3.

10. A vector comprising the nucleic acid of claim 9.

11. The vector of claim 10, wherein the vector is an expression vector, plasmid, or viral vector.

12. A host cell comprising an antibody or antigen binding fragment thereof of claim 3.

13. The host cell of claim 12, wherein the host cell is a bacterial cell or mammalian cell.

14. A pharmaceutical composition comprising an antibody or antigen binding fragment thereof of claim 3.

15-21. (canceled)

22. A method of treating a condition in a subject, comprising administering to the subject an effective amount of the antibody or antigen binding fragment thereof of claim 3, wherein the condition is selected from the group consisting of: an inflammatory skin or mucosal disease or disorder, a systemic disease or disorder associated with an inflammatory response, a systemic inflammatory drug reaction, and a CD1a-expressing malignancy.

23. A method of monitoring treatment efficacy or disease status in a subject diagnosed with a CD1a-expressing malignancy, comprising:
determining the level of binding of an antibody or antigen binding fragment thereof of claim 3 to CD1a-expressing cells in the sample obtained from the subject before treatment, or at intervals between treatments, or at time intervals in the absence of treatment;
wherein the treatment is effective, or the disease status is improving, if the tumor volume, or level of binding of the antibody or antigen binding fragment thereof to CD1a-expressing cells, is reduced after treatment or between treatment intervals or at time intervals in the absence of treatment, optionally wherein the reduction in tumor volume or level of binding of the antibody or antigen binding fragment thereof to CD1a-expressing cells is by 25% or more.

24. A method of diagnosing a subject with a condition, comprising:
using an antibody or antigen binding fragment thereof of claim 3 to determine the level of expression of CD1a in sample obtained from the subject;
comparing the level of expression of CD1a in the sample obtained from the subject with the level of expression of CD1a in a positive or negative reference sample;
iv. determining that the subject has the condition, if the level of expression of CD1a in the sample obtained from the subject is higher than the level of expression of CD1a in the negative reference sample, or equal to or higher than the level of expression of CD1a positive reference sample; or
determining that the subject does not have the condition, if the level of expression of CD1a in the sample obtained from the subject is equal to or lower than the level of expression of CD1a in the negative reference sample, or lower than the level of expression of CD1a the positive reference sample,
wherein the condition is selected from the group consisting of: an inflammatory skin or mucosal disease or disorder, a systemic disease or disorder associated with an inflammatory response, a systemic inflammatory drug reaction, and a CD1a-expressing malignancy.

25. The method of claim 22, wherein:
(a) the inflammatory skin or mucosal disease or disorder is one or more of:
(i) a predominantly neutrophilic skin disease optionally selected from the group consisting of: acne, generalized pustular psoriasis, plaque psoriasis, guttate psoriasis, palmoplantar pustulosis, SAPHO syndrome, acute febrile neutrophilic dermatosis (Sweet syndrome), histiocytoid neutrophilic dermatitis, neutrophilic dermatosis of the dorsal hands, pyoderma gangrenosum, neutrophilic eccrine hidradenitis, hidradenitis suppurativa, erythema elevatum diutinum, Behcet disease, bowel-associated dermatitis-arthritis syndrome, other infection-associated inflammation, neutrophilic urticarial dermatosis, palisading neutrophilic granulomatous dermatitis, erythema gyratum repens, neutrophilic annular erythema, acute generalised exanthematous pustulosis (AGEP), and vasculitis;
(ii) an autoimmune disorder optionally selected from the group consisting of: a connective tissue disease, lupus, dermatomyositis, scleroderma/systemic sclerosis, Churg Strauss syndrome, panniculitis, vasculitides, an autoimmune blistering condition, bullous pemphigoid, pemphigus, linear IgA disease, dermatitis herpetiformis, coeliac disease, an auto-inflammatory disease, vitiligo, alopecia areata, alopecia universalis, alopecia totalis, panniculitis, lichen planus, erythema multiforme, lichen sclerosis, a lichenoid and erythema multiforme-like diseases, vesiculation psoriatic arthritis, rheumatoid arthritis, inflammatory bowel disease, multiple sclerosis, Guillain-Barre syndrome, thyroiditis, transverse myelitis, and neurodegeneration;
(iii) a mast cell disorder and eosinophilic disorder optionally selected from the group consisting of such as Muckle Wells syndrome, eosinophilia and systemic symptoms syndrome, urticaria, angioedema, keratoconjunctivitis, food allergy, an allergy or atopy, atopic dermatitis, rhinitis, conjunctivitis, asthma, eosinophilic oesophagitis, an eosinophilic mucosal diseases, contact dermatitis, and chronic obstructive airways;
(iv) an adverse drug reaction which manifest as an inflammatory skin or mucosal disease or disorder, optionally selected from the group consisting of: Stevens Johnsons syndrome, toxic epidermal necrolysis, drug reaction with eosinophilia and systemic symptoms syndrome (DRESS) and acute generalised exanthematous pustulosis (AGEP), erythema multiforme, bullous, fixed drug eruption, and checkpoint inhibitor-associated skin inflammation;
(v) Graft vs host disease;
(vi) Pruritus and pruritic condition, optionally wherein the condition is nodular prurigo;
(b) the inflammatory drug reaction which manifests systemically is an inflammatory reaction to imiquimod; or
(c) the CD1a-expressing malignancy is one or more of Langerhans cell histiocytosis, Langerhans cell sarcoma, subsets of T cell lymphomas, subsets of thymomas or rarely-occurring instances of other malignancies, and a subset of mastocytosis.

26. The method of claim 22, wherein the antibody or antigen binding fragment thereof is administered in combination with one or more additional therapeutic agents.

27. The method of claim 26, wherein the one or more additional therapeutic agents is selected from the group consisting of: cytotoxic agents, anti-inflammatory agents such as steroids, CAR-T cells, or a cell expressing an antibody or antigen binding fragment thereof that binds to CD1a.
